(19) United States
(12) Patent Application Publication
(10) Pub. No.: US 2026/0306615 A1
(43) Pub. Date: Oct. 8, 2026

(54) Combination Vaccine

(71) Applicant: Seqirus Inc., Holly Springs, NC (US)

(72) Inventors: Cheng Chang, Holly Springs, NC (US); Michael Cheung, Holly Springs, NC (US)

(73) Assignee: Seqirus Inc., Holly Springs, NC (US)

(21) Appl. No.: 19/482,706

(22) PCT Filed: May 10, 2024
(86) PCT No.: PCT/IB2024/054549

§ 371 Date: Nov. 7, 2025

Related U.S. Application Data
(60) Provisional application No. 63/501,222, filed on May 10, 2023.

(51) Int. Cl. A61K 39/215; A61K 39/00; A61K 39/12; A61K 39/145; A61K 39/39; A61P 35/00

(52) U.S. Cl. CPC A61K 39/215; A61K 39/12; A61K 39/145; A61K 39/39; A61P 35/00; A61K 2039/53; A61K 2039/545; A61K 2039/55566; A61K 2039/575; A61K 2039/6031; A61K 2039/6075; A61K 2039/70

(57) ABSTRACT

The present disclosure relates to compositions comprising multiple antigens of interest for use in vaccine formulations. The compositions comprise (a) a polynucleotide (such as an RNA) comprising a nucleotide sequence encoding an antigen and (b) an antigen polypeptide. The compositions can be used in inducing an immune response in a subject and/or in the treatment or prevention of a disease, such as a respiratory viral infection.

### RELATED APPLICATION DATA

The present application is a national phase entry pursuant to 35 U.S.C. § 371 of International Application No. PCT/IB2024/054549, filed May 10, 2024, which claims priority to U.S. Patent Application No. 63/501,222, filed May 10, 2023, the entire contents of which are hereby incorporated by reference for any purpose.

### SEQUENCE LISTING

The present application contains a Sequence Listing, which has been submitted electronically in XML format and is hereby incorporated by reference in its entirety. Said XML copy, created on Feb. 3, 2026, is named “538860PCT-ST26 Seq listing—updated 3 Feb. 2026.xml” and is 84,581 bytes in size.

### FIELD

The present disclosure relates to compositions comprising multiple antigens of interest for use in vaccine formulations.

### BACKGROUND

Respiratory viral infections are a significant threat to human health. Infections, such as those caused by the influenza virus and severe acute respiratory syndrome coronavirus (SARS-CoV) have been known to cause global pandemics, killing millions of people worldwide. Recently, SAR-CoV-2 has been responsible for causing the ongoing worldwide pandemic of the severely infectious coronavirus disease 2019 (COVID-19). Moreover, respiratory syncytial virus (RSV) is the single most common cause of respiratory hospitalization in infants, and reinfection remains common in later life. Whilst some vaccines are available for viral infections such as influenza, SARS-CoV-2 and RSV, such as mRNA vaccines developed to treat or prevent a SARS-CoV-2 infection, further improvements can be made to increase their efficacy and/or improve treatment strategies.

Currently, egg-based manufacturing processes are the most common way that vaccines are produced. This process requires a significant amount of time to optimize virus growth in the eggs, as well as resources (i.e., eggs) to produce sufficient amounts of vaccine, particularly during a pandemic. Furthermore, given the long development time required, vaccine strain selection is conducted before the vaccine is made available, making it difficult to respond to changes in the virus. Vaccines have also been produced using cell-based manufacturing processes involving cultured mammalian cells (e.g. Madin-Darby Canine Kidney, or MDCK cells) in place of eggs, and viral-based platforms involving recombinant virus (e.g. baculovirus encoding an antigen of influenza) have also been utilised.

There remains a need for the development of specific and efficient viral vaccines that can be produced more rapidly and with broader utility than current egg-based techniques, for the treatment or prevention of respiratory viral infections, such as influenza, RSV and SARS-CoV-2. Nucleic acid-based vaccines offer distinct advantages over the current egg-based manufacturing platform, although some challenges remain. For example, the inherently labile nature of mRNA results in most RNA-based vaccines having limited ability to provide antigen at a dose and duration required to produce a strong, durable immune response.

Therefore, it will be apparent to the skilled person that there is a need in the art for compositions with broader utility and/or improved efficacy that are suitable for use as vaccines.

### SUMMARY

The present disclosure is based on the inventors' findings that combining an RNA-based antigen with a protein-based antigen in an immunogenic composition, such as a vaccine, can enhance a subject's immune response to the immunogenic composition.

The inventors additionally found that administering a coformulated RNA vaccine, e.g., a self amplifying mRNA (sa-mRNA) vaccine and an adjuvanted protein-based vaccine results in enhanced neutralization titers against the antigens of both vaccines and influence the subclasses of antigen-specific IgG induced by recombinant protein vaccines. For example, the inventors found the ratio of antigen-specific IgG1:IgG2a induced by adjuvanted protein vaccine was significantly shifted towards a balanced IgG1:IgG2a response when coformulated with a RNA vaccine, e.g., a self amplifying mRNA (sa-mRNA) vaccine. These improvements were found compared to, e.g., RNA vaccines and protein vaccines that are co administered at distal sites from one another

The inventors additionally found that a RNA vaccine and a protein vaccine locally coadministered provided similar benefits to a co-formulated RNA vaccine and protein vaccine compared to RNA vaccines and protein vaccines that are co administered at distal sites from one another.

The findings by the inventors therefore provide basis for immunogenic compositions comprising (i) a RNA comprising a nucleotide sequence encoding one or more antigens of interest selected from the group consisting of a respiratory syncytial virus (RSV), an influenza virus and/or a severe acute respiratory syndrome coronavirus (SARS-CoV-2); and (ii) a polypeptide antigen selected from the group consisting of a RSV, an influenza virus and/or a SARS-CoV-2. Furthermore, the findings by the inventors provide basis for methods of treating or preventing or delaying progression of a disease or disorder (e.g., a RSV, influenza, a SARS-COV-2 infection, COVID-19 or ARDS) in a subject by administering the immunogenic compositions of the disclosure.

Accordingly, the present disclosure provides an immunogenic composition comprising:
(i) a polynucleotide comprising a nucleotide sequence encoding an antigen selected from the group consisting of a respiratory syncytial virus (RSV), influenza and a severe acute respiratory syndrome coronavirus (SARS-CoV-2), wherein the nucleotide sequence is operably linked to a regulatory element; and
(ii) an antigen polypeptide selected from the group consisting of a RSV, influenza and a SARS-CoV-2.

When discussing an antigen selected from the group consisting of a respiratory syncytial virus (RSV), influenza and a severe acute respiratory syndrome coronavirus (SARS-CoV-2) the skilled artisan will recognize that the antigen is from the recited virus.

In an example, the immunogenic composition comprises:
a) a polynucleotide comprising a nucleotide sequence encoding an antigen from a respiratory syncytial virus (RSV), wherein the nucleotide sequence is operably linked to a regulatory element; and
b) an antigen polypeptide from a severe acute respiratory syndrome coronavirus (SARS-CoV-2).

In another example, the immunogenic composition comprises:
a) a polynucleotide comprising a nucleotide sequence encoding an antigen from a severe acute respiratory syndrome coronavirus (SARS-CoV-2), wherein the nucleotide sequence is operably linked to a regulatory element; and
b) an antigen polypeptide from a respiratory syncytial virus (RSV).

In another example, the immunogenic composition comprises:
a) a polynucleotide comprising a nucleotide sequence encoding an antigen from a severe acute respiratory syndrome coronavirus (SARS-CoV-2), wherein the nucleotide sequence is operably linked to a regulatory element; and
b) an antigen polypeptide from influenza.

In another example, the immunogenic composition comprises:
a) a polynucleotide comprising a nucleotide sequence encoding an antigen from influenza, wherein the nucleotide sequence is operably linked to a regulatory element; and
b) an antigen polypeptide from a severe acute respiratory syndrome coronavirus (SARS-CoV-2).

In another example, the immunogenic composition comprises:
a) a polynucleotide comprising a nucleotide sequence encoding an antigen from influenza, wherein the nucleotide sequence is operably linked to a regulatory element; and
b) an antigen polypeptide from a respiratory syncytial virus (RSV).

In another example, the immunogenic composition comprises:
a) a polynucleotide comprising a nucleotide sequence encoding an antigen from a respiratory syncytial virus (RSV), wherein the nucleotide sequence is operably linked to a regulatory element; and
b) an antigen polypeptide from influenza.

In another example, the immunogenic composition comprises:
a) a polynucleotide comprising a nucleotide sequence encoding an antigen from a respiratory syncytial virus (RSV), wherein the nucleotide sequence is operably linked to a regulatory element; and
b) an antigen polypeptide from a respiratory syncytial virus (RSV).

In another example, the immunogenic composition comprises:
a) a polynucleotide comprising a nucleotide sequence encoding an antigen from influenza, wherein the nucleotide sequence is operably linked to a regulatory element; and
b) an antigen polypeptide from influenza.

In another example, the immunogenic composition comprises:
a) a polynucleotide comprising a nucleotide sequence encoding an antigen from a severe acute respiratory syndrome coronavirus (SARS-CoV-2), wherein the nucleotide sequence is operably linked to a regulatory element; and
b) an antigen polypeptide from a SARS-CoV-2.

In an example, the immunogenic composition comprises:
(i) a RNA comprising a nucleotide sequence encoding an antigen selected from the group consisting of a respiratory syncytial virus (RSV), an influenza virus and/or a severe acute respiratory syndrome coronavirus (SARS-CoV-2), wherein the nucleotide sequence is operably linked to a regulatory element; and
(ii) an antigen polypeptide selected from the group consisting of a RSV, an influenza virus and/or a SARS-CoV-2.

In an example, the immunogenic composition comprises:
a) a RNA comprising a nucleotide sequence encoding an antigen from a respiratory syncytial virus (RSV), wherein the nucleotide sequence is operably linked to a regulatory element; and
b) an antigen polypeptide from a severe acute respiratory syndrome coronavirus (SARS-CoV-2).

In another example, the immunogenic composition comprises:
a) a RNA comprising a nucleotide sequence encoding an antigen from a severe acute respiratory syndrome coronavirus (SARS-CoV-2), wherein the nucleotide sequence is operably linked to a regulatory element; and
b) an antigen polypeptide from a respiratory syncytial virus (RSV).

In another example, the immunogenic composition comprises:
a) a RNA comprising a nucleotide sequence encoding an antigen from a severe acute respiratory syndrome coronavirus (SARS-CoV-2), wherein the nucleotide sequence is operably linked to a regulatory element; and
b) an antigen polypeptide from influenza.

In another example, the immunogenic composition comprises:
a) a RNA comprising a nucleotide sequence encoding an antigen from influenza, wherein the nucleotide sequence is operably linked to a regulatory element; and
b) an antigen polypeptide from a severe acute respiratory syndrome coronavirus (SARS-CoV-2).

In another example, the immunogenic composition comprises:
a) a RNA comprising a nucleotide sequence encoding an antigen from influenza, wherein the nucleotide sequence is operably linked to a regulatory element; and
b) an antigen polypeptide from a respiratory syncytial virus (RSV).

In another example, the immunogenic composition comprises:
a) a RNA comprising a nucleotide sequence encoding an antigen from a respiratory syncytial virus (RSV), wherein the nucleotide sequence is operably linked to a regulatory element; and
b) an antigen polypeptide from influenza.

In another example, the immunogenic composition comprises:
a) a RNA comprising a nucleotide sequence encoding an antigen from a respiratory syncytial virus (RSV), wherein the nucleotide sequence is operably linked to a regulatory element; and
b) an antigen polypeptide from a respiratory syncytial virus (RSV).

In another example, the immunogenic composition comprises:
a) a RNA comprising a nucleotide sequence encoding an antigen from influenza, wherein the nucleotide sequence is operably linked to a regulatory element; and
b) an antigen polypeptide from influenza.

In another example, the immunogenic composition comprises:
a) a RNA comprising a nucleotide sequence encoding an antigen from a SARS-CoV-2, wherein the nucleotide sequence is operably linked to a regulatory element; and
b) an antigen polypeptide from a SARS-CoV-2.

In another example, the immunogenic composition comprises:
(i) a cRNA comprising a nucleotide sequence encoding an antigen selected from the group consisting of a respiratory syncytial virus (RSV), an influenza virus and/or a severe acute respiratory syndrome coronavirus (SARS-CoV-2), wherein the nucleotide sequence is operably linked to a regulatory element; and
(ii) an antigen polypeptide selected from the group consisting of a RSV, an influenza virus and/or a SARS-CoV-2.

In an example, the immunogenic composition comprises:
a) a cRNA comprising a nucleotide sequence encoding an antigen from a respiratory syncytial virus (RSV), wherein the nucleotide sequence is operably linked to a regulatory element; and
b) an antigen polypeptide from a severe acute respiratory syndrome coronavirus (SARS-CoV-2).

In another example, the immunogenic composition comprises:
a) a cRNA comprising a nucleotide sequence encoding an antigen from a severe acute respiratory syndrome coronavirus (SARS-CoV-2), wherein the nucleotide sequence is operably linked to a regulatory element; and
b) an antigen polypeptide from a respiratory syncytial virus (RSV).

In another example, the immunogenic composition comprises:
a) a cRNA comprising a nucleotide sequence encoding an antigen from a severe acute respiratory syndrome coronavirus (SARS-CoV-2), wherein the nucleotide sequence is operably linked to a regulatory element; and
b) an antigen polypeptide from influenza.

In another example, the immunogenic composition comprises:
a) a cRNA comprising a nucleotide sequence encoding an antigen from influenza, wherein the nucleotide sequence is operably linked to a regulatory element; and
b) an antigen polypeptide from a severe acute respiratory syndrome coronavirus (SARS-CoV-2).

In another example, the immunogenic composition comprises:
a) a cRNA comprising a nucleotide sequence encoding an antigen from influenza, wherein the nucleotide sequence is operably linked to a regulatory element; and
b) an antigen polypeptide from a respiratory syncytial virus (RSV).

In another example, the immunogenic composition comprises:
a) a cRNA comprising a nucleotide sequence encoding an antigen from a respiratory syncytial virus (RSV), wherein the nucleotide sequence is operably linked to a regulatory element; and
b) an antigen polypeptide from influenza.

In another example, the immunogenic composition comprises:
a) a cRNA comprising a nucleotide sequence encoding an antigen from a respiratory syncytial virus (RSV), wherein the nucleotide sequence is operably linked to a regulatory element; and
b) an antigen polypeptide from a RSV.

In another example, the immunogenic composition comprises:
a) a cRNA comprising a nucleotide sequence encoding an antigen from influenza, wherein the nucleotide sequence is operably linked to a regulatory element; and
b) an antigen polypeptide from influenza.

In another example, the immunogenic composition comprises:
a) a cRNA comprising a nucleotide sequence encoding an antigen from a severe acute respiratory syndrome coronavirus (SARS-CoV-2), wherein the nucleotide sequence is operably linked to a regulatory element; and
b) an antigen polypeptide from a SARS-CoV-2.

In another example, the immunogenic composition comprises:
(i) a self-replicating RNA comprising a nucleotide sequence encoding an antigen selected from the group consisting of a respiratory syncytial virus (RSV), an influenza virus and/or a severe acute respiratory syndrome coronavirus (SARS-CoV-2), wherein the nucleotide sequence is operably linked to a regulatory element; and
(ii) an antigen polypeptide selected from the group consisting of a RSV, an influenza virus and/or a SARS-CoV-2.

In an example, the immunogenic composition comprises:
a) a self-replicating RNA comprising a nucleotide sequence encoding an antigen from a respiratory syncytial virus (RSV), wherein the nucleotide sequence is operably linked to a regulatory element; and
b) an antigen polypeptide from a severe acute respiratory syndrome coronavirus (SARS-CoV-2).

In another example, the immunogenic composition comprises:
a) a self-replicating RNA comprising a nucleotide sequence encoding an antigen from a severe acute respiratory syndrome coronavirus (SARS-CoV-2), wherein the nucleotide sequence is operably linked to a regulatory element; and
b) an antigen polypeptide from a respiratory syncytial virus (RSV).

In another example, the immunogenic composition comprises:
a) a self-replicating RNA comprising a nucleotide sequence encoding an antigen from a severe acute respiratory syndrome coronavirus (SARS-CoV-2), wherein the nucleotide sequence is operably linked to a regulatory element; and
b) an antigen polypeptide from influenza.

In another example, the immunogenic composition comprises:
a) a self-replicating RNA comprising a nucleotide sequence encoding an antigen from influenza, wherein the nucleotide sequence is operably linked to a regulatory element; and
b) an antigen polypeptide from a severe acute respiratory syndrome coronavirus (SARS-CoV-2).

In another example, the immunogenic composition comprises:
a) a self-replicating RNA comprising a nucleotide sequence encoding an antigen from influenza, wherein the nucleotide sequence is operably linked to a regulatory element; and
b) an antigen polypeptide from a respiratory syncytial virus (RSV).

In another example, the immunogenic composition comprises:
a) a self-replicating RNA comprising a nucleotide sequence encoding an antigen from a respiratory syncytial virus (RSV), wherein the nucleotide sequence is operably linked to a regulatory element; and
b) an antigen polypeptide from influenza.

In another example, the immunogenic composition comprises:
a) a self-replicating RNA comprising a nucleotide sequence encoding an antigen from a respiratory syncytial virus (RSV), wherein the nucleotide sequence is operably linked to a regulatory element; and
b) an antigen polypeptide from a RSV.

In another example, the immunogenic composition comprises:
a) a self-replicating RNA comprising a nucleotide sequence encoding an antigen from influenza, wherein the nucleotide sequence is operably linked to a regulatory element; and
b) an antigen polypeptide from influenza.

In another example, the immunogenic composition comprises:
a) a self-replicating RNA comprising a nucleotide sequence encoding an antigen from a severe acute respiratory syndrome coronavirus (SARS-CoV-2), wherein the nucleotide sequence is operably linked to a regulatory element; and
b) an antigen polypeptide from a SARS-CoV-2.

In one example, an immunogenic composition of the disclosure comprises multiple polynucleotides, wherein each polynucleotide encodes different polypeptide sequences. In another example, an immunogenic composition of the disclosure comprises multiple RNAs, wherein each RNA encodes different polypeptide sequences. In a further example, an immunogenic composition of the disclosure comprises multiple cRNAs, wherein each cRNA encodes different polypeptide sequences. In a further example, an immunogenic composition of the disclosure comprises multiple self-replicating RNAs, wherein each self-replicating RNA encodes different polypeptide sequences.

Thus, in an example, the immunogenic composition comprises:
(i) a polynucleotide comprising a nucleotide sequence encoding an antigen from respiratory syncytial virus (RSV), wherein the nucleotide sequence is operably linked to a regulatory element;
(ii) a polynucleotide comprising a nucleotide sequence encoding an antigen from influenza, wherein the nucleotide sequence is operably linked to a regulatory element; and
(iii) an antigen polypeptide from a severe acute respiratory syndrome coronavirus (SARS-CoV-2).

In an example, the immunogenic composition comprises, in order from 5′ to 3′:
(i) the polynucleotide comprising a nucleotide sequence encoding an antigen from respiratory syncytial virus (RSV);
(ii) the polynucleotide comprising a nucleotide sequence encoding an antigen from influenza.

In an example, the immunogenic composition comprises, in order from 5′ to 3′:
(i) the polynucleotide comprising a nucleotide sequence encoding an antigen from influenza, and
(ii) the polynucleotide comprising a nucleotide sequence encoding an antigen from respiratory syncytial virus (RSV).

In another example, the immunogenic composition comprises:
(i) a polynucleotide comprising a nucleotide sequence encoding an antigen from a severe acute respiratory syndrome coronavirus (SARS-CoV-2), wherein the nucleotide sequence is operably linked to a regulatory element;
(ii) a polynucleotide comprising a nucleotide sequence encoding an antigen from influenza, wherein the nucleotide sequence is operably linked to a regulatory element; and
(iii) an antigen polypeptide from a respiratory syncytial virus (RSV).

In an example, the immunogenic composition comprises, in order from 5′ to 3′:
(i) the polynucleotide comprising a nucleotide sequence encoding an antigen from a severe acute respiratory syndrome coronavirus (SARS-CoV-2);
(ii) the polynucleotide comprising a nucleotide sequence encoding an antigen from influenza.

In an example, the immunogenic composition comprises, in order from 5′ to 3′:
(i) the polynucleotide comprising a nucleotide sequence encoding an antigen from influenza; and
(ii) the polynucleotide comprising a nucleotide sequence encoding an antigen from a severe acute respiratory syndrome coronavirus (SARS-CoV-2).

In another example, the immunogenic composition comprises:
(i) a polynucleotide comprising a nucleotide sequence encoding an antigen from a severe acute respiratory syndrome coronavirus (SARS-CoV-2), wherein the nucleotide sequence is operably linked to a regulatory element;
(ii) a polynucleotide comprising a nucleotide sequence encoding an antigen from a respiratory syncytial virus (RSV), wherein the nucleotide sequence is operably linked to a regulatory element; and
(iii) an antigen polypeptide from influenza.

In an example, the immunogenic composition comprises, in order from 5′ to 3′:
(i) the polynucleotide comprising a nucleotide sequence encoding an antigen from a severe acute respiratory syndrome coronavirus (SARS-CoV-2);
(ii) the polynucleotide comprising a nucleotide sequence encoding an antigen from a respiratory syncytial virus (RSV).

In an example, the immunogenic composition comprises, in order from 5′ to 3′:
(i) the polynucleotide comprising a nucleotide sequence encoding an antigen from a respiratory syncytial virus (RSV); and
(ii) the polynucleotide comprising a nucleotide sequence encoding an antigen from a severe acute respiratory syndrome coronavirus (SARS-CoV-2).

In another example, the immunogenic composition comprises:
(i) a polynucleotide comprising a nucleotide sequence encoding an antigen from a severe acute respiratory syndrome coronavirus (SARS-CoV-2), wherein the nucleotide sequence is operably linked to a regulatory element;
(ii) a polynucleotide comprising a nucleotide sequence encoding an antigen from a severe acute respiratory syndrome coronavirus (SARS-CoV-2), wherein the nucleotide sequence is operably linked to a regulatory element; and
(iii) an antigen polypeptide from a severe acute respiratory syndrome coronavirus (SARS-CoV-2).

In another example, the immunogenic composition comprises:
(i) a polynucleotide comprising a nucleotide sequence encoding an antigen from a respiratory syncytial virus (RSV), wherein the nucleotide sequence is operably linked to a regulatory element;
(ii) a polynucleotide comprising a nucleotide sequence encoding an antigen from a respiratory syncytial virus (RSV), wherein the nucleotide sequence is operably linked to a regulatory element; and
(iii) an antigen polypeptide from a respiratory syncytial virus (RSV).

In another example, the immunogenic composition comprises:
(i) a polynucleotide comprising a nucleotide sequence encoding an antigen from influenza, wherein the nucleotide sequence is operably linked to a regulatory element;
(ii) a polynucleotide comprising a nucleotide sequence encoding an antigen from influenza, wherein the nucleotide sequence is operably linked to a regulatory element; and
(iii) an antigen polypeptide from influenza.

In an example, the immunogenic composition comprises:
(i) a polynucleotide comprising a nucleotide sequence encoding an antigen from a severe acute respiratory syndrome coronavirus (SARS-CoV-2), wherein the nucleotide sequence is operably linked to a regulatory element;
(ii) a polynucleotide comprising a nucleotide sequence encoding an antigen from a severe acute respiratory syndrome coronavirus (SARS-CoV-2), wherein the nucleotide sequence is operably linked to a regulatory element; and
(iii) an antigen polypeptide from respiratory syncytial virus (RSV).

In another example, the immunogenic composition comprises:
(i) a polynucleotide comprising a nucleotide sequence encoding an antigen from respiratory syncytial virus (RSV), wherein the nucleotide sequence is operably linked to a regulatory element;
(ii) a polynucleotide comprising a nucleotide sequence encoding an antigen from a severe acute respiratory syndrome coronavirus (SARS-CoV-2), wherein the nucleotide sequence is operably linked to a regulatory element; and
(iii) an antigen polypeptide from a severe acute respiratory syndrome coronavirus (SARS-CoV-2).

In another example, the immunogenic composition comprises:
(i) a polynucleotide comprising a nucleotide sequence encoding an antigen from respiratory syncytial virus (RSV), wherein the nucleotide sequence is operably linked to a regulatory element;
(ii) a polynucleotide comprising a nucleotide sequence encoding an antigen from respiratory syncytial virus (RSV), wherein the nucleotide sequence is operably linked to a regulatory element; and
(iii) an antigen polypeptide from a severe acute respiratory syndrome coronavirus (SARS-CoV-2).

In another example, the immunogenic composition comprises:
(i) a polynucleotide comprising a nucleotide sequence encoding an antigen from a severe acute respiratory syndrome coronavirus (SARS-CoV-2), wherein the nucleotide sequence is operably linked to a regulatory element;
(ii) a polynucleotide comprising a nucleotide sequence encoding an antigen from respiratory syncytial virus (RSV), wherein the nucleotide sequence is operably linked to a regulatory element; and
(iii) an antigen polypeptide from respiratory syncytial virus (RSV).

In an example, the immunogenic composition comprises:
(i) a polynucleotide comprising a nucleotide sequence encoding an antigen from influenza, wherein the nucleotide sequence is operably linked to a regulatory element;
(ii) a polynucleotide comprising a nucleotide sequence encoding an antigen from influenza, wherein the nucleotide sequence is operably linked to a regulatory element; and
(iii) an antigen polypeptide from respiratory syncytial virus (RSV).

In another example, the immunogenic composition comprises:
(i) a polynucleotide comprising a nucleotide sequence encoding an antigen from respiratory syncytial virus (RSV), wherein the nucleotide sequence is operably linked to a regulatory element;
(ii) a polynucleotide comprising a nucleotide sequence encoding an antigen from influenza, wherein the nucleotide sequence is operably linked to a regulatory element; and
(iii) an antigen polypeptide from influenza.

In another example, the immunogenic composition comprises:
(i) a polynucleotide comprising a nucleotide sequence encoding an antigen from respiratory syncytial virus (RSV), wherein the nucleotide sequence is operably linked to a regulatory element;
(ii) a polynucleotide comprising a nucleotide sequence encoding an antigen from respiratory syncytial virus (RSV), wherein the nucleotide sequence is operably linked to a regulatory element; and
(iii) an antigen polypeptide from influenza.

In another example, the immunogenic composition comprises:
(i) a polynucleotide comprising a nucleotide sequence encoding an antigen from influenza, wherein the nucleotide sequence is operably linked to a regulatory element;
(ii) a polynucleotide comprising a nucleotide sequence encoding an antigen from respiratory syncytial virus (RSV), wherein the nucleotide sequence is operably linked to a regulatory element; and
(iii) an antigen polypeptide from respiratory syncytial virus (RSV).

In an example, the immunogenic composition comprises:
(i) a polynucleotide comprising a nucleotide sequence encoding an antigen from a severe acute respiratory syndrome coronavirus (SARS-CoV-2), wherein the nucleotide sequence is operably linked to a regulatory element;
(ii) a polynucleotide comprising a nucleotide sequence encoding an antigen from a severe acute respiratory syndrome coronavirus (SARS-CoV-2), wherein the nucleotide sequence is operably linked to a regulatory element; and
(iii) an antigen polypeptide from influenza.

In another example, the immunogenic composition comprises:
(i) a polynucleotide comprising a nucleotide sequence encoding an antigen from influenza, wherein the nucleotide sequence is operably linked to a regulatory element;
(ii) a polynucleotide comprising a nucleotide sequence encoding an antigen from a severe acute respiratory syndrome coronavirus (SARS-CoV-2), wherein the nucleotide sequence is operably linked to a regulatory element; and
(iii) an antigen polypeptide from a severe acute respiratory syndrome coronavirus (SARS-CoV-2).

In another example, the immunogenic composition comprises:
(i) a polynucleotide comprising a nucleotide sequence encoding an antigen from influenza, wherein the nucleotide sequence is operably linked to a regulatory element;
(ii) a polynucleotide comprising a nucleotide sequence encoding an antigen from influenza, wherein the nucleotide sequence is operably linked to a regulatory element; and
(iii) an antigen polypeptide from a severe acute respiratory syndrome coronavirus (SARS-CoV-2).

In another example, the immunogenic composition comprises:
(i) a polynucleotide comprising a nucleotide sequence encoding an antigen from a severe acute respiratory syndrome coronavirus (SARS-CoV-2), wherein the nucleotide sequence is operably linked to a regulatory element;
(ii) a polynucleotide comprising a nucleotide sequence encoding an antigen from influenza, wherein the nucleotide sequence is operably linked to a regulatory element; and
(iii) an antigen polypeptide from influenza.

In an example, the immunogenic composition comprises:
(i) a RNA comprising a nucleotide sequence encoding an antigen from respiratory syncytial virus (RSV), wherein the nucleotide sequence is operably linked to a regulatory element;
(ii) a RNA comprising a nucleotide sequence encoding an antigen from influenza, wherein the nucleotide sequence is operably linked to a regulatory element; and
(iii) an antigen polypeptide from a severe acute respiratory syndrome coronavirus (SARS-CoV-2).

In an example, the immunogenic composition comprises, in order from 5′ to 3′:
(i) the RNA comprising a nucleotide sequence encoding an antigen from respiratory syncytial virus (RSV);
(ii) the RNA comprising a nucleotide sequence encoding an antigen from influenza.

In an example, the immunogenic composition comprises, in order from 5′ to 3′:
(i) the RNA comprising a nucleotide sequence encoding an antigen from influenza, and
(ii) the RNA comprising a nucleotide sequence encoding an antigen from respiratory syncytial virus (RSV).

In another example, the immunogenic composition comprises:
(i) a RNA comprising a nucleotide sequence encoding an antigen from a severe acute respiratory syndrome coronavirus (SARS-CoV-2), wherein the nucleotide sequence is operably linked to a regulatory element;
(ii) a RNA comprising a nucleotide sequence encoding an antigen from influenza, wherein the nucleotide sequence is operably linked to a regulatory element; and
(iii) an antigen polypeptide from a respiratory syncytial virus (RSV).

In an example, the immunogenic composition comprises, in order from 5′ to 3′:
(i) the RNA comprising a nucleotide sequence encoding an antigen from a severe acute respiratory syndrome coronavirus (SARS-CoV-2);
(ii) the RNA comprising a nucleotide sequence encoding an antigen from influenza.

In an example, the immunogenic composition comprises, in order from 5′ to 3′:
(i) the RNA comprising a nucleotide sequence encoding an antigen from influenza; and
(ii) the RNA comprising a nucleotide sequence encoding an antigen from a severe acute respiratory syndrome coronavirus (SARS-CoV-2).

In another example, the immunogenic composition comprises:
(i) a RNA comprising a nucleotide sequence encoding an antigen from a severe acute respiratory syndrome coronavirus (SARS-CoV-2), wherein the nucleotide sequence is operably linked to a regulatory element;
(ii) a RNA comprising a nucleotide sequence encoding an antigen from a respiratory syncytial virus (RSV), wherein the nucleotide sequence is operably linked to a regulatory element; and
(iii) an antigen polypeptide from influenza.

In an example, the immunogenic composition comprises, in order from 5′ to 3′:
(i) the RNA comprising a nucleotide sequence encoding an antigen from a severe acute respiratory syndrome coronavirus (SARS-CoV-2);
(ii) the RNA comprising a nucleotide sequence encoding an antigen from a respiratory syncytial virus (RSV).

In an example, the immunogenic composition comprises, in order from 5′ to 3′:
(i) the RNA comprising a nucleotide sequence encoding an antigen from a respiratory syncytial virus (RSV); and
(ii) the RNA comprising a nucleotide sequence encoding an antigen from a severe acute respiratory syndrome coronavirus (SARS-CoV-2).

In another example, the immunogenic composition comprises:
(i) a RNA comprising a nucleotide sequence encoding an antigen from a severe acute respiratory syndrome coronavirus (SARS-CoV-2), wherein the nucleotide sequence is operably linked to a regulatory element;
(ii) a RNA comprising a nucleotide sequence encoding an antigen from a severe acute respiratory syndrome coronavirus (SARS-CoV-2), wherein the nucleotide sequence is operably linked to a regulatory element; and
(iii) an antigen polypeptide from a severe acute respiratory syndrome coronavirus (SARS-CoV-2).

In another example, the immunogenic composition comprises:
(i) a RNA comprising a nucleotide sequence encoding an antigen from a respiratory syncytial virus (RSV), wherein the nucleotide sequence is operably linked to a regulatory element;
(ii) a RNA comprising a nucleotide sequence encoding an antigen from a respiratory syncytial virus (RSV), wherein the nucleotide sequence is operably linked to a regulatory element; and
(iii) an antigen polypeptide from a respiratory syncytial virus (RSV).

In another example, the immunogenic composition comprises:
(i) a RNA comprising a nucleotide sequence encoding an antigen from influenza, wherein the nucleotide sequence is operably linked to a regulatory element;
(ii) a RNA comprising a nucleotide sequence encoding an antigen from influenza, wherein the nucleotide sequence is operably linked to a regulatory element; and
(iii) an antigen polypeptide from influenza.

In an example, the immunogenic composition comprises:
(i) a RNA comprising a nucleotide sequence encoding an antigen from a severe acute respiratory syndrome coronavirus (SARS-CoV-2), wherein the nucleotide sequence is operably linked to a regulatory element;
(ii) a RNA comprising a nucleotide sequence encoding an antigen from a severe acute respiratory syndrome coronavirus (SARS-CoV-2), wherein the nucleotide sequence is operably linked to a regulatory element; and
(iii) an antigen polypeptide from respiratory syncytial virus (RSV).

In another example, the immunogenic composition comprises:
(i) a RNA comprising a nucleotide sequence encoding an antigen from respiratory syncytial virus (RSV), wherein the nucleotide sequence is operably linked to a regulatory element;
(ii) a RNA comprising a nucleotide sequence encoding an antigen from a severe acute respiratory syndrome coronavirus (SARS-CoV-2), wherein the nucleotide sequence is operably linked to a regulatory element; and
(iii) an antigen polypeptide from a severe acute respiratory syndrome coronavirus (SARS-CoV-2).

In another example, the immunogenic composition comprises:
(i) a RNA comprising a nucleotide sequence encoding an antigen from respiratory syncytial virus (RSV), wherein the nucleotide sequence is operably linked to a regulatory element;
(ii) a RNA comprising a nucleotide sequence encoding an antigen from respiratory syncytial virus (RSV), wherein the nucleotide sequence is operably linked to a regulatory element; and
(iii) an antigen polypeptide from a severe acute respiratory syndrome coronavirus (SARS-CoV-2).

In another example, the immunogenic composition comprises:
(i) a RNA comprising a nucleotide sequence encoding an antigen from a severe acute respiratory syndrome coronavirus (SARS-CoV-2), wherein the nucleotide sequence is operably linked to a regulatory element;
(ii) a RNA comprising a nucleotide sequence encoding an antigen from respiratory syncytial virus (RSV), wherein the nucleotide sequence is operably linked to a regulatory element; and
(iii) an antigen polypeptide from respiratory syncytial virus (RSV).

In an example, the immunogenic composition comprises:
(i) a RNA comprising a nucleotide sequence encoding an antigen from influenza, wherein the nucleotide sequence is operably linked to a regulatory element;
(ii) a RNA comprising a nucleotide sequence encoding an antigen from influenza, wherein the nucleotide sequence is operably linked to a regulatory element; and
(iii) an antigen polypeptide from respiratory syncytial virus (RSV).

In another example, the immunogenic composition comprises:
(i) a RNA comprising a nucleotide sequence encoding an antigen from respiratory syncytial virus (RSV), wherein the nucleotide sequence is operably linked to a regulatory element;
(ii) a RNA comprising a nucleotide sequence encoding an antigen from influenza, wherein the nucleotide sequence is operably linked to a regulatory element; and
(iii) an antigen polypeptide from influenza.

In another example, the immunogenic composition comprises:
(i) a RNA comprising a nucleotide sequence encoding an antigen from respiratory syncytial virus (RSV), wherein the nucleotide sequence is operably linked to a regulatory element;
(ii) a RNA comprising a nucleotide sequence encoding an antigen from respiratory syncytial virus (RSV), wherein the nucleotide sequence is operably linked to a regulatory element; and
(iii) an antigen polypeptide from influenza.

In another example, the immunogenic composition comprises:
(i) a RNA comprising a nucleotide sequence encoding an antigen from influenza, wherein the nucleotide sequence is operably linked to a regulatory element;
(ii) a RNA comprising a nucleotide sequence encoding an antigen from respiratory syncytial virus (RSV), wherein the nucleotide sequence is operably linked to a regulatory element; and
(iii) an antigen polypeptide from respiratory syncytial virus (RSV).

In an example, the immunogenic composition comprises:
(i) a RNA comprising a nucleotide sequence encoding an antigen from a severe acute respiratory syndrome coronavirus (SARS-CoV-2), wherein the nucleotide sequence is operably linked to a regulatory element;
(ii) a RNA comprising a nucleotide sequence encoding an antigen from a severe acute respiratory syndrome coronavirus (SARS-CoV-2), wherein the nucleotide sequence is operably linked to a regulatory element; and
(iii) an antigen polypeptide from influenza.

In another example, the immunogenic composition comprises:
(i) a RNA comprising a nucleotide sequence encoding an antigen from influenza, wherein the nucleotide sequence is operably linked to a regulatory element;
(ii) a RNA comprising a nucleotide sequence encoding an antigen from a severe acute respiratory syndrome coronavirus (SARS-CoV-2), wherein the nucleotide sequence is operably linked to a regulatory element; and
(iii) an antigen polypeptide from a severe acute respiratory syndrome coronavirus (SARS-CoV-2).

In another example, the immunogenic composition comprises:
(i) a RNA comprising a nucleotide sequence encoding an antigen from influenza, wherein the nucleotide sequence is operably linked to a regulatory element;
(ii) a RNA comprising a nucleotide sequence encoding an antigen from influenza, wherein the nucleotide sequence is operably linked to a regulatory element; and
(iii) an antigen polypeptide from a severe acute respiratory syndrome coronavirus (SARS-CoV-2).

In another example, the immunogenic composition comprises:
(i) a RNA comprising a nucleotide sequence encoding an antigen from a severe acute respiratory syndrome coronavirus (SARS-CoV-2), wherein the nucleotide sequence is operably linked to a regulatory element;
(ii) a RNA comprising a nucleotide sequence encoding an antigen from influenza, wherein the nucleotide sequence is operably linked to a regulatory element; and
(iii) an antigen polypeptide from influenza.

In an example, the immunogenic composition comprises:
(i) a cRNA comprising a nucleotide sequence encoding an antigen from respiratory syncytial virus (RSV), wherein the nucleotide sequence is operably linked to a regulatory element;
(ii) a cRNA comprising a nucleotide sequence encoding an antigen from influenza, wherein the nucleotide sequence is operably linked to a regulatory element; and
(iii) an antigen polypeptide from a severe acute respiratory syndrome coronavirus (SARS-CoV-2).

In an example, the immunogenic composition comprises, in order from 5′ to 3′:
(i) the cRNA comprising a nucleotide sequence encoding an antigen from respiratory syncytial virus (RSV);
(ii) the cRNA comprising a nucleotide sequence encoding an antigen from influenza.

In an example, the immunogenic composition comprises, in order from 5′ to 3′:
(i) the cRNA comprising a nucleotide sequence encoding an antigen from influenza, and
(ii) the cRNA comprising a nucleotide sequence encoding an antigen from respiratory syncytial virus (RSV).

In another example, the immunogenic composition comprises:
(i) a cRNA comprising a nucleotide sequence encoding an antigen from a severe acute respiratory syndrome coronavirus (SARS-CoV-2), wherein the nucleotide sequence is operably linked to a regulatory element;
(ii) a cRNA comprising a nucleotide sequence encoding an antigen from influenza, wherein the nucleotide sequence is operably linked to a regulatory element; and
(iii) an antigen polypeptide from a respiratory syncytial virus (RSV).

In an example, the immunogenic composition comprises, in order from 5′ to 3′:
(i) the cRNA comprising a nucleotide sequence encoding an antigen from a severe acute respiratory syndrome coronavirus (SARS-CoV-2);
(ii) the cRNA comprising a nucleotide sequence encoding an antigen from influenza.

In an example, the immunogenic composition comprises, in order from 5′ to 3′:
(i) the cRNA comprising a nucleotide sequence encoding an antigen from influenza; and
(ii) the cRNA comprising a nucleotide sequence encoding an antigen from a severe acute respiratory syndrome coronavirus (SARS-CoV-2).

In another example, the immunogenic composition comprises:
(i) a cRNA comprising a nucleotide sequence encoding an antigen from a severe acute respiratory syndrome coronavirus (SARS-CoV-2), wherein the nucleotide sequence is operably linked to a regulatory element;
(ii) a cRNA comprising a nucleotide sequence encoding an antigen from a respiratory syncytial virus (RSV), wherein the nucleotide sequence is operably linked to a regulatory element; and
(iii) an antigen polypeptide from influenza.

In an example, the immunogenic composition comprises, in order from 5′ to 3′:
(i) the cRNA comprising a nucleotide sequence encoding an antigen from a severe acute respiratory syndrome coronavirus (SARS-CoV-2);
(ii) the cRNA comprising a nucleotide sequence encoding an antigen from a respiratory syncytial virus (RSV).

In an example, the immunogenic composition comprises, in order from 5′ to 3′:
(i) the cRNA comprising a nucleotide sequence encoding an antigen from a respiratory syncytial virus (RSV); and
(ii) the cRNA comprising a nucleotide sequence encoding an antigen from a severe acute respiratory syndrome coronavirus (SARS-CoV-2).

In another example, the immunogenic composition comprises:
(i) a cRNA comprising a nucleotide sequence encoding an antigen from a severe acute respiratory syndrome coronavirus (SARS-CoV-2), wherein the nucleotide sequence is operably linked to a regulatory element;
(ii) a cRNA comprising a nucleotide sequence encoding an antigen from a severe acute respiratory syndrome coronavirus (SARS-CoV-2), wherein the nucleotide sequence is operably linked to a regulatory element; and
(iii) an antigen polypeptide from a severe acute respiratory syndrome coronavirus (SARS-CoV-2).

In another example, the immunogenic composition comprises:
(i) a cRNA comprising a nucleotide sequence encoding an antigen from a respiratory syncytial virus (RSV), wherein the nucleotide sequence is operably linked to a regulatory element;
(ii) a cRNA comprising a nucleotide sequence encoding an antigen from a respiratory syncytial virus (RSV), wherein the nucleotide sequence is operably linked to a regulatory element; and
(iii) an antigen polypeptide from a respiratory syncytial virus (RSV).

In another example, the immunogenic composition comprises:
(i) a cRNA comprising a nucleotide sequence encoding an antigen from influenza, wherein the nucleotide sequence is operably linked to a regulatory element;
(ii) a cRNA comprising a nucleotide sequence encoding an antigen from influenza, wherein the nucleotide sequence is operably linked to a regulatory element; and
(iii) an antigen polypeptide from influenza.

In an example, the immunogenic composition comprises:
(i) a cRNA comprising a nucleotide sequence encoding an antigen from a severe acute respiratory syndrome coronavirus (SARS-CoV-2), wherein the nucleotide sequence is operably linked to a regulatory element;
(ii) a cRNA comprising a nucleotide sequence encoding an antigen from a severe acute respiratory syndrome coronavirus (SARS-CoV-2), wherein the nucleotide sequence is operably linked to a regulatory element; and
(iii) an antigen polypeptide from respiratory syncytial virus (RSV).

In another example, the immunogenic composition comprises:
(i) a cRNA comprising a nucleotide sequence encoding an antigen from respiratory syncytial virus (RSV), wherein the nucleotide sequence is operably linked to a regulatory element;
(ii) a cRNA comprising a nucleotide sequence encoding an antigen from a severe acute respiratory syndrome coronavirus (SARS-CoV-2), wherein the nucleotide sequence is operably linked to a regulatory element; and
(iii) an antigen polypeptide from a severe acute respiratory syndrome coronavirus (SARS-CoV-2).

In another example, the immunogenic composition comprises:
(i) a cRNA comprising a nucleotide sequence encoding an antigen from respiratory syncytial virus (RSV), wherein the nucleotide sequence is operably linked to a regulatory element;
(ii) a cRNA comprising a nucleotide sequence encoding an antigen from respiratory syncytial virus (RSV), wherein the nucleotide sequence is operably linked to a regulatory element; and
(iii) an antigen polypeptide from a severe acute respiratory syndrome coronavirus (SARS-CoV-2).

In another example, the immunogenic composition comprises:
(i) a cRNA comprising a nucleotide sequence encoding an antigen from a severe acute respiratory syndrome coronavirus (SARS-CoV-2), wherein the nucleotide sequence is operably linked to a regulatory element;
(ii) a cRNA comprising a nucleotide sequence encoding an antigen from respiratory syncytial virus (RSV), wherein the nucleotide sequence is operably linked to a regulatory element; and
(iii) an antigen polypeptide from respiratory syncytial virus (RSV).

In an example, the immunogenic composition comprises:
(i) a cRNA comprising a nucleotide sequence encoding an antigen from influenza, wherein the nucleotide sequence is operably linked to a regulatory element;
(ii) a cRNA comprising a nucleotide sequence encoding an antigen from influenza, wherein the nucleotide sequence is operably linked to a regulatory element; and
(iii) an antigen polypeptide from respiratory syncytial virus (RSV).

In another example, the immunogenic composition comprises:
(i) a cRNA comprising a nucleotide sequence encoding an antigen from respiratory syncytial virus (RSV), wherein the nucleotide sequence is operably linked to a regulatory element;
(ii) a cRNA comprising a nucleotide sequence encoding an antigen from influenza, wherein the nucleotide sequence is operably linked to a regulatory element; and
(iii) an antigen polypeptide from influenza.

In another example, the immunogenic composition comprises:
(i) a cRNA comprising a nucleotide sequence encoding an antigen from respiratory syncytial virus (RSV), wherein the nucleotide sequence is operably linked to a regulatory element;
(ii) a cRNA comprising a nucleotide sequence encoding an antigen from respiratory syncytial virus (RSV), wherein the nucleotide sequence is operably linked to a regulatory element; and
(iii) an antigen polypeptide from influenza.

In another example, the immunogenic composition comprises:
(i) a cRNA comprising a nucleotide sequence encoding an antigen from influenza, wherein the nucleotide sequence is operably linked to a regulatory element;
(ii) a cRNA comprising a nucleotide sequence encoding an antigen from respiratory syncytial virus (RSV), wherein the nucleotide sequence is operably linked to a regulatory element; and
(iii) an antigen polypeptide from respiratory syncytial virus (RSV).

In an example, the immunogenic composition comprises:
(i) a cRNA comprising a nucleotide sequence encoding an antigen from a severe acute respiratory syndrome coronavirus (SARS-CoV-2), wherein the nucleotide sequence is operably linked to a regulatory element;
(ii) a cRNA comprising a nucleotide sequence encoding an antigen from a severe acute respiratory syndrome coronavirus (SARS-CoV-2), wherein the nucleotide sequence is operably linked to a regulatory element; and
(iii) an antigen polypeptide from influenza.

In another example, the immunogenic composition comprises:
(i) a cRNA comprising a nucleotide sequence encoding an antigen from influenza, wherein the nucleotide sequence is operably linked to a regulatory element;
(ii) a cRNA comprising a nucleotide sequence encoding an antigen from a severe acute respiratory syndrome coronavirus (SARS-CoV-2), wherein the nucleotide sequence is operably linked to a regulatory element; and
(iii) an antigen polypeptide from a severe acute respiratory syndrome coronavirus (SARS-CoV-2).

In another example, the immunogenic composition comprises:
(i) a cRNA comprising a nucleotide sequence encoding an antigen from influenza, wherein the nucleotide sequence is operably linked to a regulatory element;
(ii) a cRNA comprising a nucleotide sequence encoding an antigen from influenza, wherein the nucleotide sequence is operably linked to a regulatory element; and
(iii) an antigen polypeptide from a severe acute respiratory syndrome coronavirus (SARS-CoV-2).

In another example, the immunogenic composition comprises:
(i) a cRNA comprising a nucleotide sequence encoding an antigen from a severe acute respiratory syndrome coronavirus (SARS-CoV-2), wherein the nucleotide sequence is operably linked to a regulatory element;
(ii) a cRNA comprising a nucleotide sequence encoding an antigen from influenza, wherein the nucleotide sequence is operably linked to a regulatory element; and
(iii) an antigen polypeptide from influenza.

In an example, the immunogenic composition comprises:
(i) a self-replicating RNA comprising a nucleotide sequence encoding an antigen from respiratory syncytial virus (RSV), wherein the nucleotide sequence is operably linked to a regulatory element;
(ii) a self-replicating RNA comprising a nucleotide sequence encoding an antigen from influenza, wherein the nucleotide sequence is operably linked to a regulatory element; and
(iii) an antigen polypeptide from a severe acute respiratory syndrome coronavirus (SARS-CoV-2).

In an example, the immunogenic composition comprises, in order from 5′ to 3′:
(i) the self-replicating RNA comprising a nucleotide sequence encoding an antigen from respiratory syncytial virus (RSV);
(ii) the self-replicating RNA comprising a nucleotide sequence encoding an antigen from influenza.

In an example, the immunogenic composition comprises, in order from 5′ to 3′:
(i) the self-replicating RNA comprising a nucleotide sequence encoding an antigen from influenza, and
(ii) the self-replicating RNA comprising a nucleotide sequence encoding an antigen from respiratory syncytial virus (RSV).

In another example, the immunogenic composition comprises:
(i) a self-replicating RNA comprising a nucleotide sequence encoding an antigen from a severe acute respiratory syndrome coronavirus (SARS-CoV-2), wherein the nucleotide sequence is operably linked to a regulatory element;
(ii) a self-replicating RNA comprising a nucleotide sequence encoding an antigen from influenza, wherein the nucleotide sequence is operably linked to a regulatory element; and
(iii) an antigen polypeptide from a respiratory syncytial virus (RSV).

In an example, the immunogenic composition comprises, in order from 5′ to 3′:
(i) the self-replicating RNA comprising a nucleotide sequence encoding an antigen from a severe acute respiratory syndrome coronavirus (SARS-CoV-2);
(ii) the self-replicating RNA comprising a nucleotide sequence encoding an antigen from influenza.

In an example, the immunogenic composition comprises, in order from 5′ to 3′:
(i) the self-replicating RNA comprising a nucleotide sequence encoding an antigen from influenza; and
(ii) the self-replicating RNA comprising a nucleotide sequence encoding an antigen from a severe acute respiratory syndrome coronavirus (SARS-CoV-2).

In another example, the immunogenic composition comprises:
(i) a self-replicating RNA comprising a nucleotide sequence encoding an antigen from a severe acute respiratory syndrome coronavirus (SARS-CoV-2), wherein the nucleotide sequence is operably linked to a regulatory element;
(ii) a self-replicating RNA comprising a nucleotide sequence encoding an antigen from a respiratory syncytial virus (RSV), wherein the nucleotide sequence is operably linked to a regulatory element; and
(iii) an antigen polypeptide from influenza.

In an example, the immunogenic composition comprises, in order from 5′ to 3′:
(i) the self-replicating RNA comprising a nucleotide sequence encoding an antigen from a severe acute respiratory syndrome coronavirus (SARS-CoV-2);
(ii) the self-replicating RNA comprising a nucleotide sequence encoding an antigen from a respiratory syncytial virus (RSV).

In an example, the immunogenic composition comprises, in order from 5′ to 3′:
(i) the self-replicating RNA comprising a nucleotide sequence encoding an antigen from a respiratory syncytial virus (RSV); and
(ii) the self-replicating RNA comprising a nucleotide sequence encoding an antigen from a severe acute respiratory syndrome coronavirus (SARS-CoV-2).

In another example, the immunogenic composition comprises:
(i) a self-replicating RNA comprising a nucleotide sequence encoding an antigen from a severe acute respiratory syndrome coronavirus (SARS-CoV-2), wherein the nucleotide sequence is operably linked to a regulatory element;
(ii) a self-replicating RNA comprising a nucleotide sequence encoding an antigen from a severe acute respiratory syndrome coronavirus (SARS-CoV-2), wherein the nucleotide sequence is operably linked to a regulatory element; and
(iii) an antigen polypeptide from a severe acute respiratory syndrome coronavirus (SARS-CoV-2).

In another example, the immunogenic composition comprises:
(i) a self-replicating RNA comprising a nucleotide sequence encoding an antigen from a respiratory syncytial virus (RSV), wherein the nucleotide sequence is operably linked to a regulatory element;
(ii) a self-replicating RNA comprising a nucleotide sequence encoding an antigen from a respiratory syncytial virus (RSV), wherein the nucleotide sequence is operably linked to a regulatory element; and
(iii) an antigen polypeptide from a respiratory syncytial virus (RSV).

In another example, the immunogenic composition comprises:
(i) a self-replicating RNA comprising a nucleotide sequence encoding an antigen from influenza, wherein the nucleotide sequence is operably linked to a regulatory element;
(ii) a self-replicating RNA comprising a nucleotide sequence encoding an antigen from influenza, wherein the nucleotide sequence is operably linked to a regulatory element; and
(iii) an antigen polypeptide from influenza.

In an example, the immunogenic composition comprises:
(i) a self-replicating RNA comprising a nucleotide sequence encoding an antigen from a severe acute respiratory syndrome coronavirus (SARS-CoV-2), wherein the nucleotide sequence is operably linked to a regulatory element;
(ii) a self-replicating RNA comprising a nucleotide sequence encoding an antigen from a severe acute respiratory syndrome coronavirus (SARS-CoV-2), wherein the nucleotide sequence is operably linked to a regulatory element; and
(iii) an antigen polypeptide from respiratory syncytial virus (RSV).

In another example, the immunogenic composition comprises:
(i) a self-replicating RNA comprising a nucleotide sequence encoding an antigen from respiratory syncytial virus (RSV), wherein the nucleotide sequence is operably linked to a regulatory element;
(ii) a self-replicating RNA comprising a nucleotide sequence encoding an antigen from a severe acute respiratory syndrome coronavirus (SARS-CoV-2), wherein the nucleotide sequence is operably linked to a regulatory element; and
(iii) an antigen polypeptide from a severe acute respiratory syndrome coronavirus (SARS-CoV-2).

In another example, the immunogenic composition comprises:
(i) a self-replicating RNA comprising a nucleotide sequence encoding an antigen from respiratory syncytial virus (RSV), wherein the nucleotide sequence is operably linked to a regulatory element;
(ii) a self-replicating RNA comprising a nucleotide sequence encoding an antigen from respiratory syncytial virus (RSV), wherein the nucleotide sequence is operably linked to a regulatory element; and
(iii) an antigen polypeptide from a severe acute respiratory syndrome coronavirus (SARS-CoV-2).

In another example, the immunogenic composition comprises:
(i) a self-replicating RNA comprising a nucleotide sequence encoding an antigen from a severe acute respiratory syndrome coronavirus (SARS-CoV-2), wherein the nucleotide sequence is operably linked to a regulatory element;
(ii) a self-replicating RNA comprising a nucleotide sequence encoding an antigen from respiratory syncytial virus (RSV), wherein the nucleotide sequence is operably linked to a regulatory element; and
(iii) an antigen polypeptide from respiratory syncytial virus (RSV).

In an example, the immunogenic composition comprises:
(i) a self-replicating RNA comprising a nucleotide sequence encoding an antigen from influenza, wherein the nucleotide sequence is operably linked to a regulatory element;
(ii) a self-replicating RNA comprising a nucleotide sequence encoding an antigen from influenza, wherein the nucleotide sequence is operably linked to a regulatory element; and
(iii) an antigen polypeptide from respiratory syncytial virus (RSV).

In another example, the immunogenic composition comprises:
(i) a self-replicating RNA comprising a nucleotide sequence encoding an antigen from respiratory syncytial virus (RSV), wherein the nucleotide sequence is operably linked to a regulatory element;
(ii) a self-replicating RNA comprising a nucleotide sequence encoding an antigen from influenza, wherein the nucleotide sequence is operably linked to a regulatory element; and
(iii) an antigen polypeptide from influenza.

In another example, the immunogenic composition comprises:
(i) a self-replicating RNA comprising a nucleotide sequence encoding an antigen from respiratory syncytial virus (RSV), wherein the nucleotide sequence is operably linked to a regulatory element;
(ii) a self-replicating RNA comprising a nucleotide sequence encoding an antigen from respiratory syncytial virus (RSV), wherein the nucleotide sequence is operably linked to a regulatory element; and
(iii) an antigen polypeptide from influenza.

In another example, the immunogenic composition comprises:
(i) a self-replicating RNA comprising a nucleotide sequence encoding an antigen from influenza, wherein the nucleotide sequence is operably linked to a regulatory element;
(ii) a self-replicating RNA comprising a nucleotide sequence encoding an antigen from respiratory syncytial virus (RSV), wherein the nucleotide sequence is operably linked to a regulatory element; and
(iii) an antigen polypeptide from respiratory syncytial virus (RSV).

In an example, the immunogenic composition comprises:
(i) a self-replicating RNA comprising a nucleotide sequence encoding an antigen from a severe acute respiratory syndrome coronavirus (SARS-CoV-2), wherein the nucleotide sequence is operably linked to a regulatory element;
(ii) a self-replicating RNA comprising a nucleotide sequence encoding an antigen from a severe acute respiratory syndrome coronavirus (SARS-CoV-2), wherein the nucleotide sequence is operably linked to a regulatory element; and
(iii) an antigen polypeptide from influenza.

In another example, the immunogenic composition comprises:
(i) a self-replicating RNA comprising a nucleotide sequence encoding an antigen from influenza, wherein the nucleotide sequence is operably linked to a regulatory element;
(ii) a self-replicating RNA comprising a nucleotide sequence encoding an antigen from a severe acute respiratory syndrome coronavirus (SARS-CoV-2), wherein the nucleotide sequence is operably linked to a regulatory element; and
(iii) an antigen polypeptide from a severe acute respiratory syndrome coronavirus (SARS-CoV-2).

In another example, the immunogenic composition comprises:
(i) a self-replicating RNA comprising a nucleotide sequence encoding an antigen from influenza, wherein the nucleotide sequence is operably linked to a regulatory element;
(ii) a self-replicating RNA comprising a nucleotide sequence encoding an antigen from influenza, wherein the nucleotide sequence is operably linked to a regulatory element; and
(iii) an antigen polypeptide from a severe acute respiratory syndrome coronavirus (SARS-CoV-2).

In another example, the immunogenic composition comprises:
(i) a self-replicating RNA comprising a nucleotide sequence encoding an antigen from a severe acute respiratory syndrome coronavirus (SARS-CoV-2), wherein the nucleotide sequence is operably linked to a regulatory element;
(ii) a self-replicating RNA comprising a nucleotide sequence encoding an antigen from influenza, wherein the nucleotide sequence is operably linked to a regulatory element; and
(iii) an antigen polypeptide from influenza.

In one example, an immunogenic composition comprises multiple multicistronic self-replicating RNAs, wherein each multicistronic self-replicating RNA encodes a polypeptide of an antigen of interest. In another example, an immunogenic composition of the disclosure comprises a plurality of self-replicating monocistronic RNAs, wherein each self-replicating RNA encodes different polypeptide sequences. For example, one self-replicating RNA encodes a polypeptide of an antigen of interest and a different self-replicating RNA encodes a different polypeptide of an antigen of interest.

In one example of the present disclosure, the immunogenic composition comprises multiple polypeptide antigens, wherein each polypeptide is a different antigen of interest. In another embodiment, the different polypeptide of an antigen of interest may be from the same virus. For example, different polypeptides may be from a respiratory syncytial virus (RSV), a severe acute respiratory syndrome coronavirus (SARS-CoV-2) or influenza.

In an example, the immunogenic composition comprises:
(i) a polynucleotide comprising a nucleotide sequence encoding an antigen from a severe acute respiratory syndrome coronavirus (SARS-CoV-2), wherein the nucleotide sequence is operably linked to a regulatory element;
(ii) an antigen polypeptide from influenza; and
(iii) an antigen polypeptide from a respiratory syncytial virus (RSV).

In an example, the immunogenic composition comprises:
(i) a polynucleotide comprising a nucleotide sequence encoding an antigen from influenza, wherein the nucleotide sequence is operably linked to a regulatory element;
(ii) an antigen polypeptide from a severe acute respiratory syndrome coronavirus (SARS-CoV-2); and
(iii) an antigen polypeptide from a respiratory syncytial virus (RSV).

In an example, the immunogenic composition comprises:
(i) a polynucleotide comprising a nucleotide sequence encoding an antigen from a respiratory syncytial virus (RSV), wherein the nucleotide sequence is operably linked to a regulatory element;
(ii) an antigen polypeptide from a severe acute respiratory syndrome coronavirus (SARS-CoV-2); and
(iii) an antigen polypeptide from influenza.

In an example, the immunogenic composition comprises:
(i) a polynucleotide comprising a nucleotide sequence encoding an antigen from a respiratory syncytial virus (RSV), wherein the nucleotide sequence is operably linked to a regulatory element;
(ii) an antigen polypeptide from a respiratory syncytial virus (RSV); and
(iii) a further polypeptide from a respiratory syncytial virus (RSV).

In an example, the immunogenic composition comprises:
(i) a polynucleotide comprising a nucleotide sequence encoding an antigen from influenza, wherein the nucleotide sequence is operably linked to a regulatory element;
(ii) an antigen polypeptide from influenza; and
(iii) a further antigen polypeptide from influenza.

In an example, the immunogenic composition comprises:
(i) a polynucleotide comprising a nucleotide sequence encoding an antigen from a severe acute respiratory syndrome coronavirus (SARS-CoV-2), wherein the nucleotide sequence is operably linked to a regulatory element;
(ii) an antigen polypeptide from a severe acute respiratory syndrome coronavirus (SARS-CoV-2); and
(iii) a further antigen polypeptide from a severe acute respiratory syndrome coronavirus (SARS-CoV-2).

In an example, the immunogenic composition comprises:
(i) a RNA comprising a nucleotide sequence encoding an antigen from a severe acute respiratory syndrome coronavirus (SARS-CoV-2), wherein the nucleotide sequence is operably linked to a regulatory element;
(ii) an antigen polypeptide from influenza; and
(iii) an antigen polypeptide from a respiratory syncytial virus (RSV).

In an example, the immunogenic composition comprises:
(i) a RNA comprising a nucleotide sequence encoding an antigen from influenza, wherein the nucleotide sequence is operably linked to a regulatory element;
(ii) an antigen polypeptide from a severe acute respiratory syndrome coronavirus (SARS-CoV-2); and
(iii) an antigen polypeptide from a respiratory syncytial virus (RSV).

In an example, the immunogenic composition comprises:
(i) a RNA comprising a nucleotide sequence encoding an antigen from a respiratory syncytial virus (RSV), wherein the nucleotide sequence is operably linked to a regulatory element;
(ii) an antigen polypeptide from a severe acute respiratory syndrome coronavirus (SARS-CoV-2); and
(iii) an antigen polypeptide from influenza.

In an example, the immunogenic composition comprises:
(i) a RNA comprising a nucleotide sequence encoding an antigen from a respiratory syncytial virus (RSV), wherein the nucleotide sequence is operably linked to a regulatory element;
(ii) an antigen polypeptide from a respiratory syncytial virus (RSV); and
(iii) a further polypeptide from a respiratory syncytial virus (RSV).

In an example, the immunogenic composition comprises:
(i) a RNA comprising a nucleotide sequence encoding an antigen from influenza, wherein the nucleotide sequence is operably linked to a regulatory element;
(ii) an antigen polypeptide from influenza; and
(iii) a further antigen polypeptide from influenza.

In an example, the immunogenic composition comprises:
(i) a RNA comprising a nucleotide sequence encoding an antigen from a severe acute respiratory syndrome coronavirus (SARS-CoV-2), wherein the nucleotide sequence is operably linked to a regulatory element;
(ii) an antigen polypeptide from a severe acute respiratory syndrome coronavirus (SARS-CoV-2); and
(iii) a further antigen polypeptide from a severe acute respiratory syndrome coronavirus (SARS-CoV-2).

In an example, the immunogenic composition comprises:
(i) a cRNA comprising a nucleotide sequence encoding an antigen from a severe acute respiratory syndrome coronavirus (SARS-CoV-2), wherein the nucleotide sequence is operably linked to a regulatory element;
(ii) an antigen polypeptide from influenza; and
(iii) an antigen polypeptide from a respiratory syncytial virus (RSV).

In an example, the immunogenic composition comprises:
(i) a cRNA comprising a nucleotide sequence encoding an antigen from influenza, wherein the nucleotide sequence is operably linked to a regulatory element;
(ii) an antigen polypeptide from a severe acute respiratory syndrome coronavirus (SARS-CoV-2); and
(iii) an antigen polypeptide from a respiratory syncytial virus (RSV).

In an example, the immunogenic composition comprises:
(i) a cRNA comprising a nucleotide sequence encoding an antigen from a respiratory syncytial virus (RSV), wherein the nucleotide sequence is operably linked to a regulatory element;
(ii) an antigen polypeptide from a severe acute respiratory syndrome coronavirus (SARS-CoV-2); and
(iii) an antigen polypeptide from influenza.

In an example, the immunogenic composition comprises:
(i) a cRNA comprising a nucleotide sequence encoding an antigen from a respiratory syncytial virus (RSV), wherein the nucleotide sequence is operably linked to a regulatory element;
(ii) an antigen polypeptide from a respiratory syncytial virus (RSV); and
(iii) a further polypeptide from a respiratory syncytial virus (RSV).

In an example, the immunogenic composition comprises:
(i) a cRNA comprising a nucleotide sequence encoding an antigen from influenza, wherein the nucleotide sequence is operably linked to a regulatory element;
(ii) an antigen polypeptide from influenza; and
(iii) a further antigen polypeptide from influenza.

In an example, the immunogenic composition comprises:
(i) a cRNA comprising a nucleotide sequence encoding an antigen from a severe acute respiratory syndrome coronavirus (SARS-CoV-2), wherein the nucleotide sequence is operably linked to a regulatory element;
(ii) an antigen polypeptide from a severe acute respiratory syndrome coronavirus (SARS-CoV-2); and
(iii) a further antigen polypeptide from a severe acute respiratory syndrome coronavirus (SARS-CoV-2).

In an example, the immunogenic composition comprises:
(i) a self-replicating RNA comprising a nucleotide sequence encoding an antigen from a severe acute respiratory syndrome coronavirus (SARS-CoV-2), wherein the nucleotide sequence is operably linked to a regulatory element;
(ii) an antigen polypeptide from influenza; and
(iii) an antigen polypeptide from a respiratory syncytial virus (RSV).

In an example, the immunogenic composition comprises:
(i) a self-replicating RNA comprising a nucleotide sequence encoding an antigen from influenza, wherein the nucleotide sequence is operably linked to a regulatory element;
(ii) an antigen polypeptide from a severe acute respiratory syndrome coronavirus (SARS-CoV-2); and
(iii) an antigen polypeptide from a respiratory syncytial virus (RSV).

In an example, the immunogenic composition comprises:
(i) a self-replicating RNA comprising a nucleotide sequence encoding an antigen from a respiratory syncytial virus (RSV), wherein the nucleotide sequence is operably linked to a regulatory element;
(ii) an antigen polypeptide from a severe acute respiratory syndrome coronavirus (SARS-CoV-2); and
(iii) an antigen polypeptide from influenza.

In an example, the immunogenic composition comprises:
(i) a self-replicating RNA comprising a nucleotide sequence encoding an antigen from a respiratory syncytial virus (RSV), wherein the nucleotide sequence is operably linked to a regulatory element;
(ii) an antigen polypeptide from a respiratory syncytial virus (RSV); and
(iii) a further polypeptide from a respiratory syncytial virus (RSV).

In an example, the immunogenic composition comprises:
(i) a self-replicating RNA comprising a nucleotide sequence encoding an antigen from influenza, wherein the nucleotide sequence is operably linked to a regulatory element;
(ii) an antigen polypeptide from influenza; and
(iii) a further antigen polypeptide from influenza.

In an example, the immunogenic composition comprises:
(i) a self-replicating RNA comprising a nucleotide sequence encoding an antigen from a severe acute respiratory syndrome coronavirus (SARS-CoV-2), wherein the nucleotide sequence is operably linked to a regulatory element;
(ii) an antigen polypeptide from a severe acute respiratory syndrome coronavirus (SARS-CoV-2); and
(iii) a further antigen polypeptide from a severe acute respiratory syndrome coronavirus (SARS-CoV-2).

In an example, the compositions described herein further comprise an adjuvant.

In an example of the disclosure a protein antigen is formulated with an adjuvant.

In one example, the adjuvant is selected from the group consisting of Freund's adjuvant, incomplete Freund's adjuvants, aluminum phosphate, aluminum hydroxide, GMCSP, BCG, MDP compounds, such as thur-MDP and nor-MDP, CGP (MTP-PE), lipid A, monophosphoryl lipid A (MPL), RIBI, MPL, trehalose dimycolate (TDM), Novasomes®, QS21, Quil A (and derivatives and components thereof), calcium phosphate, calcium hydroxide, zinc hydroxide, MHC antigens, PolyI:C, MF59, glycolipid analogs, octodecyl esters of an amino acid, muramyl dipeptides, polyphosphazene, lipoproteins, ISCOM matrix, DC-Chol, ODA, cytokines, and other adjuvants and derivatives thereof. In another example, the adjuvant is MF59.

In an example, the effect of an immunogenic composition described herein, on treating, preventing or delaying progression of a disease or disorder is enhanced in the presence of an adjuvant such as MF59, when compared to the effect of an immunogenic composition described herein without an adjuvant.

In an example, the effect of an immunogenic composition described herein, on treating, preventing or delaying progression of a SARS-CoV-2 infection, COVID-19, influenza and/or respiratory syncytial virus (RSV) is enhanced in the presence of an adjuvant such as MF59, when compared to the effect of an immunogenic composition described herein without an adjuvant.

In an example, the effect of an immunogenic composition described herein, on treating, preventing or delaying progression of pneumonia, sepsis or acute respiratory distress syndrome in a subject having COVID-19 is enhanced in the presence of an adjuvant such as MF59, when compared to the effect of an immunogenic composition described herein without an adjuvant.

In an example, the effect of an immunogenic composition described herein, on treating, preventing or delaying progression of an influenza infection is enhanced in the presence of an adjuvant such as MF59, when compared to the effect of an immunogenic composition described herein without an adjuvant.

In an example, the effect of an immunogenic composition described herein, on treating, preventing or delaying progression of a RSV infection is enhanced in the presence of an adjuvant such as MF59, when compared to the effect of an immunogenic composition described herein without an adjuvant.

In an example, the effect of an immunogenic composition described herein, on inducing an immune response in a subject is enhanced in the presence of an adjuvant such as MF59, when compared to the effect of an immunogenic composition described herein without an adjuvant.

In an example, the presence of an adjuvant such as MF59 in an immunogenic composition described herein enhances the induction of the immune response of the composition by at least about 5%, at least about 10%, at least about 15%, at least about 20%, at least about 25%, at least about 30%, at least about 35%, at least about 40%, at least about 45%, at least about 50%, at least about 55% at least about 60%, at least about 65%, at least about 70%, at least about 75%, at least about 80%, at least about 85%, at least about 90%, at least about 95%, at least about 100%, at least about 200%, at least about 300%, at least about 400%, at least about 500% or more when compared to the effect of an immunogenic composition described herein without an adjuvant such as MF59.

In an example, the adjuvant is administered at the same time as an immunogenic composition of the disclosure. In another example, the adjuvant is administered sequentially to, preceding, or proceeding the administration of an immunogenic composition of the disclosure.

In one example, a composition described herein further comprises an additional polypeptide selected from the group consisting of:
(i) one or more antigens;
(ii) one or more immunopotentiating agents;
(iii) one or more chemoattractants; and/or
(iv) one or more targeting molecules.

In an example, the nucleotide sequence is operably linked to a regulatory element selected from the group consisting of a promoter, optionally a subgenomic (SG) promoter, an internal ribosome entry site (IRES) and a Kozak consensus sequence or a combination thereof. In an example, the regulatory element is a SG promoter.

For example, the polynucleotide of the immunogenic composition comprises a nucleotide sequence encoding an antigen operably linked to a regulatory element selected from a SG promoter, and IRES and/or a Kozak consensus sequence.

In another example, the RNA of the immunogenic composition comprises a nucleotide sequence encoding an antigen operably linked to a regulatory element selected from a SG promoter, and IRES and/or a Kozak consensus sequence.

In another example, the cRNA of the immunogenic composition comprises a nucleotide sequence encoding an antigen operably linked to a regulatory element selected from a SG promoter, and IRES and/or a Kozak consensus sequence.

In another example, the self-replicating RNA of the immunogenic composition comprises a nucleotide sequence encoding an antigen operably linked to a regulatory element selected from a SG promoter, and IRES and/or a Kozak consensus sequence.

Accordingly, the present disclosure provides an immunogenic composition comprising:
(i) a polynucleotide comprising: a) a first nucleotide sequence encoding an antigen from a respiratory syncytial virus (RSV); and b) a second nucleotide sequence encoding an antigen from influenza, operably linked to a regulatory element selected from the group consisting of a SG promoter and an IRES; and
(ii) an antigen polypeptide from a severe acute respiratory syndrome coronavirus (SARS-CoV-2).

In one example, the polynucleotide comprises, in order from 5′ to 3′: a) a first nucleotide sequence encoding an antigen from a respiratory syncytial virus (RSV); and b) a second nucleotide sequence encoding an antigen from influenza, operably linked to a regulatory element selected from the group consisting of a SG promoter and an IRES.

In one example, the polynucleotide comprises, in order from 5′ to 3′: a) a first nucleotide sequence encoding an antigen from influenza; and b) a second nucleotide sequence encoding an antigen from respiratory syncytial virus (RSV), operably linked to a regulatory element selected from the group consisting of a SG promoter and an IRES.

In another example, the present disclosure provides an immunogenic composition comprising:
(i) a polynucleotide comprising: a) a first nucleotide sequence encoding an antigen from a severe acute respiratory syndrome coronavirus (SARS-CoV-2); and b) a second nucleotide sequence encoding an antigen from influenza, operably linked to a regulatory element selected from the group consisting of a SG promoter and an IRES; and
(ii) an antigen polypeptide from a respiratory syncytial virus (RSV).

In one example, the polynucleotide comprises, in order from 5′ to 3′: a) a first nucleotide sequence encoding an antigen from a severe acute respiratory syndrome coronavirus (SARS-CoV-2); and b) a second nucleotide sequence encoding an antigen from influenza, operably linked to a regulatory element selected from the group consisting of a SG promoter and an IRES.

In one example, the polynucleotide comprises, in order from 5′ to 3′: a) a first nucleotide sequence encoding an antigen from influenza; and b) a second nucleotide sequence encoding an antigen from a severe acute respiratory syndrome coronavirus (SARS-CoV-2), operably linked to a regulatory element selected from the group consisting of a SG promoter and an IRES.

In another example, the present disclosure provides an immunogenic composition comprising:
(i) a polynucleotide comprising: a) a first nucleotide sequence encoding an antigen from a severe acute respiratory syndrome coronavirus (SARS-CoV-2); and b) a second nucleotide sequence encoding an antigen from a respiratory syncytial virus (RSV), operably linked to a regulatory element selected from the group consisting of a SG promoter and an IRES; and
(ii) an antigen polypeptide from influenza.

In one example, the polynucleotide comprises, in order from 5′ to 3′: a) a first nucleotide sequence encoding an antigen from a severe acute respiratory syndrome coronavirus (SARS-CoV-2); and b) a second nucleotide sequence encoding an antigen from a respiratory syncytial virus (RSV), operably linked to a regulatory element selected from the group consisting of a SG promoter and an IRES.

In one example, the polynucleotide comprises, in order from 5′ to 3′: a) a first nucleotide sequence encoding an antigen from a respiratory syncytial virus (RSV); and b) a second nucleotide sequence encoding an antigen from a severe acute respiratory syndrome coronavirus (SARS-CoV-2), operably linked to a regulatory element selected from the group consisting of a SG promoter and an IRES.

Accordingly, the present disclosure also provides an immunogenic composition comprising:
(i) a RNA comprising: a) a first nucleotide sequence encoding an antigen from a respiratory syncytial virus (RSV); and b) a second nucleotide sequence encoding an antigen from influenza, operably linked to a regulatory element selected from the group consisting of a SG promoter and an IRES; and
(ii) an antigen polypeptide from a severe acute respiratory syndrome coronavirus (SARS-CoV-2).

In one example, the RNA comprises, in order from 5′ to 3′: a) a first nucleotide sequence encoding an antigen from a respiratory syncytial virus (RSV); and b) a second nucleotide sequence encoding an antigen from influenza, operably linked to a regulatory element selected from the group consisting of a SG promoter and an IRES.

In one example, the RNA comprises, in order from 5′ to 3′: a) a first nucleotide sequence encoding an antigen from influenza; and b) a second nucleotide sequence encoding an antigen from respiratory syncytial virus (RSV), operably linked to a regulatory element selected from the group consisting of a SG promoter and an IRES.

In another example, the present disclosure provides an immunogenic composition comprising:
(i) a RNA comprising: a) a first nucleotide sequence encoding an antigen from a severe acute respiratory syndrome coronavirus (SARS-CoV-2); and b) a second nucleotide sequence encoding an antigen from influenza, operably linked to a regulatory element selected from the group consisting of a SG promoter and an IRES; and
(ii) an antigen polypeptide from a respiratory syncytial virus (RSV).

In one example, the RNA comprises, in order from 5′ to 3′: a) a first nucleotide sequence encoding an antigen from a severe acute respiratory syndrome coronavirus (SARS-CoV-2); and b) a second nucleotide sequence encoding an antigen from influenza, operably linked to a regulatory element selected from the group consisting of a SG promoter and an IRES.

In one example, the RNA comprises, in order from 5′ to 3′: a) a first nucleotide sequence encoding an antigen from influenza; and b) a second nucleotide sequence encoding an antigen from a severe acute respiratory syndrome coronavirus (SARS-CoV-2), operably linked to a regulatory element selected from the group consisting of a SG promoter and an IRES.

In another example, the present disclosure provides an immunogenic composition comprising:
(i) a RNA comprising: a) a first nucleotide sequence encoding an antigen from a severe acute respiratory syndrome coronavirus (SARS-CoV-2); and b) a second nucleotide sequence encoding an antigen from a respiratory syncytial virus (RSV), operably linked to a regulatory element selected from the group consisting of a SG promoter and an IRES; and
(ii) an antigen polypeptide from influenza.

In one example, the RNA comprises, in order from 5′ to 3′: a) a first nucleotide sequence encoding an antigen from a severe acute respiratory syndrome coronavirus (SARS-CoV-2); and b) a second nucleotide sequence encoding an antigen from a respiratory syncytial virus (RSV), operably linked to a regulatory element selected from the group consisting of a SG promoter and an IRES.

In one example, the RNA comprises, in order from 5′ to 3′: a) a first nucleotide sequence encoding an antigen from a respiratory syncytial virus (RSV); and b) a second nucleotide sequence encoding an antigen from a severe acute respiratory syndrome coronavirus (SARS-CoV-2), operably linked to a regulatory element selected from the group consisting of a SG promoter and an IRES.

Accordingly, the present disclosure also provides an immunogenic composition comprising:
(i) a cRNA comprising: a) a first nucleotide sequence encoding an antigen from a respiratory syncytial virus (RSV); and b) a second nucleotide sequence encoding an antigen from influenza, operably linked to a regulatory element selected from the group consisting of a SG promoter and an IRES; and
(ii) an antigen polypeptide from a severe acute respiratory syndrome coronavirus (SARS-CoV-2).

In one example, the cRNA comprises, in order from 5′ to 3′: a) a first nucleotide sequence encoding an antigen from a respiratory syncytial virus (RSV); and b) a second nucleotide sequence encoding an antigen from influenza, operably linked to a regulatory element selected from the group consisting of a SG promoter and an IRES.

In one example, the cRNA comprises, in order from 5′ to 3′: a) a first nucleotide sequence encoding an antigen from influenza; and b) a second nucleotide sequence encoding an antigen from respiratory syncytial virus (RSV), operably linked to a regulatory element selected from the group consisting of a SG promoter and an IRES.

In another example, the present disclosure provides an immunogenic composition comprising:
(i) a cRNA comprising: a) a first nucleotide sequence encoding an antigen from a severe acute respiratory syndrome coronavirus (SARS-CoV-2); and b) a second nucleotide sequence encoding an antigen from influenza, operably linked to a regulatory element selected from the group consisting of a SG promoter and an IRES; and
(ii) an antigen polypeptide from a respiratory syncytial virus (RSV).

In one example, the cRNA comprises, in order from 5′ to 3′: a) a first nucleotide sequence encoding an antigen from a severe acute respiratory syndrome coronavirus (SARS-CoV-2); and b) a second nucleotide sequence encoding an antigen from influenza, operably linked to a regulatory element selected from the group consisting of a SG promoter and an IRES.

In one example, the cRNA comprises, in order from 5′ to 3′: a) a first nucleotide sequence encoding an antigen from influenza; and b) a second nucleotide sequence encoding an antigen from a severe acute respiratory syndrome coronavirus (SARS-CoV-2), operably linked to a regulatory element selected from the group consisting of a SG promoter and an IRES.

In another example, the present disclosure provides an immunogenic composition comprising:
(i) a cRNA comprising: a) a first nucleotide sequence encoding an antigen from a severe acute respiratory syndrome coronavirus (SARS-CoV-2); and b) a second nucleotide sequence encoding an antigen from a respiratory syncytial virus (RSV), operably linked to a regulatory element selected from the group consisting of a SG promoter and an IRES; and
(ii) an antigen polypeptide from influenza.

In one example, the cRNA comprises, in order from 5′ to 3′: a) a first nucleotide sequence encoding an antigen from a severe acute respiratory syndrome coronavirus (SARS-CoV-2); and b) a second nucleotide sequence encoding an antigen from a respiratory syncytial virus (RSV), operably linked to a regulatory element selected from the group consisting of a SG promoter and an IRES.

In one example, the cRNA comprises, in order from 5′ to 3′: a) a first nucleotide sequence encoding an antigen from a respiratory syncytial virus (RSV); and b) a second nucleotide sequence encoding an antigen from a severe acute respiratory syndrome coronavirus (SARS-CoV-2), operably linked to a regulatory element selected from the group consisting of a SG promoter and an IRES.

Accordingly, the present disclosure also provides an immunogenic composition comprising:
(i) a self-replicating RNA comprising: a) a first nucleotide sequence encoding an antigen from a respiratory syncytial virus (RSV); and b) a second nucleotide sequence encoding an antigen from influenza, operably linked to a regulatory element selected from the group consisting of a SG promoter and an IRES; and
(ii) an antigen polypeptide from a severe acute respiratory syndrome coronavirus (SARS-CoV-2).

In one example, the self-replicating RNA comprises, in order from 5′ to 3′: a) a first nucleotide sequence encoding an antigen from a respiratory syncytial virus (RSV); and b) a second nucleotide sequence encoding an antigen from influenza, operably linked to a regulatory element selected from the group consisting of a SG promoter and an IRES.

In one example, the self-replicating RNA comprises, in order from 5′ to 3′: a) a first nucleotide sequence encoding an antigen from influenza; and b) a second nucleotide sequence encoding an antigen from respiratory syncytial virus (RSV), operably linked to a regulatory element selected from the group consisting of a SG promoter and an IRES.

In another example, the present disclosure provides an immunogenic composition comprising:
(i) a self-replicating RNA comprising: a) a first nucleotide sequence encoding an antigen from a severe acute respiratory syndrome coronavirus (SARS-CoV-2); and b) a second nucleotide sequence encoding an antigen from influenza, operably linked to a regulatory element selected from the group consisting of a SG promoter and an IRES; and
(ii) an antigen polypeptide from a respiratory syncytial virus (RSV).

In one example, the self-replicating RNA comprises, in order from 5′ to 3′: a) a first nucleotide sequence encoding an antigen from a severe acute respiratory syndrome coronavirus (SARS-CoV-2); and b) a second nucleotide sequence encoding an antigen from influenza, operably linked to a regulatory element selected from the group consisting of a SG promoter and an IRES.

In one example, the self-replicating RNA comprises, in order from 5′ to 3′: a) a first nucleotide sequence encoding an antigen from influenza; and b) a second nucleotide sequence encoding an antigen from a severe acute respiratory syndrome coronavirus (SARS-CoV-2), operably linked to a regulatory element selected from the group consisting of a SG promoter and an IRES.

In another example, the present disclosure provides an immunogenic composition comprising:
(i) a self-replicating RNA comprising: a) a first nucleotide sequence encoding an antigen from a severe acute respiratory syndrome coronavirus (SARS-CoV-2); and b) a second nucleotide sequence encoding an antigen from a respiratory syncytial virus (RSV), operably linked to a regulatory element selected from the group consisting of a SG promoter and an IRES; and
(ii) an antigen polypeptide from influenza.

In one example, the self-replicating RNA comprises, in order from 5′ to 3′: a) a first nucleotide sequence encoding an antigen from a severe acute respiratory syndrome coronavirus (SARS-CoV-2); and b) a second nucleotide sequence encoding an antigen from a respiratory syncytial virus (RSV), operably linked to a regulatory element selected from the group consisting of a SG promoter and an IRES.

In one example, the self-replicating RNA comprises, in order from 5′ to 3′: a) a first nucleotide sequence encoding an antigen from a respiratory syncytial virus (RSV); and b) a second nucleotide sequence encoding an antigen from a severe acute respiratory syndrome coronavirus (SARS-CoV-2), operably linked to a regulatory element selected from the group consisting of a SG promoter and an IRES.

The present disclosure further provides an immunogenic composition comprising
(i) a polynucleotide comprising: a) a first nucleotide sequence encoding an antigen from respiratory syncytial virus (RSV) operably linked to a regulatory element selected from the group consisting of a Kozak consensus sequence, an IRES, a SG promoter and combinations thereof, and b) a second nucleotide sequence encoding an antigen from influenza, operably linked to a regulatory element selected from the group consisting of a SG promoter and an IRES; and
(ii) an antigen polypeptide from a severe acute respiratory syndrome coronavirus (SARS-CoV-2).

In one example, the polynucleotide comprises, in order from 5′ to 3′: a) a first nucleotide sequence encoding a first antigen antigen from respiratory syncytial virus (RSV) operably linked to a regulatory element selected from the group consisting of a Kozak consensus sequence, an IRES, a SG promoter and combinations thereof, and b) a second nucleotide sequence encoding an antigen from influenza, operably linked to a regulatory element selected from the group consisting of a SG promoter and an IRES.

In one example, the polynucleotide comprises, in order from 5′ to 3′: a) a first nucleotide sequence encoding an antigen from influenza, operably linked to a regulatory element selected from the group consisting of a Kozak consensus sequence, an IRES, a SG promoter and combinations thereof, and b) a second nucleotide sequence encoding a first antigen antigen from respiratory syncytial virus (RSV) operably linked to a regulatory element selected from the group consisting of a SG promoter and an IRES.

In another example, the immunogenic composition comprises:
(i) a polynucleotide comprising: a) a first nucleotide sequence encoding an antigen from SARS-CoV-2 operably linked to a regulatory element selected from the group consisting of a Kozak consensus sequence, an IRES, a SG promoter and combinations thereof, and b) a second nucleotide sequence encoding an antigen from influenza, operably linked to a regulatory element selected from the group consisting of a SG promoter and an IRES; and
(ii) an antigen polypeptide from a respiratory syncytial virus (RSV).

In one example, the polynucleotide comprises, in order from 5′ to 3′: a) a first nucleotide sequence encoding a first antigen antigen from a severe acute respiratory syndrome coronavirus (SARS-CoV-2) operably linked to a regulatory element selected from the group consisting of a Kozak consensus sequence, an IRES, a SG promoter and combinations thereof, and b) a second nucleotide sequence encoding an antigen from influenza, operably linked to a regulatory element selected from the group consisting of a SG promoter and an IRES.

In one example, the polynucleotide comprises, in order from 5′ to 3′: a) a first nucleotide sequence encoding an antigen from influenza, operably linked to a regulatory element selected from the group consisting of a Kozak consensus sequence, an IRES, a SG promoter and combinations thereof, and b) a second nucleotide sequence encoding a first antigen antigen from a severe acute respiratory syndrome coronavirus (SARS-CoV-2) operably linked to a regulatory element selected from the group consisting of a SG promoter and an IRES.

In another example, the immunogenic composition comprises:
(i) a polynucleotide comprising: a) a first nucleotide sequence encoding an antigen from a severe acute respiratory syndrome coronavirus (SARS-CoV-2) operably linked to a regulatory element selected from the group consisting of a Kozak consensus sequence, an IRES, a SG promoter and combinations thereof, and b) a second nucleotide sequence encoding an antigen from a respiratory syncytial virus (RSV), operably linked to a regulatory element selected from the group consisting of a SG promoter and an IRES; and
(ii) an antigen polypeptide from influenza.

In one example, the polynucleotide comprises, in order from 5′ to 3′: a) a first nucleotide sequence encoding a first antigen antigen from a severe acute respiratory syndrome coronavirus (SARS-CoV-2) operably linked to a regulatory element selected from the group consisting of a Kozak consensus sequence, an IRES, a SG promoter and combinations thereof, and b) a second nucleotide sequence encoding an antigen from a respiratory syncytial virus (RSV), operably linked to a regulatory element selected from the group consisting of a SG promoter and an IRES.

In one example, the polynucleotide comprises, in order from 5′ to 3′: a) a first nucleotide sequence encoding an antigen from a respiratory syncytial virus (RSV), operably linked to a regulatory element selected from the group consisting of a Kozak consensus sequence, an IRES, a SG promoter and combinations thereof, and b) a second nucleotide sequence encoding a first antigen antigen from a severe acute respiratory syndrome coronavirus (SARS-CoV-2) operably linked to a regulatory element selected from the group consisting of a SG promoter and an IRES.

The present disclosure also provides an immunogenic composition comprising
(i) a RNA comprising: a) a first nucleotide sequence encoding an antigen from respiratory syncytial virus (RSV) operably linked to a regulatory element selected from the group consisting of a Kozak consensus sequence, an IRES, a SG promoter and combinations thereof, and b) a second nucleotide sequence encoding an antigen from influenza, operably linked to a regulatory element selected from the group consisting of a SG promoter and an IRES; and
(ii) an antigen polypeptide from a SARS-CoV-2.

In one example, the RNA comprises, in order from 5′ to 3′: a) a first nucleotide sequence encoding a first antigen antigen from respiratory syncytial virus (RSV) operably linked to a regulatory element selected from the group consisting of a Kozak consensus sequence, an IRES, a SG promoter and combinations thereof, and b) a second nucleotide sequence encoding an antigen from influenza, operably linked to a regulatory element selected from the group consisting of a SG promoter and an IRES.

In one example, the RNA comprises, in order from 5′ to 3′: a) a first nucleotide sequence encoding an antigen from influenza, operably linked to a regulatory element selected from the group consisting of a Kozak consensus sequence, an IRES, a SG promoter and combinations thereof, and b) a second nucleotide sequence encoding a first antigen antigen from respiratory syncytial virus (RSV) operably linked to a regulatory element selected from the group consisting of a SG promoter and an IRES.

In another example, the immunogenic composition comprises:
(i) a RNA comprising: a) a first nucleotide sequence encoding an antigen from a severe acute respiratory syndrome coronavirus (SARS-CoV-2) operably linked to a regulatory element selected from the group consisting of a Kozak consensus sequence, an IRES, a SG promoter and combinations thereof, and b) a second nucleotide sequence encoding an antigen from influenza, operably linked to a regulatory element selected from the group consisting of a SG promoter and an IRES; and
(ii) an antigen polypeptide from a respiratory syncytial virus (RSV).

In one example, the RNA comprises, in order from 5′ to 3′: a) a first nucleotide sequence encoding a first antigen antigen from a severe acute respiratory syndrome coronavirus (SARS-CoV-2) operably linked to a regulatory element selected from the group consisting of a Kozak consensus sequence, an IRES, a SG promoter and combinations thereof, and b) a second nucleotide sequence encoding an antigen from influenza, operably linked to a regulatory element selected from the group consisting of a SG promoter and an IRES.

In one example, the RNA comprises, in order from 5′ to 3′: a) a first nucleotide sequence encoding an antigen from influenza, operably linked to a regulatory element selected from the group consisting of a Kozak consensus sequence, an IRES, a SG promoter and combinations thereof, and b) a second nucleotide sequence encoding a first antigen antigen from a severe acute respiratory syndrome coronavirus (SARS-CoV-2) operably linked to a regulatory element selected from the group consisting of a SG promoter and an IRES.

In another example, the immunogenic composition comprises:
(i) a RNA comprising: a) a first nucleotide sequence encoding an antigen from a severe acute respiratory syndrome coronavirus (SARS-CoV-2) operably linked to a regulatory element selected from the group consisting of a Kozak consensus sequence, an IRES, a SG promoter and combinations thereof, and b) a second nucleotide sequence encoding an antigen from a respiratory syncytial virus (RSV), operably linked to a regulatory element selected from the group consisting of a SG promoter and an IRES; and
(ii) an antigen polypeptide from influenza.

In one example, the RNA comprises, in order from 5′ to 3′: a) a first nucleotide sequence encoding a first antigen antigen from a severe acute respiratory syndrome coronavirus (SARS-CoV-2) operably linked to a regulatory element selected from the group consisting of a Kozak consensus sequence, an IRES, a SG promoter and combinations thereof, and b) a second nucleotide sequence encoding an antigen from a respiratory syncytial virus (RSV), operably linked to a regulatory element selected from the group consisting of a SG promoter and an IRES.

In one example, the RNA comprises, in order from 5′ to 3′: a) a first nucleotide sequence encoding an antigen from a respiratory syncytial virus (RSV), operably linked to a regulatory element selected from the group consisting of a Kozak consensus sequence, an IRES, a SG promoter and combinations thereof, and b) a second nucleotide sequence encoding a first antigen antigen from a severe acute respiratory syndrome coronavirus (SARS-CoV-2) operably linked to a regulatory element selected from the group consisting of a SG promoter and an IRES.

The present disclosure also provides an immunogenic composition comprising
(i) a cRNA comprising: a) a first nucleotide sequence encoding an antigen from respiratory syncytial virus (RSV) operably linked to a regulatory element selected from the group consisting of a Kozak consensus sequence, an IRES, a SG promoter and combinations thereof, and b) a second nucleotide sequence encoding an antigen from influenza, operably linked to a regulatory element selected from the group consisting of a SG promoter and an IRES; and
(ii) an antigen polypeptide from a SARS-CoV-2.

In one example, the cRNA comprises, in order from 5′ to 3′: a) a first nucleotide sequence encoding a first antigen antigen from respiratory syncytial virus (RSV) operably linked to a regulatory element selected from the group consisting of a Kozak consensus sequence, an IRES, a SG promoter and combinations thereof, and b) a second nucleotide sequence encoding an antigen from influenza, operably linked to a regulatory element selected from the group consisting of a SG promoter and an IRES.

In one example, the cRNA comprises, in order from 5′ to 3′: a) a first nucleotide sequence encoding an antigen from influenza, operably linked to a regulatory element selected from the group consisting of a Kozak consensus sequence, an IRES, a SG promoter and combinations thereof, and b) a second nucleotide sequence encoding a first antigen antigen from respiratory syncytial virus (RSV) operably linked to a regulatory element selected from the group consisting of a SG promoter and an IRES.

In another example, the immunogenic composition comprises:
(i) a cRNA comprising: a) a first nucleotide sequence encoding an antigen from a severe acute respiratory syndrome coronavirus (SARS-CoV-2) operably linked to a regulatory element selected from the group consisting of a Kozak consensus sequence, an IRES, a SG promoter and combinations thereof, and b) a second nucleotide sequence encoding an antigen from influenza, operably linked to a regulatory element selected from the group consisting of a SG promoter and an IRES; and
(ii) an antigen polypeptide from a respiratory syncytial virus (RSV).

In one example, the cRNA comprises, in order from 5′ to 3′: a) a first nucleotide sequence encoding a first antigen antigen from a severe acute respiratory syndrome coronavirus (SARS-CoV-2) operably linked to a regulatory element selected from the group consisting of a Kozak consensus sequence, an IRES, a SG promoter and combinations thereof, and b) a second nucleotide sequence encoding an antigen from influenza, operably linked to a regulatory element selected from the group consisting of a SG promoter and an IRES.

In one example, the cRNA comprises, in order from 5′ to 3′: a) a first nucleotide sequence encoding an antigen from influenza, operably linked to a regulatory element selected from the group consisting of a Kozak consensus sequence, an IRES, a SG promoter and combinations thereof, and b) a second nucleotide sequence encoding a first antigen antigen from a severe acute respiratory syndrome coronavirus (SARS-CoV-2) operably linked to a regulatory element selected from the group consisting of a SG promoter and an IRES.

In another example, the immunogenic composition comprises:
(i) a cRNA comprising: a) a first nucleotide sequence encoding an antigen from a severe acute respiratory syndrome coronavirus (SARS-CoV-2) operably linked to a regulatory element selected from the group consisting of a Kozak consensus sequence, an IRES, a SG promoter and combinations thereof, and b) a second nucleotide sequence encoding an antigen from a respiratory syncytial virus (RSV), operably linked to a regulatory element selected from the group consisting of a SG promoter and an IRES; and
(ii) an antigen polypeptide from influenza.

In one example, the cRNA comprises, in order from 5′ to 3′: a) a first nucleotide sequence encoding a first antigen antigen from a severe acute respiratory syndrome coronavirus (SARS-CoV-2) operably linked to a regulatory element selected from the group consisting of a Kozak consensus sequence, an IRES, a SG promoter and combinations thereof, and b) a second nucleotide sequence encoding an antigen from a respiratory syncytial virus (RSV), operably linked to a regulatory element selected from the group consisting of a SG promoter and an IRES.

In one example, the cRNA comprises, in order from 5′ to 3′: a) a first nucleotide sequence encoding an antigen from a respiratory syncytial virus (RSV), operably linked to a regulatory element selected from the group consisting of a Kozak consensus sequence, an IRES, a SG promoter and combinations thereof, and b) a second nucleotide sequence encoding a first antigen antigen from a severe acute respiratory syndrome coronavirus (SARS-CoV-2) operably linked to a regulatory element selected from the group consisting of a SG promoter and an IRES.

The present disclosure also provides an immunogenic composition comprising
(i) a self-replicating RNA comprising: a) a first nucleotide sequence encoding an antigen from respiratory syncytial virus (RSV) operably linked to a regulatory element selected from the group consisting of a Kozak consensus sequence, an IRES, a SG promoter and combinations thereof, and b) a second nucleotide sequence encoding an antigen from influenza, operably linked to a regulatory element selected from the group consisting of a SG promoter and an IRES; and
(ii) an antigen polypeptide from a severe acute respiratory syndrome coronavirus (SARS-CoV-2).

In one example, the self-replicating RNA comprises, in order from 5′ to 3′: a) a first nucleotide sequence encoding a first antigen antigen from respiratory syncytial virus (RSV) operably linked to a regulatory element selected from the group consisting of a Kozak consensus sequence, an IRES, a SG promoter and combinations thereof, and b) a second nucleotide sequence encoding an antigen from influenza, operably linked to a regulatory element selected from the group consisting of a SG promoter and an IRES.

In one example, the self-replicating RNA comprises, in order from 5′ to 3′: a) a first nucleotide sequence encoding an antigen from influenza, operably linked to a regulatory element selected from the group consisting of a Kozak consensus sequence, an IRES, a SG promoter and combinations thereof, and b) a second nucleotide sequence encoding a first antigen antigen from respiratory syncytial virus (RSV) operably linked to a regulatory element selected from the group consisting of a SG promoter and an IRES.

In another example, the immunogenic composition comprises:
(i) a self-replicating RNA comprising: a) a first nucleotide sequence encoding an antigen from a severe acute respiratory syndrome coronavirus (SARS-CoV-2) operably linked to a regulatory element selected from the group consisting of a Kozak consensus sequence, an IRES, a SG promoter and combinations thereof, and b) a second nucleotide sequence encoding an antigen from influenza, operably linked to a regulatory element selected from the group consisting of a SG promoter and an IRES; and
(ii) an antigen polypeptide from a respiratory syncytial virus (RSV).

In one example, the self-replicating RNA comprises, in order from 5′ to 3′: a) a first nucleotide sequence encoding a first antigen antigen from a severe acute respiratory syndrome coronavirus (SARS-CoV-2) operably linked to a regulatory element selected from the group consisting of a Kozak consensus sequence, an IRES, a SG promoter and combinations thereof, and b) a second nucleotide sequence encoding an antigen from influenza, operably linked to a regulatory element selected from the group consisting of a SG promoter and an IRES.

In one example, the self-replicating RNA comprises, in order from 5′ to 3′: a) a first nucleotide sequence encoding an antigen from influenza, operably linked to a regulatory element selected from the group consisting of a Kozak consensus sequence, an IRES, a SG promoter and combinations thereof, and b) a second nucleotide sequence encoding a first antigen antigen from a severe acute respiratory syndrome coronavirus (SARS-CoV-2) operably linked to a regulatory element selected from the group consisting of a SG promoter and an IRES.

In another example, the immunogenic composition comprises:
(i) a self-replicating RNA comprising: a) a first nucleotide sequence encoding an antigen from a severe acute respiratory syndrome coronavirus (SARS-CoV-2) operably linked to a regulatory element selected from the group consisting of a Kozak consensus sequence, an IRES, a SG promoter and combinations thereof, and b) a second nucleotide sequence encoding an antigen from a respiratory syncytial virus (RSV), operably linked to a regulatory element selected from the group consisting of a SG promoter and an IRES; and
(ii) an antigen polypeptide from influenza.

In one example, the self-replicating RNA comprises, in order from 5′ to 3′: a) a first nucleotide sequence encoding a first antigen antigen from a severe acute respiratory syndrome coronavirus (SARS-CoV-2) operably linked to a regulatory element selected from the group consisting of a Kozak consensus sequence, an IRES, a SG promoter and combinations thereof, and b) a second nucleotide sequence encoding an antigen from a respiratory syncytial virus (RSV), operably linked to a regulatory element selected from the group consisting of a SG promoter and an IRES.

In one example, the self-replicating RNA comprises, in order from 5′ to 3′: a) a first nucleotide sequence encoding an antigen from a respiratory syncytial virus (RSV), operably linked to a regulatory element selected from the group consisting of a Kozak consensus sequence, an IRES, a SG promoter and combinations thereof, and b) a second nucleotide sequence encoding a first antigen antigen from a severe acute respiratory syndrome coronavirus (SARS-CoV-2) operably linked to a regulatory element selected from the group consisting of a SG promoter and an IRES.

The present disclosure provides an immunogenic composition comprising:
(i) a polynucleotide comprising: a) a first nucleotide sequence encoding an antigen from respiratory syncytial virus (RSV) operably linked to a Kozak consensus sequence; and b) a second nucleotide sequence encoding an antigen from influenza, operably linked to a regulatory element selected from the group consisting of a SG promoter and an IRES, and
(ii) an antigen polypeptide from a severe acute respiratory syndrome coronavirus (SARS-CoV-2).

In one example, the polynucleotide comprises, in order from 5′ to 3′: a) a first nucleotide sequence encoding antigen from respiratory syncytial virus (RSV) operably linked to a Kozak consensus sequence; and b) a second nucleotide sequence encoding an antigen from influenza, operably linked to a regulatory element selected from the group consisting of a SG promoter and an IRES.

In one example, the polynucleotide comprises, in order from 5′ to 3′: a) a first nucleotide sequence encoding an antigen from influenza, operably linked to a Kozak consensus sequence; and b) a second nucleotide sequence encoding an antigen from respiratory syncytial virus (RSV) operably linked to a regulatory element selected from the group consisting of a SG promoter and an IRES.

In an example, the immunogenic composition comprises:
(i) a polynucleotide comprising: a) a first nucleotide sequence encoding an antigen from a severe acute respiratory syndrome coronavirus (SARS-CoV-2) operably linked to a Kozak consensus sequence; and b) a second nucleotide sequence encoding an antigen from influenza, operably linked to a regulatory element selected from the group consisting of a SG promoter and an IRES, and
(ii) an antigen polypeptide from a respiratory syncytial virus (RSV).

In one example, the polynucleotide comprises, in order from 5′ to 3′: a) a first nucleotide sequence encoding antigen from a severe acute respiratory syndrome coronavirus (SARS-CoV-2) operably linked to a Kozak consensus sequence; and b) a second nucleotide sequence encoding an antigen from influenza, operably linked to a regulatory element selected from the group consisting of a SG promoter and an IRES.

In one example, the polynucleotide comprises, in order from 5′ to 3′: a) a first nucleotide sequence encoding an antigen from influenza, operably linked to a Kozak consensus sequence; and b) a second nucleotide sequence encoding an antigen from a severe acute respiratory syndrome coronavirus (SARS-CoV-2) operably linked to a regulatory element selected from the group consisting of a SG promoter and an IRES.

In an example, the immunogenic composition comprises:
(i) a polynucleotide comprising: a) a first nucleotide sequence encoding an antigen from a severe acute respiratory syndrome coronavirus (SARS-CoV-2) operably linked to a Kozak consensus sequence; and b) a second nucleotide sequence encoding an antigen from a respiratory syncytial virus (RSV), operably linked to a regulatory element selected from the group consisting of a SG promoter and an IRES, and
(ii) an antigen polypeptide from influenza.

In one example, the polynucleotide comprises, in order from 5′ to 3′: a) a first nucleotide sequence encoding antigen from a severe acute respiratory syndrome coronavirus (SARS-CoV-2) operably linked to a Kozak consensus sequence; and b) a second nucleotide sequence encoding an antigen from a respiratory syncytial virus (RSV), operably linked to a regulatory element selected from the group consisting of a SG promoter and an IRES.

In one example, the polynucleotide comprises, in order from 5′ to 3′: a) a first nucleotide sequence encoding an antigen from a respiratory syncytial virus (RSV), operably linked to a Kozak consensus sequence; and b) a second nucleotide sequence encoding an antigen from a severe acute respiratory syndrome coronavirus (SARS-CoV-2) operably linked to a regulatory element selected from the group consisting of a SG promoter and an IRES.

In another example, the present disclosure provides an immunogenic composition comprising:
(i) a RNA comprising: a) a first nucleotide sequence encoding an antigen from respiratory syncytial virus (RSV) operably linked to a Kozak consensus sequence; and b) a second nucleotide sequence encoding an antigen from influenza, operably linked to a regulatory element selected from the group consisting of a SG promoter and an IRES, and
(ii) an antigen polypeptide from a severe acute respiratory syndrome coronavirus (SARS-CoV-2).

In one example, the RNA comprises, in order from 5′ to 3′: a) a first nucleotide sequence encoding antigen from respiratory syncytial virus (RSV) operably linked to a Kozak consensus sequence; and b) a second nucleotide sequence encoding an antigen from influenza, operably linked to a regulatory element selected from the group consisting of a SG promoter and an IRES.

In one example, the RNA comprises, in order from 5′ to 3′: a) a first nucleotide sequence encoding an antigen from influenza, operably linked to a Kozak consensus sequence; and b) a second nucleotide sequence encoding an antigen from respiratory syncytial virus (RSV) operably linked to a regulatory element selected from the group consisting of a SG promoter and an IRES.

In an example, the immunogenic composition comprises:
(i) a RNA comprising: a) a first nucleotide sequence encoding an antigen from a severe acute respiratory syndrome coronavirus (SARS-CoV-2) operably linked to a Kozak consensus sequence; and b) a second nucleotide sequence encoding an antigen from influenza, operably linked to a regulatory element selected from the group consisting of a SG promoter and an IRES, and
(ii) an antigen polypeptide from a respiratory syncytial virus (RSV).

In one example, the RNA comprises, in order from 5′ to 3′: a) a first nucleotide sequence encoding antigen from a severe acute respiratory syndrome coronavirus (SARS-CoV-2) operably linked to a Kozak consensus sequence; and b) a second nucleotide sequence encoding an antigen from influenza, operably linked to a regulatory element selected from the group consisting of a SG promoter and an IRES.

In one example, the RNA comprises, in order from 5′ to 3′: a) a first nucleotide sequence encoding an antigen from influenza, operably linked to a Kozak consensus sequence; and b) a second nucleotide sequence encoding an antigen from a severe acute respiratory syndrome coronavirus (SARS-CoV-2) operably linked to a regulatory element selected from the group consisting of a SG promoter and an IRES.

In an example, the immunogenic composition comprises:
(i) a RNA comprising: a) a first nucleotide sequence encoding an antigen from a severe acute respiratory syndrome coronavirus (SARS-CoV-2) operably linked to a Kozak consensus sequence; and b) a second nucleotide sequence encoding an antigen from a respiratory syncytial virus (RSV), operably linked to a regulatory element selected from the group consisting of a SG promoter and an IRES, and
(ii) an antigen polypeptide from influenza.

In one example, the RNA comprises, in order from 5′ to 3′: a) a first nucleotide sequence encoding antigen from a severe acute respiratory syndrome coronavirus (SARS-CoV-2) operably linked to a Kozak consensus sequence; and b) a second nucleotide sequence encoding an antigen from a respiratory syncytial virus (RSV), operably linked to a regulatory element selected from the group consisting of a SG promoter and an IRES.

In one example, the RNA comprises, in order from 5′ to 3′: a) a first nucleotide sequence encoding an antigen from a respiratory syncytial virus (RSV), operably linked to a Kozak consensus sequence; and b) a second nucleotide sequence encoding an antigen from a severe acute respiratory syndrome coronavirus (SARS-CoV-2) operably linked to a regulatory element selected from the group consisting of a SG promoter and an IRES.

In another example, the present disclosure provides an immunogenic composition comprising:
(i) a cRNA comprising: a) a first nucleotide sequence encoding an antigen from respiratory syncytial virus (RSV) operably linked to a Kozak consensus sequence; and b) a second nucleotide sequence encoding an antigen from influenza, operably linked to a regulatory element selected from the group consisting of a SG promoter and an IRES, and
(ii) an antigen polypeptide from a severe acute respiratory syndrome coronavirus (SARS-CoV-2).

In one example, the cRNA comprises, in order from 5′ to 3′: a) a first nucleotide sequence encoding antigen from respiratory syncytial virus (RSV) operably linked to a Kozak consensus sequence; and b) a second nucleotide sequence encoding an antigen from influenza, operably linked to a regulatory element selected from the group consisting of a SG promoter and an IRES.

In one example, the cRNA comprises, in order from 5′ to 3′: a) a first nucleotide sequence encoding an antigen from influenza, operably linked to a Kozak consensus sequence; and b) a second nucleotide sequence encoding an antigen from respiratory syncytial virus (RSV) operably linked to a regulatory element selected from the group consisting of a SG promoter and an IRES.

In an example, the immunogenic composition comprises:
(i) a cRNA comprising: a) a first nucleotide sequence encoding an antigen from a severe acute respiratory syndrome coronavirus (SARS-CoV-2) operably linked to a Kozak consensus sequence; and b) a second nucleotide sequence encoding an antigen from influenza, operably linked to a regulatory element selected from the group consisting of a SG promoter and an IRES, and
(ii) an antigen polypeptide from a respiratory syncytial virus (RSV).

In one example, the cRNA comprises, in order from 5′ to 3′: a) a first nucleotide sequence encoding antigen from a severe acute respiratory syndrome coronavirus (SARS-CoV-2) operably linked to a Kozak consensus sequence; and b) a second nucleotide sequence encoding an antigen from influenza, operably linked to a regulatory element selected from the group consisting of a SG promoter and an IRES.

In one example, the cRNA comprises, in order from 5′ to 3′: a) a first nucleotide sequence encoding an antigen from influenza, operably linked to a Kozak consensus sequence; and b) a second nucleotide sequence encoding an antigen from a severe acute respiratory syndrome coronavirus (SARS-CoV-2) operably linked to a regulatory element selected from the group consisting of a SG promoter and an IRES.

In an example, the immunogenic composition comprises:
(i) a cRNA comprising: a) a first nucleotide sequence encoding an antigen from a severe acute respiratory syndrome coronavirus (SARS-CoV-2) operably linked to a Kozak consensus sequence; and b) a second nucleotide sequence encoding an antigen from a respiratory syncytial virus (RSV), operably linked to a regulatory element selected from the group consisting of a SG promoter and an IRES, and
(ii) an antigen polypeptide from influenza.

In one example, the cRNA comprises, in order from 5′ to 3′: a) a first nucleotide sequence encoding antigen from a severe acute respiratory syndrome coronavirus (SARS-CoV-2) operably linked to a Kozak consensus sequence; and b) a second nucleotide sequence encoding an antigen from a respiratory syncytial virus (RSV), operably linked to a regulatory element selected from the group consisting of a SG promoter and an IRES.

In one example, the cRNA comprises, in order from 5′ to 3′: a) a first nucleotide sequence encoding an antigen from a respiratory syncytial virus (RSV), operably linked to a Kozak consensus sequence; and b) a second nucleotide sequence encoding an antigen from a severe acute respiratory syndrome coronavirus (SARS-CoV-2) operably linked to a regulatory element selected from the group consisting of a SG promoter and an IRES.

In another example, the present disclosure provides an immunogenic composition comprising:
(i) a self-replicating RNA comprising: a) a first nucleotide sequence encoding an antigen from respiratory syncytial virus (RSV) operably linked to a Kozak consensus sequence; and b) a second nucleotide sequence encoding an antigen from influenza, operably linked to a regulatory element selected from the group consisting of a SG promoter and an IRES, and
(ii) an antigen polypeptide from a severe acute respiratory syndrome coronavirus (SARS-CoV-2).

In one example, the self-replicating RNA comprises, in order from 5′ to 3′: a) a first nucleotide sequence encoding antigen from respiratory syncytial virus (RSV) operably linked to a Kozak consensus sequence; and b) a second nucleotide sequence encoding an antigen from influenza, operably linked to a regulatory element selected from the group consisting of a SG promoter and an IRES.

In one example, the self-replicating RNA comprises, in order from 5′ to 3′: a) a first nucleotide sequence encoding an antigen from influenza, operably linked to a Kozak consensus sequence; and b) a second nucleotide sequence encoding an antigen from respiratory syncytial virus (RSV) operably linked to a regulatory element selected from the group consisting of a SG promoter and an IRES.

In an example, the immunogenic composition comprises:
(i) a self-replicating RNA comprising: a) a first nucleotide sequence encoding an antigen from a severe acute respiratory syndrome coronavirus (SARS-CoV-2) operably linked to a Kozak consensus sequence; and b) a second nucleotide sequence encoding an antigen from influenza, operably linked to a regulatory element selected from the group consisting of a SG promoter and an IRES, and
(ii) an antigen polypeptide from a respiratory syncytial virus (RSV).

In one example, the self-replicating RNA comprises, in order from 5′ to 3′: a) a first nucleotide sequence encoding antigen from a severe acute respiratory syndrome coronavirus (SARS-CoV-2) operably linked to a Kozak consensus sequence; and b) a second nucleotide sequence encoding an antigen from influenza, operably linked to a regulatory element selected from the group consisting of a SG promoter and an IRES.

In one example, the self-replicating RNA comprises, in order from 5′ to 3′: a) a first nucleotide sequence encoding an antigen from influenza, operably linked to a Kozak consensus sequence; and b) a second nucleotide sequence encoding an antigen from a severe acute respiratory syndrome coronavirus (SARS-CoV-2) operably linked to a regulatory element selected from the group consisting of a SG promoter and an IRES.

In an example, the immunogenic composition comprises:
(i) a self-replicating RNA comprising: a) a first nucleotide sequence encoding an antigen from a severe acute respiratory syndrome coronavirus (SARS-CoV-2) operably linked to a Kozak consensus sequence; and b) a second nucleotide sequence encoding an antigen from a respiratory syncytial virus (RSV), operably linked to a regulatory element selected from the group consisting of a SG promoter and an IRES, and
(ii) an antigen polypeptide from influenza.

In one example, the self-replicating RNA comprises, in order from 5′ to 3′: a) a first nucleotide sequence encoding antigen from a severe acute respiratory syndrome coronavirus (SARS-CoV-2) operably linked to a Kozak consensus sequence; and b) a second nucleotide sequence encoding an antigen from a respiratory syncytial virus (RSV), operably linked to a regulatory element selected from the group consisting of a SG promoter and an IRES.

In one example, the self-replicating RNA comprises, in order from 5′ to 3′: a) a first nucleotide sequence encoding an antigen from a respiratory syncytial virus (RSV), operably linked to a Kozak consensus sequence; and b) a second nucleotide sequence encoding an antigen from a severe acute respiratory syndrome coronavirus (SARS-CoV-2) operably linked to a regulatory element selected from the group consisting of a SG promoter and an IRES.

The present disclosure provides an immunogenic composition comprising:
(i) a polynucleotide comprising: a) a first nucleotide sequence encoding an antigen from respiratory syncytial virus (RSV) operably linked to a Kozak consensus sequence and a SG promoter; and b) a second nucleotide sequence encoding an antigen from influenza, operably linked to a regulatory element selected from the group consisting of a SG promoter and an IRES, and
(ii) an antigen polypeptide from a severe acute respiratory syndrome coronavirus (SARS-CoV-2).

In one example, the polynucleotide comprises, in order from 5′ to 3′: a) a first nucleotide sequence encoding antigen from respiratory syncytial virus (RSV) operably linked to a Kozak consensus sequence and a SG promoter; and b) a second nucleotide sequence encoding an antigen from influenza, operably linked to a regulatory element selected from the group consisting of a SG promoter and an IRES.

In one example, the polynucleotide comprises, in order from 5′ to 3′: a) a first nucleotide sequence encoding an antigen from influenza, operably linked to a Kozak consensus sequence and a SG promoter; and b) a second nucleotide sequence encoding an antigen from respiratory syncytial virus (RSV) operably linked to a regulatory element selected from the group consisting of a SG promoter and an IRES.

In an example, the immunogenic composition comprises:
(i) a polynucleotide comprising: a) a first nucleotide sequence encoding an antigen from a severe acute respiratory syndrome coronavirus (SARS-CoV-2) operably linked to a Kozak consensus sequence and a SG promoter; and b) a second nucleotide sequence encoding an antigen from influenza, operably linked to a regulatory element selected from the group consisting of a SG promoter and an IRES, and
(ii) an antigen polypeptide from a respiratory syncytial virus (RSV).

In one example, the polynucleotide comprises, in order from 5′ to 3′: a) a first nucleotide sequence encoding antigen from a severe acute respiratory syndrome coronavirus (SARS-CoV-2) operably linked to a Kozak consensus sequence and a SG promoter; and b) a second nucleotide sequence encoding an antigen from influenza, operably linked to a regulatory element selected from the group consisting of a SG promoter and an IRES.

In one example, the polynucleotide comprises, in order from 5′ to 3′: a) a first nucleotide sequence encoding an antigen from influenza, operably linked to a Kozak consensus sequence and a SG promoter; and b) a second nucleotide sequence encoding an antigen from a severe acute respiratory syndrome coronavirus (SARS-CoV-2) operably linked to a regulatory element selected from the group consisting of a SG promoter and an IRES.

In an example, the immunogenic composition comprises:
(i) a polynucleotide comprising: a) a first nucleotide sequence encoding an antigen from a severe acute respiratory syndrome coronavirus (SARS-CoV-2) operably linked to a Kozak consensus sequence and a SG promoter; and b) a second nucleotide sequence encoding an antigen from a respiratory syncytial virus (RSV), operably linked to a regulatory element selected from the group consisting of a SG promoter and an IRES, and
(ii) an antigen polypeptide from influenza.

In one example, the polynucleotide comprises, in order from 5′ to 3′: a) a first nucleotide sequence encoding antigen from a severe acute respiratory syndrome coronavirus (SARS-CoV-2) operably linked to a Kozak consensus sequence and a SG promoter; and b) a second nucleotide sequence encoding an antigen from a respiratory syncytial virus (RSV), operably linked to a regulatory element selected from the group consisting of a SG promoter and an IRES.

In one example, the polynucleotide comprises, in order from 5′ to 3′: a) a first nucleotide sequence encoding an antigen from a respiratory syncytial virus (RSV), operably linked to a Kozak consensus sequence and a SG promoter; and b) a second nucleotide sequence encoding an antigen from a severe acute respiratory syndrome coronavirus (SARS-CoV-2) operably linked to a regulatory element selected from the group consisting of a SG promoter and an IRES.

In another example, the present disclosure provides an immunogenic composition comprising:
(i) a RNA comprising: a) a first nucleotide sequence encoding an antigen from respiratory syncytial virus (RSV) operably linked to a Kozak consensus sequence and a SG promoter; and b) a second nucleotide sequence encoding an antigen from influenza, operably linked to a regulatory element selected from the group consisting of a SG promoter and an IRES, and
(ii) an antigen polypeptide from a severe acute respiratory syndrome coronavirus (SARS-CoV-2).

In one example, the RNA comprises, in order from 5′ to 3′: a) a first nucleotide sequence encoding antigen from respiratory syncytial virus (RSV) operably linked to a Kozak consensus sequence and a SG promoter; and b) a second nucleotide sequence encoding an antigen from influenza, operably linked to a regulatory element selected from the group consisting of a SG promoter and an IRES.

In one example, the RNA comprises, in order from 5′ to 3′: a) a first nucleotide sequence encoding an antigen from influenza, operably linked to a Kozak consensus sequence and a SG promoter; and b) a second nucleotide sequence encoding an antigen from respiratory syncytial virus (RSV) operably linked to a regulatory element selected from the group consisting of a SG promoter and an IRES.

In an example, the immunogenic composition comprises:
(i) a RNA comprising: a) a first nucleotide sequence encoding an antigen from a severe acute respiratory syndrome coronavirus (SARS-CoV-2) operably linked to a Kozak consensus sequence and a SG promoter; and b) a second nucleotide sequence encoding an antigen from influenza, operably linked to a regulatory element selected from the group consisting of a SG promoter and an IRES, and
(ii) an antigen polypeptide from a respiratory syncytial virus (RSV).

In one example, the RNA comprises, in order from 5′ to 3′: a) a first nucleotide sequence encoding antigen from a severe acute respiratory syndrome coronavirus (SARS-CoV-2) operably linked to a Kozak consensus sequence and a SG promoter; and b) a second nucleotide sequence encoding an antigen from influenza, operably linked to a regulatory element selected from the group consisting of a SG promoter and an IRES.

In one example, the RNA comprises, in order from 5′ to 3′: a) a first nucleotide sequence encoding an antigen from influenza, operably linked to a Kozak consensus sequence and a SG promoter; and b) a second nucleotide sequence encoding an antigen from a severe acute respiratory syndrome coronavirus (SARS-CoV-2) operably linked to a regulatory element selected from the group consisting of a SG promoter and an IRES.

In an example, the immunogenic composition comprises:
(i) a RNA comprising: a) a first nucleotide sequence encoding an antigen from a severe acute respiratory syndrome coronavirus (SARS-CoV-2) operably linked to a Kozak consensus sequence and a SG promoter; and b) a second nucleotide sequence encoding an antigen from a respiratory syncytial virus (RSV), operably linked to a regulatory element selected from the group consisting of a SG promoter and an IRES, and
(ii) an antigen polypeptide from influenza.

In one example, the RNA comprises, in order from 5′ to 3′: a) a first nucleotide sequence encoding antigen from a severe acute respiratory syndrome coronavirus (SARS-CoV-2) operably linked to a Kozak consensus sequence and a SG promoter; and b) a second nucleotide sequence encoding an antigen from a respiratory syncytial virus (RSV), operably linked to a regulatory element selected from the group consisting of a SG promoter and an IRES.

In one example, the RNA comprises, in order from 5′ to 3′: a) a first nucleotide sequence encoding an antigen from a respiratory syncytial virus (RSV), operably linked to a Kozak consensus sequence and a SG promoter; and b) a second nucleotide sequence encoding an antigen from a severe acute respiratory syndrome coronavirus (SARS-CoV-2) operably linked to a regulatory element selected from the group consisting of a SG promoter and an IRES.

In another example, the present disclosure provides an immunogenic composition comprising:
(i) a cRNA comprising: a) a first nucleotide sequence encoding an antigen from respiratory syncytial virus (RSV) operably linked to a Kozak consensus sequence and a SG promoter; and b) a second nucleotide sequence encoding an antigen from influenza, operably linked to a regulatory element selected from the group consisting of a SG promoter and an IRES, and
(ii) an antigen polypeptide from a severe acute respiratory syndrome coronavirus (SARS-CoV-2).

In one example, the cRNA comprises, in order from 5′ to 3′: a) a first nucleotide sequence encoding antigen from respiratory syncytial virus (RSV) operably linked to a Kozak consensus sequence and a SG promoter; and b) a second nucleotide sequence encoding an antigen from influenza, operably linked to a regulatory element selected from the group consisting of a SG promoter and an IRES.

In one example, the cRNA comprises, in order from 5′ to 3′: a) a first nucleotide sequence encoding an antigen from influenza, operably linked to a Kozak consensus sequence and a SG promoter; and b) a second nucleotide sequence encoding an antigen from respiratory syncytial virus (RSV) operably linked to a regulatory element selected from the group consisting of a SG promoter and an IRES.

In an example, the immunogenic composition comprises:
(i) a cRNA comprising: a) a first nucleotide sequence encoding an antigen from SARS-CoV-2 operably linked to a Kozak consensus sequence and a SG promoter; and b) a second nucleotide sequence encoding an antigen from influenza, operably linked to a regulatory element selected from the group consisting of a SG promoter and an IRES, and
(ii) an antigen polypeptide from a respiratory syncytial virus (RSV).

In one example, the cRNA comprises, in order from 5′ to 3′: a) a first nucleotide sequence encoding antigen from a severe acute respiratory syndrome coronavirus (SARS-CoV-2) operably linked to a Kozak consensus sequence and a SG promoter; and b) a second nucleotide sequence encoding an antigen from influenza, operably linked to a regulatory element selected from the group consisting of a SG promoter and an IRES.

In one example, the cRNA comprises, in order from 5′ to 3′: a) a first nucleotide sequence encoding an antigen from influenza, operably linked to a Kozak consensus sequence and a SG promoter; and b) a second nucleotide sequence encoding an antigen from a severe acute respiratory syndrome coronavirus (SARS-CoV-2) operably linked to a regulatory element selected from the group consisting of a SG promoter and an IRES.

In an example, the immunogenic composition comprises:
(i) a cRNA comprising: a) a first nucleotide sequence encoding an antigen from SARS-CoV-2 operably linked to a Kozak consensus sequence and a SG promoter; and b) a second nucleotide sequence encoding an antigen from a respiratory syncytial virus (RSV), operably linked to a regulatory element selected from the group consisting of a SG promoter and an IRES, and
(ii) an antigen polypeptide from influenza.

In one example, the cRNA comprises, in order from 5′ to 3′: a) a first nucleotide sequence encoding antigen from a severe acute respiratory syndrome coronavirus (SARS-CoV-2) operably linked to a Kozak consensus sequence and a SG promoter; and b) a second nucleotide sequence encoding an antigen from a respiratory syncytial virus (RSV), operably linked to a regulatory element selected from the group consisting of a SG promoter and an IRES.

In one example, the cRNA comprises, in order from 5′ to 3′: a) a first nucleotide sequence encoding an antigen from a respiratory syncytial virus (RSV), operably linked to a Kozak consensus sequence and a SG promoter; and b) a second nucleotide sequence encoding an antigen from a severe acute respiratory syndrome coronavirus (SARS-CoV-2) operably linked to a regulatory element selected from the group consisting of a SG promoter and an IRES.

In another example, the present disclosure provides an immunogenic composition comprising:
(i) a self-replicating RNA comprising: a) a first nucleotide sequence encoding an antigen from respiratory syncytial virus (RSV) operably linked to a Kozak consensus sequence and a SG promoter; and b) a second nucleotide sequence encoding an antigen from influenza, operably linked to a regulatory element selected from the group consisting of a SG promoter and an IRES, and
(ii) an antigen polypeptide from a severe acute respiratory syndrome coronavirus (SARS-CoV-2).

In one example, the self-replicating RNA comprises, in order from 5′ to 3′: a) a first nucleotide sequence encoding antigen from respiratory syncytial virus (RSV) operably linked to a Kozak consensus sequence and a SG promoter; and b) a second nucleotide sequence encoding an antigen from influenza, operably linked to a regulatory element selected from the group consisting of a SG promoter and an IRES.

In one example, the self-replicating RNA comprises, in order from 5′ to 3′: a) a first nucleotide sequence encoding an antigen from influenza, operably linked to a Kozak consensus sequence and a SG promoter; and b) a second nucleotide sequence encoding an antigen from respiratory syncytial virus (RSV) operably linked to a regulatory element selected from the group consisting of a SG promoter and an IRES.

In an example, the immunogenic composition comprises:
(i) a self-replicating RNA comprising: a) a first nucleotide sequence encoding an antigen from a severe acute respiratory syndrome coronavirus (SARS-CoV-2) operably linked to a Kozak consensus sequence and a SG promoter; and b) a second nucleotide sequence encoding an antigen from influenza, operably linked to a regulatory element selected from the group consisting of a SG promoter and an IRES, and
(ii) an antigen polypeptide from a respiratory syncytial virus (RSV).

In one example, the self-replicating RNA comprises, in order from 5′ to 3′: a) a first nucleotide sequence encoding antigen from a severe acute respiratory syndrome coronavirus (SARS-CoV-2) operably linked to a Kozak consensus sequence and a SG promoter; and b) a second nucleotide sequence encoding an antigen from influenza, operably linked to a regulatory element selected from the group consisting of a SG promoter and an IRES.

In one example, the self-replicating RNA comprises, in order from 5′ to 3′: a) a first nucleotide sequence encoding an antigen from influenza, operably linked to a Kozak consensus sequence and a SG promoter; and b) a second nucleotide sequence encoding an antigen from a severe acute respiratory syndrome coronavirus (SARS-CoV-2) operably linked to a regulatory element selected from the group consisting of a SG promoter and an IRES.

In an example, the immunogenic composition comprises:
(i) a self-replicating RNA comprising: a) a first nucleotide sequence encoding an antigen from a severe acute respiratory syndrome coronavirus (SARS-CoV-2) operably linked to a Kozak consensus sequence and a SG promoter; and b) a second nucleotide sequence encoding an antigen from a respiratory syncytial virus (RSV), operably linked to a regulatory element selected from the group consisting of a SG promoter and an IRES, and
(ii) an antigen polypeptide from influenza.

In one example, the self-replicating RNA comprises, in order from 5′ to 3′: a) a first nucleotide sequence encoding antigen from a severe acute respiratory syndrome coronavirus (SARS-CoV-2) operably linked to a Kozak consensus sequence and a SG promoter; and b) a second nucleotide sequence encoding an antigen from a respiratory syncytial virus (RSV), operably linked to a regulatory element selected from the group consisting of a SG promoter and an IRES.

In one example, the self-replicating RNA comprises, in order from 5′ to 3′: a) a first nucleotide sequence encoding an antigen from a respiratory syncytial virus (RSV), operably linked to a Kozak consensus sequence and a SG promoter; and b) a second nucleotide sequence encoding an antigen from a severe acute respiratory syndrome coronavirus (SARS-CoV-2) operably linked to a regulatory element selected from the group consisting of a SG promoter and an IRES.

The present disclosure provides an immunogenic composition comprising:
(i) a polynucleotide comprising: a) a first nucleotide sequence encoding an antigen from respiratory syncytial virus (RSV) operably linked to a Kozak consensus sequence and an IRES; and b) a second nucleotide sequence encoding an antigen from influenza, operably linked to a regulatory element selected from the group consisting of a SG promoter and an IRES, and
(ii) an antigen polypeptide from a severe acute respiratory syndrome coronavirus (SARS-CoV-2).

In one example, the polynucleotide comprises, in order from 5′ to 3′: a) a first nucleotide sequence encoding antigen from respiratory syncytial virus (RSV) operably linked to a Kozak consensus sequence and an IRES; and b) a second nucleotide sequence encoding an antigen from influenza, operably linked to a regulatory element selected from the group consisting of a SG promoter and an IRES.

In one example, the polynucleotide comprises, in order from 5′ to 3′: a) a first nucleotide sequence encoding an antigen from influenza, operably linked to a Kozak consensus sequence and an IRES; and b) a second nucleotide sequence encoding an antigen from respiratory syncytial virus (RSV) operably linked to a regulatory element selected from the group consisting of a SG promoter and an IRES.

In an example, the immunogenic composition comprises:
(i) a polynucleotide comprising: a) a first nucleotide sequence encoding an antigen from a severe acute respiratory syndrome coronavirus (SARS-CoV-2) operably linked to a Kozak consensus sequence and an IRES; and b) a second nucleotide sequence encoding an antigen from influenza, operably linked to a regulatory element selected from the group consisting of a SG promoter and an IRES, and
(ii) an antigen polypeptide from a respiratory syncytial virus (RSV).

In one example, the polynucleotide comprises, in order from 5′ to 3′: a) a first nucleotide sequence encoding antigen from a severe acute respiratory syndrome coronavirus (SARS-CoV-2) operably linked to a Kozak consensus sequence and an IRES; and b) a second nucleotide sequence encoding an antigen from influenza, operably linked to a regulatory element selected from the group consisting of a SG promoter and an IRES.

In one example, the polynucleotide comprises, in order from 5′ to 3′: a) a first nucleotide sequence encoding an antigen from influenza, operably linked to a Kozak consensus sequence and an IRES; and b) a second nucleotide sequence encoding an antigen from a severe acute respiratory syndrome coronavirus (SARS-CoV-2) operably linked to a regulatory element selected from the group consisting of a SG promoter and an IRES.

In an example, the immunogenic composition comprises:
(i) a polynucleotide comprising: a) a first nucleotide sequence encoding an antigen from a severe acute respiratory syndrome coronavirus (SARS-CoV-2) operably linked to a Kozak consensus sequence and an IRES; and b) a second nucleotide sequence encoding an antigen from a respiratory syncytial virus (RSV), operably linked to a regulatory element selected from the group consisting of a SG promoter and an IRES, and
(ii) an antigen polypeptide from influenza.

In one example, the polynucleotide comprises, in order from 5′ to 3′: a) a first nucleotide sequence encoding antigen from a severe acute respiratory syndrome coronavirus (SARS-CoV-2) operably linked to a Kozak consensus sequence and an IRES; and b) a second nucleotide sequence encoding an antigen from a respiratory syncytial virus (RSV), operably linked to a regulatory element selected from the group consisting of a SG promoter and an IRES.

In one example, the polynucleotide comprises, in order from 5′ to 3′: a) a first nucleotide sequence encoding an antigen from a respiratory syncytial virus (RSV), operably linked to a Kozak consensus sequence and an IRES; and b) a second nucleotide sequence encoding an antigen from a severe acute respiratory syndrome coronavirus (SARS-CoV-2) operably linked to a regulatory element selected from the group consisting of a SG promoter and an IRES.

In another example, the present disclosure provides an immunogenic composition comprising:
(i) a RNA comprising: a) a first nucleotide sequence encoding an antigen from respiratory syncytial virus (RSV) operably linked to a Kozak consensus sequence and an IRES; and b) a second nucleotide sequence encoding an antigen from influenza, operably linked to a regulatory element selected from the group consisting of a SG promoter and an IRES, and
(ii) an antigen polypeptide from a severe acute respiratory syndrome coronavirus (SARS-CoV-2).

In one example, the RNA comprises, in order from 5′ to 3′: a) a first nucleotide sequence encoding antigen from respiratory syncytial virus (RSV) operably linked to a Kozak consensus sequence and an IRES; and b) a second nucleotide sequence encoding an antigen from influenza, operably linked to a regulatory element selected from the group consisting of a SG promoter and an IRES.

In one example, the RNA comprises, in order from 5′ to 3′: a) a first nucleotide sequence encoding an antigen from influenza, operably linked to a Kozak consensus sequence and an IRES; and b) a second nucleotide sequence encoding an antigen from respiratory syncytial virus (RSV) operably linked to a regulatory element selected from the group consisting of a SG promoter and an IRES.

In an example, the immunogenic composition comprises:
(i) a RNA comprising: a) a first nucleotide sequence encoding an antigen from a severe acute respiratory syndrome coronavirus (SARS-CoV-2) operably linked to a Kozak consensus sequence and an IRES; and b) a second nucleotide sequence encoding an antigen from influenza, operably linked to a regulatory element selected from the group consisting of a SG promoter and an IRES, and
(ii) an antigen polypeptide from a respiratory syncytial virus (RSV).

In one example, the RNA comprises, in order from 5′ to 3′: a) a first nucleotide sequence encoding antigen from a severe acute respiratory syndrome coronavirus (SARS-CoV-2) operably linked to a Kozak consensus sequence and an IRES; and b) a second nucleotide sequence encoding an antigen from influenza, operably linked to a regulatory element selected from the group consisting of a SG promoter and an IRES.

In one example, the RNA comprises, in order from 5′ to 3′: a) a first nucleotide sequence encoding an antigen from influenza, operably linked to a Kozak consensus sequence and an IRES; and b) a second nucleotide sequence encoding an antigen from a SARS-CoV-2 operably linked to a regulatory element selected from the group consisting of a SG promoter and an IRES.

In an example, the immunogenic composition comprises:
(i) a RNA comprising: a) a first nucleotide sequence encoding an antigen from a severe acute respiratory syndrome coronavirus (SARS-CoV-2) operably linked to a Kozak consensus sequence and an IRES; and b) a second nucleotide sequence encoding an antigen from a respiratory syncytial virus (RSV), operably linked to a regulatory element selected from the group consisting of a SG promoter and an IRES, and
(ii) an antigen polypeptide from influenza.

In one example, the RNA comprises, in order from 5′ to 3′: a) a first nucleotide sequence encoding antigen from a severe acute respiratory syndrome coronavirus (SARS-CoV-2) operably linked to a Kozak consensus sequence and an IRES; and b) a second nucleotide sequence encoding an antigen from a respiratory syncytial virus (RSV), operably linked to a regulatory element selected from the group consisting of a SG promoter and an IRES.

In one example, the RNA comprises, in order from 5′ to 3′: a) a first nucleotide sequence encoding an antigen from a respiratory syncytial virus (RSV), operably linked to a Kozak consensus sequence and an IRES; and b) a second nucleotide sequence encoding an antigen from a severe acute respiratory syndrome coronavirus (SARS-CoV-2) operably linked to a regulatory element selected from the group consisting of a SG promoter and an IRES.

In another example, the present disclosure provides an immunogenic composition comprising:
(i) a cRNA comprising: a) a first nucleotide sequence encoding an antigen from respiratory syncytial virus (RSV) operably linked to a Kozak consensus sequence and an IRES; and b) a second nucleotide sequence encoding an antigen from influenza, operably linked to a regulatory element selected from the group consisting of a SG promoter and an IRES, and
(ii) an antigen polypeptide from a severe acute respiratory syndrome coronavirus (SARS-CoV-2).

In one example, the cRNA comprises, in order from 5′ to 3′: a) a first nucleotide sequence encoding antigen from respiratory syncytial virus (RSV) operably linked to a Kozak consensus sequence and an IRES; and b) a second nucleotide sequence encoding an antigen from influenza, operably linked to a regulatory element selected from the group consisting of a SG promoter and an IRES.

In one example, the cRNA comprises, in order from 5′ to 3′: a) a first nucleotide sequence encoding an antigen from influenza, operably linked to a Kozak consensus sequence and an IRES; and b) a second nucleotide sequence encoding an antigen from respiratory syncytial virus (RSV) operably linked to a regulatory element selected from the group consisting of a SG promoter and an IRES.

In an example, the immunogenic composition comprises:
(i) a cRNA comprising: a) a first nucleotide sequence encoding an antigen from a severe acute respiratory syndrome coronavirus (SARS-CoV-2) operably linked to a Kozak consensus sequence and an IRES; and b) a second nucleotide sequence encoding an antigen from influenza, operably linked to a regulatory element selected from the group consisting of a SG promoter and an IRES, and
(ii) an antigen polypeptide from a respiratory syncytial virus (RSV).

In one example, the cRNA comprises, in order from 5′ to 3′: a) a first nucleotide sequence encoding antigen from a severe acute respiratory syndrome coronavirus (SARS-CoV-2) operably linked to a Kozak consensus sequence and an IRES; and b) a second nucleotide sequence encoding an antigen from influenza, operably linked to a regulatory element selected from the group consisting of a SG promoter and an IRES.

In one example, the cRNA comprises, in order from 5′ to 3′: a) a first nucleotide sequence encoding an antigen from influenza, operably linked to a Kozak consensus sequence and an IRES; and b) a second nucleotide sequence encoding an antigen from a severe acute respiratory syndrome coronavirus (SARS-CoV-2) operably linked to a regulatory element selected from the group consisting of a SG promoter and an IRES.

In an example, the immunogenic composition comprises:
(i) a cRNA comprising: a) a first nucleotide sequence encoding an antigen from a severe acute respiratory syndrome coronavirus (SARS-CoV-2) operably linked to a Kozak consensus sequence and an IRES; and b) a second nucleotide sequence encoding an antigen from a respiratory syncytial virus (RSV), operably linked to a regulatory element selected from the group consisting of a SG promoter and an IRES, and
(ii) an antigen polypeptide from influenza.

In one example, the cRNA comprises, in order from 5′ to 3′: a) a first nucleotide sequence encoding antigen from a severe acute respiratory syndrome coronavirus (SARS-CoV-2) operably linked to a Kozak consensus sequence and an IRES; and b) a second nucleotide sequence encoding an antigen from a respiratory syncytial virus (RSV), operably linked to a regulatory element selected from the group consisting of a SG promoter and an IRES.

In one example, the cRNA comprises, in order from 5′ to 3′: a) a first nucleotide sequence encoding an antigen from a respiratory syncytial virus (RSV), operably linked to a Kozak consensus sequence and an IRES; and b) a second nucleotide sequence encoding an antigen from a severe acute respiratory syndrome coronavirus (SARS-CoV-2) operably linked to a regulatory element selected from the group consisting of a SG promoter and an IRES.

In another example, the present disclosure provides an immunogenic composition comprising:
(i) a self-replicating RNA comprising: a) a first nucleotide sequence encoding an antigen from respiratory syncytial virus (RSV) operably linked to a Kozak consensus sequence and an IRES; and b) a second nucleotide sequence encoding an antigen from influenza, operably linked to a regulatory element selected from the group consisting of a SG promoter and an IRES, and
(ii) an antigen polypeptide from a severe acute respiratory syndrome coronavirus (SARS-CoV-2).

In one example, the self-replicating RNA comprises, in order from 5′ to 3′: a) a first nucleotide sequence encoding antigen from respiratory syncytial virus (RSV) operably linked to a Kozak consensus sequence and an IRES; and b) a second nucleotide sequence encoding an antigen from influenza, operably linked to a regulatory element selected from the group consisting of a SG promoter and an IRES.

In one example, the self-replicating RNA comprises, in order from 5′ to 3′: a) a first nucleotide sequence encoding an antigen from influenza, operably linked to a Kozak consensus sequence and an IRES; and b) a second nucleotide sequence encoding an antigen from respiratory syncytial virus (RSV) operably linked to a regulatory element selected from the group consisting of a SG promoter and an IRES.

In an example, the immunogenic composition comprises:
(i) a self-replicating RNA comprising: a) a first nucleotide sequence encoding an antigen from a severe acute respiratory syndrome coronavirus (SARS-CoV-2) operably linked to a Kozak consensus sequence and an IRES; and b) a second nucleotide sequence encoding an antigen from influenza, operably linked to a regulatory element selected from the group consisting of a SG promoter and an IRES, and
(ii) an antigen polypeptide from a respiratory syncytial virus (RSV).

In one example, the self-replicating RNA comprises, in order from 5′ to 3′: a) a first nucleotide sequence encoding antigen from a severe acute respiratory syndrome coronavirus (SARS-CoV-2) operably linked to a Kozak consensus sequence and an IRES; and b) a second nucleotide sequence encoding an antigen from influenza, operably linked to a regulatory element selected from the group consisting of a SG promoter and an IRES.

In one example, the self-replicating RNA comprises, in order from 5′ to 3′: a) a first nucleotide sequence encoding an antigen from influenza, operably linked to a Kozak consensus sequence and a SG promoter; and b) a second nucleotide sequence encoding an antigen from a severe acute respiratory syndrome coronavirus (SARS-CoV-2) operably linked to a regulatory element selected from the group consisting of a SG promoter and an IRES.

In an example, the immunogenic composition comprises:
(i) a self-replicating RNA comprising: a) a first nucleotide sequence encoding an antigen from a severe acute respiratory syndrome coronavirus (SARS-CoV-2) operably linked to a Kozak consensus sequence and an IRES; and b) a second nucleotide sequence encoding an antigen from a respiratory syncytial virus (RSV), operably linked to a regulatory element selected from the group consisting of a SG promoter and an IRES, and
(ii) an antigen polypeptide from influenza.

In one example, the self-replicating RNA comprises, in order from 5′ to 3′: a) a first nucleotide sequence encoding antigen from a severe acute respiratory syndrome coronavirus (SARS-CoV-2) operably linked to a Kozak consensus sequence and an IRES; and b) a second nucleotide sequence encoding an antigen from a respiratory syncytial virus (RSV), operably linked to a regulatory element selected from the group consisting of a SG promoter and an IRES.

In one example, the self-replicating RNA comprises, in order from 5′ to 3′: a) a first nucleotide sequence encoding an antigen from a respiratory syncytial virus (RSV), operably linked to a Kozak consensus sequence and an IRES; and b) a second nucleotide sequence encoding an antigen from a severe acute respiratory syndrome coronavirus (SARS-CoV-2) operably linked to a regulatory element selected from the group consisting of a SG promoter and an IRES.

The present disclosure provides an immunogenic composition comprising:
(i) a polynucleotide comprising: a) a first nucleotide sequence encoding an antigen from respiratory syncytial virus (RSV) operably linked to a SG promoter; and b) a second nucleotide sequence encoding an antigen from influenza, operably linked to a regulatory element selected from the group consisting of a SG promoter and an IRES, and
(ii) an antigen polypeptide from a severe acute respiratory syndrome coronavirus (SARS-CoV-2).

In one example, the polynucleotide comprises, in order from 5′ to 3′: a) a first nucleotide sequence encoding antigen from respiratory syncytial virus (RSV) operably linked to a SG promoter; and b) a second nucleotide sequence encoding an antigen from influenza, operably linked to a regulatory element selected from the group consisting of a SG promoter and an IRES.

In one example, the polynucleotide comprises, in order from 5′ to 3′: a) a first nucleotide sequence encoding an antigen from influenza, operably linked to a SG promoter; and b) a second nucleotide sequence encoding an antigen from respiratory syncytial virus (RSV) operably linked to a regulatory element selected from the group consisting of a SG promoter and an IRES.

In an example, the immunogenic composition comprises:
(i) a polynucleotide comprising: a) a first nucleotide sequence encoding an antigen from a severe acute respiratory syndrome coronavirus (SARS-CoV-2) operably linked to a SG promoter; and b) a second nucleotide sequence encoding an antigen from influenza, operably linked to a regulatory element selected from the group consisting of a SG promoter and an IRES, and
(ii) an antigen polypeptide from a respiratory syncytial virus (RSV).

In one example, the polynucleotide comprises, in order from 5′ to 3′: a) a first nucleotide sequence encoding antigen from SARS-CoV-2 operably linked to a SG promoter; and b) a second nucleotide sequence encoding an antigen from influenza, operably linked to a regulatory element selected from the group consisting of a SG promoter and an IRES.

In one example, the polynucleotide comprises, in order from 5′ to 3′: a) a first nucleotide sequence encoding an antigen from influenza, operably linked to a SG promoter; and b) a second nucleotide sequence encoding an antigen from a severe acute respiratory syndrome coronavirus (SARS-CoV-2) operably linked to a regulatory element selected from the group consisting of a SG promoter and an IRES.

In an example, the immunogenic composition comprises:
(i) a polynucleotide comprising: a) a first nucleotide sequence encoding an antigen from a severe acute respiratory syndrome coronavirus (SARS-CoV-2) operably linked to a SG promoter; and b) a second nucleotide sequence encoding an antigen from a respiratory syncytial virus (RSV), operably linked to a regulatory element selected from the group consisting of a SG promoter and an IRES, and
(ii) an antigen polypeptide from influenza.

In one example, the polynucleotide comprises, in order from 5′ to 3′: a) a first nucleotide sequence encoding antigen from a severe acute respiratory syndrome coronavirus (SARS-CoV-2) operably linked to a SG promoter; and b) a second nucleotide sequence encoding an antigen from a respiratory syncytial virus (RSV), operably linked to a regulatory element selected from the group consisting of a SG promoter and an IRES.

In one example, the polynucleotide comprises, in order from 5′ to 3′: a) a first nucleotide sequence encoding an antigen from a respiratory syncytial virus (RSV), operably linked to a SG promoter; and b) a second nucleotide sequence encoding an antigen from a severe acute respiratory syndrome coronavirus (SARS-CoV-2) operably linked to a regulatory element selected from the group consisting of a SG promoter and an IRES.

In another example, the present disclosure provides an immunogenic composition comprising:
(i) a RNA comprising: a) a first nucleotide sequence encoding an antigen from respiratory syncytial virus (RSV) operably linked to a SG promoter; and b) a second nucleotide sequence encoding an antigen from influenza, operably linked to a regulatory element selected from the group consisting of a SG promoter and an IRES, and
(ii) an antigen polypeptide from a severe acute respiratory syndrome coronavirus (SARS-CoV-2).

In one example, the RNA comprises, in order from 5′ to 3′: a) a first nucleotide sequence encoding antigen from respiratory syncytial virus (RSV) operably linked to a SG promoter; and b) a second nucleotide sequence encoding an antigen from influenza, operably linked to a regulatory element selected from the group consisting of a SG promoter and an IRES.

In one example, the RNA comprises, in order from 5′ to 3′: a) a first nucleotide sequence encoding an antigen from influenza, operably linked to a SG promoter; and b) a second nucleotide sequence encoding an antigen from respiratory syncytial virus (RSV) operably linked to a regulatory element selected from the group consisting of a SG promoter and an IRES.

In an example, the immunogenic composition comprises:
(i) a RNA comprising: a) a first nucleotide sequence encoding an antigen from a severe acute respiratory syndrome coronavirus (SARS-CoV-2) operably linked to a SG promoter; and b) a second nucleotide sequence encoding an antigen from influenza, operably linked to a regulatory element selected from the group consisting of a SG promoter and an IRES, and
(ii) an antigen polypeptide from a respiratory syncytial virus (RSV).

In one example, the RNA comprises, in order from 5′ to 3′: a) a first nucleotide sequence encoding antigen from a severe acute respiratory syndrome coronavirus (SARS-CoV-2) operably linked to a SG promoter; and b) a second nucleotide sequence encoding an antigen from influenza, operably linked to a regulatory element selected from the group consisting of a SG promoter and an IRES.

In one example, the RNA comprises, in order from 5′ to 3′: a) a first nucleotide sequence encoding an antigen from influenza, operably linked to a SG promoter; and b) a second nucleotide sequence encoding an antigen from a severe acute respiratory syndrome coronavirus (SARS-CoV-2) operably linked to a regulatory element selected from the group consisting of a SG promoter and an IRES.

In an example, the immunogenic composition comprises:
(i) a RNA comprising: a) a first nucleotide sequence encoding an antigen from a severe acute respiratory syndrome coronavirus (SARS-CoV-2) operably linked to a SG promoter; and b) a second nucleotide sequence encoding an antigen from a respiratory syncytial virus (RSV), operably linked to a regulatory element selected from the group consisting of a SG promoter and an IRES, and
(ii) an antigen polypeptide from influenza.

In one example, the RNA comprises, in order from 5′ to 3′: a) a first nucleotide sequence encoding antigen from a severe acute respiratory syndrome coronavirus (SARS-CoV-2) operably linked to a SG promoter; and b) a second nucleotide sequence encoding an antigen from a respiratory syncytial virus (RSV), operably linked to a regulatory element selected from the group consisting of a SG promoter and an IRES.

In one example, the RNA comprises, in order from 5′ to 3′: a) a first nucleotide sequence encoding an antigen from a respiratory syncytial virus (RSV), operably linked to a SG promoter; and b) a second nucleotide sequence encoding an antigen from a severe acute respiratory syndrome coronavirus (SARS-CoV-2) operably linked to a regulatory element selected from the group consisting of a SG promoter and an IRES.

In another example, the present disclosure provides an immunogenic composition comprising:
(i) a cRNA comprising: a) a first nucleotide sequence encoding an antigen from respiratory syncytial virus (RSV) operably linked to a SG promoter; and b) a second nucleotide sequence encoding an antigen from influenza, operably linked to a regulatory element selected from the group consisting of a SG promoter and an IRES, and
(ii) an antigen polypeptide from a severe acute respiratory syndrome coronavirus (SARS-CoV-2).

In one example, the cRNA comprises, in order from 5′ to 3′: a) a first nucleotide sequence encoding antigen from respiratory syncytial virus (RSV) operably linked to a SG promoter; and b) a second nucleotide sequence encoding an antigen from influenza, operably linked to a regulatory element selected from the group consisting of a SG promoter and an IRES.

In one example, the cRNA comprises, in order from 5′ to 3′: a) a first nucleotide sequence encoding an antigen from influenza, operably linked to a SG promoter; and b) a second nucleotide sequence encoding an antigen from respiratory syncytial virus (RSV) operably linked to a regulatory element selected from the group consisting of a SG promoter and an IRES.

In an example, the immunogenic composition comprises:
(i) a cRNA comprising: a) a first nucleotide sequence encoding an antigen from a severe acute respiratory syndrome coronavirus (SARS-CoV-2) operably linked to a SG promoter; and b) a second nucleotide sequence encoding an antigen from influenza, operably linked to a regulatory element selected from the group consisting of a SG promoter and an IRES, and
(ii) an antigen polypeptide from a respiratory syncytial virus (RSV).

In one example, the cRNA comprises, in order from 5′ to 3′: a) a first nucleotide sequence encoding antigen from a severe acute respiratory syndrome coronavirus (SARS-CoV-2) operably linked to a SG promoter; and b) a second nucleotide sequence encoding an antigen from influenza, operably linked to a regulatory element selected from the group consisting of a SG promoter and an IRES.

In one example, the cRNA comprises, in order from 5′ to 3′: a) a first nucleotide sequence encoding an antigen from influenza, operably linked to a SG promoter; and b) a second nucleotide sequence encoding an antigen from a severe acute respiratory syndrome coronavirus (SARS-CoV-2) operably linked to a regulatory element selected from the group consisting of a SG promoter and an IRES.

In an example, the immunogenic composition comprises:
(i) a cRNA comprising: a) a first nucleotide sequence encoding an antigen from a severe acute respiratory syndrome coronavirus (SARS-CoV-2) operably linked to a SG promoter; and b) a second nucleotide sequence encoding an antigen from a respiratory syncytial virus (RSV), operably linked to a regulatory element selected from the group consisting of a SG promoter and an IRES, and
(ii) an antigen polypeptide from influenza.

In one example, the cRNA comprises, in order from 5′ to 3′: a) a first nucleotide sequence encoding antigen from a severe acute respiratory syndrome coronavirus (SARS-CoV-2) operably linked to a SG promoter; and b) a second nucleotide sequence encoding an antigen from a respiratory syncytial virus (RSV), operably linked to a regulatory element selected from the group consisting of a SG promoter and an IRES.

In one example, the cRNA comprises, in order from 5′ to 3′: a) a first nucleotide sequence encoding an antigen from a respiratory syncytial virus (RSV), operably linked to a SG promoter; and b) a second nucleotide sequence encoding an antigen from a severe acute respiratory syndrome coronavirus (SARS-CoV-2) operably linked to a regulatory element selected from the group consisting of a SG promoter and an IRES.

In another example, the present disclosure provides an immunogenic composition comprising:
(i) a self-replicating RNA comprising: a) a first nucleotide sequence encoding an antigen from respiratory syncytial virus (RSV) operably linked to a SG promoter; and b) a second nucleotide sequence encoding an antigen from influenza, operably linked to a regulatory element selected from the group consisting of a SG promoter and an IRES, and
(ii) an antigen polypeptide from a severe acute respiratory syndrome coronavirus (SARS-CoV-2).

In one example, the self-replicating RNA comprises, in order from 5′ to 3′: a) a first nucleotide sequence encoding antigen from respiratory syncytial virus (RSV) operably linked to a SG promoter; and b) a second nucleotide sequence encoding an antigen from influenza, operably linked to a regulatory element selected from the group consisting of a SG promoter and an IRES.

In one example, the self-replicating RNA comprises, in order from 5′ to 3′: a) a first nucleotide sequence encoding an antigen from influenza, operably linked to a SG promoter; and b) a second nucleotide sequence encoding an antigen from respiratory syncytial virus (RSV) operably linked to a regulatory element selected from the group consisting of a SG promoter and an IRES.

In an example, the immunogenic composition comprises:
(i) a self-replicating RNA comprising: a) a first nucleotide sequence encoding an antigen from a severe acute respiratory syndrome coronavirus (SARS-CoV-2) operably linked to a SG promoter; and b) a second nucleotide sequence encoding an antigen from influenza, operably linked to a regulatory element selected from the group consisting of a SG promoter and an IRES, and
(ii) an antigen polypeptide from a respiratory syncytial virus (RSV).

In one example, the self-replicating RNA comprises, in order from 5′ to 3′: a) a first nucleotide sequence encoding antigen from a severe acute respiratory syndrome coronavirus (SARS-CoV-2) operably linked to a SG promoter; and b) a second nucleotide sequence encoding an antigen from influenza, operably linked to a regulatory element selected from the group consisting of a SG promoter and an IRES.

In one example, the self-replicating RNA comprises, in order from 5′ to 3′: a) a first nucleotide sequence encoding an antigen from influenza, operably linked to a SG promoter; and b) a second nucleotide sequence encoding an antigen from a severe acute respiratory syndrome coronavirus (SARS-CoV-2) operably linked to a regulatory element selected from the group consisting of a SG promoter and an IRES.

In an example, the immunogenic composition comprises:
(i) a self-replicating RNA comprising: a) a first nucleotide sequence encoding an antigen from a severe acute respiratory syndrome coronavirus (SARS-CoV-2) operably linked to a SG promoter; and b) a second nucleotide sequence encoding an antigen from a respiratory syncytial virus (RSV), operably linked to a regulatory element selected from the group consisting of a SG promoter and an IRES, and
(ii) an antigen polypeptide from influenza.

In one example, the self-replicating RNA comprises, in order from 5′ to 3′: a) a first nucleotide sequence encoding antigen from a severe acute respiratory syndrome coronavirus (SARS-CoV-2) operably linked to a SG promoter; and b) a second nucleotide sequence encoding an antigen from a respiratory syncytial virus (RSV), operably linked to a regulatory element selected from the group consisting of a SG promoter and an IRES.

In one example, the self-replicating RNA comprises, in order from 5′ to 3′: a) a first nucleotide sequence encoding an antigen from a respiratory syncytial virus (RSV), operably linked to a SG promoter; and b) a second nucleotide sequence encoding an antigen from a severe acute respiratory syndrome coronavirus (SARS-CoV-2) operably linked to a regulatory element selected from the group consisting of a SG promoter and an IRES.

The present disclosure provides an immunogenic composition comprising:
(i) a polynucleotide comprising: a) a first nucleotide sequence encoding an antigen from respiratory syncytial virus (RSV) operably linked to an IRES; and b) a second nucleotide sequence encoding an antigen from influenza, operably linked to a regulatory element selected from the group consisting of a SG promoter and an IRES, and
(ii) an antigen polypeptide from a severe acute respiratory syndrome coronavirus (SARS-CoV-2).

In one example, the polynucleotide comprises, in order from 5′ to 3′: a) a first nucleotide sequence encoding antigen from respiratory syncytial virus (RSV) operably linked to an IRES; and b) a second nucleotide sequence encoding an antigen from influenza, operably linked to a regulatory element selected from the group consisting of a SG promoter and an IRES.

In one example, the polynucleotide comprises, in order from 5′ to 3′: a) a first nucleotide sequence encoding an antigen from influenza, operably linked to an IRES; and b) a second nucleotide sequence encoding an antigen from respiratory syncytial virus (RSV) operably linked to a regulatory element selected from the group consisting of a SG promoter and an IRES.

In an example, the immunogenic composition comprises:
(i) a polynucleotide comprising: a) a first nucleotide sequence encoding an antigen from a severe acute respiratory syndrome coronavirus (SARS-CoV-2) operably linked to an IRES; and b) a second nucleotide sequence encoding an antigen from influenza, operably linked to a regulatory element selected from the group consisting of a SG promoter and an IRES, and
(ii) an antigen polypeptide from a respiratory syncytial virus (RSV).

In one example, the polynucleotide comprises, in order from 5′ to 3′: a) a first nucleotide sequence encoding antigen from a severe acute respiratory syndrome coronavirus (SARS-CoV-2) operably linked to an IRES; and b) a second nucleotide sequence encoding an antigen from influenza, operably linked to a regulatory element selected from the group consisting of a SG promoter and an IRES.

In one example, the polynucleotide comprises, in order from 5′ to 3′: a) a first nucleotide sequence encoding an antigen from influenza, operably linked to an IRES; and b) a second nucleotide sequence encoding an antigen from a severe acute respiratory syndrome coronavirus (SARS-CoV-2) operably linked to a regulatory element selected from the group consisting of a SG promoter and an IRES.

In an example, the immunogenic composition comprises:
(i) a polynucleotide comprising: a) a first nucleotide sequence encoding an antigen from a severe acute respiratory syndrome coronavirus (SARS-CoV-2) operably linked to an IRES; and b) a second nucleotide sequence encoding an antigen from a respiratory syncytial virus (RSV), operably linked to a regulatory element selected from the group consisting of a SG promoter and an IRES, and
(ii) an antigen polypeptide from influenza.

In one example, the polynucleotide comprises, in order from 5′ to 3′: a) a first nucleotide sequence encoding antigen from a severe acute respiratory syndrome coronavirus (SARS-CoV-2) operably linked to an IRES; and b) a second nucleotide sequence encoding an antigen from a respiratory syncytial virus (RSV), operably linked to a regulatory element selected from the group consisting of a SG promoter and an IRES.

In one example, the polynucleotide comprises, in order from 5′ to 3′: a) a first nucleotide sequence encoding an antigen from a respiratory syncytial virus (RSV), operably linked to an IRES; and b) a second nucleotide sequence encoding an antigen from a severe acute respiratory syndrome coronavirus (SARS-CoV-2) operably linked to a regulatory element selected from the group consisting of a SG promoter and an IRES.

In another example, the present disclosure provides an immunogenic composition comprising:
(i) a RNA comprising: a) a first nucleotide sequence encoding an antigen from respiratory syncytial virus (RSV) operably linked to an IRES; and b) a second nucleotide sequence encoding an antigen from influenza, operably linked to a regulatory element selected from the group consisting of a SG promoter and an IRES, and
(ii) an antigen polypeptide from a severe acute respiratory syndrome coronavirus (SARS-CoV-2).

In one example, the RNA comprises, in order from 5′ to 3′: a) a first nucleotide sequence encoding antigen from respiratory syncytial virus (RSV) operably linked to an IRES; and b) a second nucleotide sequence encoding an antigen from influenza, operably linked to a regulatory element selected from the group consisting of a SG promoter and an IRES.

In one example, the RNA comprises, in order from 5′ to 3′: a) a first nucleotide sequence encoding an antigen from influenza, operably linked to an IRES; and b) a second nucleotide sequence encoding an antigen from respiratory syncytial virus (RSV) operably linked to a regulatory element selected from the group consisting of a SG promoter and an IRES.

In an example, the immunogenic composition comprises:
(i) a RNA comprising: a) a first nucleotide sequence encoding an antigen from a severe acute respiratory syndrome coronavirus (SARS-CoV-2) operably linked to an IRES; and b) a second nucleotide sequence encoding an antigen from influenza, operably linked to a regulatory element selected from the group consisting of a SG promoter and an IRES, and
(ii) an antigen polypeptide from a respiratory syncytial virus (RSV).

In one example, the RNA comprises, in order from 5′ to 3′: a) a first nucleotide sequence encoding antigen from a severe acute respiratory syndrome coronavirus (SARS-CoV-2) operably linked to an IRES; and b) a second nucleotide sequence encoding an antigen from influenza, operably linked to a regulatory element selected from the group consisting of a SG promoter and an IRES.

In one example, the RNA comprises, in order from 5′ to 3′: a) a first nucleotide sequence encoding an antigen from influenza, operably linked to an IRES; and b) a second nucleotide sequence encoding an antigen from a severe acute respiratory syndrome coronavirus (SARS-CoV-2) operably linked to a regulatory element selected from the group consisting of a SG promoter and an IRES.

In an example, the immunogenic composition comprises:
(i) a RNA comprising: a) a first nucleotide sequence encoding an antigen from a severe acute respiratory syndrome coronavirus (SARS-CoV-2) operably linked to an IRES; and b) a second nucleotide sequence encoding an antigen from a respiratory syncytial virus (RSV), operably linked to a regulatory element selected from the group consisting of a SG promoter and an IRES, and
(ii) an antigen polypeptide from influenza.

In one example, the RNA comprises, in order from 5′ to 3′: a) a first nucleotide sequence encoding antigen from a severe acute respiratory syndrome coronavirus (SARS-CoV-2) operably linked to an IRES; and b) a second nucleotide sequence encoding an antigen from a respiratory syncytial virus (RSV), operably linked to a regulatory element selected from the group consisting of a SG promoter and an IRES.

In one example, the RNA comprises, in order from 5′ to 3′: a) a first nucleotide sequence encoding an antigen from a respiratory syncytial virus (RSV), operably linked to an IRES; and b) a second nucleotide sequence encoding an antigen from a severe acute respiratory syndrome coronavirus (SARS-CoV-2) operably linked to a regulatory element selected from the group consisting of a SG promoter and an IRES.

In another example, the present disclosure provides an immunogenic composition comprising:
(i) a cRNA comprising: a) a first nucleotide sequence encoding an antigen from respiratory syncytial virus (RSV) operably linked to an IRES; and b) a second nucleotide sequence encoding an antigen from influenza, operably linked to a regulatory element selected from the group consisting of a SG promoter and an IRES, and
(ii) an antigen polypeptide from a severe acute respiratory syndrome coronavirus (SARS-CoV-2).

In one example, the cRNA comprises, in order from 5′ to 3′: a) a first nucleotide sequence encoding antigen from respiratory syncytial virus (RSV) operably linked to an IRES; and b) a second nucleotide sequence encoding an antigen from influenza, operably linked to a regulatory element selected from the group consisting of a SG promoter and an IRES.

In one example, the cRNA comprises, in order from 5′ to 3′: a) a first nucleotide sequence encoding an antigen from influenza, operably linked to an IRES; and b) a second nucleotide sequence encoding an antigen from respiratory syncytial virus (RSV) operably linked to a regulatory element selected from the group consisting of a SG promoter and an IRES.

In an example, the immunogenic composition comprises:
(i) a cRNA comprising: a) a first nucleotide sequence encoding an antigen from a severe acute respiratory syndrome coronavirus (SARS-CoV-2) operably linked to an IRES; and b) a second nucleotide sequence encoding an antigen from influenza, operably linked to a regulatory element selected from the group consisting of a SG promoter and an IRES, and
(ii) an antigen polypeptide from a respiratory syncytial virus (RSV).

In one example, the cRNA comprises, in order from 5′ to 3′: a) a first nucleotide sequence encoding antigen from a severe acute respiratory syndrome coronavirus (SARS-CoV-2) operably linked to an IRES; and b) a second nucleotide sequence encoding an antigen from influenza, operably linked to a regulatory element selected from the group consisting of a SG promoter and an IRES.

In one example, the cRNA comprises, in order from 5′ to 3′: a) a first nucleotide sequence encoding an antigen from influenza, operably linked to an IRES; and b) a second nucleotide sequence encoding an antigen from a severe acute respiratory syndrome coronavirus (SARS-CoV-2) operably linked to a regulatory element selected from the group consisting of a SG promoter and an IRES.

In an example, the immunogenic composition comprises:
(i) a cRNA comprising: a) a first nucleotide sequence encoding an antigen from a severe acute respiratory syndrome coronavirus (SARS-CoV-2) operably linked to an IRES; and b) a second nucleotide sequence encoding an antigen from a respiratory syncytial virus (RSV), operably linked to a regulatory element selected from the group consisting of a SG promoter and an IRES, and
(ii) an antigen polypeptide from influenza.

In one example, the cRNA comprises, in order from 5′ to 3′: a) a first nucleotide sequence encoding antigen from a severe acute respiratory syndrome coronavirus (SARS-CoV-2) operably linked to an IRES; and b) a second nucleotide sequence encoding an antigen from a respiratory syncytial virus (RSV), operably linked to a regulatory element selected from the group consisting of a SG promoter and an IRES.

In one example, the cRNA comprises, in order from 5′ to 3′: a) a first nucleotide sequence encoding an antigen from a respiratory syncytial virus (RSV), operably linked to an IRES; and b) a second nucleotide sequence encoding an antigen from a severe acute respiratory syndrome coronavirus (SARS-CoV-2) operably linked to a regulatory element selected from the group consisting of a SG promoter and an IRES.

In another example, the present disclosure provides an immunogenic composition comprising:
(i) a self-replicating RNA comprising: a) a first nucleotide sequence encoding an antigen from respiratory syncytial virus (RSV) operably linked to an IRES; and b) a second nucleotide sequence encoding an antigen from influenza, operably linked to a regulatory element selected from the group consisting of a SG promoter and an IRES, and
(ii) an antigen polypeptide from a severe acute respiratory syndrome coronavirus (SARS-CoV-2).

In one example, the self-replicating RNA comprises, in order from 5′ to 3′: a) a first nucleotide sequence encoding antigen from respiratory syncytial virus (RSV) operably linked to an IRES; and b) a second nucleotide sequence encoding an antigen from influenza, operably linked to a regulatory element selected from the group consisting of a SG promoter and an IRES.

In one example, the self-replicating RNA comprises, in order from 5′ to 3′: a) a first nucleotide sequence encoding an antigen from influenza, operably linked to an IRES; and b) a second nucleotide sequence encoding an antigen from respiratory syncytial virus (RSV) operably linked to a regulatory element selected from the group consisting of a SG promoter and an IRES.

In an example, the immunogenic composition comprises:
(i) a self-replicating RNA comprising: a) a first nucleotide sequence encoding an antigen from a severe acute respiratory syndrome coronavirus (SARS-CoV-2) operably linked to an IRES; and b) a second nucleotide sequence encoding an antigen from influenza, operably linked to a regulatory element selected from the group consisting of a SG promoter and an IRES, and
(ii) an antigen polypeptide from a respiratory syncytial virus (RSV).

In one example, the self-replicating RNA comprises, in order from 5′ to 3′: a) a first nucleotide sequence encoding antigen from a severe acute respiratory syndrome coronavirus (SARS-CoV-2) operably linked to an IRES; and b) a second nucleotide sequence encoding an antigen from influenza, operably linked to a regulatory element selected from the group consisting of a SG promoter and an IRES.

In one example, the self-replicating RNA comprises, in order from 5′ to 3′: a) a first nucleotide sequence encoding an antigen from influenza, operably linked to an IRES; and b) a second nucleotide sequence encoding an antigen from a severe acute respiratory syndrome coronavirus (SARS-CoV-2) operably linked to a regulatory element selected from the group consisting of a SG promoter and an IRES.

In an example, the immunogenic composition comprises:
(i) a self-replicating RNA comprising: a) a first nucleotide sequence encoding an antigen from a severe acute respiratory syndrome coronavirus (SARS-CoV-2) operably linked to an IRES; and b) a second nucleotide sequence encoding an antigen from a respiratory syncytial virus (RSV), operably linked to a regulatory element selected from the group consisting of a SG promoter and an IRES, and
(ii) an antigen polypeptide from influenza.

In one example, the self-replicating RNA comprises, in order from 5′ to 3′: a) a first nucleotide sequence encoding antigen from a severe acute respiratory syndrome coronavirus (SARS-CoV-2) operably linked to an IRES; and b) a second nucleotide sequence encoding an antigen from a respiratory syncytial virus (RSV), operably linked to a regulatory element selected from the group consisting of a SG promoter and an IRES.

In one example, the self-replicating RNA comprises, in order from 5′ to 3′: a) a first nucleotide sequence encoding an antigen from a respiratory syncytial virus (RSV), operably linked to an IRES; and b) a second nucleotide sequence encoding an antigen from a severe acute respiratory syndrome coronavirus (SARS-CoV-2) operably linked to a regulatory element selected from the group consisting of a SG promoter and an IRES.

In one example, the disclosure additionally provides an immunogenic composition comprising:
(i) a F protein from RSV; and
(ii) a sa-mRNA encoding a HA from influenza.

In one example, the disclosure additionally provides an immunogenic composition comprising:
(i) a F protein from RSV; and
(ii) a sa-mRNA encoding a HA and a NA from influenza.

In one example, the disclosure additionally provides an immunogenic composition comprising:
(i) a F protein from RSV; and
(ii) a plurality of sa-mRNAs each sa-mRNA encoding a HA from influenza.

In one example, the disclosure additionally provides an immunogenic composition comprising:
(i) a F protein from RSV; and
(ii) a plurality of sa-mRNAs each sa-mRNA encoding a HA and a NA from influenza.

In one example, the disclosure additionally provides an immunogenic composition comprising:
(i) a F protein from RSV; and
(ii) three or more sa-mRNAs each sa-mRNA encoding a HA from influenza.

In one example, the disclosure additionally provides an immunogenic composition comprising:
(i) a F protein from RSV; and
(ii) three or more sa-mRNAs each sa-mRNA encoding a HA and a NA from influenza.
(i) a F protein from RSV; and
(ii) four or more sa-mRNAs each sa-mRNA encoding a HA from influenza.

In one example, the disclosure additionally provides an immunogenic composition comprising:
(i) a F protein from RSV; and
(ii) four or more sa-mRNAs each sa-mRNA encoding a HA and a NA from influenza.

In some examples, e.g., where multiple sequence encoding antigens are included in a single sa-mRNA, the first nucleotide sequence is operably linked to a SG promoter and the second nucleotide sequence is operably linked to a SG promoter.

In one example, the SG promoter is a native SG promoter. For example, a native SG promoter is a promoter that is native to the RNA virus from which it is derived and/or based on (e.g., an alphavirus). In one example, the native SG promoter is a native alphavirus SG promoter.

In some examples, the SG promoter is encoded by a sequence comprising or consisting of SEQ ID NO: 32.

In one example, the SG promoter is a minimal SG promoter or an extended SG promoter.

In one example, the SG promoter is a minimal SG promoter. In one example, the native SG promoter is a minimal SG promoter. For example, the minimal SG promoter is the minimal sequence required for initiation of transcription. In one example, the minimal native SG promoter is 49 nucleotides in length. In one example, the minimal SG promoter is 49 nucleotides in length. In one example, the minimal native SG promoter is encoded by a sequence comprising or consisting of a sequence set forth in SEQ ID NO: 13. In one example, the minimal SG promoter is encoded by a sequence comprising or consisting of a sequence set forth in SEQ ID NO: 13.

In one example, the SG promoter is an extended SG promoter. In one example, the native SG promoter is an extended SG promoter. For example, the extended SG promoter is extended at the 5′ end with nucleotides occurring in a sequence encoding a non-structural protein (e.g., NSP4) of the RNA virus (e.g., an alphavirus). In one example, the extended SG promoter is extended at the 5′ end with nucleotides occurring in a sequence encoding an alphavirus NSP4. The addition of nucleotides to the 5′ end of the SG promoter sequence did not interfere with expression of the non-structural protein and viral replicase, e.g., alphavirus NSP4.

In one example, the SG promoter is extended at the 5′ end by 51 or fewer nucleotides occurring in a sequence encoding a non-structural protein (e.g., an alphavirus NSP4). In one example, the extended SG promoter is a minimal SG promoter extended at the 5′ end by no more than 51 nucleotides occurring in a sequence encoding a non-structural protein (e.g., an alphavirus NSP4). In one example, the extended SG promoter is encoded by a sequence comprising or consisting of a sequence set forth in SEQ ID NO: 13 extended at the 5′ end by no more than 51 nucleotides occurring in a sequence encoding a non-structural protein (e.g., an alphavirus NSP4). For example, the extended SG promoter is no more than 100 nucleotides in length. In one example, the extended SG promoter is encoded by a sequence comprising or consisting of nucleotides 2 to 101 of SEQ ID NO: 17.

In one example, the SG promoter is extended at the 5′ end by about 5 nucleotides to about 20 nucleotides, for example by about 5 nucleotides, or about 10 nucleotides, or about 12, or about 15 nucleotides, or about 20 nucleotides, occurring in a sequence encoding a non-structural protein (e.g., an alphavirus NSP4). In another example, the SG promoter is extended at the 5′ end by about 20 to about 35 nucleotides, for example, by about 25 nucleotides or about 27 nucleotides, or about 30 nucleotides, or about 35 nucleotides, occurring in a sequence encoding a non-structural protein (e.g., an alphavirus NSP4).

In one example, the SG promoter is extended at the 5′ end by about 12 nucleotides occurring in a sequence encoding a non-structural protein (e.g., an alphavirus NSP4). In one example, the extended SG promoter is encoded by a sequence set forth in SEQ ID NO: 13 extended at the 5′ end by 12 nucleotides occurring in a sequence encoding a non-structural protein (e.g., an alphavirus NSP4). For example, the extended SG promoter is no more than 61 nucleotides in length. In one example, the extended SG promoter is encoded by a sequence comprising or consisting of nucleotides 41 to 101 of SEQ ID NO: 17. In another example, the extended SG promoter is encoded by a sequence comprising or consisting of a sequence set forth in SEQ ID NO: 14.

In one example, the SG promoter is extended at the 5′ end by about 31 nucleotides occurring in a sequence encoding a non-structural protein (e.g., an alphavirus NSP4). In one example, the extended SG promoter is encoded by a sequence set forth in SEQ ID NO: 13 extended at the 5′ end by 31 nucleotides occurring in a sequence encoding a non-structural protein (e.g., an alphavirus NSP4). For example, the extended SG promoter is no more than 80 nucleotides in length. In one example, the extended SG promoter is encoded by a sequence comprising or consisting of nucleotides 22 to 101 of SEQ ID NO: 17. In another example, the extended SG promoter is encoded by a sequence comprising or consisting of a sequence set forth in SEQ ID NO: 15.

In one example, the extended SG promoter comprises a repeat sequence corresponding to nucleotides 66 to 75 of SEQ ID NO: 17. For example, the extended SG promoter is encoded by a sequence comprising nucleotides 50 to 75 of SEQ ID NO: 17 and nucleotides 66 to 101 of SEQ ID NO: 17. For example, the extended SG promoter is encoded by a sequence set forth in SEQ ID NO: 27.

In one example, the IRES is an IRES from encephalomyocarditis virus (EMCV), poliovirus (PV), human enterovirus, foot-and-mouth disease virus (FMDV), hepatitis C virus (HCV), classical swine fever virus (CSFV), murine leukemia virus (MLV), simian immunodeficiency virus (SIV), Eukaryotic translation initiation factor 4G (elF4G), Death-associated protein 5 (DAP5), cellular Myc (c-Myc), NF-xB-repressing factor (NRF), vascular endothelial growth factor (VEGF), fibroblast growth factor (FGF-2), platelet-derived growth factor B (PDGF B), Antennapedia, X-linked inhibitor of apoptosis (XIAP or Apaf-1), immunoglobulin heavy-chain binding protein BiP, or fibroblast growth factor 1a (FGF1A), GTX, or a combination thereof.

In one example, the IRES is a wild-type IRES derived from encephalomyocarditis virus (EMCV). For example, the wild-type EMCV IRES comprises a sequence set forth in SEQ ID NO: 16.

In one example, the nucleotide sequence is codon optimized.

In one example, the G/C content of the nucleotide sequence is modified.

In one example, the G/C content of the nucleotide sequence is increased by at least 5% compared to the G/C content of the unmodified sequence. For example, the G/C content of the first and/or second nucleotide sequence and/or the one or more additional nucleotide sequences is increased by at least 10%, or 15%, or 20%, or 25%, or 30%, or 35%, or 40% compared to the G/C content of the unmodified sequence.

In one example, the polynucleotide comprises at least one chemically modified nucleotide.

In one example, the chemically modified nucleotide is selected from the group consisting of N6,2′-O-dimethyl-adenosine (m6Am), 5-methyluridine (m5U), N4-acetylcytidine (ac4C), 2-thiocytidine (s2C), 2-thiouridine (s2U), 5-methylcytidine (m5C), N6-methyladenosine (m6a), pseudouridine (ψ), 1-methylpseudouridine (m1ψ), and combinations thereof. For example, the chemically modified nucleotide is N6,2′-O-dimethyl-adenosine (m6Am). For example, the chemically modified nucleotide is 5-methyluridine (m5U). For example, the chemically modified nucleotide is N4-acetylcytidine (ac4C). For example, the chemically modified nucleotide is 2-thiocytidine (s2C). For example, the chemically modified nucleotide is 2-thiouridine (s2U). For example, the chemically modified nucleotide is 5-methylcytidine (m5C). For example, the chemically modified nucleotide is N6-methyladenosine (m6a). For example, the chemically modified nucleotide is pseudouridine (ψ). For example, the chemically modified nucleotide is 1-methylpseudouridine (m1ψ).

In one example, the nucleotide sequence comprises the 5′-UTR of haptoglobin (HP), fibrinogen beta chain (FGB), haptoglobin-related protein (HPR), albumin (ALB), complement component 3 (C3), fibrinogen alpha chain (FGA), alpha 6 collagen (Col6A), alpha-1-antitrypsin (SERPINA1), alpha-1-antichymotrypsin (SERPINA3) a fragment and/or a variant thereof.

In one example, the 5′UTR is a 5′UTR of a Venezuelan equine encephalitis virus (VEEV) or modified forms thereof. For example, the 5′UTR comprises a sequence set forth in SEQ ID NO: 25.

In one example, the 5′-UTR, the fragment and/or the variant thereof is between 40 and 2000 nucleotides in length. For example, the 5′-UTR, the fragment and/or the variant thereof is between 40 and 100 nucleotides in length. For example, the 5′-UTR, the fragment and/or the variant thereof is between 100 and 250 nucleotides in length. For example, the 5′-UTR, the fragment and/or the variant thereof is between 250 and 500 nucleotides in length. For example, the 5′-UTR, the fragment and/or the variant thereof is between 500 and 750 nucleotides in length. For example, the 5′-UTR, the fragment and/or the variant thereof is between 750 and 1000 nucleotides in length. For example, the 5′-UTR, the fragment and/or the variant thereof is between 1000 and 1250 nucleotides in length. For example, the 5′-UTR, the fragment and/or the variant thereof is between 1250 and 1500 nucleotides in length. For example, the 5′-UTR, the fragment and/or the variant thereof is between 1500 and 1750 nucleotides in length. For example, the 5′-UTR, the fragment and/or the variant thereof is between 1750 and 2000 nucleotides in length.

In one example, the 5′-UTR, the fragment and/or the variant thereof comprises a nucleotide sequence at least 90% identical to a nucleotide sequence set forth in any one of SEQ ID NO: 21 to 26. For example, the 5′-UTR, the fragment and/or the variant thereof comprises a nucleotide sequence 90%, or 91%, or 92%, or 93%, or 94%, or 95%, or 96%, or 97%, or 98%, or 99% identical to a nucleotide sequence set forth in any one of SEQ ID NO: 21 to 26.

In one example, the polynucleotide comprises a combination of two or more 5′-UTRs, fragments and/or variants thereof. In one example, the two or more 5′-UTRs are the same. In one example, the two or more 5′-UTRs are different.

In one example, the nucleotide sequence comprising the 5′UTR comprises at least one microRNA binding site, an AU rich element (ARE), a GC-rich element, a stem loop, and combinations thereof. In one example, the nucleotide sequence comprises a microRNA binding site. In one example, the nucleotide sequence comprises an AU rich element (ARE). In one example, the nucleotide comprises a GC-rich element. In one example, the nucleotide sequence comprises a stem loop. For example, the stem loop is a histone stem loop.

In one example, the polynucleotide further comprises a nucleotide sequence comprising a 3′UTR. In one example, the nucleotide sequence comprising the 3′UTR is located 3′ of the second or the one or more additional nucleotide sequences. For example, the nucleotide sequence comprising the 3′UTR is located 3′ of the second nucleotide sequence. In one example, the 3′UTR comprises a 3′-UTR of arachidonate 5-lipoxygenase (ALOX5), alpha I collagen (COL1A1), tyrosine hydroxylase (TH) gene, amino-terminal enhancer of split (AES), human mitochondrial 12S rRNA (mtRNR1), a fragment and/or a variant thereof.

In one example, the 3′UTR is a 3′UTR of a Sindbis virus (SINV) or modified forms thereof. For example, the 3′UTR comprises a sequence set forth in SEQ ID NO: 26.

In one example, the 3′UTR, the fragment and/or the variant thereof is between 40 and 400 nucleotides in length. For example, the 3′-UTR is between 40 and 50, or 50 and 60, or 60 and 70, or 70 and 80, or 80 and 90, or 90 and 100, or 100 and 125, or 125 and 150, or 150 and 175, or 175 and 200, or 200 and 225, or 225 and 250, or 250 and 275, or 275 and 300, or 300 and 325, or 325 and 350, or 350 and 375, or 375 and 400 nucleotides in length. For example, the 3′-UTR, the fragment and/or the variant thereof is between 40 and 50 nucleotides in length. For example, the 3′-UTR, the fragment and/or the variant thereof is between 50 and 60 nucleotides in length. For example, the 3′-UTR, the fragment and/or the variant thereof is between 60 and 70 nucleotides in length. For example, the 3′-UTR, the fragment and/or the variant thereof is between 70 and 80 nucleotides in length. For example, the 3′-UTR, the fragment and/or the variant thereof is between 80 and 90 nucleotides in length. For example, the 3′-UTR, the fragment and/or the variant thereof is between 90 and 100 nucleotides in length. For example, the 3′-UTR, the fragment and/or the variant thereof is between 100 and 125 nucleotides in length. For example, the 3′-UTR, the fragment and/or the variant thereof is between 125 and 150 nucleotides in length. For example, the 3′-UTR, the fragment and/or the variant thereof is between 150 and 175 nucleotides in length. For example, the 3′-UTR, the fragment and/or the variant thereof is between 175 and 200 nucleotides in length. For example, the 3′-UTR, the fragment and/or the variant thereof is between 200 and 225 nucleotides in length. For example, the 3′-UTR, the fragment and/or the variant thereof is between 225 and 250 nucleotides in length. For example, the 3′-UTR, the fragment and/or the variant thereof is between 250 and 275 nucleotides in length. For example, the 3′-UTR, the fragment and/or the variant thereof is between 275 and 300 nucleotides in length. For example, the 3′-UTR, the fragment and/or the variant thereof is between 300 and 325 nucleotides in length. For example, the 3′-UTR, the fragment and/or the variant thereof is between 325 and 350 nucleotides in length. For example, the 3′-UTR, the fragment and/or the variant thereof is between 350 and 375 nucleotides in length. For example, the 3′-UTR, the fragment and/or the variant thereof is between 375 and 400 nucleotides in length.

In one example, the polynucleotide comprises a combination of two or more 3′-UTRs, fragments and/or variants thereof. In one example, the two or more 3′-UTRs are the same. In one example, the two or more 3′-UTRs are different.

In one example, the nucleotide sequence comprising the 3′UTR, the fragment and/or variant thereof comprises at least one microRNA binding site, an AU rich element (ARE), a GC-rich element, a triple helix, a stem loop, one or more stop codons and combinations thereof. In one example, the nucleotide sequence comprises a microRNA binding site. In one example, the nucleotide sequence comprises an AU rich element (ARE). In one example, the nucleotide sequence comprises a GC-rich element. In one example, the nucleotide sequence comprises a triple helix. In one example, the nucleotide sequence comprises a stem loop. For example, the stem loop is a histone stem loop. In one example, the nucleotide sequence comprises one or more stop codons. For example, the one or more stop codons are located at the 5′end of the 3′-UTR.

In one example, the polynucleotide comprises a nucleotide sequence comprising one or more 3′ tailing sequences located at the 3′end of the nucleotide sequence comprising the 3′UTR. In one example, the one or more 3′ tailing sequences are selected from the group consisting of a poly-A sequence, polyadenylation signal, a G-quadruplex, a poly-C sequence, a stem loop and combinations thereof. For example, the 3′ tailing sequence comprises a poly-A sequence. In one example, the 3′ tailing sequence comprises a polyadenylation signal. In one example, the 3′ tailing sequence comprises a G-quadruplex. In one example, the 3′ tailing sequence comprises a poly-C sequence. In one example, the 3′ tailing sequence comprises a stem loop. For example, the stem loop is a histone stem loop. In one example, the 3′ tailing sequence comprises a poly-A sequence and a G-quadruplex. In one example, the 3′ tailing sequence comprises a stem loop (e.g., a histone stem loop) and a poly-A sequence.

In one example, the one or more 3′ tailing sequences comprises one or more poly-A sequences each comprising between 10 and 300 consecutive adenosine nucleotides. For example, the poly-A sequences each comprises between 10 and 20, or 20 and 30, or 30 and 40, or 40 and 50, or 50 and 60, or 60 and 70, or 70 and 80, or 80 and 90, or 90 and 100, or 100 and 125, or 125 and 150, or 150 and 175, or 175 and 200, or 200 and 225, or 225 and 250, or 250 and 275, or 275 and 300 consecutive adenosine nucleotides. For example, the one or more poly-A sequences each comprises between 10 and 20 consecutive adenosine nucleotides. For example, the one or more poly-A sequences each comprises between 20 and 30 consecutive adenosine nucleotides. For example, the one or more poly-A sequences each comprises between 30 and 40 consecutive adenosine nucleotides. For example, the one or more poly-A sequences each comprise 36 consecutive adenosine nucleotides. For example, the one or more poly-A sequences each comprises between 40 and 50 consecutive adenosine nucleotides. For example, the one or more poly-A sequences each comprises between 50 and 60 consecutive adenosine nucleotides. For example, the one or more poly-A sequences each comprises between 60 and 70 consecutive adenosine nucleotides. For example, the one or more poly-A sequences each comprises between 70 and 80 consecutive adenosine nucleotides. For example, the one or more poly-A sequences each comprises between 80 and 90 consecutive adenosine nucleotides. For example, the one or more poly-A sequences each comprises between 90 and 100 consecutive adenosine nucleotides. For example, the one or more poly-A sequences each comprises between 100 and 125 consecutive adenosine nucleotides. For example, the one or more poly-A sequences each comprises between 125 and 150 consecutive adenosine nucleotides. For example, the one or more poly-A sequences each comprises between 150 and 175 consecutive adenosine nucleotides. For example, the one or more poly-A sequences each comprises between 175 and 200 consecutive adenosine nucleotides. For example, the one or more poly-A sequences each comprises between 200 and 225 consecutive adenosine nucleotides. For example, the one or more poly-A sequences each comprises between 225 and 250 consecutive adenosine nucleotides. For example, the one or more poly-A sequences each comprises between 250 and 275 consecutive adenosine nucleotides. For example, the one or more poly-A sequences each comprises between 275 and 300 consecutive adenosine nucleotides.

In one example, the one or more poly-A sequence each comprises 10, or 20, or 30, or 40, or 50, or 60, or 70, or 80, or 90, or 100, or 125, or 150, or 175, or 200, or 225, or 250, or 275, or 300 consecutive adenosine nucleotides. For example, the one or more poly-A sequence each comprises 10 consecutive adenosine nucleotides. For example, the one or more poly-A sequence each comprises 20 consecutive adenosine nucleotides. For example, the one or more poly-A sequence each comprises 30 consecutive adenosine nucleotides. For example, the one or more poly-A sequence each comprises 40 consecutive adenosine nucleotides. For example, the one or more poly-A sequence each comprises 50 consecutive adenosine nucleotides. For example, the one or more poly-A sequence each comprises 60 consecutive adenosine nucleotides. For example, the one or more poly-A sequence each comprises 70 consecutive adenosine nucleotides. For example, the one or more poly-A sequence each comprises 80 consecutive adenosine nucleotides. For example, the one or more poly-A sequence each comprises 90 consecutive adenosine nucleotides. For example, the one or more poly-A sequence each comprises 100 consecutive adenosine nucleotides. For example, the one or more poly-A sequence each comprises 125 consecutive adenosine nucleotides. For example, the one or more poly-A sequence each comprises 150 consecutive adenosine nucleotides. For example, the one or more poly-A sequence each comprises 175 consecutive adenosine nucleotides. For example, the one or more poly-A sequence each comprises 200 consecutive adenosine nucleotides. For example, the one or more poly-A sequence each comprises 225 consecutive adenosine nucleotides. For example, the one or more poly-A sequence each comprises 250 consecutive adenosine nucleotides. For example, the one or more poly-A sequence each comprises 275 consecutive adenosine nucleotides. For example, the one or more poly-A sequence each comprises 300 consecutive adenosine nucleotides.

In one example, the poly-A sequence comprises 36 consecutive adenosine nucleotides. For example, the poly-A sequence comprises a sequence set forth in SEQ ID NO: 28.

In one example, the one or more poly-A sequences is separated by an interrupting linker. For example, the 3′tailing sequence comprises, in order of 5′ to 3′: a poly-A sequence comprising consecutive adenosine nucleotides, an interrupting linker, and a further poly-A sequence comprising consecutive adenosine nucleotides.

In one example, the interrupting linker is from 10 to 50, or 50 to 100, or 100 to 150 nucleotides in length. For example, the interrupting linker is from 10 to 50 nucleotides in length. For example, the interrupting linker is from 50 to 100 nucleotides in length. For example, the interrupting linker is from 100 to 150 nucleotides in length.

In one example, the interrupting linker is 1, or 2, or 3, or 4, or 5, or 6, or 7, or 8, or 9, or 10, or 11, or 12, or 13, or 14, or 15, or 16, or 17, or 18, or 19, or 20, or 25, or 30, or 35, or 40, or 45, or 50, or 55, or 60, or 65, or 70, or 75, or 80, or 85, or 90, or 95, or 100, or 110, or 120, or 130, or 140, or 150 nucleotides in length. For example, the interrupting linker is 1 nucleotide in length. For example, the interrupting linker is 2 nucleotides in length. For example, the interrupting linker is 3 nucleotides in length. For example, the interrupting linker is 4 nucleotides in length. For example, the interrupting linker is 5 nucleotides in length. For example, the interrupting linker is 6 nucleotides in length. For example, the interrupting linker is 7 nucleotides in length. For example, the interrupting linker is 8 nucleotides in length. For example, the interrupting linker is 9 nucleotides in length. For example, the interrupting linker is 10 nucleotides in length. For example, the interrupting linker is 11 nucleotides in length. For example, the interrupting linker is 12 nucleotides in length. For example, the interrupting linker is 13 nucleotides in length. For example, the interrupting linker is 14 nucleotides in length. For example, the interrupting linker is 15 nucleotides in length. For example, the interrupting linker is 16 nucleotides in length. For example, the interrupting linker is 17 nucleotides in length. For example, the interrupting linker is 18 nucleotides in length. For example, the interrupting linker is 19 nucleotides in length. For example, the interrupting linker is 20 nucleotides in length. For example, the interrupting linker is 25 nucleotides in length. For example, the interrupting linker is 30 nucleotides in length. For example, the interrupting linker is 35 nucleotides in length. For example, the interrupting linker is 40 nucleotides in length. For example, the interrupting linker is 45 nucleotides in length. For example, the interrupting linker is 50 nucleotides in length. For example, the interrupting linker is 55 nucleotides in length. For example, the interrupting linker is 60 nucleotides in length. For example, the interrupting linker is 65 nucleotides in length. For example, the interrupting linker is 70 nucleotides in length. For example, the interrupting linker is 75 nucleotides in length. For example, the interrupting linker is 80 nucleotides in length. For example, the interrupting linker is 85 nucleotides in length. For example, the interrupting linker is 90 nucleotides in length. For example, the interrupting linker is 95 nucleotides in length. For example, the interrupting linker is 100 nucleotides in length. For example, the interrupting linker is 110 nucleotides in length. For example, the interrupting linker is 120 nucleotides in length. For example, the interrupting linker is 130 nucleotides in length. For example, the interrupting linker is 140 nucleotides in length. For example, the interrupting linker is 150 nucleotides in length.

In one example, the interrupting linker is 10 nucleotides in length. In one example, the interrupting linker comprises or consists of the nucleotide sequence set forth in SEQ ID NO: 20. For example, the interrupting linker comprises or consists of a nucleotide sequence GCAUAUGACU.

In one example, the 3′ tailing sequence comprises, in order of 5′ to 3′: a poly-A sequence comprising 30 consecutive adenosine nucleotides, an interrupting linker of 10 nucleotides, and a further poly-A sequence comprising 70 consecutive adenosine nucleotides.

In one example, the 3′ tailing sequence comprises, in order of 5′ to 3′: a poly-A sequence comprising 30 consecutive adenosine nucleotides, an interrupting linker comprising or consisting of the nucleotide sequence set forth in SEQ ID NO: 20, and a further poly-A sequence comprising 70 consecutive adenosine nucleotides.

In one example, the RNA further comprises a 5′ terminal cap structure.

In one example, the 5′ terminal cap structure is an endogenous cap or analogue thereof. For example, the 5′terminal cap structure is an endogenous cap. For example, the 5′terminal cap structure is an analogue of an endogenous cap.

In one example, the 5′ terminal cap structure comprise a guanine or guanine analogue thereof. For example, the 5′ terminal cap structure comprise a guanine. For example, the 5′ terminal cap structure comprise a guanine analogue of a guanine.

In one example, the 5′ terminal cap structure is selected from a group consisting of anti-reverse cap analogue (ARCA), N7,2′-O-dimethyl-guanosine (mCAP), inosine, N1-methyl-guanosine, 2′fluoro-guanosine, 7-deaza-guanosine, 8-oxo-guanosine, 2-amino-guanosine, LNA-guanosine, 2-azido-guanosine, N6,2′-O-dimethyladenosine, 7-methylguanosine (m7G), Cap1, and Cap2. For example, the 5′ terminal cap structure is anti-reverse cap analogue (ARCA). For example, the 5′ terminal cap structure is N7,2′-0-dimethyl-guanosine (mCAP). For example, the 5′ terminal cap structure is inosine. For example, the 5′ terminal cap structure is N1-methyl-guanosine. For example, the 5′ terminal cap structure is 2′fluoro-guanosine. For example, the 5′ terminal cap structure is 7-deaza-guanosine. For example, the 5′ terminal cap structure is 8-oxo-guanosine. For example, the 5′ terminal cap structure is 2-amino-guanosine. For example, the 5′ terminal cap structure is LNA-guanosine. For example, the 5′ terminal cap structure is 2-azido-guanosine. For example, the 5′ terminal cap structure is N6,2′-O-dimethyladenosine. For example, the 5′ terminal cap structure is 7-methylguanosine (m7G). For example, the 5′ terminal cap structure is Cap1. For example, the 5′ terminal cap structure is Cap2.

In one example, the 5′terminal cap structure is linked to the 5′ end of the RNA by a 5′-5′-triphosphate linkage or a 5′-5′ phosphorothioate linkage. For example, the 5′terminal cap structure is linked to the 5′ end of the RNA by a 5′-5′-triphosphate linkage. For example, the 5′terminal cap structure is linked to the 5′ end of the RNA by a 5′-5′ phosphorothioate linkage.

In one example, the self-replicating RNA is from an alphavirus. For example, the alphavirus is selected from the group consisting of Semliki Forest virus (SFV), Sindbis virus (SIN), and Venezuelan equine encephalitis virus (VEE) and combinations thereof.

In one example, the self-replicating RNA is from a Semliki Forest virus (SFV).

In one example, the self-replicating RNA is from a Sindbis virus (SIN).

In one example, the self-replicating RNA is from a Venezuelan equine encephalitis virus (VEE).

In one example, the self-replicating RNA comprises or consists of a polynucleotide sequence according to SEQ ID NO: 30.

In one example, the antigen is selected from the group consisting of a respiratory syncytial virus (RSV), an influenza virus and/or a SARS-CoV-2.

In an example, the antigen is from a respiratory syncytial virus (RSV).

In an example, the antigen is from influenza.

In an example, the antigen is from a SARS-CoV-2.

In one example, the antigens are from a single strain of an influenza virus (i.e., monovalent) or from multiple strains (i.e., multivalent). For example, the immunogenic composition includes antigens from one or more (e.g., 1 or 2 or 3) influenza virus strains.

In one example, the composition comprises first and second influenza viral antigens from different strains of the influenza virus. In an example, the composition comprises first, second and third influenza viral antigens from different strains of the influenza virus. For example, the composition comprises (i) a RNA comprising a nucleotide sequence encoding a first influenza antigen; and (ii) a polypeptide antigen from a different strain of influenza. In another example, the composition comprises (i) a RNA comprising a nucleotide sequence encoding a first influenza antigen; (ii) a RNA comprising a nucleotide sequence encoding a second influenza antigen and (iii) a polypeptide of a third influenza antigen. In another example, the composition comprises (i) a RNA comprising a nucleotide sequence encoding a first influenza antigen from influenza A; (ii) a RNA comprising a nucleotide sequence encoding a second influenza antigen from influenza B and (iii) a polypeptide of a third influenza antigen from influenza C.

In one example, the antigens are from an influenza A virus strain. For example, the antigens are an influenza A virus hemagglutinin (HA) protein, a neuraminidase (NA) protein, a matrix (M) protein, a nucleoprotein (NP), a non-structural (NS) protein, or an immunogenic fragment or variant thereof. In one example, the antigens are an influenza A hemagglutinin (HA) subtype H1, H2, H3, H4, H5, H6, H7, H8, H9, H10, H11, H12, H13, H14, H15 or H16 and/or an influenza A neuraminidase (NA) subtype N1, N2, N3, N4, N5, N6, N7, N8 or N9 and/or an influenza A matrix (M) protein subtype M1 or M2 and/or an influenza A non-structural (NS) protein subtype NS1 or NS2.

In one example, the influenza viral antigens are from different subtypes of the influenza virus. For example, different hemagglutinin subtypes and/or different neuraminidase subtypes and/or matrix protein subtypes, and/or nucleoprotein subtypes and/or non-structural protein subtypes.

The skilled person will be aware that pandemic strains of the influenza virus are commonly H1, H2, H3, H5, H6, H7 or H9 subtype influenza A virus strains. For example, H1N1, H2N2, H3N2, H5N1, H5N3, H6N1, H7N2, H7N3, H7N7, H7N9 and H9N2, strains. In one example, the antigen is a H1N1 antigen from a A/Delaware/55/2019 virus strain. In one example, the antigen is a H1 antigen from a A/Delaware/55/2019 virus strain.

In one example, the antigens are from influenza A virus strains having the same hemagglutinin subtypes. In another example, the antigens are influenza A virus strains having different hemagglutinin subtypes. In one example, the antigens are H1, H2, H3, H5, H6, H7 or H9 subtype influenza A virus strains. For example, the antigens are a H1 hemagglutinin, or a H2 hemagglutinin, or a H3 hemagglutinin, or a H5 hemagglutinin, or a H6 hemagglutinin, or a H7 hemagglutinin or a H9 hemagglutinin. For example, the antigens are a H5 subtype influenza A virus strain (i.e., a H5 hemagglutinin). In one example, the H5 hemagglutinin is an A/turkey/Turkey/1/2005 virus strain. In one example, the H3 hemagglutinin is an A/Delaware/39/2019 virus strain

In one example, the antigens are influenza A virus strains having the same neuraminidase subtypes. In another example, the antigens are influenza A virus strains having different neuraminidase subtypes. In one example, the antigens are N1, N2, N3, N7 or N9 subtype influenza A virus strains. For example, the antigens are a N1 neuraminidase, or a N2 neuraminidase, or a N3 neuraminidase, or a N7 neuraminidase, or a N9 neuraminidase. For example, the antigens are a N1 neuraminidase subtype influenza A virus strain. In one example, the N1 neuraminidase is an A/turkey/Turkey/1/2005 strain. In one example, the N2 neuraminidase is an A/Delaware/39/2019 virus strain.

In one example, the antigens are a H5 hemagglutinin protein and/or a N1 neuraminidase protein. For example, the first antigen is a H5 hemagglutinin subtype influenza A virus strain and the second antigen is a N1 neuraminidase subtype influenza A virus strain.

In one example, the antigens are an influenza B virus strain. The skilled person will be aware that influenza B viruses are not divided into subtypes but are classified into two lineages, namely, B/Yamagata and B/Victoria.

In one example, the antigens are a B/Yamagata influenza B virus strain. For example, the influenza B virus strain is a B/Singapore/INFTT 16 0610/16 (By) virus strain. In another example, the antigens are a B/Victoria influenza B virus strain. In one example, the antigens are influenza B virus strains in the same lineage. In another example, the antigens are influenza B virus strains in different lineages.

In one example, the antigens are an influenza B virus Hyam protein and/or a Nyam protein. For example, antigens are an influenza B virus Hyam protein. In another example, the antigens are an influenza B virus Nyam protein. In a further example, the antigens are an influenza B virus Hyam and Nyam protein.

In one example, the antigen is a H1N1 antigen from influenza A and is encoded by a sequence that is at least about 70%, at least about 75%, at least about 80%, at least about 85%, at least about 90% at least about 95%, at least about 96%, at least about 97%, at least about 98%, at least about 99% or 100% identical to SEQ ID NO: 5. In another example, the antigen is a H5N1 antigen from influenza A and is encoded by a sequence that is at least about 70%, at least about 75%, at least about 80%, at least about 85%, at least about 90% at least about 95%, at least about 96%, at least about 97%, at least about 98%, at least about 99% or 100% identical to SEQ ID NO: 7.

In one example, the antigen is a H1N1 antigen from influenza A and has an amino acid sequence that is at least about 70%, at least about 75%, at least about 80%, at least about 85%, at least about 90% at least about 95%, at least about 96%, at least about 97%, at least about 98%, at least about 99% or 100% identical to SEQ ID NO: 6. In another example, the antigen is a H5N1 antigen from influenza A and has an amino acid sequence that is at least about 70%, at least about 75%, at least about 80%, at least about 85%, at least about 90% at least about 95%, at least about 96%, at least about 97%, at least about 98%, at least about 99% or 100% identical to SEQ ID NO: 8.

In one example, the antigen is from influenza B.

In one example, the antigen is from influenza C.

In one example, the antigens are from a single strain of a SARS-CoV-2 (i.e., monovalent) or from multiple strains of a SARS-CoV-2 (i.e., multivalent). For example, the immunogenic composition includes antigens from one or more (e.g., 1 or 2 or 3) SARS-CoV-2 strains.

In one example, the composition comprises first and second SARS-CoV-2 antigens from different strains of a SARS-CoV-2. In an example, the composition comprises first, second and third SARS-CoV-2 antigens from different strains of a SARS-CoV-2 virus. For example, the composition comprises (i) a RNA comprising a nucleotide sequence encoding a first SARS-CoV-2 antigen; and (ii) a polypeptide antigen from a different strain of a SARS-CoV-2. In another example, the composition comprises (i) a RNA comprising a nucleotide sequence encoding a first SARS-CoV-2 antigen; (ii) a RNA comprising a nucleotide sequence encoding a second SARS-CoV-2 antigen and (iii) a polypeptide of a third SARS-CoV-2 antigen. In another example, the composition comprises (i) a RNA comprising a nucleotide sequence encoding a first SARS-CoV-2 antigen from a delta strain of SARS-CoV-2; (ii) a RNA comprising a nucleotide sequence encoding a second SARS-CoV-2 antigen from an Omicron strain of SARS-CoV-2 and (iii) a polypeptide of a third SARS-CoV-2 antigen from a Wuhan (original) strain of SARS-CoV-2. In yet another example, the antigens are from one or more of the Alpha (B.1.1.7) strain, the Beta (B.1.351) strain, the Gamma (P1) strain, the Epsilon (B.1.429) strain, the Delta (B.1.617.2) mutant, the Kappa (B.1.617.1) mutant, the Wuhan (original) strain and/or the Omicron (B.1.1.529) strain of a SARS-CoV-2.

In one example, the antigens are a spike (S) protein and/or a nucleocapsid (N) protein of a SARS-CoV-2. For example, the antigens are a SARS-CoV-2 N protein and/or a S protein from the Alpha (B.1.1.7) strain, the Beta (B.1.351) strain, the Gamma (P1) strain, the Epsilon (B.1.429) strain, the Delta (B.1.617.2) mutant, the Kappa (B.1.617.1) mutant, the Wuhan (original) strain and/or the Omicron (B.1.1.529) strain. In one example, the antigens are a SARS-CoV-2 N protein and/or a S protein from the Wuhan (original) SARS-CoV-2 strain. In one example, the antigens are a SARS-CoV-2 N protein and/or a S protein from the delta SARS-CoV-2 strain. In one example, the antigens are a SARS-CoV-2 N protein and/or a S protein from the delta SARS-CoV-2 Omicron strain.

In one example, the antigen is a SARS-CoV-2 N protein.

In another example, the antigen is an ectodomain of the S protein from the Omicron strain of SARS-CoV-2.

For example, the antigen is an ectodomain of the S protein of a SARS-CoV-2 encoded by a sequence that is at least about 70%, at least about 75%, at least about 80%, at least about 85%, at least about 90% at least about 95%, at least about 96%, at least about 97%, at least about 98%, at least about 99% or 100% identical to the sequence set forth in SEQ ID NO: 9 or 11.

In another example, the antigen is an ectodomain of the S protein of a SARS-CoV-2 spike protein has a sequence that is at least about 70%, at least about 75%, at least about 80%, at least about 85%, at least about 90% at least about 95%, at least about 96%, at least about 97%, at least about 98%, at least about 99% or 100% identical to the sequence set forth in SEQ ID NO: 10 or 12.

In another example, the S protein is a mutant S protein.

In one example, a mutant S protein comprises a mutation in the receptor binding domain. For example, the mutation is selected from the group consisting of S438F, N439K, N440K, L441I, K444R, V445A, V445I, G446V, G446S, N450K, L452R, L452P, L455F, K458N, N460T, D467V, I468F, I468T, I468V, E4710, 1472V, A475V, G476S, S477G, S477I, S477N, S477R, T478I, P479L, P479L, P479S, N481D, N481H, V483F, V483A, E484D, E484K, E484K, E4840, G485S, Y489H, Y489D, Y489F, Y489C, Y489N, F490L, F490S, P491R, Q493L, S494P, Y495N, T500N, N501S and Y505H, Y508H. In one example, a mutant S protein comprises a mutation in the receptor binding domain selected from the group consisting of N439K, N439L, L452R, S477N, T478I, V483A and E484D.

In one example, a mutant S protein comprises a mutation in the receptor binding domain. For example, the mutation is selected from the group consisting of R346K, K417N, K417T, S438F, N439K, N440K, L441I, K444R, V445A, V445I, G446V, G446S, N450K, L452R, L452P, L455F, K458N, N460T, D467V, I468F, I468T, I468V, E4710, 1472V, A475V, G476S, S477G, S477I, S477N, S477R, T478I, T478K, P479L, P479S, N481D, N481H, V483F, V483A, E484D, E484K, E484K, E4840, G485S, Y489H, Y489D, Y489F, Y489C, Y489N, F490L, F490S, P491R, Q493L, S494P, Y495N, T500N, N501S, N501Y, Y505H and Y508H. In one example, a mutant S protein comprises a mutation in the receptor binding domain selected from the group consisting of R346K, K417N, K417T, N439K, N439L, L452R, S477N, T478I, V483A, E484D, E484K and N501Y.

In one example, a mutant S protein comprises a mutation selected from the group consisting of P337S, F338L, F338C, G339D, E340K, V341I, A344S, T345S, R346K, A348S, A348T, W353R, N354D, N354K, N354S, S359N, D364Y, V367F, S373L, V382L, P384L, P384S, T385A, T393P, V395I, F400C, R403K, R403S, D405V, R408I, Q414E, Q414K, Q414P, Q414R, T415S, K417R, K417N, 1418V, Y421S, Y423C, Y423F, Y423S, D427Y, R509K, V510L, V511E, V512L, L518I, H5190, A520S, A520V, P521R, P521S, A522P, A522S and D614G.

In one example, a mutant S protein comprises a mutation selected from the group consisting of L18F, D80A, T95I, Y144S, Y145N, D215G, P337S, F338L, F338C, G339D, E340K, V341I, A344S, T345S, R346K, A348S, A348T, W353R, N354D, N354K, N354S, S359N, D364Y, V367F, S373L, V382L, P384L, P384S, T385A, T393P, V395I, F400C, R403K, R403S, D405V, R408I, Q414E, Q414K, Q414P, Q414R, T415S, K417N, K417T, K417R, I418V, Y421S, Y423C, Y423F, Y423S, D427Y, S438F, N439K, N440K, L441I, K444R, V445A, V445I, G446V, G446S, N450K, L452R, L452P, L455F, K458N, N460T, D467V, I468F, I468T, I468V, E4710, I472V, A475V, G476S, S477G, S477I, S477N, S477R, T478I, T478K, P479L, P479S, N481D, N481H, V483F, V483A, E484D, E484K, E484K, E4840, G485S, Y489H, Y489D, Y489F, Y489C, Y489N, F490L, F490S, P491R, Q493L, S494P, Y495N, T500N, N501S, N501Y, Y505H, Y508H, R509K, V510L, V511E, V512L, L518I, H5190, A520S, A520V, P521R, P521S, A522P, A522S, A570D, D614G, P680H, P681H, A701V, T716I and D950N.

In one example, the S protein lacks a furin cleavage site at the S1/S2 boundary and/or the S2′ site.

In one example, the mutant S protein comprises mutations (e.g., substitutions) that stabilize the S protein in a prefusion conformation. For example, the S protein comprises substitutions K986P and V987P.

In one example, the antigens are from a single strain of a respiratory syncytial virus (RSV) (i.e., monovalent) or from multiple strains (i.e., multivalent) of a RSV. For example, the immunogenic composition includes antigens from one or more (e.g., 1 or 2 or 3) RSV strains.

In one example, the composition comprises first and second RSV antigens from different strains of RSV. In an example, the composition comprises first, second and third RSV antigens from different strains of RSV. For example, the composition comprises (i) a RNA comprising a nucleotide sequence encoding a first RSV antigen; and (ii) a polypeptide antigen from a different strain of RSV. In another example, the composition comprises (i) a RNA comprising a nucleotide sequence encoding a first RSV antigen; (ii) a RNA comprising a nucleotide sequence encoding a second RSV antigen and (iii) a polypeptide of a third RSV antigen from RSV.

For example, the antigen is selected from a RSV surface glycoprotein including a Fusion (F), Glycoprotein (G), Small Hydrophobic protein (SH), the matrix proteins M and M2, the nucleocapsid proteins N, P and L, and the nonstructural proteins NS1 and NS2. In certain examples, the first and second antigens are each an RSV-F antigen. In certain examples, the first, second and third antigens are each an RSV-F antigen.

In one example, the antigen is from a Pre F protein of a RSV encoded by a sequence that is at least about 70%, at least about 75%, at least about 80%, at least about 85%, at least about 90% at least about 95%, at least about 96%, at least about 97%, at least about 98%, at least about 99% or 100% identical to SEQ ID NO: 1 or 3.

In one example, the antigen is from a Pre F protein of a RSV has a sequence that is at least about 70%, at least about 75%, at least about 80%, at least about 85%, at least about 90% at least about 95%, at least about 96%, at least about 97%, at least about 98%, at least about 99% or 100% identical to SEQ ID NO: 2 or 4.

In one example, the antigen is from a G protein of a RSV.

In an example, the composition further comprises one or more additional antigens. In some examples, the composition will therefore comprise a third antigen. In another example, the composition will comprise a fourth antigen. In an example, the one or more additional, or third or fourth antigens are viral antigens. For example, the viral antigen is from a respiratory virus. In one example, the respiratory virus is selected from the group consisting of influenza virus, respiratory syncytial virus, parainfluenza viruses, metapneumovirus, rhinovirus, coronaviruses, adenoviruses and bocaviruses.

In one example, the one or more additional viral antigens are from an influenza virus.

In one example, the one or more additional viral antigens are from a respiratory syncytial virus.

In one example, the one or more additional viral antigens are from a parainfluenza virus.

In one example, the one or more additional viral antigens are from a metapneumovirus.

In one example, the one or more additional viral antigens are from a rhinovirus.

In one example, the one or more additional viral antigens are from a coronavirus.

In one example, the one or more additional viral antigens are from an adenovirus.

In one example, the one or more additional viral antigens are from a bocavirus.

In one example, the one or more additional antigens are viral antigens from an influenza virus or a coronavirus.

In one example, the first, second, third and/or fourth antigens are expressed at substantially the same level. For example, the antigens have a level of expression within about 10%, or about 5% or about 1% of each other. In another example, the first, second, third and/or fourth antigens are expressed at different levels. For example, the antigens have a level of expression greater than about 10%, or about 15% or about 20% of each other. Methods for determining the level of expression are known in the art and/or are described herein.

The present disclosure also provides a pharmaceutical composition comprising an immunogenic composition of the present disclosure and a pharmaceutically acceptable carrier. Pharmaceutically acceptable carriers suitable for use in the present disclosure will be apparent to the skilled person and/or are described herein.

In one example, the pharmaceutical composition further comprises a lipid nanoparticle (LNP), a polymeric microparticle, and an oil-in-water emulsion. For example, the polynucleotide, the RNA, the cRNA or the self-replicating RNA is encapsulated in, bound to or adsorbed on a LNP, a polymeric microparticle, and an oil-in-water emulsion. In one example, the polynucleotide is encapsulated in, bound to or adsorbed on a LNP, a polymeric microparticle, and an oil-in-water emulsion. In another example, the RNA is encapsulated in, bound to or adsorbed on a LNP, a polymeric microparticle, and an oil-in-water emulsion. For example, the cRNA is encapsulated in, bound to or adsorbed on a LNP, a polymeric microparticle, and an oil-in-water emulsion. For example, the self-replicating RNA is encapsulated in, bound to or adsorbed on a LNP, a polymeric microparticle, and an oil-in-water emulsion.

In one example, the pharmaceutical composition further comprises a LNP. For example, the polynucleotide is encapsulated in a LNP. In another example, the RNA is encapsulated in a LNP. For example, the cRNA is encapsulated in a LNP. For example, the self-replicating RNA is encapsulated in a LNP. For example, the polynucleotide is bound to a LNP. In another example, the RNA is bound to a LNP. For example, the cRNA is bound to a LNP. In another example, the self-replicating RNA is bound to a LNP. For example, the polynucleotide is adsorbed on to a LNP. In another example, the RNA is adsorbed on to a LNP. For example, the cRNA is adsorbed on to a LNP. In a further example, the self-replicating RNA is adsorbed on to a LNP. In another example, wherein each RNA is formulated together in the LNP. In another example, wherein each RNA is formulated separately in the LNP.

In one example, where multiple RNAs are contemplated, the RNAs are contained within the same LNP. In another example, the multiple RNAs are contained within different LNPs.

In one example, the composition further comprises an additional RNA encoding:
(i) one or more antigens;
(ii) one or more immunopotentiating agents;
(iii) one or more chemoattractants; and/or
(iv) one or more targeting molecules.

In one example, the additional RNA is contained within the same LNP as the RNA encoding the antigen or is contained within a LNP separate to the RNA encoding the antigen.

In one example, the LNP comprises a PEG-lipid, a structural lipid and/or a neutral lipid. For example, the LNP comprises a PEG-lipid, a structural lipid and a neutral lipid. In another example, the LNP comprises a PEG-lipid, a structural lipid or a neutral lipid.

In one example, the LNP further comprises a cationic lipid. In another example, the LNP does not comprise a cationic lipid.

In one example, the pharmaceutical composition further comprises a polymeric microparticle. For example, the polynucleotide is encapsulated in a polymeric microparticle. In another example, the RNA is encapsulated in a polymeric microparticle. For example, the cRNA is encapsulated in a polymeric microparticle. For example, the self-replicating RNA is encapsulated in a polymeric microparticle. For example, the polynucleotide is bound to a polymeric microparticle. In another example, the RNA is bound to a polymeric microparticle. For example, the cRNA is bound to a polymeric microparticle. In another example, the self-replicating RNA is bound to a polymeric microparticle. For example, the polynucleotide is adsorbed on to a polymeric microparticle. In another example, the RNA is adsorbed on to a polymeric microparticle. For example, the cRNA is adsorbed on to a polymeric microparticle. In a further example, the self-replicating RNA is adsorbed on to a polymeric microparticle.

In one example, the pharmaceutical composition further comprises an oil-in-water emulsion. For example, the polynucleotide is encapsulated in an oil-in-water emulsion. In another example, the RNA is encapsulated in an oil-in-water emulsion. For example, the cRNA is encapsulated in an oil-in-water emulsion. For example, the self-replicating RNA is encapsulated in an oil-in-water emulsion. For example, the polynucleotide is bound to an oil-in-water emulsion. In another example, the RNA is bound to an oil-in-water emulsion. For example, the cRNA is bound to an oil-in-water emulsion. In another example, the self-replicating RNA is bound to an oil-in-water emulsion. In a further example, the self-replicating RNA is adsorbed on to an oil-in-water emulsion. In a further example, the self-replicating RNA is resuspended in an oil-in-water emulsion.

The present disclosure also provides an immunogenic composition or a pharmaceutical composition of the disclosure for use as a vaccine.

In one example, the polynucleotide is DNA. In one example, the DNA encodes a cRNA described herein. In one example, the DNA encodes a self-replicating RNA vaccine described herein.

In one example, the DNA is a plasmid.

The present disclosure provides a method of treating or preventing or delaying progression of a disease or condition in a subject, the method comprising administering the immunogenic composition or the pharmaceutical composition of the present disclosure to a subject in need thereof. In one example, the disclosure provides a method of treating a disease or condition in a subject, the method comprising administering the immunogenic composition or the pharmaceutical composition of the present disclosure to a subject in need thereof. In another example, the disclosure provides a method of preventing a disease or condition in a subject, the method comprising administering the immunogenic composition or the pharmaceutical composition of the present disclosure to a subject in need thereof. In a further example, the disclosure provides a method of delaying progression of a disease or condition in a subject, the method comprising administering the immunogenic composition or the pharmaceutical composition of the present disclosure to a subject in need thereof.

In one example, the subject suffers from a disease or condition. In one example, the subject has been diagnosed as suffering from a disease or condition. In one example, the subject is receiving treatment for a disease or condition.

In one example, the disease or condition is selected from the group consisting of influenza, COVID-19, a SARS-CoV-2, respiratory syncytial virus (RSV) and acute respiratory distress syndrome (ARDS) or a combination thereof. In another example, the disease or condition is influenza and COVID-19. In another example, the disease or condition is influenza and RSV. In another example, the disease or condition is RSV and COVID-19. In another example, the disease or condition is influenza, RSV and COVID-19. In another example, the disease or condition is influenza and a SARS-CoV-2. In another example, the disease or condition is a SARS-CoV-2 and RSV. In another example, the disease or condition is a SARS-CoV-2. In another example, the disease or condition is a RSV. In another example, the disease or condition is influenza. In another example, the disease or condition is COVID-19. In another example, the disease or condition is ARDS.

A skilled person will understand that where the treatment, prevention or delaying progression of influenza is contemplated, the immunogenic compositions described herein will include at least one polynucleotide comprising a nucleotide sequence encoding an antigen from influenza or an antigen polypeptide from influenza. The composition may comprise additional polynucleotide or polypeptide antigens, for instance from RSV or a SARS-CoV-2, as described herein.

In an example, an immunogenic composition suitable for the treatment, prevention or delaying progression of influenza comprises:
a) a RNA comprising a nucleotide sequence encoding an antigen from a SARS-CoV-2, wherein the nucleotide sequence is operably linked to a regulatory element; and
b) an antigen polypeptide from influenza.

In another example, an immunogenic composition suitable for the treatment, prevention or delaying progression of influenza comprises:
a) a RNA comprising a nucleotide sequence encoding an antigen from influenza, wherein the nucleotide sequence is operably linked to a regulatory element; and
b) an antigen polypeptide from a SARS-CoV-2.

In another example, an immunogenic composition suitable for the treatment, prevention or delaying progression of influenza comprises:
a) a RNA comprising a nucleotide sequence encoding an antigen from influenza, wherein the nucleotide sequence is operably linked to a regulatory element; and
b) an antigen polypeptide from influenza.

In another example, an immunogenic composition suitable for the treatment, prevention or delaying progression of influenza comprises:
a) a RNA comprising a nucleotide sequence encoding an antigen from influenza, wherein the nucleotide sequence is operably linked to a regulatory element; and
b) an antigen polypeptide from a respiratory syncytial virus (RSV).

In another example, an immunogenic composition suitable for the treatment, prevention or delaying progression of influenza comprises:
a) a RNA comprising a nucleotide sequence encoding an antigen from a respiratory syncytial virus (RSV), wherein the nucleotide sequence is operably linked to a regulatory element; and
b) an antigen polypeptide from influenza.

In another example, an immunogenic composition suitable for the treatment, prevention or delaying progression of influenza comprises:
(i) a RNA comprising a nucleotide sequence encoding an antigen from respiratory syncytial virus (RSV), wherein the nucleotide sequence is operably linked to a regulatory element;
(ii) a RNA comprising a nucleotide sequence encoding an antigen from influenza, wherein the nucleotide sequence is operably linked to a regulatory element; and
(iii) an antigen polypeptide from a SARS-CoV-2.

In another example, an immunogenic composition suitable for the treatment, prevention or delaying progression of influenza comprises:
(i) a RNA comprising a nucleotide sequence encoding an antigen from a SARS-CoV-2, wherein the nucleotide sequence is operably linked to a regulatory element;
(ii) a RNA comprising a nucleotide sequence encoding an antigen from influenza, wherein the nucleotide sequence is operably linked to a regulatory element; and
(iii) an antigen polypeptide from a respiratory syncytial virus (RSV).

In another example, an immunogenic composition suitable for the treatment, prevention or delaying progression of influenza comprises:
(i) a RNA comprising a nucleotide sequence encoding an antigen from a SARS-CoV-2, wherein the nucleotide sequence is operably linked to a regulatory element;
(ii) a RNA comprising a nucleotide sequence encoding an antigen from respiratory syncytial virus (RSV), wherein the nucleotide sequence is operably linked to a regulatory element; and
(iii) an antigen polypeptide from influenza.

In another example, an immunogenic composition suitable for the treatment, prevention or delaying progression of influenza comprises:
(i) a RNA comprising a nucleotide sequence encoding an antigen from a SARS-CoV-2, wherein the nucleotide sequence is operably linked to a regulatory element;
(ii) an antigen polypeptide from influenza; and
(iii) an antigen polypeptide from respiratory syncytial virus (RSV).

In another example, an immunogenic composition suitable for the treatment, prevention or delaying progression of influenza comprises:
(i) a RNA comprising a nucleotide sequence encoding an antigen from influenza, wherein the nucleotide sequence is operably linked to a regulatory element;
(ii) an antigen polypeptide from a SARS-CoV-2; and
(iii) an antigen polypeptide from respiratory syncytial virus (RSV).

In another example, an immunogenic composition suitable for the treatment, prevention or delaying progression of influenza comprises:
(i) a RNA comprising a nucleotide sequence encoding an antigen from a respiratory syncytial virus (RSV), wherein the nucleotide sequence is operably linked to a regulatory element;
(ii) an antigen polypeptide from a SARS-CoV-2; and
(iii) an antigen polypeptide from influenza.

A skilled person will understand that where the treatment, prevention or delaying progression of a SARS-CoV-2 infection or COVID-19 is contemplated, the immunogenic compositions described herein will include at least one polynucleotide comprising a nucleotide sequence encoding an antigen from a SARS-CoV-2 or an antigen polypeptide from a SARS-CoV-2. The composition may comprise additional polynucleotide or polypeptide antigens, for instance from influenza or a RSV, as described herein.

In an example, an immunogenic composition suitable for the treatment, prevention or delaying progression of a SARS-CoV-2 infection or COVID-19 comprises:
a) a RNA comprising a nucleotide sequence encoding an antigen from a SARS-CoV-2, wherein the nucleotide sequence is operably linked to a regulatory element; and
b) an antigen polypeptide from influenza.

In another example, an immunogenic composition suitable for the treatment, prevention or delaying progression of a SARS-CoV-2 infection or COVID-19 comprises:
a) a RNA comprising a nucleotide sequence encoding an antigen from influenza, wherein the nucleotide sequence is operably linked to a regulatory element; and
b) an antigen polypeptide from a SARS-CoV-2.

In another example, an immunogenic composition suitable for the treatment, prevention or delaying progression of a SARS-CoV-2 infection or COVID-19 comprises:
a) a RNA comprising a nucleotide sequence encoding an antigen from a SARS-CoV-2, wherein the nucleotide sequence is operably linked to a regulatory element; and
b) an antigen polypeptide from a SARS-CoV-2.

In another example, an immunogenic composition suitable for the treatment, prevention or delaying progression of a SARS-CoV-2 infection or COVID-19 comprises:
a) a RNA comprising a nucleotide sequence encoding an antigen from a SARS-CoV-2, wherein the nucleotide sequence is operably linked to a regulatory element; and
b) an antigen polypeptide from a respiratory syncytial virus (RSV).

In another example, an immunogenic composition suitable for the treatment, prevention or delaying progression of a SARS-CoV-2 infection or COVID-19 comprises:
a) a RNA comprising a nucleotide sequence encoding an antigen from a respiratory syncytial virus (RSV), wherein the nucleotide sequence is operably linked to a regulatory element; and
b) an antigen polypeptide from a SARS-CoV-2.

In another example, an immunogenic composition suitable for the treatment, prevention or delaying progression of a SARS-CoV-2 infection or COVID-19 comprises:
(i) a RNA comprising a nucleotide sequence encoding an antigen from respiratory syncytial virus (RSV), wherein the nucleotide sequence is operably linked to a regulatory element;
(ii) a RNA comprising a nucleotide sequence encoding an antigen from influenza, wherein the nucleotide sequence is operably linked to a regulatory element; and
(iii) an antigen polypeptide from a SARS-CoV-2.

In another example, an immunogenic composition suitable for the treatment, prevention or delaying progression of a SARS-CoV-2 infection or COVID-19 comprises:
(i) a RNA comprising a nucleotide sequence encoding an antigen from a SARS-CoV-2, wherein the nucleotide sequence is operably linked to a regulatory element;
(ii) a RNA comprising a nucleotide sequence encoding an antigen from influenza, wherein the nucleotide sequence is operably linked to a regulatory element; and
(iii) an antigen polypeptide from a respiratory syncytial virus (RSV).

In another example, an immunogenic composition suitable for the treatment, prevention or delaying progression of a SARS-CoV-2 infection or COVID-19 comprises:
(i) a RNA comprising a nucleotide sequence encoding an antigen from a SARS-CoV-2, wherein the nucleotide sequence is operably linked to a regulatory element;
(ii) a RNA comprising a nucleotide sequence encoding an antigen from respiratory syncytial virus (RSV), wherein the nucleotide sequence is operably linked to a regulatory element; and
(iii) an antigen polypeptide from influenza.

In another example, an immunogenic composition suitable for the treatment, prevention or delaying progression of a SARS-CoV-2 infection or COVID-19 comprises:
(i) a RNA comprising a nucleotide sequence encoding an antigen from a SARS-CoV-2, wherein the nucleotide sequence is operably linked to a regulatory element;
(ii) an antigen polypeptide from influenza; and
(iii) an antigen polypeptide from respiratory syncytial virus (RSV).

In another example, an immunogenic composition suitable for the treatment, prevention or delaying progression of a SARS-CoV-2 infection or COVID-19 comprises:
(i) a RNA comprising a nucleotide sequence encoding an antigen from influenza, wherein the nucleotide sequence is operably linked to a regulatory element;
(ii) an antigen polypeptide from a SARS-CoV-2; and
(iii) an antigen polypeptide from respiratory syncytial virus (RSV).

In another example, an immunogenic composition suitable for the treatment, prevention or delaying progression of a SARS-CoV-2 infection or COVID-19 comprises:
(i) a RNA comprising a nucleotide sequence encoding an antigen from a respiratory syncytial virus (RSV), wherein the nucleotide sequence is operably linked to a regulatory element;
(ii) an antigen polypeptide from a SARS-CoV-2; and
(iii) an antigen polypeptide from influenza.

A skilled person will understand that where the treatment, prevention or delaying progression of RSV is contemplated, the immunogenic compositions described herein will include at least one polynucleotide comprising a nucleotide sequence encoding an antigen from a RSV or an antigen polypeptide from a RSV. The composition may comprise additional polynucleotide or polypeptide antigens, for instance from influenza or a SARS-CoV-2, as described herein.

In an example, an immunogenic composition suitable for the treatment, prevention or delaying progression of RSV comprises:
a) a RNA comprising a nucleotide sequence encoding an antigen from a SARS-CoV-2, wherein the nucleotide sequence is operably linked to a regulatory element; and
b) an antigen polypeptide from RSV.

In another example, an immunogenic composition suitable for the treatment, prevention or delaying progression of RSV comprises:
a) a RNA comprising a nucleotide sequence encoding an antigen from RSV, wherein the nucleotide sequence is operably linked to a regulatory element; and
b) an antigen polypeptide from a SARS-CoV-2.

In another example, an immunogenic composition suitable for the treatment, prevention or delaying progression of RSV comprises:
a) a RNA comprising a nucleotide sequence encoding an antigen from RSV, wherein the nucleotide sequence is operably linked to a regulatory element; and
b) an antigen polypeptide from RSV.

In another example, an immunogenic composition suitable for the treatment, prevention or delaying progression of RSV comprises:
a) a RNA comprising a nucleotide sequence encoding an antigen from influenza, wherein the nucleotide sequence is operably linked to a regulatory element; and
b) an antigen polypeptide from a respiratory syncytial virus (RSV).

In another example, an immunogenic composition suitable for the treatment, prevention or delaying progression of RSV comprises:
a) a RNA comprising a nucleotide sequence encoding an antigen from a respiratory syncytial virus (RSV), wherein the nucleotide sequence is operably linked to a regulatory element; and
b) an antigen polypeptide from influenza.

In another example, an immunogenic composition suitable for the treatment, prevention or delaying progression of RSV comprises:
(i) a RNA comprising a nucleotide sequence encoding an antigen from respiratory syncytial virus (RSV), wherein the nucleotide sequence is operably linked to a regulatory element;
(ii) a RNA comprising a nucleotide sequence encoding an antigen from influenza, wherein the nucleotide sequence is operably linked to a regulatory element; and
(iii) an antigen polypeptide from a SARS-CoV-2.

In another example, an immunogenic composition suitable for the treatment, prevention or delaying progression of RSV comprises:
(i) a RNA comprising a nucleotide sequence encoding an antigen from a SARS-CoV-2, wherein the nucleotide sequence is operably linked to a regulatory element;
(ii) a RNA comprising a nucleotide sequence encoding an antigen from influenza, wherein the nucleotide sequence is operably linked to a regulatory element; and
(iii) an antigen polypeptide from a respiratory syncytial virus (RSV).

In another example, an immunogenic composition suitable for the treatment, prevention or delaying progression of RSV comprises:
(i) a RNA comprising a nucleotide sequence encoding an antigen from a SARS-CoV-2, wherein the nucleotide sequence is operably linked to a regulatory element;
(ii) a RNA comprising a nucleotide sequence encoding an antigen from respiratory syncytial virus (RSV), wherein the nucleotide sequence is operably linked to a regulatory element; and
(iii) an antigen polypeptide from influenza.

In another example, an immunogenic composition suitable for the treatment, prevention or delaying progression of RSV comprises:
(i) a RNA comprising a nucleotide sequence encoding an antigen from a SARS-CoV-2, wherein the nucleotide sequence is operably linked to a regulatory element;
(ii) an antigen polypeptide from influenza; and
(iii) an antigen polypeptide from respiratory syncytial virus (RSV).

In another example, an immunogenic composition suitable for the treatment, prevention or delaying progression of RSV comprises:
(i) a RNA comprising a nucleotide sequence encoding an antigen from influenza, wherein the nucleotide sequence is operably linked to a regulatory element;
(ii) an antigen polypeptide from a SARS-CoV-2; and
(iii) an antigen polypeptide from respiratory syncytial virus (RSV).

In another example, an immunogenic composition suitable for the treatment, prevention or delaying progression of RSV comprises:
(i) a RNA comprising a nucleotide sequence encoding an antigen from a respiratory syncytial virus (RSV), wherein the nucleotide sequence is operably linked to a regulatory element;
(ii) an antigen polypeptide from a SARS-CoV-2; and
(iii) an antigen polypeptide from influenza.

The present disclosure provides a method of treating or preventing or delaying progression of a disease or condition in a subject, the method comprising locally coadministering
(i) a polynucleotide comprising a nucleotide sequence encoding an antigen selected from the group consisting of a respiratory syncytial virus (RSV), influenza and a severe acute respiratory syndrome coronavirus (SARS-CoV-2), wherein the nucleotide sequence is operably linked to a regulatory element; and
(ii) an antigen polypeptide selected from the group consisting of a RSV, influenza and a SARS-CoV-2.

In one example, the subject suffers from a disease or condition. In one example, the subject has been diagnosed as suffering from a disease or condition. In one example, the subject is receiving treatment for a disease or condition.

In another example, the method is for prophylaxis, e.g., to prevent or reduce the risk of contracting a disease or condition.

In one example, the disease or condition is selected from the group consisting of influenza, COVID-19, a SARS-CoV-2, respiratory syncytial virus (RSV) and acute respiratory distress syndrome (ARDS) or a combination thereof. In another example, the disease or condition is influenza and COVID-19. In another example, the disease or condition is influenza and RSV. In another example, the disease or condition is RSV and COVID-19. In another example, the disease or condition is influenza, RSV and COVID-19. In another example, the disease or condition is influenza and a SARS-CoV-2. In another example, the disease or condition is a SARS-CoV-2 and RSV. In another example, the disease or condition is a SARS-CoV-2. In another example, the disease or condition is a RSV. In another example, the disease or condition is influenza. In another example, the disease or condition is COVID-19. In another example, the disease or condition is ARDS.

A skilled person will understand that where the treatment, prevention or delaying progression of influenza is contemplated, the method described herein will include administering at least one polynucleotide comprising a nucleotide sequence encoding an antigen from influenza or an antigen polypeptide from influenza. The composition may comprise additional polynucleotide or polypeptide antigens, for instance from RSV or a SARS-CoV-2, as described herein.

In an example, a method for the treatment, prevention or delaying progression of an infection comprises locally co-administering:
a) a RNA comprising a nucleotide sequence encoding an antigen from a SARS-CoV-2, wherein the nucleotide sequence is operably linked to a regulatory element; and
b) an antigen polypeptide from influenza.

In another example, the method comprises administering:
a) a RNA comprising a nucleotide sequence encoding an antigen from influenza, wherein the nucleotide sequence is operably linked to a regulatory element; and
b) an antigen polypeptide from a SARS-CoV-2.

In another example, the method comprises administering:
a) a RNA comprising a nucleotide sequence encoding an antigen from influenza, wherein the nucleotide sequence is operably linked to a regulatory element; and
b) an antigen polypeptide from influenza.

In another example, the method comprises administering:
a) a RNA comprising a nucleotide sequence encoding an antigen from influenza, wherein the nucleotide sequence is operably linked to a regulatory element; and
b) an antigen polypeptide from a respiratory syncytial virus (RSV).

In another example, the method comprises administering:
a) a RNA comprising a nucleotide sequence encoding an antigen from a respiratory syncytial virus (RSV), wherein the nucleotide sequence is operably linked to a regulatory element; and
b) an antigen polypeptide from influenza.

In another example, the method comprises administering:
(i) a RNA comprising a nucleotide sequence encoding an antigen from respiratory syncytial virus (RSV), wherein the nucleotide sequence is operably linked to a regulatory element;
(ii) a RNA comprising a nucleotide sequence encoding an antigen from influenza, wherein the nucleotide sequence is operably linked to a regulatory element; and
(iii) an antigen polypeptide from a SARS-CoV-2.

In another example, the method comprises administering:
(i) a RNA comprising a nucleotide sequence encoding an antigen from a SARS-CoV-2, wherein the nucleotide sequence is operably linked to a regulatory element;
(ii) a RNA comprising a nucleotide sequence encoding an antigen from influenza, wherein the nucleotide sequence is operably linked to a regulatory element; and
(iii) an antigen polypeptide from a respiratory syncytial virus (RSV).

In another example, the method comprises administering:
(i) a RNA comprising a nucleotide sequence encoding an antigen from a SARS-CoV-2, wherein the nucleotide sequence is operably linked to a regulatory element;
(ii) a RNA comprising a nucleotide sequence encoding an antigen from respiratory syncytial virus (RSV), wherein the nucleotide sequence is operably linked to a regulatory element; and
(iii) an antigen polypeptide from influenza.

In another example, the method comprises administering:
(i) a RNA comprising a nucleotide sequence encoding an antigen from a SARS-CoV-2, wherein the nucleotide sequence is operably linked to a regulatory element;
(ii) an antigen polypeptide from influenza; and
(iii) an antigen polypeptide from respiratory syncytial virus (RSV).

In another example, the method comprises administering:
(i) a RNA comprising a nucleotide sequence encoding an antigen from influenza, wherein the nucleotide sequence is operably linked to a regulatory element;
(ii) an antigen polypeptide from a SARS-CoV-2; and
(iii) an antigen polypeptide from respiratory syncytial virus (RSV).

In another example, the method comprises administering:
(i) a RNA comprising a nucleotide sequence encoding an antigen from a respiratory syncytial virus (RSV), wherein the nucleotide sequence is operably linked to a regulatory element;
(ii) an antigen polypeptide from a SARS-CoV-2; and
(iii) an antigen polypeptide from influenza.

A skilled person will understand that where the treatment, prevention or delaying progression of a an influenza infection and/or a RSV infection and/or a SARS-CoV-2 infection or COVID-19 is contemplated.

Suitable combinations of antigens and/or antigens per se are described herein in relation to immunogenic compostions and shall be taken to apply mutatis mutandis to the methods of treatment described herein.

In one example, the disclosure provides a method of treating, preventing or delaying progression of an infection by influenza and/or RSV comprising locally coadministering:
(i) a F protein from RSV; and
(ii) a sa-mRNA encoding a HA from influenza.

In one example, the disclosure provides a method of treating, preventing or delaying progression of an infection by influenza and/or RSV comprising locally coadministering:
(i) a F protein from RSV; and
(ii) a sa-mRNA encoding a HA and a NA from influenza.

In one example, the disclosure provides a method of treating, preventing or delaying progression of an infection by influenza and/or RSV comprising locally coadministering:
(i) a F protein from RSV; and
(ii) a plurality of sa-mRNAs each sa-mRNA encoding a HA from influenza.

In one example, the disclosure provides a method of treating, preventing or delaying progression of an infection by influenza and/or RSV comprising locally coadministering:
(i) a F protein from RSV; and
(ii) a plurality of sa-mRNAs each sa-mRNA encoding a HA and a NA from influenza.

In one example, the disclosure provides a method of treating, preventing or delaying progression of an infection by influenza and/or RSV comprising locally coadministering:
(i) a F protein from RSV; and
(ii) three or more sa-mRNAs each sa-mRNA encoding a HA from influenza.

In one example, the disclosure provides a method of treating, preventing or delaying progression of an infection by influenza and/or RSV comprising locally coadministering:
(i) a F protein from RSV; and
(ii) three or more sa-mRNAs each sa-mRNA encoding a HA and a NA from influenza.
(i) a F protein from RSV; and
(ii) four or more sa-mRNAs each sa-mRNA encoding a HA from influenza.

In one example, the disclosure provides a method of treating, preventing or delaying progression of an infection by influenza and/or RSV comprising locally coadministering:
(i) a F protein from RSV; and
(ii) four or more sa-mRNAs each sa-mRNA encoding a HA and a NA from influenza.

In an example, the present disclosure provides a method of inducing an immune response in a subject, the method comprising administering a pharmaceutical composition disclosed herein, an immunogenic composition disclosed herein or a vaccine disclosed herein to a subject in need thereof or performing a method described herein.

In an example, the present disclosure provides use of a pharmaceutical composition disclosed herein, an immunogenic composition disclosed herein or a vaccine disclosed herein in the manufacture of a medicament for inducing an immune response in a subject in need thereof.

In an example, the present disclosure provides a pharmaceutical composition disclosed herein, an immunogenic composition disclosed herein or a vaccine disclosed herein for use in inducing an immune response in a subject in need thereof.

In one example, the composition or method of the disclosure induces a humoral immune response in the subject. For example, the humoral immune response is an antibody-mediated immune response. For example, production of neutralizing antibodies. In another example, the composition induces a cell-mediated immune response. For example, the cell-mediated immune response includes activation of antigen-specific cytotoxic T cells. For example, the T cells are CD4 T cells and/or CD8 T cells. In one example, the T cells are CD4 T cells. In another example, the T cells are CD8 T cells. In a further example, the T cells are CD4 and CD8 T cells.

In one example, administration of a pharmaceutical composition disclosed herein, an immunogenic composition disclosed herein or a vaccine of the present disclosure induces a CD4 T cell mediated immune response.

In one example, administration of a pharmaceutical composition, an immunogenic composition disclosed herein or a vaccine of the present disclosure induces a CD8 T cell mediated immune response.

In one example, administration of a pharmaceutical composition disclosed herein, an immunogenic composition disclosed herein or a vaccine of the present disclosure induces a CD4 and a CD8 T cell mediated immune response.

In one example, the CD4 T cell mediated immune response is a Th0, a Th1 and/or a Th2 response. For example, the CD4 T cell mediated immune response is a Th0 response. In another example, the CD4 T cell mediated immune response is a Th1 response. In a further example, the CD4 T cell mediated immune response is a Th2 response. In one example, the CD4 T cell mediated immune response is a Th0 and Th1 response. In another example, the CD4 T cell mediated immune response is a Th0 and Th2 response. In a further example, the CD4 T cell mediated immune response is a Th1 and Th2 response. In another example, the CD4 T cell mediated immune response is a Th0, Th1 and Th2 response.

In one example, the CD8 T cell mediated immune response is a Th0, a Th1 and/or a Th2 response. For example, the CD8 T cell mediated immune response is a Th0 response. In another example, the CD8 T cell mediated immune response is a Th1 response. In a further example, the CD8 T cell mediated immune response is a Th2 response. In one example, the CD8 T cell mediated immune response is a Th0 and Th1 response. In another example, the CD8 T cell mediated immune response is a Th0 and Th2 response. In a further example, the CD8 T cell mediated immune response is a Th1 and Th2 response. In another example, the CD8 T cell mediated immune response is a Th0, Th1 and Th2 response.

In one example, the compositions described herein are capable of modifying the Th2:Th1 ratio. In one example, the compositions described herein decrease the Th2:Th1 ratio.

In one example, the Th0 response cytokines express interleukin 2 (IL2+) and/or tumor necrosis factor alpha (TNFa+); and/or are negative for interferon gamma (IFNg−), IL5− and/or IL13−. For example, the cytokine is IL2+. In another example, the cytokine is TNFa+. In one example, the cytokine is IFNg−. In another example, the cytokine is IL5−. In a further example, the cytokine is IL13−.

In one example, the Th1 response cytokines express interferon gamma (IFNg+); and/or are negative for IL5− and/or IL13−. For example, the cytokine is IFNg+. In another example, the cytokine is IL5−. In a further example, the cytokine is IL13−.

In one example, the Th2 response cytokines express IL5+ and/or IL13+; and/or are negative for IFNg. For example, the cytokine is IL5+. In a further example, the cytokine is IL13+. For example, the cytokine is IFNg−.

In an example, the present disclosure provides a method for inducing a Th1 immune response in a subject in need thereof, comprising administering a pharmaceutical composition disclosed herein, an immunogenic composition disclosed herein or a vaccine disclosed herein to the subject or performing a method described herein.

In an example, the present disclosure provides use of a pharmaceutical composition disclosed herein, an immunogenic composition disclosed herein or a vaccine disclosed herein in the preparation of a medicament for inducing a Th1 immune response in a subject.

In an example, the present disclosure provides a pharmaceutical composition disclosed herein, an immunogenic composition disclosed herein or a vaccine disclosed herein for use in inducing a Th1 immune response in a subject.

In an example, the present disclosure provides a method for reducing viral load in a subject having a viral infection comprising administering a pharmaceutical composition disclosed herein, an immunogenic composition disclosed herein or a vaccine disclosed herein to a subject having a viral infection or performing a method described herein.

In an example, the present disclosure provides use of a pharmaceutical composition disclosed herein, an immunogenic composition disclosed herein or a vaccine disclosed herein in the preparation of a medicament for reducing viral load in a subject having a viral infection.

In an example, the present disclosure provides a pharmaceutical composition disclosed herein, an immunogenic composition disclosed herein or a vaccine disclosed herein for use in reducing viral load in a subject having a viral infection.

In an example, the subject is a human of 18 years of age or older. In another example, the subject is a human of any age, e.g., from about 1 month to 100 years old, e.g., from about 2 months to about 80 years old, from about 6 months of age to about 3 years old, from about 3 years to about 18 years old, from about 12 years to about 18 years old, from about 18 years to about 55 years old, from about 50 years to about 75 years old, from about 40 years to about 65 years old. In another example, the subject is a human from 2 years of age. In another example, subject is a human from 18 years of age, a human from 30 years of age, a human from 40 years of age, a human from 50 years of age, a human from 60 years of age, a human from 70 years of age, a human from 80 years of age or a human from about 90 years of age. In another example, the subject is less than 2 years of age, less than 18 months of age, less than 12 months of age, less than 6 months of age or less than 3 months of age.

In an example, the immunogenic compositions described herein are administered to an infant subject. In an example, the infant subject has, or is suspected of having RSV, influenza and/or a SARS-CoV-2 infection. In an example, the infant is between about 1 month and 12 months of age, or between 2 months and 11 months of age, or between 3 months and 10 months of age, or between 4 months and 9 months of age, or between 5 months and 8 months of age or between 6 months and 7 months of age. In an example, the infant is between about 1 month and 11 months of age, between about 1 month and 10 months of age, between about 1 month and 9 months of age, between about 1 month and 8 months of age, between about 1 month and 7 months of age, between about 1 month and 6 months of age, between about 1 month and 5 months of age, between about 1 month and 4 months of age, between about 1 month and 3 months of age or between about 1 month and 2 months of age. In another example the infant is about 1 month of age, about 2 months of age, about 3 months of age, about 4 months of age, about 5 months of age, about 6 months of age, about 7 months of age, about 8 months of age, about 9 months of age, about 10 months of age, about 11 months of age or about 12 months of age.

Thus, in an example, there is provided a method of treating, preventing or delaying progress of a respiratory syncytial virus (RSV) in an infant comprising administering an immunogenic composition, pharmaceutical composition or vaccine disclosed herein to the infant or performing a method described herein.

In another example, there is provided use of an immunogenic composition, pharmaceutical composition or vaccine disclosed herein in the preparation of a medicament for treating, preventing or delaying progress of a respiratory syncytial virus (RSV) in an infant.

In another example, there is provided an immunogenic composition, pharmaceutical composition or vaccine disclosed herein for use in treating, preventing or delaying progress of a respiratory syncytial virus (RSV) in an infant.

In another example, there is provided a method of inducing an immune response to a respiratory syncytial virus (RSV) in an infant comprising administering an immunogenic composition, pharmaceutical composition or vaccine disclosed herein to the infant or performing a method described herein.

In another example, there is provided use of an immunogenic composition, pharmaceutical composition or vaccine disclosed herein in the preparation of a medicament for inducing an immune response to a respiratory syncytial virus (RSV) in an infant.

In another example, there is provided an immunogenic composition, pharmaceutical composition or vaccine disclosed herein for use in inducing an immune response to a respiratory syncytial virus (RSV) in an infant.

Where treatment, prevention or delaying progression of RSV in an infant is contemplated, a skilled person will be aware of suitable immunogenic compositions described herein for administration to the infant.

In an example, the immunogenic composition suitable for administration to an infant comprises at least one polynucleotide comprising a nucleotide sequence encoding an antigen from respiratory syncytial virus (RSV) and/or an antigen polypeptide from a RSV. The composition may comprise additional polynucleotide or polypeptide antigens, for instance from influenza or a SARS-CoV-2, as described herein.

In an example, an immunogenic composition suitable for treatment of RSV in infant comprises:
(i) a RNA comprising a nucleotide sequence encoding an antigen from a respiratory syncytial virus (RSV), wherein the nucleotide sequence is operably linked to a regulatory element; and
(ii) an antigen polypeptide selected from the group consisting of a RSV, an influenza virus and/or a SARS-CoV-2.

In another example, an immunogenic composition suitable for treatment of RSV in infant comprises:
(i) a RNA comprising a nucleotide sequence encoding an antigen selected from the group consisting of a RSV, an influenza virus and/or a SARS-CoV-2, wherein the nucleotide sequence is operably linked to a regulatory element; and
(ii) an antigen polypeptide selected from a RSV.

In another example, an immunogenic composition suitable for treatment of RSV in infant comprises:
(i) a RNA comprising a nucleotide sequence encoding an antigen from respiratory syncytial virus (RSV), wherein the nucleotide sequence is operably linked to a regulatory element;
(ii) a RNA comprising a nucleotide sequence encoding an antigen selected from the group consisting of RSV, influenza or SARS-CoV-2, wherein the nucleotide sequence is operably linked to a regulatory element; and
(iii) an antigen polypeptide selected from the group consisting of RSV, influenza or SARS-CoV-2.

In another example, an immunogenic composition suitable for treatment of RSV in infant comprises:
(i) a RNA comprising a nucleotide sequence encoding an antigen from influenza or SARS-CoV-2, wherein the nucleotide sequence is operably linked to a regulatory element;
(ii) a RNA comprising a nucleotide sequence encoding an antigen from influenza or SARS-CoV-2, wherein the nucleotide sequence is operably linked to a regulatory element; and
(iii) an antigen polypeptide from a respiratory syncytial virus (RSV).

In one example, the immunogenic composition comprises:
(i) a F protein from RSV; and
(ii) a sa-mRNA encoding a HA from influenza.

In one example, the immunogenic composition comprises:
(i) a F protein from RSV; and
(ii) a sa-mRNA encoding a HA and a NA from influenza.

In one example, the immunogenic composition comprises:
(i) a F protein from RSV; and
(ii) a plurality of sa-mRNAs each sa-mRNA encoding a HA from influenza.

In one example, the immunogenic composition comprises:
(i) a F protein from RSV; and
(ii) a plurality of sa-mRNAs each sa-mRNA encoding a HA and a NA from influenza.

In one example, the immunogenic composition comprises:
(i) a F protein from RSV; and
(ii) three or more sa-mRNAs each sa-mRNA encoding a HA from influenza.

In one example, the immunogenic composition comprises:
(i) a F protein from RSV; and
(ii) three or more sa-mRNAs each sa-mRNA encoding a HA and a NA from influenza.
(i) a F protein from RSV; and
(ii) four or more sa-mRNAs each sa-mRNA encoding a HA from influenza.

In one example, the immunogenic composition comprises:
(i) a F protein from RSV; and
(ii) four or more sa-mRNAs each sa-mRNA encoding a HA and a NA from influenza.

In an example, a composition or vaccine described herein is administered in a one dose regimen. In another example, the composition is administered in a two, three or four dose regimen, wherein the doses are administered about 1, 2 or 3 months apart.

The present disclosure also provides a polynucleotide that encodes a self-replicating RNA described herein. For example, the polynucleotide is a recombinant DNA. In one example, the recombinant DNA is a plasmid. In one example, the plasmid comprises a sequence set forth in SEQ ID NO: 31.

The present disclosure also provides a kit comprising at least one immunogenic composition of the disclosure, optionally in a delivery system and/or a pharmaceutically acceptable carrier or diluent, packaged with instructions for use in treating or preventing or delaying progression of a disease or disorder in a subject.

The present disclosure also provides a kit comprising at least one immunogenic composition of the disclosure, optionally in a delivery system and/or a pharmaceutically acceptable carrier or diluent, packaged with instructions to administer the immunogenic composition to a subject who is suffering from, or at risk of suffering from, a disease or disorder.

In one example, an immunogenic composition, a pharmaceutical composition of the disclosure or a vaccine of the disclosure is supplied in a vial. In another example, an immunogenic composition, a pharmaceutical composition of the disclosure or a vaccine of the disclosure is supplied in a syringe.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### BRIEF DESCRIPTION OF FIGURES

FIG. 1A. Effect of sa-mRNA on adjuvanted protein vaccine MN and HAI neutralising titers. MN titers were determined by fluorescent foci assay (FFA) microneutralisation test on MDCK cells. GMT titers represent the EC50 values from a non-linear regression with 95% confidence interval. B. Effect of sa-mRNA on adjuvanted protein vaccine IgG titers. IgG titers were determined by Indirect ELISA against Monobulk-H1N1: A/Delaware/55/2019. The geometric mean of the titers for each group are displayed. C. Dose effect of sa-mRNA on IgG subclass. IgG1 and IgG2a titers were determined by Indirect ELISA against Monobulk-H1N1: A/Delaware/55/2019. The geometric mean of the titers for each group are displayed. D. Effect of sa-mRNA on IgG1:IgG2 ratio.

FIG. 2A. Effect of sa-mRNA on CD4 T-cell cytokines at day 42 in BALB/c mice. B. Effect of sa-mRNA on CD8 T-cell cytokines at day 42 in BALB/c mice. Peptide A is IYSTVASSL influenza A hemagglutinin epitope ID 29690.

FIG. 3A. Effect of sa-mRNA on adjuvanted protein vaccine MN neutralising titers. B. Effect of sa-mRNA on adjuvanted protein vaccine IgG titers. C. Dose effect of sa-mRNA on IgG subclass. D. Effect of sa-mRNA on IgG1:IgG2a ratio.

FIG. 4A. Effect of sa-mRNA on the Th response. B. Effect of sa-mRNA on CD4 T cell responses C. Effect of sa-mRNA on CD8 T cell responses. D. Effect of sa-mRNA on T helper cell response.

FIG. 5A. Effect of sa-mRNA on influenza MN, NA and IgG titers. B. Effect of sa-mRNA on influenza IgG1 and IgG2a responses C. Effect of sa-mRNA on omicron MN titers and ACE2 binding. D. Effect of sa-mRNA on omicron IgG1 and IgG2a responses.

FIG. 6A. Effect of sa-mRNA and protein based vaccine on CD4 T cell responses. B. Effect of sa-mRNA on T helper cell response. The ratio was set to 50 is zero Th1 cells were detected. The ratios for S protein were excluded if no/low responses were detected. C. Effect of sa-mRNA on CD8 T cell response.

FIG. 7A. Effect of sa-mRNA and protein based vaccine on H1N1 specific IgG response. B. Effect of sa-mRNA on IgG subclass C. Effect of sa-mRNA on H1N1 HAI titer. D. Effect of sa-mRNA on H1N1 MN titer. E. Effect of sa-mRNA on N1H1 NAI titer

FIG. 8A. Effect of sa-mRNA and protein based vaccine in presence of LNP adjuvant on neutralising titer. B. Effect of sa-mRNA and protein based vaccine in presence of LNP adjuvant on Pre F specific IgG1 response. C. Effect of sa-mRNA and protein based vaccine in presence of LNP adjuvant on Pre F specific IgG2a response. D-E. Effect of sa-mRNA and protein based vaccine in presence of LNP adjuvant on Pre F specific IgG1:IgG2a ratio. F. Effect of sa-mRNA and protein based vaccine in presence of LNP adjuvant on neutralising titer.

FIG. 9A. Effect of sa-mRNA and protein based vaccine on the Th1 response.

FIG. 10A. Effect of sa-mRNA on H1N1 HAI, FFA and IgG titers. B-C. Effect of sa-mRNA on B subunit antibody response.

FIG. 11A. Effect of sa-mRNA on the nSP1 Th1 response. B. Effect of sa-mRNA on the H1 Th1 response. C. Effect of sa-mRNA on the B/Yamagata Th2 response. D. Ratio of Th2/Th1 bias by antigen.

FIG. 12 includes a series of Figures. FIG. 12A. Depicts a study design to examine co-formulation and local or distal co-administration of sa-mRNA and unadjuvanted or adjuvanted protein vaccines. FIGS. 12B-D. Effect of unadjuvanted or adjuvanted protein on sa-mRNA vaccine neutralizing antibody response in context of co-formulation, local co-administration and distal co-administration was determined by influenza HAI, NAI and FFA-based MN assays. FIG. 12E. Effect of sa-mRNA on unadjuvanted or adjuvanted protein vaccine neutralizing antibody response in context of co-formulation, local co-administration and distal co-administration was determined by RSV FFA-based MN assay. FIGS. 12F-G. Effect of co-formulation and local or distal co-administration on IgG subclass response was measured by influenza- or RSV-binding IgG1 and IgG2a ELISA and data shown as a ratio of the antigen-specific IgG1 to IgG2a titers.

KEY TO SEQUENCE LISTING

| | SEQ |
|---|---|
| | ID |
| Sequence | NO |
| mRNA sequence of RSV F protein antigen | 1 |
| Amino acid sequence of RSV F protein antigen | 2 |
| mRNA sequence of RSV Pre F protein antigen | 3 |
| Amino acid sequence of RSV Pre F protein antigen | 4 |
| mRNA sequence of influenza antigen H1N1 | 5 |
| Amino acid sequence of influenza antigen H1N1 | 6 |
| mRNA sequence of influenza antigen H5N1 | 7 |
| Amino acid sequence of influenza antigen H5N1 | 8 |
| mRNA sequence of Omicron strain of SARS-CoV-2 S protein | 9 |
| ectodomain antigen | |
| Amino acid sequence of Omicron strain of SARS-CoV-2 S protein | 10 |
| ectodomain antigen | |
| mRNA sequence of Omicron strain of SARS-CoV-2 S protein | 11 |
| ectodomain antigen | |
| Amino acid sequence of Omicron strain of SARS-CoV-2 S protein | 12 |
| ectodomain antigen | |
| Nucleotide sequence of alphavirus native subgenomic | 13 |
| promoter (v1) | |
| Nucleotide sequence of extended subgenomic promoter (v2) | 14 |
| Nucleotide sequence of extended subgenomic promoter (v3) | 15 |
| Nucleotide sequence of wild-type EMCV IRES | 16 |
| Nucleotide sequence of extended subgenomic promoter (v4) | 17 |
| Intentionally skipped | 18 |
| Intentionally skipped | 19 |
| Nucleotide sequence of an interrupting linker | 20 |
| Nucleotide sequence of a GC-rich element | 21 |
| Nucleotide sequence of a GC-rich element | 22 |
| Intentionally skipped | 23 |
| Nucleotide sequence of a histone stem loop | 24 |
| Nucleotide sequence of 5′UTR of VEEV | 25 |
| Nucleotide sequence of 3′UTR of SINV | 26 |
| Nucleotide sequence of extended subgenomic promoter | 27 |
| Poly-A sequence | 28 |
| DNA sequence of self-replicating VEEV mRNA-H5 | 29 |
| RNA sequence of self-replicating VEEV mRNA-H5 | 30 |
| Plasmid sequence of self-replicating VEEV mRNA-H5 | 31 |
| Nucleotide sequence of a subgenomic promoter | 32 |

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise. Stated another way, any specific example of the present disclosure may be combined with any other specific example of the disclosure (except where mutually exclusive).

Any example of the present disclosure disclosing a specific feature or group of features or method or method steps will be taken to provide explicit support for disclaiming the specific feature or group of features or method or method steps.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein the term “derived from” shall be taken to indicate that a specified integer may be obtained from a particular source albeit not necessarily directly from that source. Similarly, the term “based on” shall be taken to indicate that a specified integer may be developed or used from a particular source albeit not necessarily directly from that source.

### Selected Definitions

As used herein, the term “self-replicating RNA” refers to a construct based on an RNA virus that has been engineered to allow expression of heterologous RNA and proteins. Self-replicating RNA (e.g., in the form of naked RNA) can amplify in host cells leading to expression of the desired gene product in the host cell.

As used herein, the term “conventional mRNA” or “cRNA” or “non-amplifying RNA” refers to a construct that allows expression of heterologous RNA and proteins but the RNA that cannot amplify in host cells.

As used herein, the term “monocistronic” in reference to the self-replicating RNA, refers to a RNA that encodes one polypeptide.

As used herein, the term “multicistronic” (also known as “polycistronic”) in reference to the polynucleotide, RNA, cRNA and/or self-replicating RNA, refers to a RNA that encodes two or more polypeptides. The term encompasses “bicistronic” (or “dicistronic”; i.e., encoding two polypeptides), “tricistronic” (i.e., encoding three polypeptides) molecules as well as RNA that is capable of encoding more than three polypeptides. By “bicistronic” is meant a single nucleic acid that is capable of encoding two distinct polypeptides from different regions of the nucleic acid.

The term “naked” as used herein refers to nucleic acids that are substantially free of other macromolecules, such as lipids, polymers and proteins. A “naked” nucleic acid, such as a self-replicating RNA, is not formulated with other macromolecules to improve cellular uptake. Accordingly, a naked nucleic acid is not encapsulated in, absorbed on, or bound to a LNP, a liposome, a polymeric microparticle or an oil-in-water emulsion.

As used herein, the term “nucleotide sequence” or “nucleic acid sequence” will be understood to mean a series of contiguous nucleotides (or bases) covalently linked to a phosphodiester backbone. By convention, sequences are presented from the 5′ end to the 3′ end, unless otherwise specified. To facilitate a clear description of the nucleic acids, particular sequence components are referred to as e.g., a “first nucleotide sequence” and a “second nucleotide sequence”. It is to be understood that the first and second sequences can appear in any desired order or orientation, unless otherwise specified, and that no particular order or orientation is intended by the words “first”, “second” etc.

As used herein, the term “antigen” refers to a molecule or structure containing one or more epitopes that induce, elicit, augment or boost a cellular and/or humoral immune response. Antigens can include, for example, proteins and peptides from a pathogen such as a virus, bacteria, fungus, protozoan, plant or from a tumour.

The term “polypeptide” or “polypeptide chain” will be understood to mean a series of contiguous amino acids linked by peptide bonds. For example, a protein shall be taken to include a single polypeptide chain i.e., a series of contiguous amino acids linked by peptide bonds or a series of polypeptide chains covalently or non-covalently linked to one another (i.e., a polypeptide complex). The series of polypeptide chains can be covalently linked using a suitable chemical or a disulfide bond. Examples of non-covalent bonds include hydrogen bonds, ionic bonds, Van der Waals forces, and hydrophobic interactions.

As used herein, the term “operably linked to” means positioning a subgenomic promoter or regulatory element (e.g., an IRES) relative to a nucleic acid such that expression of the nucleic acid is controlled or regulated by the element. For example, a subgenomic promoter can be operably linked to numerous nucleic acids, e.g., through another regulatory element, such as an internal ribosome entry site (IRES).

As used herein, the term “subgenomic promoter” (also known as ‘junction region’ promoter) refers to a promoter that directs the expression of a heterologous nucleotide sequence, regulating protein expression.

As used herein, the term “internal ribosome entry site” or “IRES” refers to a sequence of nucleotides within a mRNA to which a ribosome or a component thereof, e.g., a 40S subunit of a ribosome, is capable of binding. An IRES need not necessarily comprise nucleic acid that induces translation of a mRNA (e.g., a start codon; AUG).

The term “recombinant” shall be understood to mean the product of artificial genetic recombination.

As used herein, the terms “disease”, “disorder” or “condition” refers to a disruption of or interference with normal function, and is not to be limited to any specific condition, and will include diseases or disorders.

As used herein, a subject “at risk” of developing a disease or condition may or may not have detectable disease or symptoms of disease, and may or may not have displayed detectable disease or symptoms of disease prior to the treatment according to the present disclosure. “At risk” denotes that a subject has one or more risk factors, which are measurable parameters that correlate with development of the disease or condition, as known in the art and/or described herein.

As used herein, the terms “treating”, “treat” or “treatment” include administering a RNA or composition described herein to thereby reduce or eliminate at least one symptom of a specified disease or condition.

As used herein, the term “preventing”, “prevent” or “prevention” includes providing prophylaxis with respect to occurrence or recurrence of a specified disease or condition in an individual. An individual may be predisposed to or at risk of developing the disease but has not yet been diagnosed with the disease.

As used herein, the phrase “delaying progression of” includes reducing or slowing down the progression of the disease or condition in an individual and/or at least one symptom of a disease or condition.

An “effective amount” refers to at least an amount effective, at dosages and for periods of time necessary, to achieve the desired result. For example, the desired result may be a therapeutic or prophylactic result. An effective amount can be provided in one or more administrations. In some examples of the present disclosure, the term “effective amount” is meant an amount necessary to effect treatment of a disease or condition as hereinbefore described. In some examples of the present disclosure, the term “effective amount” is meant an amount necessary to effect a change associated with a disease or condition as hereinbefore described. The effective amount may vary according to the disease or condition to be treated or factor to be altered and also according to the weight, age, racial background, sex, health and/or physical condition and other factors relevant to the mammal being treated. Typically, the effective amount will fall within a relatively broad range (e.g. a “dosage” range) that can be determined through routine trial and experimentation by a medical practitioner. Accordingly, this term is not to be construed to limit the disclosure to a specific quantity, e.g., weight or number of RNA. The effective amount can be administered in a single dose or in a dose repeated once or several times over a treatment period.

A “therapeutically effective amount” is at least the minimum concentration required to effect a measurable improvement of a particular disease or condition. A therapeutically effective amount herein may vary according to factors such as the disease state, age, sex, and weight of the patient, and the ability of a composition of the present disclosure to elicit a desired response in the individual. A therapeutically effective amount is also one in which any toxic or detrimental effects of a composition are outweighed by the therapeutically beneficial effects.

As used herein, the term “prophylactically effective amount” shall be taken to mean a sufficient quantity of a composition of the disclosure to prevent or inhibit or delay the onset of one or more detectable symptoms of a disease or disorder as described herein.

As used herein, the term “subject” shall be taken to mean any animal including humans, for example a mammal. Exemplary subjects include but are not limited to humans and non-human primates. For example, the subject is a human.

As used herein, “locally coadministering” shall be understood to mean that two or more compositions are administered at substantially the same time, e.g., within 10 minutes or 5 minutes or 4 minutes or 3 minutes or 2 minutes or 1 minute of one another and at a similar site in a subject, e.g., within the same limb or within the same muscle or subcutaneously at a site covering the same muscle in a subject.

As used herein, “co-administered distally” shall be understood to mean that two or more compositions are administered at substantially the same time, e.g., within 10 minutes or 5 minutes or 4 minutes or 3 minutes or 2 minutes or 1 minute of one another and at distal sites in a subject, e.g., within different limbs of the subject.

### Antigens

Antigens suitable for use in the compositions described herein will be apparent to the skilled person.

In an example, the immunogenic compositions described herein comprise (i) an antigen selected from the group consisting of a respiratory syncytial virus (RSV), an influenza virus and/or a SARS-CoV-2 that is encoded by a nucleotide sequence and (ii) an antigen polypeptide selected from the group consisting of a respiratory syncytial virus (RSV), an influenza virus and/or a SARS-CoV-2.

In this regard, the phrase “an antigen selected from the group consisting of RSV, an influenza virus and a SARS-CoV-2” refers to an antigen from a virus selected from the group consisting of RSV, an influenza virus and a SARS-CoV-2. Without being bound by theory, it is envisaged that the compositions and vaccines of the present disclosure provide for an enhanced immune response when compared to the effect of a vaccine or composition comprising RNA antigens or polypeptide antigens alone. In particular, whilst protein based vaccines and RNA based vaccines tend to induce TH-2 and TH-1 immune responses respectively, the vaccines or compositions of the present disclosure are capable of inducing a TH-1 immune response, despite the presence of a polypeptide antigen. This type of vaccine or composition therefore has utility in particular settings where the induction of a TH-2 immune response is unwanted. For example, RSV protein vaccines are not administered to infants due to the belief that TH-2 dominated responses can lead to vaccine-enhanced disease(s). Thus, the administration of a vaccine of the disclosure could make the vaccine safer in this population by shifting the immune response towards a TH-1 response and/or providing a broader immune response.

### Influenza

Influenza, also known as “the flu”, is an infectious disease caused by an influenza virus. It will be apparent to the skilled person that there are currently four influenza viruses—A, B, C and D. Influenza A virus is the most common flu virus infecting humans, animals, and birds, whilst influenza B virus infection mostly occurs in humans. Infection of influenza C virus does not cause any severe symptom in human or mammals and influenza D, to date, has only infected pigs and cattle.

In one example, the antigen is from an influenza A virus strain. For example, the antigen is an influenza A virus hemagglutinin (HA) protein, a neuraminidase (NA) protein, a matrix (M) protein, a nucleoprotein (NP), a non-structural (NS) protein, or an immunogenic fragment or variant thereof. In one example, the antigen is an influenza A hemagglutinin (HA) subtype H1, H2, H3, H4, H5, H6, H7, H8, H9, H10, H11, H12, H13, H14, H15 or H16 and/or an influenza A neuraminidase (NA) subtype N1, N2, N3, N4, N5, N6, N7, N8 or N9 and/or an influenza A matrix (M) protein subtype M1 or M2 and/or an influenza A non-structural (NS) protein subtype NS1 or NS2.

In one example, the influenza viral antigen is from different subtypes of the influenza virus. For example, different hemagglutinin subtypes and/or different neuraminidase subtypes and/or matrix protein subtypes, and/or nucleoprotein subtypes and/or non-structural protein subtypes.

The skilled person will be aware that pandemic strains of the influenza virus are commonly H1, H2, H3, H5, H6, H7 or H9 subtype influenza A virus strains. For example, H1N1, H2N2, H3N2, H5N1, H5N3, H6N1, H7N2, H7N3, H7N7, H7N9 and H9N2, strains. In one example, the antigen is a H1N1 antigen from a A/Delaware/55/2019 virus strain. In one example, the antigen is a H1 antigen from a A/Delaware/55/2019 virus strain.

In one example, the antigen is from an influenza A virus strain having the same hemagglutinin subtypes. In another example, the antigen is an influenza A virus strains having different hemagglutinin subtypes. In one example, the antigen is a H1, H2, H3, H5, H6, H7 or H9 subtype influenza A virus strain. For example, the antigen is a H1 hemagglutinin, or a H2 hemagglutinin, or a H3 hemagglutinin, or a H5 hemagglutinin, or a H6 hemagglutinin, or a H7 hemagglutinin or a H9 hemagglutinin. For example, the antigen is a H5 subtype influenza A virus strain (i.e., a H5 hemagglutinin). In one example, the H5 hemagglutinin is an A/turkey/Turkey/1/2005 virus strain. In one example, the H3 hemagglutinin is an A/Delaware/39/2019 virus strain.

In one example, the antigen is an influenza A virus strain having the same neuraminidase subtypes. In another example, the antigen is an influenza A virus strain having different neuraminidase subtypes. In one example, the antigen is an N1, N2, N3, N7 or N9 subtype influenza A virus strain. For example, the antigen is a N1 neuraminidase, or a N2 neuraminidase, or a N3 neuraminidase, or a N7 neuraminidase, or a N9 neuraminidase. For example, the antigen is a N1 neuraminidase subtype influenza A virus strain. In one example, the N1 neuraminidase is an A/turkey/Turkey/1/2005 strain. In one example, the N2 neuraminidase is an A/Delaware/39/2019 virus strain.

In one example, the antigen is a H5 hemagglutinin protein and/or a N1 neuraminidase protein. For example, the first antigen is a H5 hemagglutinin subtype influenza A virus strain and the second antigen is a N1 neuraminidase subtype influenza A virus strain.

In one example, the antigen is an influenza B virus strain. The skilled person will be aware that influenza B viruses are not divided into subtypes but are classified into two lineages, namely, B/Yamagata and B/Victoria.

In one example, the antigen is a B/Yamagata influenza B virus strain. For example, the influenza B virus strain is a B/Singapore/INFTT 16 0610/16 (By) virus strain. In another example, the antigens are a B/Victoria influenza B virus strain. In one example, the antigens are influenza B virus strains in the same lineage. In another example, the antigens are influenza B virus strains in different lineages.

In one example, the antigen is an influenza B virus Hyam protein and/or a Nyam protein. For example, antigen is an influenza B virus Hyam protein. In another example, the antigen is an influenza B virus Nyam protein. In a further example, the antigen is an influenza B virus Hyam and Nyam protein.

### Severe Acute Respiratory Syndrome Coronavirus 2 (SARS-CoV-2)

COVID-19 is an infectious disease caused by SARS-CoV-2. It was first identified in December 2019 in Wuhan, Hubei, China, and has resulted in an ongoing pandemic.

In one example, the antigens are from a single strain of a SARS-CoV-2 (i.e., monovalent) or from multiple strains of a SARS-CoV-2 (i.e., multivalent). For example, the immunogenic composition includes antigens from one or more (e.g., 1 or 2 or 3) SARS-CoV-2 strains.

In one example, the composition comprises first and second SARS-CoV-2 antigens from different strains of a SARS-CoV-2. In an example, the composition comprises first, second and third SARS-CoV-2 antigens from different strains of a SARS-CoV-2 virus. For example, the composition comprises (i) a RNA comprising a nucleotide sequence encoding a first SARS-CoV-2 antigen; and (ii) a polypeptide antigen from a different strain of a SARS-CoV-2. In another example, the composition comprises (i) a RNA comprising a nucleotide sequence encoding a first SARS-CoV-2 antigen; (ii) a RNA comprising a nucleotide sequence encoding a second SARS-CoV-2 antigen and (iii) a polypeptide of a third SARS-CoV-2 antigen. In another example, the composition comprises (i) a RNA comprising a nucleotide sequence encoding a first SARS-CoV-2 antigen from a delta strain of SARS-CoV-2; (ii) a RNA comprising a nucleotide sequence encoding a second SARS-CoV-2 antigen from an omicron strain of SARS-CoV-2 and (iii) a polypeptide of a third SARS-CoV-2 antigen from a Wuhan (original) strain of SARS-CoV-2. In yet another example, the antigens are from one or more of the Alpha (B.1.1.7) strain, the Beta (B.1.351) strain, the Gamma (P1) strain, the Epsilon (B.1.429) strain, the Delta (B.1.617.2) mutant, the Kappa (B.1.617.1) mutant, the Wuhan (original) strain and/or the Omicron (B.1.1.529) strain of a SARS-CoV-2.

In one example, the antigens are a spike (S) protein and/or a nucleocapsid (N) protein of a SARS-CoV-2. For example, the antigens are a SARS-CoV-2 N protein and/or a S protein from the Alpha (B.1.1.7) strain, the Beta (B.1.351) strain, the Gamma (P1) strain, the Epsilon (B.1.429) strain, the Delta (B.1.617.2) mutant, the Kappa (B.1.617.1) mutant, the Wuhan (original) strain and/or the Omicron (B.1.1.529) strain. In one example, the antigens are a SARS-CoV-2 N protein and/or a S protein from the Wuhan (original) SARS-CoV-2 strain. In one example, the antigens are a SARS-CoV-2 N protein and/or a S protein from the delta SARS-CoV-2 strain. In one example, the antigens are a SARS-CoV-2 N protein and/or a S protein from the delta SARS-CoV-2 Omicron strain. In another example, the antigen is an ectodomain of the S protein from the Omicron strain of SARS-CoV-2.

### Respiratory Syncytial Virus (RSV)

RSV is an enveloped non-segmented negative-strand RNA virus in the family Paramyxoviridae, genus Pneumovirus. To infect a host cell, paramyxoviruses such as RSV, like other enveloped viruses such as influenza virus require fusion of the viral membrane with a host cell's membrane. For RSV, the conserved fusion protein (RSV-F glycoprotein) fuses the viral and cellular membranes by coupling irreversible protein refolding with juxtaposition of the membranes. Based on paramyxovirus studies, the RSV-F protein initially folds into a metastable pre-fusion conformation. During cell entry, the pre-fusion conformation undergoes refolding and conformational changes to its stable post-fusion conformation.

In an example, the one or more of the antigens are from RSV. For example, the antigen is a RSV surface glycoprotein selected from the Fusion (F), Glycoprotein (G), Small Hydrophobic protein (SH), the matrix proteins M and M2, the nucleocapsid proteins N, P and L, and the nonstructural proteins NS1 and NS2. In certain examples, two antigens are each an RSV-F antigen. In another example, the antigen is from a Pre F protein of a RSV. The F-protein presents two different conformations, a lollipop-shaped prefusion (preF), present on the virus surface before virus-cell interaction, and a crutch-shaped postfusion (postF) state which is acquired following the fusion between the virus and cell membrane or by unknown mechanisms that spontaneously initiate the rearrangement from the highly metastable preF into the energetically favorable postF conformation. The two forms are antigenically distinct, and both are considered as potential vaccine candidates.

The F glycoprotein of RSV is a type I single-pass integral membrane protein having four general domains: N-terminal ER-translocating signal sequence (SS), ectodomain (ED), transmembrane domain (TM), and a cytoplasmic tail (CT). CT contains a single palmitoylated cysteine residue. The sequence of F protein is highly conserved among RSV isolates but evolves over time. Unlike most paramyxoviruses, the F protein in RSV can mediate entry and syncytium formation independent of the other viral proteins (HN is usually necessary in addition to F in other paramyxoviruses).

The RSV-F glycoprotein is translated from mRNA into an approximately 574 amino acid protein designated F0. Post-translational processing of F0 includes removal of an N-terminal signal peptide by a signal peptidase in the endoplasmic reticulum. F0 is also cleaved at two sites (approximately 109/110 and approximately 136/137) by cellular proteases (in particular furin) in the trans-Golgi. This cleavage results in the removal of a short intervening sequence and generates two subunits designated F1 (~50 kDa; C-terminal; approximately residues 137-574) and F2 (~20 kDa; N-terminal; approximately residues 1-109) that remain associated with each other. F1 contains a hydrophobic fusion peptide at its N-terminus and also two amphipathic heptad-repeat regions (HRA and HRB). HRA is near the fusion peptide and HRB is near the transmembrane domain. Three F1-F2 heterodimers are assembled as homotrimers of F1-F2 in the virion.

RSV-F antigens suitable for inclusion in the immunogenic compositions described herein, either in RNA form or as polypeptides, include RSV-F glycoprotein and RSV-F glycoprotein variants. Suitable RSV-F glycoprotein variants include, for example, full length F protein and truncated variants such as soluble ecto-domains, each optionally containing one or more mutations, such as furin-cleavage mutations, trypsin-cleavage mutations, fusion peptide mutations (e.g., deletions in whole or in part), mutations that stabilize the HRB trimer, and mutations that destabilize the HRA trimer.

Full length and truncated RSV-F glycoproteins, including those with one or more such mutations in a variety of combinations are well known in the art and are disclosed for example in WO2011/008974, the disclosure of which is incorporated herein by reference in its entirety.

In an example, the composition described herein comprises additional antigens. In an example, the additional antigen is a virus, bacteria, a fungus or a protozoan.

### Viral Antigens

In one example, the polynucleotides of the present disclosure further comprise an additional viral antigen.

Additional viral antigens that can be encoded by a RNA according to the present disclosure, or provided in the form of a polypeptide, will be apparent to the skilled person and include, for example, proteins and peptides from a Orthomyxoviruses (e.g., Influenza A, B and C), Paramyxoviridae viruses (Pneumoviruses (e.g., Respiratory syncytial virus (RSV), Bovine respiratory syncytial virus, Pneumonia virus of mice, and Turkey rhinotracheitis virus), Paramyxovirus types 1-4 (PIV), Mumps, Sendai viruses, Simian virus 5)), Bovine parainfluenza virus, Nipahvirus, Henipavirus and Newcastle disease virus), Poxviridae (e.g., Variola vera, including but not limited to, Variola major and Variola minor, Metapneumoviruses, such as human metapneumovirus (hMPV) and avian metapneumoviruses (aMPV)), Morbilliviruses (e.g., Measles), Picornaviruses (e.g., Enteroviruses, Rhinoviruses, Heparnavirus, Parechovirus, Cardioviruses and Aphthoviruses), Enteroviruseses (e.g., Poliovirus types 1, 2 or 3, Coxsackie A virus types 1 to 22 and 24, Coxsackie B virus types 1 to 6, Echovirus (ECHO) virus types 1 to 9, 11 to 27 and 29 to 34 and Enterovirus 68 to 71), Bunyaviruses (e.g., California encephalitis virus), Phlebovirus (e.g., Rift Valley Fever virus), Nairovirus (e.g., Crimean-Congo hemorrhagic fever virus), Heparnaviruses (e.g., Hepatitis A virus (HAV)), Togaviruses (e.g., Rubivirus, an Alphavirus, or an Arterivirus), Flaviviruses (e.g., Tick-borne encephalitis (TBE) virus, Dengue (types 1, 2, 3 or 4) virus, Yellow Fever virus, Japanese encephalitis virus, Kyasanur Forest Virus, West Nile encephalitis virus, St. Louis encephalitis virus, Russian spring-summer encephalitis virus, Powassan encephalitis virus), Pestiviruses (e.g., Bovine viral diarrhea (BVDV), Classical swine fever (CSFV) or Border disease (BDV)), Hepadnaviruses (e.g., Hepatitis B virus, Hepatitis C virus), Rhabdoviruses (e.g., Lyssavirus (Rabies virus) and Vesiculovirus (VSV)), Caliciviridae (e.g., Norwalk virus, and Norwalk-like Viruses (e.g., Hawaii Virus and Snow Mountain Virus); Coronaviruses (e.g., severe acute respiratory syndrome (SARS) coronavirus (SARS-CoV), SARS coronavirus 2 (SARS-CoV-2), Middle East respiratory syndrome (MERS) coronavirus (MERS-CoV), Avian infectious bronchitis (IBV), Mouse hepatitis virus (MHV), and Porcine transmissible gastroenteritis virus (TGEV)), Retroviruses (e.g., Oncovirus, a Lentivirus or a Spumavirus), Reoviruses (e.g., Orthoreo virus, a Rotavirus, an Orbivirus, or a Coltivirus), Parvoviruses (e.g., Parvovirus B 19), Delta hepatitis virus (HDV), Hepatitis E virus (HEV), Human Herpesviruses (e.g., Herpes Simplex Viruses (HSV), Varicella-zoster virus (VZV), Epstein-Barr virus (EBV), Cytomegalovirus (CMV), Human Herpesvirus 6 (HHV6), Human Herpesvirus 7 (HHV7), and Human Herpesvirus 8 (HHV8)), Papovaviruses (e.g., Papillomaviruses and Polyomaviruses), Adenoviruess and Arenaviruses.

In one example, the additional viral antigen is from a parainfluenza virus.

In one example, the additional viral antigen is from a metapneumovirus.

In one example, the additional viral antigen is from a rhinovirus.

In one example, the additional viral antigen is from a coronavirus. In this example, the coronavirus may be SARS-CoV2 and the antigen is a RNA encoding a SARS-CoV2 spike glycoprotein peptide or fragment thereof, a RNA encoding a SARS-CoV2 nucleocapsid phosphoprotein peptide or fragment thereof, a RNA encoding a SARS-CoV2 membrane glycoprotein peptide or fragment thereof, a RNA encoding a SARS-CoV2 orf3a or fragment thereof, a RNA encoding a SARS-CoV2 orflab or fragment thereof or a variant of the above.

In one example, the additional viral antigen is from an adenovirus.

In one example, the additional viral antigen is from a bocavirus.

In one example, the additional antigen is from a single strain of an influenza virus (i.e., monovalent) or from multiple strains (i.e., multivalent).

In one example, the additional antigen is from an influenza A, B and/or C virus strain.

In one example, the additional antigen is from an influenza A virus strain. For example, the antigen is an influenza A virus hemagglutinin (HA) protein, a neuraminidase (NA) protein, a matrix (M) protein, a nucleoprotein (NP), a non-structural (NS) protein, or an immunogenic fragment or variant thereof. In one example, the additional antigen is an influenza A hemagglutinin (HA) subtype H1, H2, H3, H4, H5, H6, H7, H8, H9, H10, H11, H12, H13, H14, H15 or H16 and/or an influenza A neuraminidase (NA) subtype N1, N2, N3, N4, N5, N6, N7, N8 or N9 and/or an influenza A matrix (M) protein subtype M1 or M2 and/or an influenza A non-structural (NS) protein subtype NS1 or NS2.

In one example, the influenza viral antigens are from different subtypes of the influenza virus. For example, different hemagglutinin subtypes and/or different neuraminidase subtypes and/or matrix protein subtypes, and/or nucleoprotein subtypes and/or non-structural protein subtypes.

The skilled person will be aware that pandemic strains of the influenza virus are commonly H1, H2, H3, H5, H6, H7 or H9 subtype influenza A virus strains. For example, H1N1, H2N2, H3N2, H5N1, H5N3, H6N1, H7N2, H7N3, H7N7, H7N9 and H9N2, strains.

In one example, the additional antigen is from influenza A virus strain having the same hemagglutinin subtypes. In another example, the antigen is from influenza A virus strain having different hemagglutinin subtypes. In one example, the antigen is H1, H2, H3, H5, H6, H7 or H9 subtype influenza A virus strain. For example, the antigen is a H1 hemagglutinin, or a H2 hemagglutinin, or a H3 hemagglutinin, or a H5 hemagglutinin, or a H6 hemagglutinin, or a H7 hemagglutinin or a H9 hemagglutinin. For example, the antigen is a H5 subtype influenza A virus strain (i.e., a H5 hemagglutinin). In one example, the H5 hemagglutinin is an A/turkey/Turkey/1/2005 virus strain. In one example, the H3 hemagglutinin is an A/Delaware/39/2019 virus strain.

In one example, the additional antigen is from influenza A virus strain having the same neuraminidase subtypes. In another example, the antigen is from influenza A virus strains having different neuraminidase subtypes. In one example, the antigen is N1, N2, N3, N7 or N9 subtype influenza A virus strain. For example, the antigen is a N1 neuraminidase, or a N2 neuraminidase, or a N3 neuraminidase, or a N7 neuraminidase, or a N9 neuraminidase. For example, the antigen is a N1 neuraminidase subtype influenza A virus strain. In one example, the N1 neuraminidase is an A/turkey/Turkey/1/2005 strain. In one example, the N2 neuraminidase is an A/Delaware/39/2019 virus strain.

In an example, the antigens are a H5 hemagglutinin protein and a N1 neuraminidase protein. For example, the first antigen is a H5 hemagglutinin subtype influenza A virus strain and the second antigen is a N1 neuraminidase subtype influenza A virus strain.

Infections such as influenza and coronavirus infection are leading causes of ARDS. Accordingly, in one example of the present disclosure, the ARDS is associated with an influenza, RSV or a SARS-CoV-2 infection. In one example, the ARDS is associated with a SARS-CoV-2 infection. Thus, a skilled person will understand that antigens targeting a SARS-CoV-2 infection or influenza, including those listed above, may be antigens suitable for the treatment of ARDS.

### Bacterial Antigens

In one example, the additional antigen is a bacterial antigen.

Bacterial antigens that can be encoded by the RNA in accordance with the present disclosure will be apparent to the skilled person and include, for example, proteins and peptides from a Neisseria meningitides, Streptococcus pneumoniae, Streptococcus pyogenes, Moraxella catarrhalis, Bordetella pertussis, Burkholderia sp. (e.g., Burkholderia mallei, Burkholderia pseudomallei and Burkholderia cepacia), Staphylococcus aureus, Haemophilus influenzae, Clostridium tetani (Tetanus), Clostridium perfringens, Clostridium botulinum, Corynebacterium diphtheriae (Diphtheria), Pseudomonas aeruginosa, Legionella pneumophila, Coxiella burnetii, Brucella sp. (e.g., B. abortus, B. canis, B. melitensis, B. neotomae, B. ovis, B. suis and B. pinnipediae), Francisella sp. (e.g., F. novicida, F. philomiragia and F. tularensis), Streptococcus agalactiae, Neisseria gonorrhoeae, Chlamydia trachomatis, Treponema pallidum (Syphilis), Haemophilus ducreyi, Enterococcus faecalis, Enterococcus faecium, Helicobacter pylori, Staphylococcus saprophyticus, Yersinia enterocolitica, E. coli, Bacillus anthracis (anthrax), Yersinia pestis (plague), Mycobacterium tuberculosis, Rickettsia, Listeria, Chlamydia pneumoniae, Vibrio cholerae, Salmonella typhi (typhoid fever), Borrelia burgdorferi, Porphyromonas sp, Klebsiella sp.

### Fungal Antigens

In one example, the additional antigen is a fungal antigen.

Fungal antigens that can be encoded by a RNA according to the present disclosure or provided in the form of a polypeptide will be apparent to the skilled person and include, for example, proteins and peptides from Dermatophytes (including Epidermophyton floccusum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleini, Trichophyton tonsurans, Trichophyton verrucosum, T verrucosum var. album, var. discoides, var. ochraceum, Trichophyton violaceum, and/or Trichophyton faviforme), Aspergillus fumigatus, Aspergillus flavus, Aspergillus niger, Aspergillus nidulans, Aspergillus terreus, Aspergillus sydowii, Aspergillus flavatus, Aspergillus glaucus, Blastoschizomyces capitatus, Candida albicans, Candida enolase, Candida tropicalis, Candida glabrata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides immitis, Blastomyces dermatidis, Cryptococcus neoformans, Geotrichum clavatum, Histoplasma capsulatum, Klebsiella pneumoniae, Microsporidia, Encephalitozoon spp., Septata intestinalis and Enterocytozoon bieneusi.

### Protazoan Antigens

In one example, the additional antigen is a protazoan antigen.

Protazoan antigens that can be encoded by a RNA according to the present disclosure, or provided in the form of a polypeptide will be apparent to the skilled person and include, for example, proteins and peptides from Entamoeba histolytica, Giardia lambli, Cryptosporidium parvum, Cyclospora cayatanensis and Toxoplasma.

### Polypeptides

The compositions and vaccines described herein comprise at least one polypeptide antigen selected from the group consisting of a RSV, influenza and a SARS-CoV-2, including those described herein.

As used herein, the term “polypeptide antigen” refers to a polypeptide comprising one or more epitopes (either linear, conformational or both), that elicits an immunological response. Polypeptide antigens include, for example, a naturally-occurring protein, a mutational variant of a naturally-occurring protein (e.g., a protein that has amino acid substitution(s), addition(s), or deletion(s)), a truncated form of a naturally-occurring protein (e.g., an intracellular domain or extracellular domain of a membrane-anchored protein), as well as a fusion protein (a protein that is derived from at least two different naturally occurring proteins or polypeptide chains). In addition, polypeptide antigens also encompass polypeptides that comprise one or more amino acid stereoisomers, derivatives, or analogues. For example, amino acid derivatives include, e.g., chemical modifications of amino acids such as alkylation, acylation, carbamylation, iodination, etc. Amino acid analogues include, e.g., compounds that have the same basic chemical structure as a naturally occurring amino acid, such as homoserine, norleucine, methionine sulfoxide, methionine methyl sulfonium. Polypeptide antigens also encompass polypeptides that are modified post-translationally (such as acetylated, phosphorylated, or glycosylated polypeptides). Therefore, an epitope of a polypeptide antigen is not limited to a peptide. For example, an epitope of a glycosylated polypeptide may be a saccharide group that is attached to the polypeptide chain.

It will be appreciated that the present disclosure is directed to analogs of polypeptide antigens in other organisms and is not limited to the human analog. Homologs can be found in other species by methods known in the art. For example, sequence similarity may be determined by conventional algorithms, which typically allow introduction of a small number of gaps in order to achieve the best fit. In particular, “percent identity” of two polypeptides or two nucleic acid sequences is determined using the algorithm of Karlin and Altschul (Proc. Natl. Acad. Sci. USA 87:2264-2268, 1993). Such an algorithm is incorporated into the BLASTN and BLASTX programs of Altschul et al. (J. Mol. Biol. 215:403-410, 1990). BLAST nucleotide searches may be performed with the BLASTN program to obtain nucleotide sequences homologous to a nucleic acid molecule described herein.

Equally, BLAST protein searches may be performed with the BLASTX program to obtain amino acid sequences that are homologous to a polypeptide described herein. To obtain gapped alignments for comparison purposes, Gapped BLAST is utilized as described in Altschul et al. (Nucleic Acids Res. 25:3389-3402, 1997). When utilizing BLAST and Gapped BLAST programs, the default parameters of the respective programs (e.g., BLASTX and BLASTN) are employed.

In certain embodiments, the polypeptide antigen may be PEGylated for improved systemic half-life and reduced dosage frequency. In an embodiment, PEG may be added to the RSV, influenza and/or a SARS-CoV-2 polypeptide antigen. As such, a composition of the disclosure may comprise RSV, influenza and/or a SARS-CoV-2 polypeptide antigen comprising PEG. In an embodiment, PEG may be selected from the group consisting of PEG-10K, PEG-20K and PEG-40K. Methods of conjugating PEG to a protein are standard in the art. For example, see Kolate et al, Journal of Controlled Release 2014; 192(28): 67-81, which is hereby incorporated by reference in its entirety.

### Polynucleotides

As used herein, the term “polynucleotide” refers a molecular chain of nucleotides chemically bonded by a series of ester linkages between the phosphoryl group of one nucleotide and the hydroxyl group of the sugar in an adjacent nucleotide. In one example, the polynucleotide is a DNA. In one example, the polynucleotide is a RNA, e.g., mRNA. For example, the mRNA is a conventional mRNA (cRNA) or a self-replicating RNA.

As used herein, the term “fragment” refers to a portion of a nucleotide sequence or polypeptide of a reference nucleotide sequence or polypeptide disclosed herein which maintains a defined activity of the full length nucleotide sequence or polypeptide.

By way of non-limiting example, where the conventional mRNA or self-replicating RNA comprises a polynucleotide encoding more than one antigen, wherein said more than one antigen may be expressed by a monocistronic polynucleotide, or each of said antigens may be expressed by polycistronic (or multicistronic) polynucleotides. For example, the antigens may be expressed by a monocistronic polynucleotide or by polycistronic polynucleotides.

As used herein, the term “variant” refers to a nucleotide sequence with one or more substitutions, insertions, deletions and/or other modifications compared to the unmodified sequence. It will be apparent to the skilled person that any variant described herein will have the same or similar expression of the encoded protein. For example, the variant is a functional variant. Exemplary modifications to the nucleotide sequence and/or polypeptide will be apparent to the skilled person and/or described herein.

In one example, a modification is a chemical modification of one or more nucleotide(s) of the nucleotide sequence. For example, at least one naturally occurring nucleotide of the polynucleotide is replaced with a chemically modified nucleotide (e.g. pseudouridine (ψ), and 1-methylpseudouridine (m1ψ)).

In one example, the modification comprises increasing the G/C content of the nucleotide sequence.

In one example, the modification comprises codon optimization of the nucleotide sequence.

In one example, the substitution is a conservative substitution. A skilled person will appreciate that a conservative substitution with reference to a polypeptide involves replacement of an amino acid in the polypeptide with a different amino acid with similar biochemical properties (e.g. charge, hydrophobicity and size). In one example, the substitution is a non-conservative substitution.

As used herein, the term “encode”, “encodes” or “encoding” refers to a region of a polynucleotide capable of undergoing translation into a polypeptide.

A polynucleotide described herein includes DNA and RNA (e.g. mRNA).

### Deoxyribonucleic Acid (DNA)

In one example, the polynucleotide is a DNA (e.g. DNA vector).

It will be apparent to the skilled person that a DNA may further comprise an endonuclease restriction site at the 3′ end of the 3′UTR. The skilled person will appreciate that endonuclease restriction site allows for the insertion of one or more nucleotide sequence(s) (e.g. encoding an antigen of interest, a fragment and/or a variant thereof) without disrupting the remainder of the DNA.

As used herein, the term “restriction endonuclease site” refers to a sequence of DNA that binds to a restriction endonuclease. Typically, the restriction endonuclease site is short sequence (e.g. of approximately 4-8 base pairs) recognised and cleaved by the restriction endonuclease.

As used herein, the term “restriction enzymes” or “restriction endonucleases” refers to a class of enzyme that occur naturally in bacteria and in some viruses. Restriction endonuclease bind specifically to and cleave double-stranded DNA at specific sites within or adjacent to a restriction endonuclease site. Exemplary restriction endonuclease include, for example, BciVI (Bful), Bcul (Spel), EcoRI, Aatll, AgeI (BshTI), Apal, BamHI, BglII, Blpl (Bpu1102I), BsrGI (Bsp1407), Clal (Bsu15I), EcoRI, EcoRV (Eco32I), Eam1104I (EarI), Hindlll, Kpnl, Mlul, Neol, Ndel, Nhel, Notl, Nsil, Mph 1103I), Pstl, Pvul, Pvull, SacI, SalI, ScaI, SpeI, Xbal, Xhol, Sacll (Cfr42I) and Xbal.

In one example, the present disclosure provides a transcribable polynucleotide comprising nucleotide sequences encoding antigens of interest operably linked to a regulatory element such as a SG promoter and an IRES. For example, the polynucleotide is the DNA plasmid comprising the first and second nucleotide sequences.

In one example, the DNA comprises a nucleotide sequence comprising a restriction endonuclease site located 3′ of the 3′UTR. The presence of the restriction endonuclease site located 3′ of the 3′UTR allows for production of a linearised DNA. Linearisation of DNA ensures defined termination of in vitro transcribed DNA to produce mRNA.

### Ribonucleic Acid (RNA)

As used herein, the term “RNA (ribonucleic acid)” refers to a single stranded molecular chain of nucleotides chemically bonded by a series of ester linkages between the phosphoryl group of one nucleotide and the hydroxyl group of the sugar in an adjacent nucleotide. Suitable forms of RNA will be apparent to the skilled person. In one example, the RNA is messenger RNA (mRNA). In one example, the mRNA encoding the antigen is a monocistronic mRNA. For example, the monocistronic mRNA is a conventional mRNA (cRNA) or a self-replicating RNA. In another example, the mRNA encoding the antigen is a multicistronic mRNA. For example, the multicistronic mRNA is a conventional mRNA (cRNA) or a self-replicating RNA.

### Conventional (Non-Replicating) RNA

RNAs suitable for use in accordance with the present disclosure encompass a non-replicating mRNA (also referred to as conventional mRNA (cRNA) or non-amplifying). The skilled person will understand that cRNA may comprise in order from 5′ to 3′: a 5′cap structure, a 5′-UTR, a fragment and/or a variant thereof, a first nucleotide sequence encoding a first antigen of interest, optionally a second nucleotide sequence encoding a second antigen of interest, a 3′-UTR and a 3′tailing sequence (e.g. a polyadenylation signal or one or more poly-A tails). The cRNA may further comprise a translation internal ribosome entry site (e.g. Kozak consensus sequence or IRES) operably linked to the antigen of interest. In an example, the present disclosure provides a monocistronic cRNA. In an example, the present disclosure provides a multicistronic cRNA.

### Self-Replicating RNA

Compositions of the present disclosure encompass a self-replicating RNA (also known as self-amplifying RNA or sa-mRNA). In an example, the RNA is a self-replicating RNA (also known as a replicon). In an example, the RNA is a monocistronic self-replicating RNA. In an example, the RNA is a multicistronic self-replicating RNA.

The skilled person will understand that the self-replicating RNA described herein is based on the genomic RNA of RNA viruses. The RNA should be positive (+)-stranded so that it can be directly translated after delivery to a cell without the need for intervening replication steps (e.g., reverse transcription). Translation of the RNA results in the production of non-structural proteins (NSPs) which combine to form a replicase complex (i.e., an RNA-dependent RNA polymerase). The complex then amplifies the original RNA, producing both antisense and sense transcripts, resulting in production of multiple daughter RNAs which may subsequently be translated and transcribed, enhancing overall protein expression.

In one example, the self-replicating RNA comprises the non-structural proteins of the RNA virus, the 5′ and 3′ untranslated regions (UTRs) and the native subgenomic promoter.

In one example, the self-replicating RNA comprises one or more non-structural proteins of the RNA virus. For example, the RNA comprises at least one or more genes selected from the group consisting of a viral replicase (or viral polymerase), a viral protease, a viral helicase and other non-structural viral proteins. For example, the self-replicating RNA comprises a viral replicase (or viral polymerase).

It will be apparent to the skilled person that RNA suitable for use in the present disclosure may also include a 5′ untranslated region (5′-UTR), a 3′ untranslated region (3′UTR), and/or a coding or translating sequence. In addition, the RNA may comprise a 5′ cap structure, a chain terminating nucleotide, a stem loop (e.g., a histone stem loop), a 3′ tailing sequence (e.g., a polyadenylation signal or one or more polyA tails. In another example, the self-replicating RNA comprises a 5′- and a 3′-end UTR of the RNA virus. It will be apparent to the skilled person that the terms 5′ and a 3′UTR also encompasses the terms 5′ and 3′ conserved sequence elements (CSE). In one example, the self-replicating RNA comprises a 5′- and a 3′-end CSE.

The self-replicating RNA described herein cannot induce production of infectious viral particles. For example, the self-replicating RNA does not comprise viral genes encoding structural proteins necessary for production of viral particles.

In one example, the self-replicating RNA is derived from, or based on, an alphavirus. Suitable alphaviruses will be apparent to the skilled person and/or described herein.

In another example, the self-replicating RNA is derived from, or based on, a virus other than an alphavirus, for example, a positive-stranded RNA virus. Suitable positive-stranded RNA viruses suitable for use in the present disclosure will be apparent to the skilled person and include, for example, a picornavirus, a flavivirus, a rubivirus, a pestivirus, a hepacivirus, a calicivirus, or a coronavirus.

### Alphavirus

In one example, the self-replicating RNA described herein is derived from (or based on) an alphavirus.

Alphaviruses are the sole genus in the Togaviridae family and are an enveloped virus with a positive-sense, single-stranded RNA genome. The skilled person will understand that the alphavirus genome comprises two open reading frames (ORFs), non-structural and structural. The first ORF encodes four non-structural proteins (NSP1, NSP2, NSP3 and NSP4) necessary for transcription and replication of viral RNA. The second encodes three structural proteins: the core nucleocapsid protein C, and the envelope proteins P62 and E1, which associate as a heterodimer. The viral membrane-anchored surface glycoproteins are responsible for receptor recognition and entry into target cells through membrane fusion.

In one example, the self-replicating RNA comprises a viral replicase (or viral polymerase). For example, the viral replicase is an alphavirus replicase, such as an alphavirus protein NSP4.

In one example, the self-replicating RNA does not encode one or more alphavirus structural proteins (e.g., capsid and/or envelope glycoproteins). For example, the self-replicating RNA is unable to produce RNA-containing alphavirus virions (i.e., infectious viral particles).

In one example, the self-replicating RNA comprises a native alphavirus SG promoter. For example, the native alphavirus SG promoter is a minimal SG promoter (i.e., the minimal sequence required for initiation of transcription) and comprises a sequence set forth in SEQ ID NO: 13.

The skilled person will be aware of alphaviruses suitable for use in the present disclosure. Exemplary alphaviruses include, but are not limited to, Venezuelan equine encephalitis virus (VEE; e.g., Trinidad donkey, TC83CR), Semliki Forest virus (SFV), Sindbis virus (SIN), Ross River virus, Western equine encephalitis virus, Eastern equine encephalitis virus, Chikungunya virus, S.A. AR86 virus, Everglades virus, Mucambo virus, Barmah Forest virus, Middelburg virus, Pixuna virus, O'nyong-nyong virus, Getah virus, Sagiyama virus, Bebaru virus, Mayaro virus, Una virus, Aura virus, Whataroa virus, Banbanki virus, Kyzylagach virus, Highlands J virus, Fort Morgan virus, Ndumu virus, and Buggy Creek virus. The term alphavirus may also include chimeric alphaviruses (e.g., as described by Perri et al, (2003) J. Virol. 77(19): 10394-403) that contain genome sequences from more than one alphavirus.

### Regulatory Elements

The present disclosure provides the present disclosure provides an immunogenic composition comprising:
(i) a polynucleotide comprising: a) a first nucleotide sequence encoding an antigen from a respiratory syncytial virus (RSV), influenza and/or SARS-CoV-2; and b) a second nucleotide sequence encoding an antigen from influenza, SARS-CoV-2 and/or RSV operably linked to a regulatory element selected from the group consisting of a SG promoter and an IRES; and
(ii) an antigen polypeptide from a SARS-CoV-2, RSV and/or influenza.
   In this example, the nucleotide sequences are operably linked to a regulatory element selected from a promoter, IRES or Kozak consensus sequence. In one example, the promoter is a subgenomic (SG) promoter.

### Subgenomic Promoter

SG promoters (also known as ‘junction region’ promoters) suitable for use in the present disclosure will be apparent to the skilled person and/or are described herein.

In one example, the SG promoter is derived from or based on an alphavirus SG promoter. For example, the SG promoter is a native alphavirus SG promoter. In one example, the native SG promoter is a minimal SG promoter. For example, the minimal SG promoter is the minimal sequence required for initiation of transcription. In one example, the native SG promoter is an extended SG promoter. For example, the extended SG promoter is a minimal SG promoter extended at the 5′ end with nucleotides occurring in a sequence encoding a non-structural protein (e.g., NSP4) of the RNA virus (e.g., an alphavirus). In one example, the extended SG promoter is a minimal SG promoter extended at the 5′ end with nucleotides occurring in a sequence encoding an alphavirus NSP4.

In one example, the polynucleotide comprises a SG promoter from any alphavirus. For example, the RNA (e.g., cRNA or self-replicating RNA) comprises a SG promoter from any alphavirus.

In one example, the self-replicating RNA comprises a SG promoter from any alphavirus.

In one example, the polynucleotide is a multicistronic mRNA that comprises two or more nucleotide sequences. In one example, the two or more nucleotide sequences are each operably linked to SG promoters. When two or more SG promoters are present in the RNA, the promoters can be the same or different. For example, the two or more SG promoters are derived from the same alphavirus. In another example, the two or more SG promoters are derived from different alphaviruses.

When two or more SG promoters are present in the self-replicating RNA, the promoters can be the same or different. For example, the two or more SG promoters are derived from the same alphavirus. In another example, the two or more SG promoters are derived from different alphaviruses.

### Internal Ribosomal Entry Site (IRES)

IRES suitable for use in the present disclosure will be apparent to the skilled person and/or are described herein.

In one example, the IRES is derived from encephalomyocarditis virus (EMCV). For example, the IRES is a wild-type IRES from EMCV.

In one example, the IRES is derived from a fibroblast growth factor 1A (FGF1A) IRES.

In addition, synthetic IRES elements have been described, which can be designed, according to methods know in the art to mimic the function of naturally occurring IRES elements (see Chappell, S A et al. Proc. Natl Acad. Sci. USA (2000) 97(4): 1536-41).

### Kozak Consensus Sequence

As used herein, the term “Kozak consensus sequence” refers to a nucleotide sequence identified in eukaryotic genes that facilitates the translation of the gene by containing a start codon (also referred to as a translation initiation codon) which is recognised by a ribosome.

Exemplary Kozak consensus sequence are known in the art and/or described herein. In one example, the Kozak consensus sequence is GCCACC. In another example, the Kozak consensus sequence is ACCATGG. In one example, the Kozak consensus sequence is ACCATGG. In another example, the Kozak consensus sequence is ACCATG.

### 5′ Untranslated Region (5′UTR)

In one example, the self-replicating RNA comprises a 5′-UTR of the RNA virus.

As used herein, the term “5′-untranslated region” or “5′-UTR” refers to a non-coding region of an mRNA located at the 5′end of the translation initiation sequence (AUG).

Exemplary 5′-UTRs include, for example, 5′-UTR of haptoglobin (HP), fibrinogen beta chain (FGB), haptoglobin-related protein (HPR), albumin (ALB), complement component 3 (C3), fibrinogen alpha chain (FGA), alpha 6 collagen (Col6A), alpha-1-antitrypsin (SERPINA1), alpha-1-antichymotrypsin (SERPINA3) a fragment and/or a variant thereof.

In one example, the 5′UTR is a 5′UTR of a Venezuelan equine encephalitis virus (VEEV) or modified forms thereof. For example, the 5′UTR comprises a sequence set forth in SEQ ID NO: 25.

In one example, the 5′UTR comprises at least one microRNA binding site, an AU rich element (ARE), a GC-rich element, a stem loop, and combinations thereof.

microRNA Binding Site

As used herein, the term “microRNA binding site” refers to a sequence within a polynucleotide (e.g. within a DNA or RNA transcript) that has sufficient complementarity to all or one region of a miRNA to interact, associate or bind to the microRNA (miRNA).

As used herein, the term “microRNA” or “miRNA” refers to 19-25 nucleotide long non-coding RNAs that bind to the 5′-UTR of polynucleotides and down-regulate gene expression (e.g. by inhibiting translation). The presence of microRNA binding site(s) in the 5′UTR can function to inhibit translation of the 5′-UTR.

Suitable miRNA binding sites for use in the present disclosure will be apparent to the skilled person and/or described herein.

In one example, the miRNA binding site comprises a binding site for tissue specific microRNA or those regulating biological processes. For example, miRNA of the liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-id, miR-149), kidney (miR-192, miR-194, miR-204), and lung epithelial cells (let-7, miR-133, miR-126). For example, microRNA that regulate biological processes such as angiogenesis (miR-132). Further exemplifying miRNA and miRNA binding sites are disclosed in US patent application U.S. Ser. No. 14/043,927.

### AU Rich Element (ARE)

As used herein, the term “AU rich element (ARE)” or “AU rich elements (AREs)” refers to a region of a nucleotide sequence comprising stretches of Adenosine (A) and Uridine (U). Exemplary AREs include, for example, ARE from cytoplasmic myc (c-myc), myoblast determination protein 1 (myoD), c-Jun, Myogenin, granulocyte-macrophage colony-stimulating factor (GM-CSF) and tumour necrosis factor alpha (TNF-α), or a combination thereof.

In one example, the ARE comprises a human antigen R or “HuR” (also known as Elavl1) specific binding site. HuR is known to bind AREs increasing the stability of the mRNA.

### GC-Rich Element

As used herein, the term “GC-rich element” refers to a nucleotide sequence with a high amount of Guanine (G) and/or Cytosine (C) compared to Adenine (A) and Thymine(T)/Uracil(U). The presence of GC-rich elements in a polynucleotide (e.g. mRNA) can stabilise the mRNA.

In one example, the GC-rich element comprises a sequence of 3, or 4, or 5, or 6, or 7, or 8, or 9, or 10, or 11, or 12, or 13, or 14, or 15, or 16, or 17, or 18, or 19, or 20, or 21, or 22, or 23, or 24, or 25, or 26, or 27, or 28, or 29, or 30 nucleotides in length.

In one example, the GC-rich element comprises between 30% and 40%, or 40% and 50%, or 50% and 60%, or 60% and 70% cytosine. For example, the GC-rich element comprises between 30% and 40% cytosine. For example, the GC-rich element comprises between 40% and 50% cytosine. For example, the GC-rich element comprises between 50% and 60% cytosine. For example, the GC-rich element comprises between 60% and 70% cytosine.

In one example, the GC-rich element comprises 30%, or 40%, or 50%, or 60%, or 70% cytosine. For example, the GC-rich element comprise 30% cytosine. For example, the GC-rich element comprises 40% cytosine. For example, the GC-rich element comprises 50% cytosine. For example, the GC-rich element comprises 60% cytosine. For example, the GC-rich element comprises 60% cytosine. For example, the GC-rich element comprises 70% cytosine.

In one example, the GC-rich element is at least 50% cytosine.

In one example, the GC-rich element is at least 60% cytosine.

In one example, the GC-rich element is at least 70% cytosine.

In one example, the GC-rich element comprises a nucleotide sequence CCCCGGCGCC (SEQ ID NO: 22). In another example, the GC-rich element comprises a nucleotide sequence CCCCGGC. In a further example, the GC-rich element comprises a nucleotide sequence GCGCCCCGCGGCGCCCCGCG (SEQ ID NO: 33).

In one example, the GC-rich element comprises a nucleotide sequence set forth in SEQ ID NO: 21 or SEQ ID NO: 22. In one example, the GC-rich element comprises a nucleotide sequence set forth in SEQ ID NO: 21. In another example, the GC-rich element comprises a nucleotide sequence set forth in SEQ ID NO: 22. In a further example, the GC-rich element comprises a nucleotide sequence of CCCCGGC.

### Stem Loop

As used herein, the term “stem loop” refers to a nucleotide sequence comprising an intramolecular base pairing of two neighboured entirely or partially reverse complementary sequences to form a stem-loop. A stem-loop can occur in single-stranded DNA or, more commonly, in RNA. The stem loop can also be referred to as a hairpin or hairpin loop which usually consists of a stem and a terminal loop within a consecutive sequence, wherein the stem is formed by two neighboured entirely or partially reverse complementary sequences separated by a short sequence which builds the loop into a stem-loop structure.

The stability of the paired stem loop is determined by the length, the number of mismatched or bulges it contains, and the nucleotide composition of the paired region.

In one example, a loop of the stem loop is between 3 and 10 nucleotides in length. For example, the loop of the stem loop is between 3 and 8, or 3 and 7, or 3 and 6, or 4 and 5 nucleotides in length.

In one example, the loop of the stem loop is 4 nucleotides in length.

In one example, the stem loop is a histone stem loop. For example, the histone stem loop comprises or consist of a nucleotide sequence set for in SEQ ID NO: 24.

### 3′ Untranslated Region (3′-UTR)

In one example, an RNA described herein comprises a 3′-untranslated region (3′-UTR).

As used herein, the term “3′-UTR” refers to a region of an mRNA located at the 3′end of the translation termination codon (i.e. stop codon).

Exemplary 3′-UTRs include, for example, a 3′-UTR of arachidonate 5-lipoxygenase (ALOX5), alpha I collagen (COL1A1), tyrosine hydroxylase (TH) gene, amino-terminal enhancer of split (AES), human mitochondrial 12S rRNA (mtRNR1), a fragment and/or a variant thereof.

In one example, the 3′UTR is a 3′UTR of a Sindbis virus (SINV) or modified forms thereof. For example, the 3′UTR comprises a sequence set forth in SEQ ID NO: 26.

In one example, the 3′-UTR comprises or consists of a nucleotide sequence derived from a 3′-UTR of an albumin gene. In one example, the 3′-UTR comprises or consists of a nucleotide sequence derived from a 3′-UTR of a vertebrate α-globin gene. For example, the 3′-UTR comprises or consists of a nucleotide sequence derived from a 3′-UTR of a mammalian α-globin gene. For example, the 3′-UTR comprises or consists of a nucleotide sequence derived from a 3′-UTR of a human α-globin gene.

In one example, the 3′-UTR further comprises at least one microRNA binding site, an AU rich element (ARE), a GC-rich element, a triple helix, a stem loop, one or more stop codons or a combination thereof.

### Stop Codon

As used herein, the term “stop codon” refers to a trinucleotide sequence within a mRNA that signals the stop of protein synthesis by a ribosome.

In one example, the polynucleotide comprises at least one stop codon at the 5′end of a 3′-UTR. For example, the stop codon is selected from UAG, UAA, and UGA.

In one example, the polynucleotide comprises two consecutive stop codons comprising a sequence UGAUGA.

In one example, the polynucleotide comprises two consecutive stop codons comprising a sequence UAAUAG.

### 3′ Tailing Sequence

The RNA described herein may comprise one or more 3′ tailing sequences located at the 3′end of the 3′UTR.

As described herein, the term “3′ tailing sequence” or “3′ tailing sequences” refers to a nucleotide sequence (e.g. polyadenylation signal) which induces the addition of non-encoded nucleotides to the 3′end of a mRNA or a nucleotide sequence (e.g. poly-A sequence) located at the 3′ end of a mRNA. A skilled person will appreciate that the 3′tailing sequence and/or products of the 3′tailing sequence in a mRNA functions to stabilise the mRNA and/or prevent the mRNA from degradation.

As used herein, the term “interrupting linker” in reference to a poly-A or poly-C sequence refers to a single nucleotide or nucleotide sequence which are linked to, and interrupt, a stretch of consecutive adenosine or cytosine nucleotides in the poly-A or poly-C sequence. For example, the interrupting linker in a poly-A sequence is a single nucleotide or a nucleotide sequence consisting or comprising a nucleotide other than an adenosine nucleotide. For example, the interrupting linker in a poly-C sequence is a single nucleotide or a nucleotide sequence consisting or comprising a nucleotide other than a cytosine nucleotide.

In one example, the one or more 3′ tailing sequences are selected from the group consisting of a poly-A sequence, polyadenylation signal, a G-quadruplex, a poly-C sequence, a stem loop and combinations thereof.

### Poly-A Sequence

As used herein, the term “polyA sequence” refers to a nucleotide sequence of Adenine (A) located at the 3′end of a mRNA. In the context of the present disclosure, the polyA sequence may be located within the mRNA or DNA (e.g. a DNA plasmid serving as a template for generating the mRNA by transcription of the vector).

Suitable poly-A sequences for use in the present disclosure will be apparent to the skilled person and/or are described herein. In one example, the poly-A sequence comprises consecutive (i.e. one after the other) adenosine nucleotides of any length (e.g. to 10 to 300). For example, the poly-A sequence comprises 36 consecutive adenosine nucleotides. In one example, the poly-A sequence comprises a sequence set forth in SEQ ID NO: 28.

In one example, the poly-A sequence comprises consecutive adenosine nucleotides separated by one or more interrupting linkers. In one example, the poly-A sequence comprises consecutive adenosine nucleotides without an interrupting linker.

### Polyadenylation Signal

As used herein, the term “polyadenylation signal” refers to a nucleotide sequence which induces polyadenylation. Polyadenylation is typically understood to be the addition of a polyA sequence to a RNA (e.g. to a premature mRNA to generate a mature mRNA). The polyadenylation signal may be located within a nucleotide sequence at the 3′-end of the polynucleotide (e.g. mRNA) to be polyadenylated.

Suitable polyadenylation signal for use in the present disclosure will be apparent to the skilled person and/or described herein.

In one example, the polyadenylation signal comprises a hexamer consisting of Adenine and Uracil/Thymidine nucleotides. In one example, the hexamer sequence comprises or consists of AAUAAA.

In one example, the 3′tailing sequence comprises a polyadenylation signal but does not comprise a polyA sequence.

### G-Quadruplex

As used herein, the term “G-quadruplex” or “G4” refers to a nucleotide sequence rich in guanine residues which forms a four stranded secondary structure. For example, the G-quadruplex is a cyclic hydrogen bonded array of four guanine nucleotides formed by G-rich sequences in both DNA and RNA.

In one example, the 3′ tailing sequence comprises a polyA sequence and a G-quadruplex. For example, the 3′ tailing sequence comprises a polyA sequence linked to a G-quadruplex to produce a polyA-G quartet.

### Poly-C Sequence

As used herein, the term “poly-C sequence” refers to a nucleotide sequence of Cytosine (C) located at the 3′end of a mRNA. In the context of the present disclosure, the polyC sequence may be located within the mRNA or DNA (e.g. a DNA plasmid serving as a template for generating the mRNA by transcription of the vector).

Suitable poly-C sequence for use in the present disclosure will be apparent to the skilled person and/or are described herein.

In one example, the one or more 3′ tailing sequences comprises one or more poly-C sequences each comprising between 10 and 300 consecutive cytosine nucleotides. For example, the one or more poly-C sequences each comprises between 10 and 20, or 20 and 30, or 30 and 40, or 40 and 50, or 50 and 60, or 60 and 70, or 70 and 80, or 80 and 90, or 90 and 100, or 100 and 125, or 125 and 150, or 150 and 175, or 175 and 200, or 200 and 225, or 225 and 250, or 250 and 275, or 275 and 300 consecutive cytosine nucleotides. For example, the one or more poly-C sequence each comprises 10, or 20, or 30, or 40, or 50, or 60, or 70, or 80, or 90, or 100, or 125, or 150, or 175, or 200, or 225, or 250, or 275, or 300 consecutive cytosine nucleotides.

In one example, the one or more poly-C sequences is separated by an interrupting linker. For example, the fourth nucleotide sequence comprising the one or more 3′tailing sequences comprises, in order of 5′ to 3′: consecutive cytosine nucleotides, an interrupting linker, and further consecutive cytosine nucleotides.

In one example, the interrupting linker is from 10 to 50, or 50 to 100, or 100 to 150 nucleotides in length. For example, the interrupting linker is 1, or 2, or 3, or 4, or 5, or 6, or 7, or 8, or 9, or 10, or 11, or 12, or 13, or 14, or 15, or 16, or 17, or 18, or 19, or 20, or 25, or 30, or 35, or 40, or 45, or 50, or 55, or 60, or 65, or 70, or 75, or 80, or 85, or 90, or 95, or 100, or 110, or 120, or 130, or 140, or 150 nucleotides in length.

### 5′Cap

In one example, an mRNA according to the present disclosure comprises a 5′terminal cap structure.

As used herein, the term “5′cap structure” refers to a structure at the 5′ terminal end of a mRNA involved in nuclear export and binds a mRNA Cap Binding Protein (CBP). The 5′cap structure is known to stabilise mRNA through association of CBP with poly(A) binding protein to form a mature mRNA. Accordingly, the presence of a 5′cap structure in the mRNA can further increase the stability of the mRNA compared to a mRNA without the 5′cap.

Exemplary 5′cap structure includes, for example, anti-reverse cap analogue (ARCA), N7,2′-O-dimethyl-guanosine (mCAP), inosine, N1-methyl-guanosine, 2′fluoro-guanosine, 7-deaza-guanosine, 8-oxo-guanosine, 2-amino-guanosine, LNA-guanosine, 2-azido-guanosine, N6,2′-O-dimethyladenosine, 7-methylguanosine (m7G), Cap1, and Cap2.

Typically, an endogenous mRNA is 5′capped with a guanosine through a (5)′-ppp-(5)′-triphosphate linkage attached to the 5′terminal nucleotide of the mRNA. The guanosine cap can then be methylated to a 7-methylguanosine (m7G) generating a 7mG(5′)ppp(5′)N,pN2p (Cap0 structure), where N represents the first and second 5′terminal nucleotide of the mRNA. The cap0 structure can be further 2′-O-methylated to produce 7mG(5′)ppp(5′)NlmpNp (Cap1), and/or 7mG(5′)-ppp(5′)NlmpN2mp (Cap2).

In one example, the polynucleotide comprises an endogenous cap.

As used herein, the term “endogenous cap” refers to a 5′cap synthesised in a cell. For example, endogenous cap is a natural 5′cap or a wild-type 5′cap. For example, the endogenous cap is a Cap0, Cap1, or Cap2 structure.

In one example, the polynucleotide comprises an analog of an endogenous cap (also referred to as cap analog).

As used herein, the term “analogue thereof” in the context of an endogenous cap or “cap analog” refers to a synthetic 5′cap. The cap analog can be used to produce 5′capped mRNA in in vitro transcription reactions. Cap analogs may be chemically (i.e. non-ezymatically) or enzymatically synthesized and/or linked to a nucleotide (e.g. 5′terminal nucleotide of an mRNA). Exemplary cap analogs are commercially available and include, for example, 3″-O-Me-m7G(5′)ppp(5′)G, G(5′)ppp(5′)A, G(5′)ppp(5′)G, m7G(5′)ppp(5′)A, m7G(5′)ppp(5′)G (New England BioLabs). In one example, the cap analog is N7,3′-O-dimethyl-guanosine-5′-triphosphate-5′-guanosine (i.e. anti-reverse cap analogue (ARCA)).

In one example, the 5′cap structure is a non-hydrolyzable cap structure. The non-hydrolyzable cap structure can prevent decapping of the mRNA and increase the half-life of the mRNA.

In one example, the non-hydrolyzable cap structure comprises a modified nucleotide selected from a group consisting or a α-thio-guanosine nucleotide, α-methyl-phosphonate, seleno-phosphate, and a combination thereof. In one example, the modified nucleotide is linked to the 5′end of the mRNA through an α-phosphorothiate linkage. Methods of linking the modified nucleotide to the 5′end of the mRNA will be apparent to the skilled person. For example, using a Vaccinia Capping Enzyme (New England Biolabs).

### Modifications

In one example, the polynucleotide comprises one or more modification(s). Typically, modifications are introduced into a polynucleotide (e.g. mRNA) to increase the translation efficiency and/or stability of the polynucleotide. Suitable modifications to the polynucleotide will be apparent to the skilled person and/or described herein.

In one example, the first nucleotide sequence comprising the 5′-UTR and/or the fragment thereof is modified. Modification of the first nucleotide sequences comprising the 5′-UTR and/or the fragment thereof results in a variant of the 5′-UTR and/or the fragment thereof.

In one example, one or more nucleotide sequence(s) of the polynucleotide are codon optimized. Method of codon optimization will be apparent to the skilled person and/or described herein. For example, tools for codon optimization of polynucleotide include, for example, GeneArt GeneOptimizer (Thermofisher®) or GenSmart® (GeneScript®).

In one example, the polynucleotide is modified to increase the amount of Guanine (G) and/or Cytosine (C) in the polynucleotide. The amount of G/C in the polynucleotide (i.e. G/C content) can influence the stability of the polynucleotide. Accordingly, polynucleotide comprising an increased amount of G/C nucleotides can be functionally more stable than polynucleotides containing a large amount of Adenine (A) and Thymine (T) or Uracil (U) nucleotides. The G/C content is increased by substituting A or T nucleotides with G or C nucleotides.

In one example, the G/C content is increased in the nucleotide sequences encoding the antigens of interest. In one example, the G/C content is increased in the nucleotide sequence encoding the antigens of interest and/or the one or more additional nucleotide sequences encoding the one or more antigens of interest. The modification(s) in the nucleotide sequences takes advantage of the ability of substituting codons that contain less favourable combinations of nucleotides (in terms of mRNA stability) with alternative codons encoding the same amino acid, or encoding amino acid(s) of similar chemistry (e.g. conserved amino acid substitution). For example, the G/C content is increased by substituting codons containing A or T nucleotides with codons containing G or C nucleotides that encode for the same amino acid. For example, the G/C content is increased by substituting codons containing A or T nucleotides with codons containing G or C nucleotides that encode for an amino acid of similar chemistry.

In one example, the G/C content is increased in one or more nucleotide sequences of the polynucleotide which do not encode the antigen of interest. For example, the G/C content is increased in the 5′-UTR, the fragment and/or the variant thereof. For example, the G/C content is increased in the 3′-UTR, the fragment and/or the variant thereof.

In one example, the polynucleotide comprises at least one chemically modified nucleotide.

As used herein, the term “chemical modification” or “chemical modified” in the context of a nucleotide refers to a naturally occurring nucleotides (i.e. A, T, C, G, U) which are modified by replacement, insertion or removal of individual or several atoms or atomic groups compared to the naturally occurring nucleotides. In one example, at least one naturally occurring nucleotide of the polynucleotide is replaced with a chemically modified nucleotide. In one example, at least 10%, or 20%, or 30%, or 40%, or 50%, or 60%, or 70%, or 80%, or 90%, or 100% of naturally occurring nucleotides of the polynucleotide is replaced with a chemically modified nucleotides. Suitable chemical modified nucleotides for use in the present disclosure will be apparent to the skilled person and/or described herein. Exemplary chemically modified nucleotides include, for example, N6,2′-O-dimethyl-adenosine (m6Am), 5-methyluridine (m5U), N4-acetylcytidine (ac4C), 2-thiocytidine (s2C), 2-thiouridine (s2U), 5-methylcytidine (m5C), N6-methyladenosine (m6a), pseudouridine (ψ), and 1-methylpseudouridine (m1ψ).

### Pharmaceutical Compositions

The present disclosure provides an immunogenic composition comprising a polynucleotide described herein and a polypeptide antigen described herein.

The present disclosure provides an immunogenic composition comprising a RNA described herein and a polypeptide antigen described herein.

The present disclosure also provides an immunogenic composition comprising a cRNA described herein and a polypeptide antigen described herein.

The present disclosure further provides an immunogenic composition comprising a self-replicating RNA described herein and a polypeptide antigen described herein.

The present disclosure also provides a pharmaceutical composition comprising an immunogenic composition of the present disclosure and a pharmaceutically acceptable carrier.

It will be apparent to the skilled person and/or described herein, that the polynucleotide, RNA, cRNA and/or self-replicating RNA may be present as naked polynucleotides, RNA, cRNA and/or self-replicating RNA, or in combination with lipids, polymers or other delivery system that facilitates entry into the cells.

### Delivery Systems

In one example, the pharmaceutical composition of the present disclosure further comprises a LNP, a polymeric microparticle and an oil-in-water emulsion. For example, the polynucleotide, the cRNA and/or the self-replicating RNA is encapsulated in, bound to or adsorbed on a LNP, a polymeric microparticle, or an oil-in-water emulsion.

### Lipid Nanoparticles

In one example, the pharmaceutical composition of the present disclosure further comprises a LNP.

It will be apparent that the term “lipid nanoparticle” or “LNP” refers to any lipid composition, including, but not limited to, liposomes or vesicles, where an aqueous volume is encapsulated by amphipathic lipid bilayers (e.g., single; unilamellar or multiple; multilamellar) micelle-like lipid nanoparticles having a non-aqueous core and solid lipid nanoparticles, wherein solid lipid nanoparticles lack lipid bilayers.

Lipid nanoparticles suitable for use in the present disclosure will be apparent to the skilled person and/or are described herein. The lipids can have an anionic, cationic or zwitterionic hydrophilic head group.

In one example, the lipid nanoparticle comprises a PEG-lipid, a sterol structural lipid and/or a neutral lipid. In one example, the lipid nanoparticle further comprises a cationic lipid. In one example, the lipid nanoparticle does not comprise a cationic lipid.

In one example, the LNP comprises a PEG-lipid. For example, the PEG-lipid is selected from the group consisting of PEG-c-DMG, PEG-DMG, PEG-DLPE, PEG-DMPE, PEG-DPPC, a PEG-DSPE lipid and combinations thereof.

In one example, the LNP comprises a structural lipid. For example, the structural lipid is selected from the group consisting of cholesterol fecosterol, sitosterol, campesterol, stigmasterol, brassicasterol, ergosterol, tomatidine, tomatine, ursolic acid and alpha-tocopherol and combinations thereof.

In one example, the LNP comprises a neutral lipid. Exemplary phospholipids (anionic or zwitterionic) for use in the present disclosure include, for example, phosphatidylethanolamines, phosphatidylcholines, phosphatidylserines, and phosphatidylglycerols. For example, the neutral lipid is selected from the group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine (DSPE), 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin and combinations thereof.

In one example, the LNP comprises a cationic lipid. Exemplary cationic lipids include, but are not limited to, dioleoyl trimethylammonium propane (DOTAP), 1,2-distearyloxy-N,N-dimethyl-3-aminopropane (DSDMA), 1,2-dioleyloxy-N,Ndimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA), 2,5-bis((9z,12z)-octadeca-9,12,dien-1-yloxyl)benzyl-4-(dimethylamino)butnoate (LKY750). In one example, the phospholipid is 2,5-bis((9z,12z)-octadeca-9,12,dien-1-yloxyl)benzyl-4-(dimethylamino)butanoate (LKY750). Exemplary zwitterionic lipids include, but are not limited to, acyl zwitterionic lipids and ether zwitterionic lipids, such as dipalmitoylphosphatidylcholine (DPPC), dioleoylphosphatidylcholine (DOPC) and dodecylphosphocholine. The lipids can be saturated or unsaturated.

### Polymeric Microparticles

In one example, the pharmaceutical composition of the present disclosure further comprises a polymeric microparticle.

The skilled person will be aware that various polymers can form microparticles to encapsulate or adsorb the polynucleotide, the cRNA and/or the self-replicating RNA described herein. It will be apparent that use of a substantially non-toxic polymer means that particles are safe, and the use of a biodegradable polymer means that the particles can be metabolised after delivery to avoid long-term persistence. Useful polymers are also sterilisable, to assist in the preparation of pharmaceutical grade formulations.

Exemplary non-toxic and biodegradable polymers include, but are not limited to, poly(α-hydroxy acids), polyhydroxy butyric acids, polylactones (including polycaprolactones), polydioxanones, polyvalerolactone, polyorthoesters, polyanhydrides, polycyanoacrylates, tyrosine-derived polycarbonates, polyvinyl-pyrrolidinones or polyester-amides, and combinations thereof.

### Oil-In-Water Cationic Emulsions

In one example, the pharmaceutical composition of the present disclosure further comprises an oil-in-water cationic emulsion.

Suitable oils for use in an oil-in-water emulsion will be apparent to the skilled person and/or are described herein. For example, the emulsion comprises one or more oils derived, for example, from an animal (e.g., fish) or a vegetable source (e.g., nuts, seeds, grains). The skilled person will recognise that biocompatible and biodegradable oils are preferentially used. Exemplary animal oils (i.e., fish oils) include cod liver oil, shark liver oils, and whale oil. Exemplary vegetable oils include peanut oil, coconut oil, olive oil, soybean oil, jojoba oil, safflower oil, cottonseed oil, sunflower seed oil, sesame seed oil, corn oil.

In addition to the oil, the oil-in-water emulsion also comprises a cationic lipid to facilitate formation and stabilisation of the emulsion. Suitable cationic lipids will be apparent to the skilled person and/or are described herein. Exemplary cationic lipids include, but are not limited to, limited to: 1,2-dioleoyloxy-3-(trimethylammonio)propane (DOTAP), 3′-[N—(N′,N′-Dimethylaminoethane)-carbamoyl] Cholesterol (DC Cholesterol), dimethyldioctadecyl-ammonium (DDA), 1,2-Dimyristoyl-3-Trimethyl-AmmoniumPropane (DMTAP), dipalmitoyl[C16:0]trimethyl ammonium propane (DPTAP) and distearoyltrimethylammonium propane (DSTAP).

In some examples, the oil-in-water emulsion also comprises a non-ionic surfactant and/or a zwitterionic surfactant. The skilled person will be aware of surfactants suitable for use in the present disclosure. Exemplary surfactants include, but are not limited to: the polyoxyethylene sorbitan esters surfactants (e.g., polysorbate 20 and polysorbate 80) and copolymers of ethylene oxide (EO), propylene oxide (PO), and/or butylene oxide (BO).

### Pharmaceutically Acceptable Carrier

Suitably, a composition described herein comprises a pharmaceutically acceptable carrier as is understood in the art. Accordingly, one example of the present disclosure provides a composition (e.g., a pharmaceutical composition) comprising the polynucleotide, RNA, cRNA and/or the self-replicating RNA described herein (and any delivery system), and a polypeptide antigen, combined with a pharmaceutically acceptable carrier.

In general terms, by “carrier” is meant a solid or liquid filler, binder, diluent, encapsulating substance, emulsifier, wetting agent, solvent, suspending agent, coating or lubricant that may be safely administered to any subject, e.g., a human. Depending upon the particular route of administration, a variety of acceptable carriers, known in the art may be used, as for example described in Remington's Pharmaceutical Sciences (Mack Publishing Co. N.J. USA, 1991).

The compositions of the present disclosure are useful for parenteral, topical, oral, or local administration, intramuscular administration, aerosol administration, or transdermal administration, for prophylactic or for therapeutic treatment. In one example, the composition is administered parenterally, such as intramuscularly, subcutaneously or intravenously. For example, composition may be administered intramuscularly. In another example, the composition is administered parenterally, such as intramuscularly, subcutaneously or intravenously.

Formulation of a polynucleotide, RNA, cRNA and/or the self-replicating RNA and a polypeptide antigen to be administered in a composition will vary according to the route of administration and formulation (e.g., solution, emulsion, capsule) selected. An appropriate pharmaceutical composition comprising a polynucleotide, RNA, cRNA and/or the self-replicating RNA to be administered can be prepared in a physiologically acceptable carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. A variety of appropriate aqueous carriers are known to the skilled artisan, including water, buffered water, buffered saline, polyols (e.g., glycerol, propylene glycol, liquid polyethylene glycol), dextrose solution and glycine. Intravenous vehicles can include various additives, preservatives, or fluid, nutrient or electrolyte replenishers (See, generally, Remington's Pharmaceutical Science, 16th Edition, Mack, Ed. 1980). The compositions can optionally contain pharmaceutically acceptable auxiliary substances as required to approximate physiological conditions such as pH adjusting and buffering agents and toxicity adjusting agents, for example, sodium acetate, sodium chloride, potassium chloride, calcium chloride and sodium lactate. The polynucleotides, RNA, cRNA and/or the self-replicating RNA can be stored in the liquid stage or can be lyophilized for storage and reconstituted in a suitable carrier prior to use according to art-known lyophilization and reconstitution techniques.

The optimum concentration of the active ingredient(s) in the chosen medium can be determined empirically, according to procedures known to the skilled artisan, and will depend on the ultimate pharmaceutical formulation desired.

Upon formulation, compositions of the present disclosure will be administered in a manner compatible with the dosage formulation and in such amount as is therapeutically/prophylactically effective. The dosage ranges for the administration of the polynucleotides, RNA, cRNA and/or the self-replicating RNA and polypeptide antigen described herein are those large enough to produce the desired effect. For example, the composition comprises an effective amount of the polynucleotides, RNA, cRNA and/or the self-replicating RNA and polypeptide antigen. In one example, the composition comprises a therapeutically effective amount of the self-replicating RNA and polypeptide antigen. In another example, the composition comprises a prophylactically effective amount of the self-replicating RNA and polypeptide antigen. In one example, the composition comprises an effective amount of the RNA and polypeptide antigen. In one example, the composition comprises a therapeutically effective amount of the RNA and polypeptide antigen. In another example, the composition comprises a prophylactically effective amount of the RNA and polypeptide antigen.

The dosage should not be so large as to cause adverse side effects. Generally, the dosage will vary with the age, condition, sex and extent of the disease in the patient and can be determined by one of skill in the art. The dosage can be adjusted by the individual physician in the event of any complication.

Dosage can vary from about 0.1 mg/kg to about 300 mg/kg, e.g., from about 0.2 mg/kg to about 200 mg/kg, such as, from about 0.5 mg/kg to about 20 mg/kg, in one or more dose administrations daily, for one or several days.

In some examples, the polynucleotide, RNA, cRNA or the self-replicating RNA and polypeptide antigen is used at an initial (or loading) dose which is higher than subsequent (maintenance doses). For example, the polynucleotide, RNA, cRNA and/or the self-replicating RNA is used at an initial dose of between about 10 mg/kg to about 30 mg/kg. The polynucleotide, RNA, cRNA or the self-replicating RNA is then used at a maintenance dose of between about 0.0001 mg/kg to about 10 mg/kg. The maintenance doses may be administered every 7-35 days, such as, every 7 or 14 or 28 days.

In some examples, a dose escalation regime is used, in which the polynucleotide, RNA, cRNA or the self-replicating RNA and polypeptide antigen is initially used at a lower dose than used in subsequent doses. This dosage regime is useful in the case of subject's initially suffering adverse events

In the case of a subject that is not adequately responding to treatment, multiple doses in a week may be administered. Alternatively, or in addition, increasing doses may be administered.

A subject may be retreated with the compositions or vaccines of the present disclosure. A subject may be retreated with the compositions by being given more than one exposure or set of doses, such as at least about two exposures of the binding protein, for example, from about 2 to 60 exposures, and more particularly about 2 to 40 exposures, most particularly, about 2 to 20 exposures.

In one example, any retreatment may be given when signs or symptoms of disease return.

In another example, any retreatment may be given at defined intervals. For example, subsequent exposures may be administered at various intervals, such as, for example, about 24-28 weeks or 48-56 weeks or longer. For example, such exposures are administered at intervals each of about 24-26 weeks or about 38-42 weeks, or about 50-54 weeks.

In the case of a subject that is not adequately responding to treatment, multiple doses in a week may be administered. Alternatively, or in addition, increasing doses may be administered.

In another example, for subjects experiencing an adverse reaction, the initial (or loading) dose may be split over numerous days in one week or over numerous consecutive days.

Administration of the compositions or vaccines according to the methods of the present disclosure can be continuous or intermittent, depending, for example, on the recipient's physiological condition, whether the purpose of the administration is therapeutic or prophylactic, and other factors known to skilled practitioners. The administration of the compositions or vaccines may be essentially continuous over a preselected period of time or may be in a series of spaced doses, e.g., either during or after development of a condition.

Adjuvants and/or Additional Components

Pharmaceutical compositions according to the present disclosure can comprise additional adjuvants or immunopotentiating agents. An adjuvant or immunopotentiating agent is a substance that modifies the action of the principal ingredient. Thus, a skilled person will understand that the terms “adjuvant” or “immunopotentiating agent” may be used interchangeably. In an example, an adjuvant or immunopotentiating agent is a substance that enhances a subject's immune response to an antigen. Suitable adjuvants or immunopotentiating agents will be apparent to the skilled person and include, for example, aluminium-containing adjuvants (e.g. amorphous aluminum hydroxyphosphate sulfate, aluminum hydroxide, aluminum phosphate, and potassium aluminum sulfate), AS04, MF59, AS01_{B}, or CpG 1018.

In an example, the immunogenic compositions described herein comprise an adjuvant for enhancing immunogenicity. In an example, the adjuvant is selected from the group consisting of Freund's adjuvant, incomplete Freund's adjuvants, aluminum phosphate, aluminum hydroxide, GMCSP, BCG, MDP compounds, such as thur-MDP and nor-MDP, CGP (MTP-PE), lipid A, monophosphoryl lipid A (MPL), RIBI, MPL, trehalose dimycolate (TDM), Novasomes®, QS21, Quil A (and derivatives and components thereof), calcium phosphate, calcium hydroxide, zinc hydroxide, MHC antigens, PolyI:C, MF59, glycolipid analogs, octodecyl esters of an amino acid, muramyl dipeptides, polyphosphazene, lipoproteins, ISCOM matrix, DC-Chol, ODA, cytokines, and other adjuvants and derivatives thereof. In an example, the adjuvant is MF59. In an example, MF59 is administered at the same time as the administration of a composition or vaccine of the disclosure. In another example, MF59 is administered sequentially to, preceding, or proceeding the administration of a composition or vaccine of the disclosure.

In an example, an adjuvant has one or more of the following effects:
increasing the magnitude or function of the antibody response;
increasing cell mediated immunity;
inducing mucosal immunity; and/or
reducing antigen dose.

In an example, a LNP described herein has the capability of acting as an adjuvant. In another example, where use of an LNP is contemplated, MF59 is capable of restoring a reduced immunogenic effect induced by a LNP when compared to a composition without MF59. This is particularly advantageous where an improved immunogenic effect is desirable.

In one example, compositions according to the disclosure comprise an additional RNA encoding or an additional polypeptide including: (i) one or more antigens; (ii) one or more immunopotentiating agents; (iii) one or more chemoattractants; and/or (iv) one or more targeting molecules.

As used herein, “immunopotentiating agent” means any molecule capable of enhancing an immune response in a subject. Examples of immunopotentiating agents include cytokines, chemokines, and immune potentiators. In example, the one or more immunopotentiating agents are selected from the group consisting of interleukin 12, interleukin 7, interleukin 15, and interleukin 21.

As used herein, “targeting molecule” means any molecule capable of targeting an immune cell surface marker, for example, a dendritic cell or an antigen presenting cell. In one example, the targeting molecule targets a dendritic cell surface marker. In an example, the targeting molecule is selected from the group consisting of DEC-205, Clec-9A, DC-SIGN, CD11c, DCIR2, Dectin-1/2, CD80/86, F4/80, CIRE, mannose, and CD36. In an example, the targeting molecule is DEC-205.

In another example, the targeting molecule is an antibody. In one example, the targeting molecule is a monoclonal antibody.

### Methods of Production

Suitable methods for the production of polynucleotides, RNA, cRNA or the self-replicating RNA according to the present disclosure will be apparent to the skilled person and/or described herein.

In one example, the cRNA is produced using a plasmid DNA. In one example, the self-replicating RNA is produced using a plasmid DNA. The skilled person will understand that plasmid DNA is relatively stable. Briefly, competent bacterial cells (e.g., Escherichia coli) cells are transformed with a DNA plasmid encoding a self-replicating RNA described herein. Individual bacterial colonies are isolated and the resultant plasmid DNA amplified in E. co/i cultures.

In one example, the plasmid DNA is isolated following fermentation. For example, the plasmid DNA is isolated using a commercially available kit (e.g., Maxiprep DNA kit), or other routine methods known to the skilled person. Following isolation, plasmid DNA is linearized by restriction digest (i.e., using a restricting enzyme). Restriction enzymes are removed using methods known in the art, including for example phenol/chloroform extraction and ethanol precipitation.

In one example, mRNA is made by in vitro transcription from a linearized DNA template using an RNA polymerase (e.g., T7 RNA polymerase). Following in vitro transcription, the DNA template is removed by DNase digestion. The skilled person will understand that synthetic mRNA capping is performed to correct mRNA processing and contribute to stabilization of the mRNA. In one example, the mRNA is enzymatically 5′-capped. For example, the 5′ cap is a cap0 structure or a cap1 structure. In one example, the 5′ cap is a cap0 structure, for example, the 5′-cap (i.e., cap0) consists of an inverted 7-methylguanosine connected to the rest of the mRNA via a 5′-5′ triphosphate bridge. In one example, the 5′ cap is a cap1 structure, for example, the 5′-cap (i.e., cap1) consists of the cap0 with an additional methylation of the 2′O position of the initiating nucleotide.

In one example, the mRNA is purified. Various methods for purifying mRNA will be apparent to the skilled person. For example, the mRNA is purified using lithium chloride (LiCl) precipitation. In another example, the mRNA is purified using tangential flow filtration (TFF). Following purification, the mRNA is resuspended in e.g., nuclease-free water.

### Screening Assays

Suitable methods for selecting a polynucleotide, RNA, cRNA or the self-replicating RNA described herein are available to those skilled in the art. Assays may be conducted to assess the efficiency and efficacy of the RNA including, for example, serology and immune responses.

### Polypeptide Expression

In one example, the self-replicating RNA is assessed for expression of the antigen and of interest. In another example, the presence of the polypeptide antigen is detected by suitable means in the art.

For example, antigen expression is detected using antibodies against the antigen of interest. In one example, the number of cells positive for antigen expression is measured by e.g., fluorescence-activated cell sorting (FACS). In another example the mean fluorescence intensity (MFI) is determined using e.g., FACS. In a further example, the specific potency value or the probability of successful transfection per unit mass of RNA is calculated.

### Microneutralization Assay

In one example, the self-replicating RNA (naked and/or formulated) and/or polypeptide antigen is assessed for antibody responses. For example, the self-replicating RNA and/or polypeptide antigen is assessed using a microneutralisation assay. Methods of performing a microneutralization assay will be apparent to the skilled person. In one example, the microneutralization assay is a short form assay. For one example, a virus fluorescent focus-based microneutralization assay is performed. In another example, the microneutralization assay is a long form assay.

### Hemagglutination Inhibition (HAI) Assay

In one example, the self-replicating RNA (naked and/or formulated) is assessed for antibody responses. For example, the self-replicating RNA is assessed using a hemagglutination inhibition (HAI) assay. Methods of performing a HAI assay will be apparent to the skilled person and/or described, for example, in WHO (2011) Manual for the laboratory diagnosis and virological surveillance of influenza: WHO Press, World Health Organization.

### Antigen Specific T Cell Responses

In one example, the self-replicating RNA is assessed for its ability to induce antigen specific T cell responses. Methods of assessing induction of antigen specific T cell responses will be apparent to the skilled person and/or are described herein.

For example, antigen-specific T cell detection is performed on splenic cultures. Briefly, splenocyte cultures are established in T cell medium and cell cultures are either stimulated with antigenic peptides or unstimulated. In one example, antigen-specific T cell responses are determined using flow cytometry.

### Neutralising Assays

The self-replicating RNA and/or polypeptide antigen described herein may be screened in vitro for their ability to bind to an antigen e.g., SARS-CoV-2 S protein RBD and neutralise binding of the S protein RBD to ACE2. Suitable assays will be apparent to the skilled person and include, for example, a Vero microneutralisation assay, a sVNT assay, or a psuedovirus neutralisation assay (using e.g., HEK-293T cells or HeLa-ACE2 cells).

In one example, the neutralization assay is a Vero microneutralization assay. Briefly, SARS-Cov-2 wild-type virus is passaged in Vero cells (i.e., the Vero lineage isolated from kidney epithelial cells extracted from an African green monkey). Serial two-fold dilutions of a test protein are incubated with 100 TCID₅₀ (i.e., median tissue culture infectious dose) of SARS-CoV-2 for 1 hour and residual virus infectivity is assessed in Vero cells; viral cytopathic effect is read, for example, on day 5. The neutralising antibody titre is calculated using the Reed/Muench method as previously described (Houser et al., 2016; Subbarao et al 2004).

In one example, the neutralization assay is a surrogate neutralization test (sVNT). Briefly, the wells of a plate are coated with hACE2 protein in carbonate-bicarbonate coating buffer (e.g., pH 9.6). HRP-conjugated SARS-CoV-2 and HRP-conjugated SARS-CoV-RBD pre-incubated with test proteins is added to the hACE2 at different concentrations and incubated, for example, for 1 h at room temperature. Unbound HRP conjugated antigens are removed by washing. Colorimetric signal is developed on the enzymatic reaction of HRP with chromogenic substrate, e.g., 3,3′,5,5′-tetramethylbenzidine (TMB). In one example, the absorbance reading at 450 nm and 570 nm is acquired.

In one example, the neutralisation is a psuedovirus neutralisation assay. For example, HIV reporter virus pseudotyped with SARS-2-Spike protein is produced by co-transfection of SARS-2-COV-2 spike plasmids together with a viral backbone plasmid (e.g., pDR-NL Δenv FLUC) into e.g., HEK-293T cells. Pseudovirus is harvested post transfection and clarified by filtration. Virus stock titres, reported as Relative Luciferase Units infectious dose (RLU), are calculated by limiting dilution infections in Hela-hACE2 cells measuring luciferase activity as a read-out for viral infection.

### Methods of Treatment or Prevention

The present disclosure provides methods of using the immunogenic composition or the pharmaceutical composition of the present disclosure as a vaccine.

The present disclosure also provides methods of treating or preventing a disease or condition in a subject comprising administering the immunogenic composition or the pharmaceutical composition of the present disclosure. For example, the disease or condition is a respiratory virus infection, such as influenza, a SARS-CoV-2 infection, COVID-19, or respiratory syncytial virus (RSV). In another example, the disease or condition is acute respiratory distress syndrome (ARDS).

### Influenza

Influenza, also known as “the flu”, is an infectious disease caused by an influenza virus. Symptoms can be mild to severe and the most common symptoms include high fever, runny nose, sore throat, muscle and joint pain, headache, coughing, and feeling tired. Symptoms typically begin two days after exposure to the virus and most last less than a week. Complications of influenza may include viral pneumonia, secondary bacterial pneumonia, sinus infections, and worsening of previous health problems such as asthma or heart failure. Viral pneumonia may also lead to acute respiratory distress syndrome (ARDS).

It will be apparent to the skilled person that there are currently four influenza viruses—A, B, C and D. Influenza A virus is the most common flu virus infecting humans, animals, and birds, whilst influenza B virus infection mostly occurs in humans. Infection of influenza C virus does not cause any severe symptom in human or mammals and influenza D, to date, has only infected pigs and cattle.

Thus, in some examples of the present disclosure, the subject has an influenza virus infection. In one example, the subject has influenza. In particular, the influenza is associated with ARDS. In one example, the methods of the present disclosure can be used to treat or prevent ARDS in a subject suffering from an influenza virus infection. In one example, the methods of the present disclosure can be used to treat or prevent ARDS in a subject suffering from influenza.

In an example, the methods described herein comprise the identification of a subject having or suspected of having influenza. In this example, the subject may have one or more of the above symptoms and may be classified as having mild or severe influenza.

### Coronavirus Disease 2019 (COVID-19)

The present disclosure provides, for example, methods of treating or preventing COVID-19. The present disclosure also provides, for example, methods of treating or preventing SARS-CoV-2 infection. In some examples of the present disclosure the subject has a SARS-CoV-2 infection but does not have clinically diagnosed COVID-19.

COVID-19 is an infectious disease caused by SARS-CoV-2. It was first identified in December 2019 in Wuhan, Hubei, China, and has resulted in an ongoing pandemic. Common symptoms include fever, cough, fatigue, shortness of breath, and loss of smell and taste. While the majority of cases result in mild symptoms, some progress to ARDS. The time from exposure to onset of symptoms is typically around five days, but may range from two to fourteen days.

Thus, in some examples, the subject has a SARS-CoV-2 infection. In one example, the subject has COVID-19, for example, severe COVID-19. In particular, severe COVID-19 often results in ARDS. The methods of the present disclosure can be used to treat or prevent ARDS in a subject suffering from severe COVID-19.

In an example, the methods described herein comprise the identification of a subject having or suspected of having SARS-CoV-2. In this example, the subject may have one or more of the above symptoms and may be classified as having mild or severe SARS-CoV-2.

### Acute Respiratory Distress Syndrome (ARDS)

The present disclosure provides, for example, methods of treating or preventing ARDS in a subject.

ARDS is a life-threatening condition characterized by bilateral pulmonary infiltrates, severe hypoxemia, and disruption of the alveolar-capillary membrane barrier (i.e., pulmonary vascular leak), leading to non-cardiogenic pulmonary edema. There is currently no effective pharmacological therapy.

Infectious etiologies, including influenza and coronavirus infection, are leading causes of ARDS. Accordingly, in one example of the present disclosure, the ARDS is associated with an influenza or a coronavirus infection. For example, the ARDS is associated with influenza. In another example, the ARDS is associated with a coronavirus infection, such as a SARS-COV infection. In one example, the ARDS is associated with a SARS-CoV-2 infection.

ARDS is classified according to the Berlin Definition, which includes:
(1) presentation within 1 week of clinical insult or onset of respiratory symptoms;
(2) acute hypoxemic respiratory failure, as determined by a PaO2/FiO2 ratio of 300 mmHg or less on at least 5 cm of continuous positive airway pressure (CPAP) or positive end expiratory pressure (PEEP), where PaO2 is the partial pressure of oxygen in arterial blood and the FiO2 is the fraction of inspired oxygen;
(3) bilateral opacities on lung radiographs not fully explained by effusions, consolidation, or atelectasis; and
(4) edema/respiratory failure not fully explained by cardiac failure or fluid overload.

In one example, the subject has or suffers from ARDS (i.e., the subject satisfies the Berlin definition of ARDS). For example, the subject is in need of treatment (i.e., in need thereof).

In one example, the subject has or suffers from a symptom associated with ARDS. Symptoms associated with ARDS and methods of identifying subjects at risk of developing ARDS will be apparent to the skilled person and/or are described herein. For example, the subject has one or more or all of the following symptoms:
a) a respiratory frequency of greater than 30 breaths per minute;
b) an oxygen saturation (SpO₂) of 93% or less on room air;
c) a ratio of arterial partial pressure of oxygen to fraction of inspired oxygen (PaO₂/FiO₂) of less than 300 mmHg;
d) a SpO₂/FiO₂ ratio of less than 218; and
e) radiographic lung infiltrates in an amount of greater than 50%.

Currently, ARDS is classified as mild, moderate or severe with an associated increased mortality. The severity of ARDS can be categorized according to the Berlin definition as follows:
(i) Mild ARDS: PaO₂/FiO₂ of 200-300 mmHg on at least 5 cm CPAP or PEEP;
(ii) Moderate ARDS: PaO₂/FiO₂ of 100-200 mmHg on at least 5 cm PEEP; and
(iii) Severe ARDS: PaO₂/FiO₂ of less than or equal to 100 mmHg on at least 5 cm PEEP.

In one example, the ARDS is mild ARDS. In another example, the ARDS is moderate ARDS. In a further example, the ARDS is severe ARDS.

In an example, the methods described herein comprise the identification of a subject having or suspected of having ARDS. In this example, the subject may have one or more of the above symptoms and may be classified as having mild or severe ARDS.

The methods of the present disclosure can, in addition to treatment of existing ARDS, be used to prevent the onset of ARDS. Thus, in one example, the subject does not have ARDS.

### Respiratory Syncytial Virus (RSV)

The present disclosure provides, for example, methods of treating or preventing or delaying progress of RSV. RSV is an enveloped non-segmented negative-strand RNA virus in the family Paramyxoviridae, genus Pneumovirus. To infect a host cell, paramyxoviruses such as RSV, like other enveloped viruses such as influenza virus, require fusion of the viral membrane with a host cell's membrane.

In one example, the subject has or suffers from a symptom associated with RSV. Symptoms associated with RSV and methods of identifying subjects at risk of developing RSV will be apparent to the skilled person and/or are described herein. For example, the subject has one or more or all of the following symptoms indicative of mild RSV:
a) congested or runny nose;
b) dry cough;
c) low-grade fever;
d) sore throat;
e) sneezing;
f) headache;
   or in severe cases:
a) short, shallow and rapid breathing;
b) struggling to breathe i.e., chest muscles and skin pull inward with each breath;
c) cough;
d) poor feeding;
e) unusual tiredness (lethargy);
f) irritability.

Thus, in one example, the RSV is mild RSV. In a further example, the RSV is severe RSV.

In an example, the methods described herein comprise the identification of a subject having or suspected of having RSV. In this example, the subject may have one or more of the above symptoms and may be classified as having mild or severe RSV.

The methods of the present disclosure can, in addition to treatment of existing RSV, be used to prevent the onset of RSV. Thus, in one example, the subject does not have RSV.

In another example, there is provided a method of treating, preventing or delaying progress of a respiratory syncytial virus (RSV) in an infant comprising administering an immunogenic composition, pharmaceutical composition or vaccine disclosed herein to the infant.

An infant having or suspected of having RSV may display one or more or all of the following symptoms indicative of RSV:
a) irritability;
b) decreased activity;
c) decreased appetite;
d) apnea;
e) fever;
f) runny nose;
g) cough, which progressed to wheezing or difficulty breathing.

In an example, the methods described herein comprise the identification of an infant having or suspected of having RSV. In this example, the infant may have one or more of the above symptoms.

### Kits

Another example of the disclosure provides kits containing a composition or vaccine of the present disclosure useful for the treatment or prevention of a disease or disorder as described above (e.g., a SARS-CoV-2 infection, COVID-19, RSV, influenza, ARDS).

Another example of the disclosure provides kits containing a composition or vaccine of the present disclosure useful for the treatment or prevention of a disease or disorder as described above.

In one example, the kit comprises (a) a container comprising a composition or vaccine of the disclosure; and (b) a package insert with instructions for treating or preventing a disease or disorder (e.g., influenza, COVID-19 or RSV) in a subject.

In accordance with this example of the disclosure, the package insert is on or associated with the container. Suitable containers include, for example, bottles, vials, syringes, etc. The containers may be formed from a variety of materials such as glass or plastic. The container holds or contains a composition that is effective for a disease or disorder of the disclosure and may have a sterile access port (for example, the container may be an intravenous solution bag or a vial having a stopper pierceable by a hypodermic injection needle). At least one active agent in the composition is the self-replicating RNA, RNA, cRNA or polynucleotide. The label or package insert indicates that the composition is used for treating a subject eligible for treatment, e.g., one having or predisposed to developing influenza, an influenza virus infection, a SARS-CoV-2 infection, COVID-19 and/or RSV, with specific guidance regarding dosing amounts and intervals of treatment and any other medicament being provided. The kit may further comprise an additional container comprising a pharmaceutically acceptable diluent buffer, such as bacteriostatic water for injection (BWFI), phosphate-buffered saline, Ringer's solution, and/or dextrose solution. The kit may further include other materials desirable from a commercial and user standpoint, including other buffers, diluents, filters, needles, and syringes.

The disclosure contains the following non-limiting examples.

### EXAMPLES

### Example 1: Methods

### Generation of the Self-Replicating RNA

DNA templates encoding self-replicating RNAs described herein were produced in competent Escherichia coli cells transformed with a DNA plasmid. Individual bacterial colonies were isolated and the resultant plasmid DNA amplified in E. coli cultures. Following fermentation, the plasmid DNA was isolated using Maxiprep DNA kit and linearized by restriction digest. Restriction enzymes were removed using phenol/chloroform extraction and ethanol precipitation.

mRNA was made by in vitro transcription from the linearized DNA template using a T7 RNA polymerase. Subsequently, the DNA template was removed by DNase digestion. Enzymatic capping was performed with Cap0 to provide functional mRNA. The resultant mRNA was then purified and resuspended in nuclease-free water.

### In Vitro Characterisation of the Compositions

The self-replicating RNAs were assessed for expression of the genes of interest that are expressed in the form of an antigen. Two-fold serial dilutions of unformulated (naked) or LNP-formulated self-amplifying mRNA constructs were either electroporated or transfected into a Baby Hamster Kidney (BHK) cell line. After about 17-19 hrs, cells were harvested and stained for antigen expression using suitable antibodies. The number of cells positive for antigen expression and the mean fluorescence intensities (MFIs) was measured by FACS. Data was analysed to calculate the specific potency values (the probability of successful transfection per unit of mass of RNA) and the MFI generated.

In vitro activity and potency of unformulated RNA and LNPs was determined by FACs based on antigen co-expression and expressed in read-outs such as FACS potentcy, encapsulation efficiency, SAM recovery, size, PDI, Zeta potential, conductivity, concentration and endotoxin levels.

### Antibody Responses

To assess antibody responses, serum was collected at the end of study and tested by microneutralization assays and hemagglutination inhibition assay.

For all serological assays, sera was treated in the same way, for example with Vibrio cholerae neuraminidase, also known as receptor-destroying enzyme (RDE) (Denka Seiken Co. Ltd., Tokyo, Japan) and diluted to a starting dilution of 1:10 with PBS. Sheep serum to H5N1 virus (FDA/CBER Kensington lot nu. H5-Ag-1115) can be used as positive control sera.

### Microneutralization Assays

Microneutralization assays, short and long form was performed in a qualified mammalian cell line (proprietary 33016-PF Madin-Darby Canine Kidney (MDCK)).

### Microneutralization Assay Short Form (MNAssay SF)

Virus fluorescent focus-based microneutralization (FFA MN) assay was performed using an in house developed protocol. RDE treated test mouse samples and positive control sera was heat inactivated, diluted to a starting dilution of 1:40 with PBS, and fourfold serial diluted using the U-Bottom 96 well plate (BD Falcon) in neutralization medium (comprised of minimum essential medium D-MEM (GIBCO), supplemented with 1% BSA (Rockland, BSA-30), 100 U/mL penicillin and 100 μg/mL streptomycin (GIBCO)). A/turkey/Turkey/1/2005 (H5N1) virus was diluted to ~1,000-1,500 fluorescent focus-forming units (FFU)/well (20,000-30,000 FFU/mL) in neutralization medium and added in a 1:1 ratio to diluted serum.

After incubation for 2 h at 37° C., 5% CO₂, plates (Half Area 96 well plate, Corning) containing MDCK 33016-PF cells were inoculated with this mixture and incubated overnight for 16-18 h at 37° C. with 5% CO₂. MDCK 33016-PF cells are seeded as 3.0E4/well (3.0E6/plate) at 6-8 h earlier in the cell growth medium (comprised of D-MEM, supplemented with 10% HyClone fetal bovine serum—FBS (Gibco), 100 U/mL penicillin and 100 μg/mL streptomycin). Following the overnight incubation and prior to immunostaining, cells were fixed with cold mixture of acetone and methanol.

The virus was visualized using separate 1 h incubations at room temperature of monoclonal antibodies specific to the virus proteins of interest and Alexa Fluor 488 Goat Anti-Mouse IgG (H+L) Ab (Invitrogen cat. no. A11001) diluted in PBS buffer containing 0.05% tween-20 (Sigma) and 2% BSA (Fraction V, Calbiochem, 2960, 1194C175). Viral protein was quantified by a CTL Immunospot analyzer (Cellular Technology Limited, Shaker Heights, Cleveland, OH), using a fluorescein isothiocyanate (FITC) fluorescence filter set with excitation and emission wavelengths of 482 and 536 nm. Fluorescent foci were enumerated by use of software Immunospot 7.0.12.1 professional analyzer DC, using a custom analysis module.

### Microneutralization Assay Long Form (MN Assay LF)

MN assay LF was performed using an in house developed protocol. RDE treated test mouse samples and positive control sera were heat inactivated, diluted to a starting dilution of 1:40 with PBS, and twofold serial diluted using the U-Bottom 96 well plate (BD Falcon) in neutralization medium (comprised of the 30% spent growth media (Irvine Scientific) and 70% infective media (protein free media—33016 MDCK PFM; GIBCO) supplemented with 100 U/mL penicillin, 100 μg/mL streptomycin (GIBCO), and 0.33 ug/mL TPCK-trypsin (TPCK treated, Tosyl phenylalanyl chloromethyl ketone, Sigma). A virus of interest was diluted to 100TCID (tissue culture infectious dose) per well in neutralization medium and added in a 1:1 ratio to diluted serum. Serially pre-diluted serum samples were incubated with the virus and allowed to react for 1 h at 37° C., 5% CO2. In the inoculation step, plates (Cell Culture 96-well plate, Costar) containing MDCK 33016-PF cells were seeded at 3.0^{E4}/well (3.0^{E6}/plate) a day before in the antibiotic free cell growth medium (Irvine Scientific) and washed with sterile PBS, then infected with this mixture and incubated for 1 h at 37° C. with 5% CO₂. Infection was stopped by aspiration of antibody/virus mixture and cells washed with sterile PBS, inoculated with neutralizing media (100 ul/well) containing twofold serially diluted antibodies and then incubated for 5 days at 37° C. with 5% CO₂. In the final “read-out” step, detection of virus was performed by quantification of the virus using 0.5% turkey red blood cells (Lampire Biological Laboratories). The absence of infectivity constitutes a positive neutralization reaction and indicates the presence of virus-specific antibodies in the serum sample.

### Hemagglutination Inhibition (HAI) Assay

HAI assays were performed as previously described (WHO (2011) Manual for the laboratory diagnosis and virological surveillance of influenza: WHO Press, World Health Organization, 20 Avenue Appia, 1211 Geneva 27, Switzerland). Briefly, RDE treated test mouse samples and positive control sera were heat inactivated, diluted to a starting dilution of 1:10 with PBS, and twofold serial diluted samples (25 μl) were incubated with equal volumes of viruses (4 hemagglutinating units [HAU]) of A/turkey/Turkey/1/2005 (H5N1) at room temperature (RT) for 30 minutes. Then, an equal volume of 0.5% turkey red blood cells (Lampire Biological Laboratories) was added and incubated at RT for 30 minutes. The HAI titer was expressed as the reciprocal of the highest dilution of the samples inhibiting hemagglutination.

### Effect of the Compositions on Cell-Mediated Immunity

The compositions described herein were assessed for their ability to induce antigen specific T cell responses. Antigen-specific T cell detection was performed on splenic cultures. Briefly, splenocytes are dissociated in dissociation solution (MACS BSA stock 1:20 with autoMACS rinsing solution) and concentrated at 4^{E7} cells/ml. Briefly, splenocyte cultures were established in 96 well plates in T cell medium containing RPMI, NEAA, pen/strep and PME) and cultured at 37° C./5% CO₂. Anti-CD28 (clone 37.51; BD Biosciences #553294) and anti-CD107a (clone #1D4B; Biolegend #121618) are added to each well. Cell cultures are either stimulated or unstimulated. To stimulate cultures NA pep mix (JPT Peptide Technologies GmbH; PM-INFA-NATur), HA pep mix (JPT Peptide Technologies GmbH; PM-INFA-HAIndo) is added. Following 2 hours of stimulation, Golgi Plug (with brefeldin A; BD Biosciences #555029) was added to each well. Cells are incubated at 37° C. for a total of 6 hours after which the cells are transferred to 4° C. and stored overnight.

Antigen-specific T cell responses were determined using flow cytometry. Briefly, Fc block mixture (clone 2.4G2; BD Biosciences #553142) was added to each well, followed by extracellular stain (comprising Brilliant stain buffer plus (BD Biosciences #566385), ICOS BV711 (clone C398.4A; Biolegend #313548), CD44 BUV395 (clone IM7; BD Biosciences #740215), CD3 BV786 (clone 145-2C11; BD Biosciences #564379), CD4 APC-H7 (clone GK1.5, BD Biosciences #560181), CD8 AF700 (clone 53-6.7, BD Biosciences #557959) and staining buffer). Cells were stained with UltraComp eBeads (eBiosciences #01-222-42) according to the manufacturer's protocol and incubated at 4° C. for 30 mins, protected from the light. Cells were washed with staining buffer, centrifuged, resuspended in staining buffer and data acquired using a flow cytometer.

### IgG Subclass

To characterize the type of immune response generated, i.e. Th1 vs Th2 type responses, the S specific IgG1 and IgG2a IgG subclasses were evaluated by ELISA. In addition, the ratio of IgG1/IgG2a antibodies was also assessed.

Assessment of the Effect of Immunization with the Compositions

To evaluate the protective effect of immunization, mice were immunized with compositions described herein at the indicated doses. In homologous antigen experiments Balb/c mice were immunised with either a coformulation of monovalent influenza sa-mRNA vaccine and homologous monovalent cell derived influenza subunit vaccine; or RSV F protein sa-mRNA vaccine and homologous recombinant RSV F protein subunit vaccine. In heterologous antigen experiments Balb/c mice were immunised with either a coformulation of monovalent influenza sa-mRNA vaccine and recombinant RSV F protein subunit vaccine or monovalent influenza sa-mRNA vaccine and recombinant SARS-CoV-2 S protein subunit vaccine. sa-mRNA and protein vaccines alone were included as controls. Antibody response to vaccination were evaluated by measuring neutralizing antibody titers as well as antigen binding IgG and IgG subclasses. Cell-mediated immune responses were measured by flow cytometric analysis for antigen-specific T cells, as outlined above.

### Example 2: Effect of Homologous Influenza Antigen mRNA and Protein Composition in Mice

Balb/c mice were immunised with a coformulation of monovalent influenza sa-mRNA vaccine and homologous monovalent cell derived influenza subunit vaccine according to Table 1.

TABLE 1
Experimental mouse groups

| | | | | Vaccine | |
|---|---|---|---|---|---|
| | Vaccine component | | | component 2 | |
| Mice/group | 1 (protein) | Dose (ug) | Adjuvant | (RNA) | Dose (ug) |
| 10 | Monobulk - H1N1¹ | 10 | MF59 | — | — |
| | Monobulk - H1N1 | 10 | | sa-mRNA - H1N1² | 1.0 |
| | Monobulk - H1N1 | 10 | | sa-mRNA - H1N1 | 0.01 |
| | Monobulk - H1N1 | 10 | | sa-mRNA - H1N1 | 0.001 |
| | Monobulk - H1N1 | 1.0 | | — | |
| | Monobulk - H1N1 | 1.0 | | sa-mRNA - H1N1 | 1.0 |
| | Monobulk - H1N1 | 1.0 | | sa-mRNA - H1N1 | 0.01 |
| | Monobulk - H1N1 | 1.0 | | sa-mRNA - H1N1 | 0.001 |
| | Monobulk - H1N1 | 0.1 | | — | |
| | Monobulk - H1N1 | 0.1 | | sa-mRNA - H1N1 | 1.0 |
| | Monobulk - H1N1 | 0.1 | | sa-mRNA - H1N1 | 0.01 |
| | Monobulk - H1N1 | 0.1 | | sa-mRNA - H1N1 | 0.001 |
| | — | — | — | sa-mRNA - H1N1 | 1.0 |
| | — | — | — | sa-mRNA - H1N1 | 0.01 |
| | — | — | — | sa-mRNA - H1N1 | 0.001 |

¹Monobulk-H1N1: A/Delaware/55/2019^{;}
²sa-mRNA - H1: Monocistronic (A/Delaware/55/2019) # F641.

Mice were subjected to two vaccinations with sa-mRNA formulated in lipid nanoparticles according to the following.

| | | | | | | | | | |
|---|---|---|---|---|---|---|---|---|---|
| | | | Ionizable | FACS | Encapsulation | | | Endotoxin | Osmolality^{b} |
| | LNP | | Cationic | Potency | Efficiency | Size | | Level^{a} | (mmol/kg) |
| Groups | Lot# | Vector | Lipid | (ng⁻¹) | (%) | (nm) | PDI | (EU/mL) | Vac. 1 |

Biophysical characterization of CRL-05 LNPs Vaccination1

| | | | | | | | | | |
|---|---|---|---|---|---|---|---|---|---|
| 2, 3, 4, 6, | TY040 | F602 | LKY750 | 1649 | 87.0 | 123.4 | 0.231 | <1.0 | 212, 240, 240, 261 |
| 7, 8, 10, 11, | | | | | | | | | 286, 284, 263, 290 |
| 12, 13, 14 & 15 | | | | | | | | | 290, 245, 271 & 272 |

| | | |
|---|---|---|
| 1, 5, 9 | N/A | 250, 288, 290 |

Biophysical characterization of CRL-05 LNPs Vaccination2

| | | | | | | | | | |
|---|---|---|---|---|---|---|---|---|---|
| 2, 3, 4, 6, | TY040 | F602 | LKY750 | 1649 | 87.0 | 123.4 | 0.231 | <1.0 | 213, 241, 240, 259 |
| 7, 8, 10, 11, | | | | | | | | | 284, 283, 265, 291 |
| 12, 13, 14 & 15 | | | | | | | | | 289, 246, 270 & 270 |

| | | |
|---|---|---|
| 1, 5, 9 | N/A | 243, 285, 292 |

^{a}Endotoxin level measured from original stock
^{b}Osmolality measured from final vaccine sample

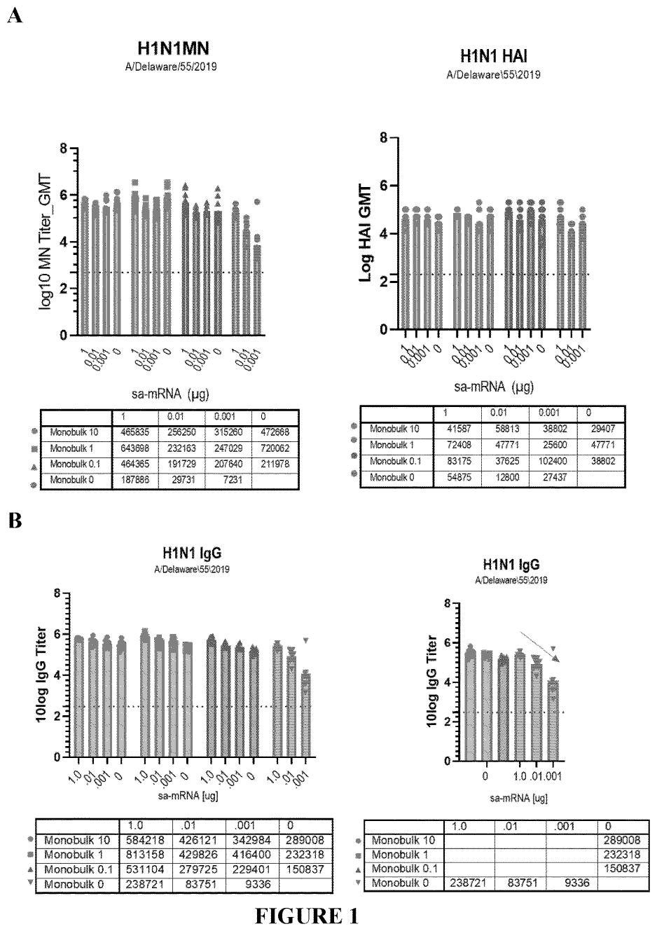

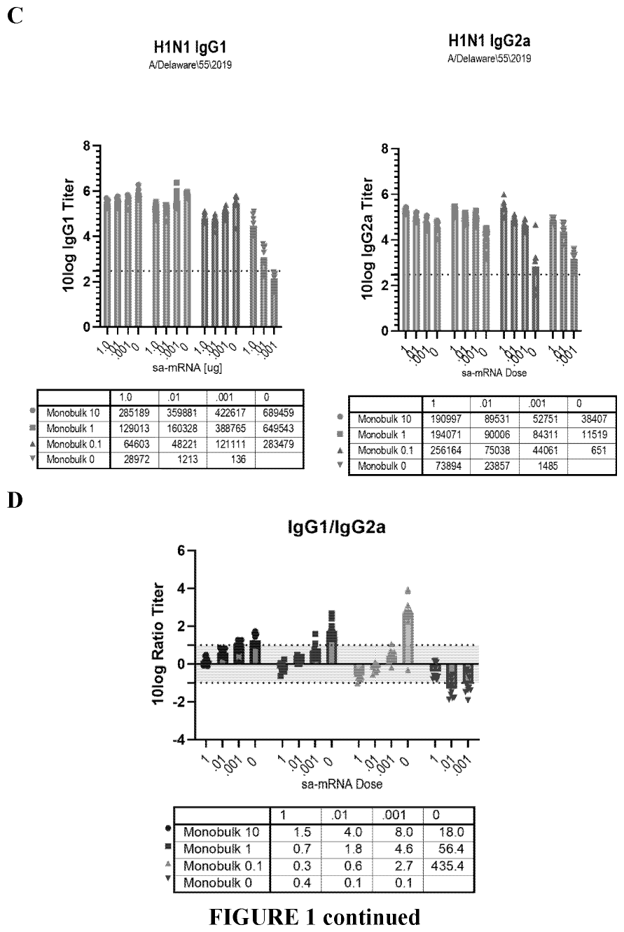

As shown in FIG. 1A, the addition of sa-mRNA to the protein based vaccine did not have a significant effect on neutralising titers at day 42. Adding the sa-mRNA to the protein based vaccine did not significantly increase total IgG titers (FIG. 1B). There was however a trend for the sa-mRNA to effect IgG subclass as shown in FIG. 1C.

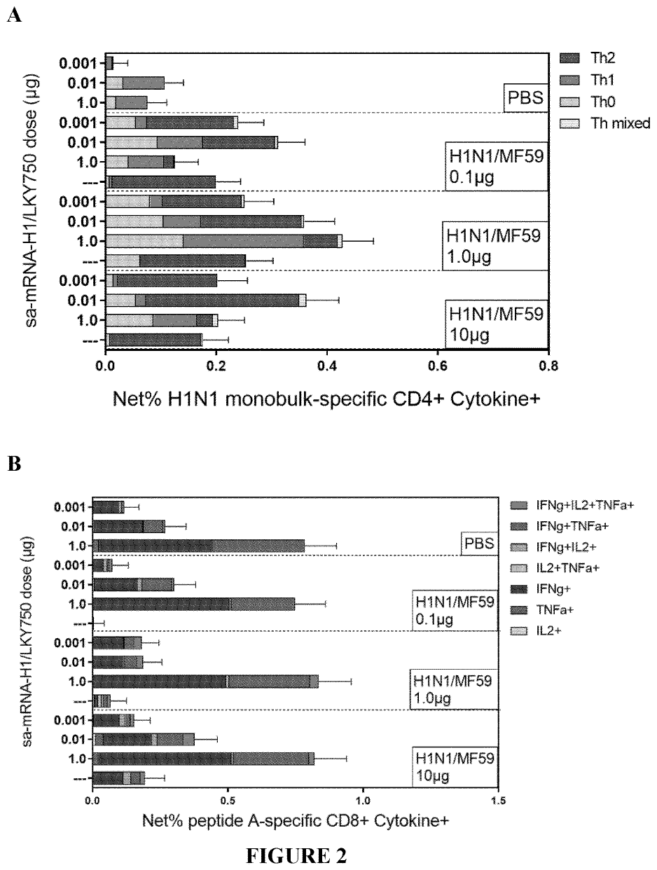

In the presence of a high dose of sa-mRNA, the Th response of the adjuvanted protein subunit vaccine was modified to a Th1 response (FIG. 1D). FIGS. 2A and 2B show the proportions of CD4 and CD8 T cell cytokines at day 42. FIG. 2A and Table 2 demonstrate CD4 T cells having a predominant Th1 phenotype when mice were vaccinated with a sa-mRNA formulated in a LNP (LKY750), and a predominant Th2 phenotype when mice were vaccinated with protein based vaccine adjuvanted with MF59. However, with increasing doses of sa-mRNA formulated in a LNP, the response is skewed toward a Th1 response. FIG. 2B demonstrates that the CD8 T cell response is predominantly IFNg+. An equivalent CD8 T cell cytokine response was observed when the sa-mRNA-formulated in a LNP was combined with PBS or any dose of H1N1/MF59. The CD8 T cell cytokine response was sa-mRNA dose dependent.

TABLE 2
CD4 T-cell cytokine summary

| H1N1/MF59 | sa-mRNA/LKY750 | % Th1:% Th2 |
|---|---|---|
| (μg) | (μg) | Ratio |
| 10 | 0 | 0.00 |
| | 1.0 | 2.78 |
| | 0.01 | 0.07 |
| | 0.001 | 0.05 |
| 1 | 0 | 0.00 |
| | 1.0 | 3.61 |
| | 0.01 | 0.38 |
| | 0.001 | 0.16 |
| 0.1 | 0 | 0.03 |
| | 1.0 | 3.61 |
| | 0.01 | 0.64 |
| | 0.001 | 0.13 |
| 0 | 1.0 | zero Th2 response |
| | 0.01 | |
| | 0.001 | |

### Example 3: Effect of Homologous RSV Antigen mRNA and Protein Composition in Mice

BALB/c mice were immunised with a coformulation of monovalent RSV sa-mRNA vaccine and homologous monovalent cell derived RSV subunit vaccine according to Table 3.

TABLE 3
Experimental mouse groups

| | | Pre F protein | |
|---|---|---|---|
| Group | Mice/Group | (μg) + MF59 | sa-mRNA-Pre-F (μg) |
| 1 | 10 | 10 | — |
| 2 | | 10 | 1.0 |
| 3 | | 10 | 0.01 |
| 4 | | 10 | 0.001 |
| 5 | | 1.0 | — |
| 6 | | 1.0 | 1.0 |
| 7 | | 1.0 | 0.01 |
| 8 | | 1.0 | 0.001 |
| 9 | | 0.1 | — |
| 10 | | 0.1 | 1.0 |
| 11 | | 0.1 | 0.01 |
| 12 | | 0.1 | 0.001 |
| 13 | | — | 1.0 |
| 14 | | — | 0.01 |
| 15 | | — | 0.001 |

Mice were subjected to two vaccinations with sa-mRNA formulated in lipid nanoparticles according to the following.

| | | | Ionizable | FACS | Encapsulation | | | Endotoxin | Osmolality^{b} |
|---|---|---|---|---|---|---|---|---|---|
| | LNP | | Cationic | Potency | Efficiency | Size | | Level^{a} | (mmol/kg) |
| Groups | Lot# | Vector | Lipid | (ng⁻¹) | (%) | (nm) | PDI | (EU/mL) | Vac. 1 |
| 2, 3, 4, 6, | JG095 | R003 | SL02 | 6012 | 87.0 | 96.3 | 0.076 | <1.0 | 219, 264, 265, 243 |
| 7, 8, 10, 11, | | | | | | | | | 288, 287, 249, 293 |
| 12, 13, 14 & 15 | | | | | | | | | 292, 228, 270 & 276 |
| 1, 5, 9 | | N/A | | | | | | | 266, 289, 293 |
| 2, 3, 4, 6, | JG095 | R003 | SL02 | 6012 | 87.0 | 96.3 | 0.076 | <1.0 | 225, 267, 268, 246 |
| 7, 8, 10, 11, | | | | | | | | | 293, 292, 253, 293 |
| 12, 13, 14 & 15 | | | | | | | | | 292, 229, 273 & 273 |

| | | |
|---|---|---|
| 1, 5, 9 | N/A | 273, 291, 294 |

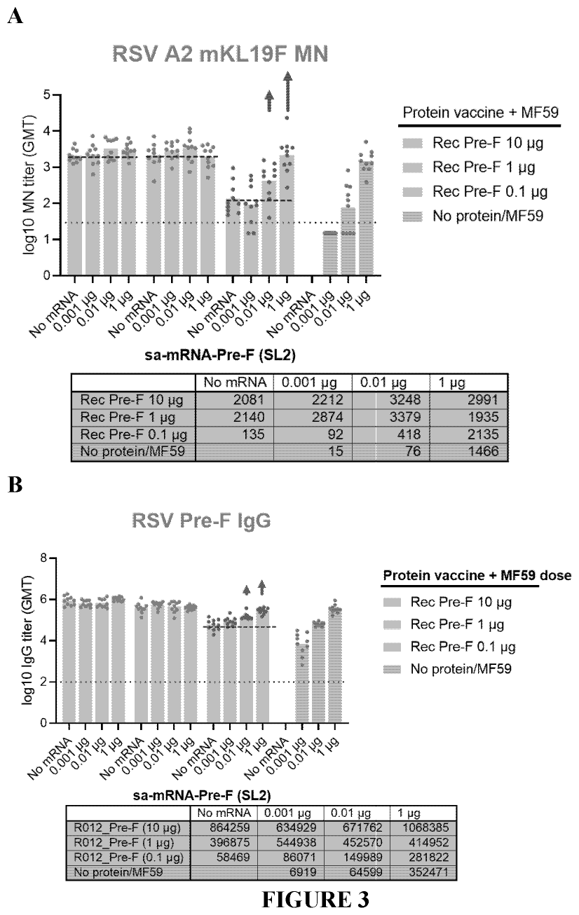

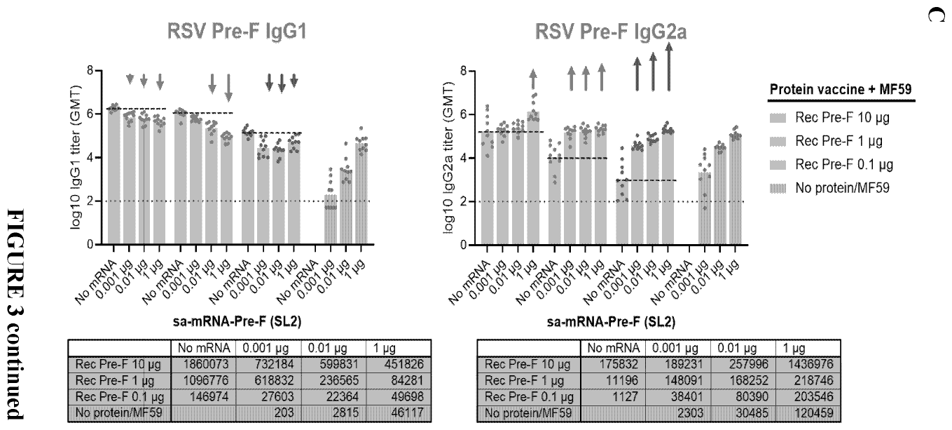

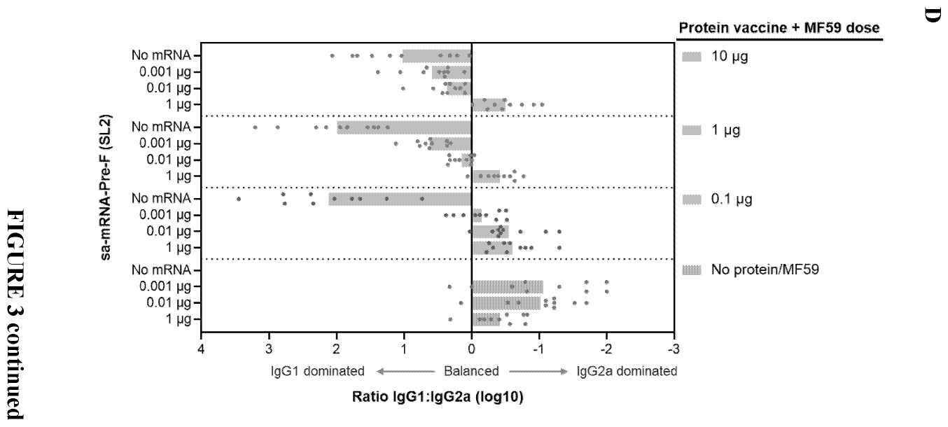

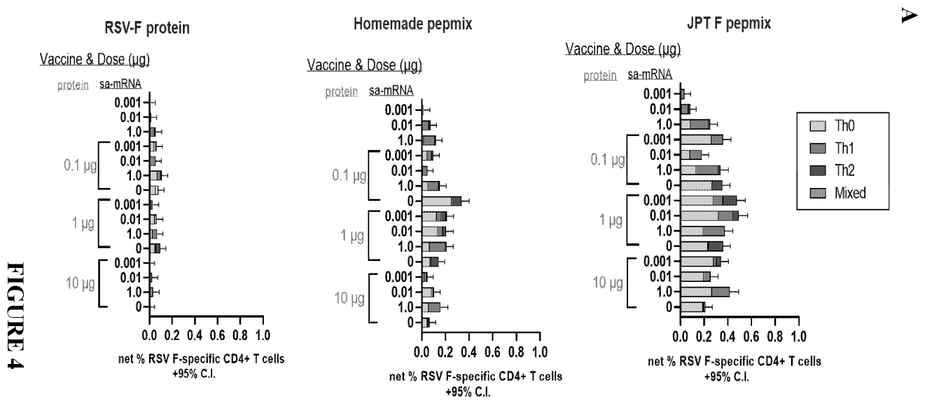

As shown in FIG. 3A, the addition of sa-mRNA to the protein based vaccine enhances neutralising titers at day 42 at lower doses of protein based vaccine. Similarly, adding the sa-mRNA to the protein based vaccine increased total IgG titers at lower doses of protein based vaccine (FIG. 3B). The administration of the sa-mRNA also reduced anti-Pre-F IgG1 subclass and increased IgG2a subclass, as shown in FIGS. 3C and 3D. FIG. 3D demonstrates that the sa-mRNA vaccine reduced anti-Pre-F IgG1:IgG2a ratio in a dose dependent manner and the effect was stronger at lower doses of protein.

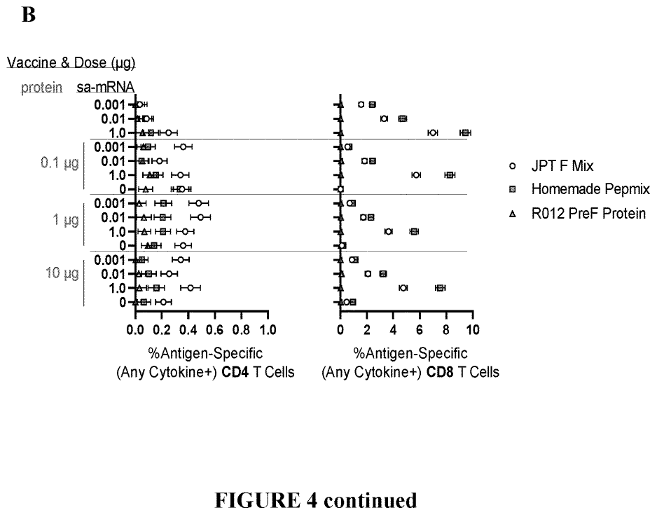

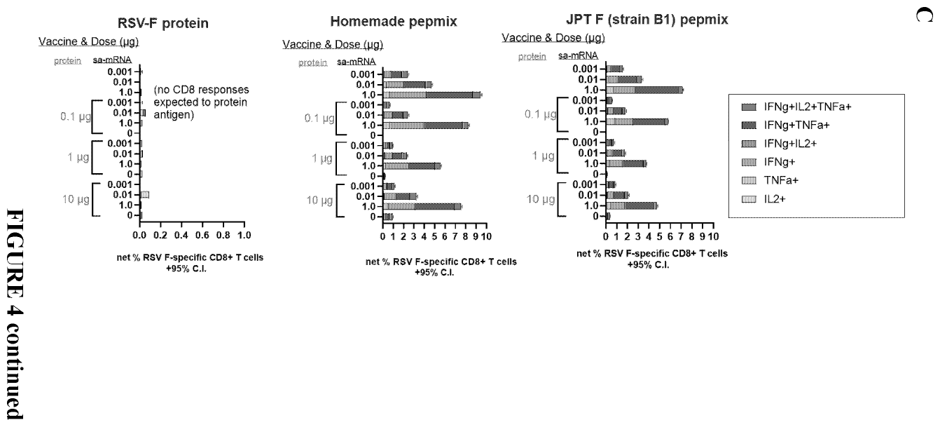

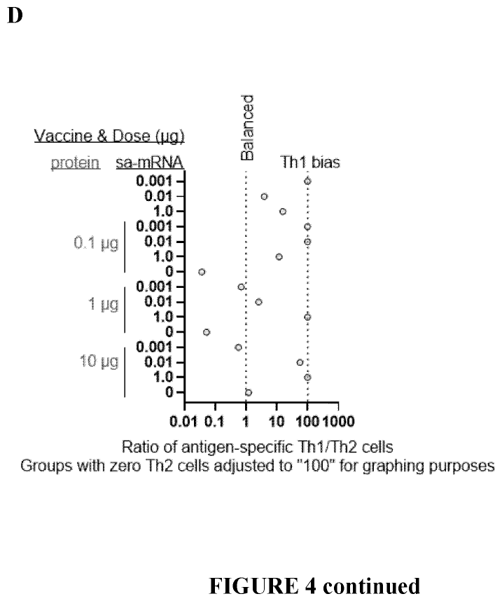

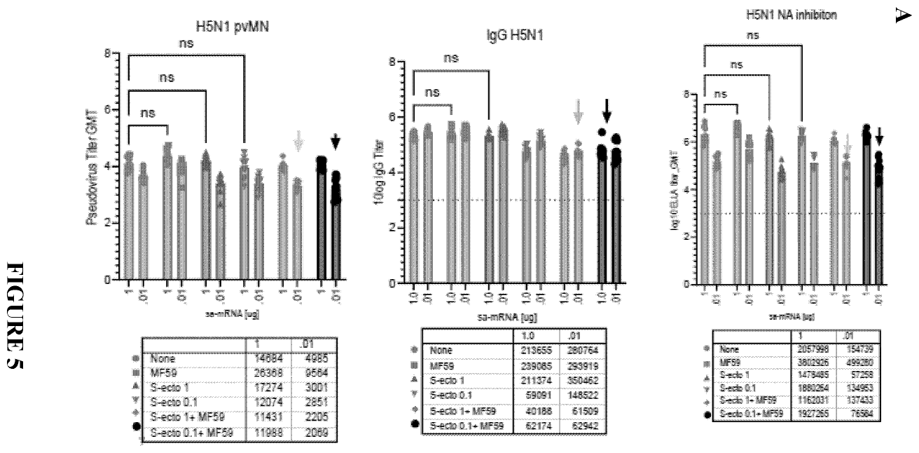

Whilst peptides used in the literature stimulated CD8 T cells, they did not provide an insight into the magnitudes of CD4 T cell responses. For this purpose, T cells were stimulated with a homemade peptide mix, or a “JPT peptide mix” which includes RSV F from strain B1, and RSV pre F-R012 recombinant protein. This peptide mix includes the following peptides: FYQSTCSAV (SEQ ID NO: 34; F32-40); WYTSVITI (SEQ ID NO: 35; F52-59); KYKNAVTEL (SEQ ID NO: 36; F85-92); EVNKIKSAL (SEQ ID NO: 37; F163-171); IKSALLSTN (SEQ ID NO: 38; F167-175) and TYMLTNSEL (SEQ ID NO: 39; F249-258). As shown in FIG. 4A, in the presence of the sa-mRNA alone, a Th1/Th0 response is induced, whilst in the presence of adjuvanted protein alone, a Th2/Th0 response is induced. FIG. 4B demonstrates that the JPT peptide mix is a better CD4 stimulus but the homemade peptide mix is a better CD8 stimulus. Consistently, FIG. 4C demonstrates that magnitude of CD8 responses are relatively high but as expected the adjuvanted protein based vaccine did not induce a strong CD8 response. The results demonstrate that the protein based vaccine does not strongly modulate the CD8 response to the sa-mRNA based vaccine. FIG. 4D demonstrates that T helper cells have a Th2 phenotype in the presence of protein based vaccine and a Th1 phenotype in the presence of sa-mRNA vaccine alone. The data also demonstrate a Th1 phenotype in the presence of both the sa-mRNA vaccine and protein based vaccine, in a dose dependent manner.

### Example 4: Effect of Heterologous Antigen mRNA and Protein Composition in Mice

BALB/c mice were immunised with a coformulation of monovalent H5N1 sa-mRNA vaccine and heterologous monovalent cell derived Spike-ecto protein subunit vaccine according to Table 4.

TABLE 4
Experimental mouse groups

| | | | Dose per | | Dose per | |
|---|---|---|---|---|---|---|
| Group | Mice/Group | Component #1 | animal (ug) | Component #2 | animal (ug) | ID |
| 1 | 10 | Sa-mRNA H5N1 | 1 | na | na | A |
| 2 | | (F602) | 1 | MF59 | [symbol] | B |
| 3 | | | 1 | S-ecto | 1 | C |
| 4 | | | 1 | S-ecto | 0.1 | D |
| 5 | | | 1 | S-ecto + MF59 [symbol] | 1 | E |
| 6 | | | 1 | S-ecto + MF59 1x | 0.1 | F |
| 7 | | | 0.01 | na | na | G |
| 8 | | | 0.01 | MF59 | [symbol] | H |
| 9 | | | 0.01 | S-ecto | 1 | I |
| 10 | | | 0.01 | S-ecto | 0.1 | J |
| 11 | | | 0.01 | S-ecto + MF59 [symbol] | 1 | K |
| 12 | | | 0.01 | S-ecto + MF59 [symbol] | 0.1 | L |
| 13 | | S-ecto Omicron [symbol] | 1 | na | na | M |
| 14 | | ( [symbol] ) | 1 | MF59 | [symbol] | N |
| 15 | | | 1 | empty LNP | [symbol] RNA 1.0 μg | O |
| 16 | | | 1 | Unformulated | 1 | P |
| | | | | RNA (F602) | | |
| 17 | | | 0.1 | na | na | Q |
| 18 | | | 0.1 | MF59 | [symbol] | R |
| 19 | | | 0.1 | empty LNP | [symbol] RNA 1.0 μg | S |
| 20 | | | 0.1 | Unformulated | 1 | T |
| | | | | RNA (F602) | | |

[symbol]

indicates data missing or illegible when filed

Mice were subjected to two vaccinations with sa-mRNA formulated in lipid nanoparticles according to the following.

Biophysical cheracterization of LNP in formulation

| | | | | | | | | | | sa- | Endo- | Vac. 1 | Vac. 2 |
|---|---|---|---|---|---|---|---|---|---|---|---|---|---|
| | | | | | | Encap- | | | | mRNA | toxin | Osmo- | Osmo- |
| | | LNP | | sa- | FACS | sulation | | | | Concen- | Level [symbol] | lality^{b} | lality^{b} |
| | | FD | LNP | mRNA | Potency | Effi- | | Size | | tration | (EU/ | (mmol/ | (mmol/ |
| Group # | Vacc. | Lot# | Type | vector | (ng − 1) | ciency(%) | Recovery | (nm) | PDI | (ug/mL) | mL) | kg) | kg) |
| Group #1 | H5N1 LNP | CY007 | NP082 | F602 | 2487 | 94.3 | 41.9 | 103.1 | 0.209 | 55 | <1 | 231 | 227 |
| Group #2 | H5N1 LNP + | | | | | | | | | | | 247 | 245 |
| | MF59 | | | | | | | | | | | | |
| Group #3 | H5N1 LNP + | | | | | | | | | | | 224 | 253 |
| | S-ecto | | | | | | | | | | | | |
| Group #4 | HSN1 LNP + | | | | | | | | | | | 224 | 245 |
| | S-ecto | | | | | | | | | | | | |
| Group #5 | H5N1 LNP + | | | | | | | | | | | 243 | 267 |
| | S-ecto + | | | | | | | | | | | | |
| | MF59 | | | | | | | | | | | | |
| Group #6 | H5N1 LNP + | | | | | | | | | | | 247 | 268 |
| | S-ecto + | | | | | | | | | | | | |
| | MF59 | | | | | | | | | | | | |
| Group #7 | H5N1 LNP | | | | | | | | | | | 274 | 304 |
| Group #8 | H5N1 LNP | | | | | | | | | | | 294 | 319 |
| Group #9 | H5N1 LNP + | | | | | | | | | | | 272 | 296 |
| | S-ecto | | | | | | | | | | | | |
| Group #10 | HSN1 LNP + | | | | | | | | | | | 273 | 288 |
| | S-ecto | | | | | | | | | | | | |
| Group #11 | H5N1 LNP + | | | | | | | | | | | 296 | 312 |
| | S-ecto + | | | | | | | | | | | | |
| | MF59 | | | | | | | | | | | | |
| Group #12 | H5N1 LNP + | | | | | | | | | | | 296 | 313 |
| | S-ecto + | | | | | | | | | | | | |
| | MF59 | | | | | | | | | | | | |
| Group #13 | S-ecto | | | | | | | | | | | 275 | 292 |
| Group #14 | S-ecto + | | | | | | | | | | | 295 | 314 |
| | MF59 | | | | | | | | | | | | |
| Group #15 | S-ecto + | CY010 | Empty | — | — | — | 41.5 | 98.11 | 0.205 | 100 | <1 | 248 | 263 |
| | empty LNP | | | | | | | | | | | | |
| Group #16 | S-ecto + | | | | | | | | | | | 272 | 283 |
| | sa-mRNA | | | | | | | | | | | | |
| Group #17 | S-ecto | | | | | | | | | | | 273 | 291 |
| Group #18 | S-acto + | | | | | | | | | | | 301 | 305 |
| | MF59 | | | | | | | | | | | | |
| Group #19 | S-ecto + | CY010 | Empty | — | — | — | 41.5 | 98.11 | 0.205 | 100 | <1 | 248 | 257 |
| | empty LNP | | | | | | | | | | | | |
| Group #20 | S-ecto + | | | | | | | | | | | 265 | 282 |
| | sa-mRNA | | | | | | | | | | | | |

[symbol]

indicates data missing or illegible when filed

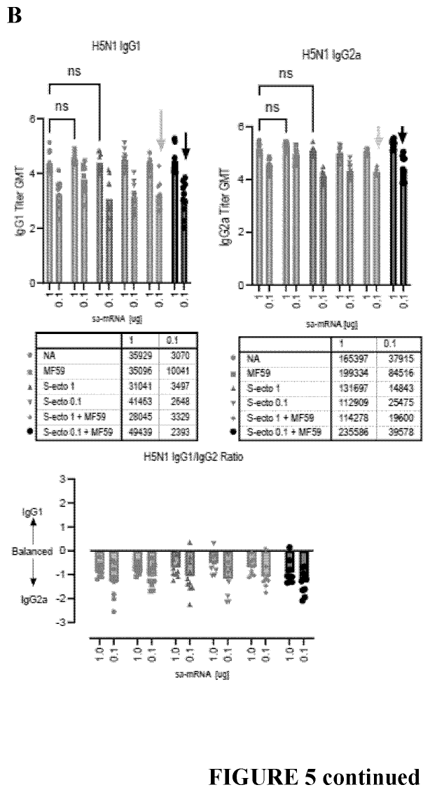

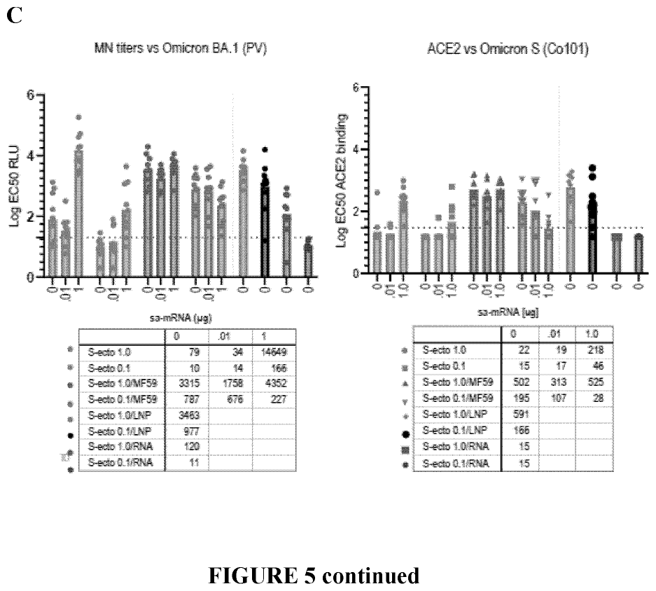

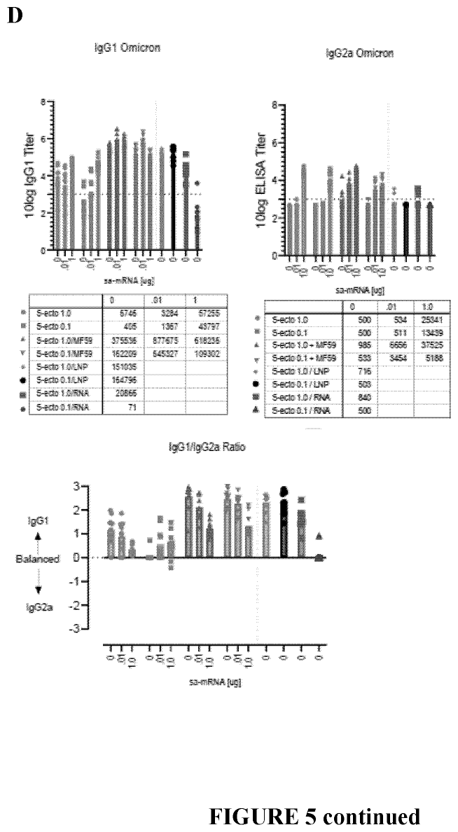

As shown in FIG. 5A, whilst the protein-based vaccine did not effect neutralising titers, the addition of sa-mRNA to the protein based vaccine showed a trend for reduced titers at lower doses. There was also no effect on IgG subclass as shown in FIG. 5B, although high doses of sa-mRNA boosted levels of IgG2a demonstrating a Th1 predominant response. As shown in FIG. 5C, a high dose of sa-mRNA enhanced the unadjuvanted protein-based vaccine response as illustrated through Omicron MN titer and ACE2-binding. Moreover, the immunogenicity achieved by using MF59 could also be achieved by delivery of the LNP. FIG. 5D demonstrates a predominant IgG1 Omicron Th1 response.

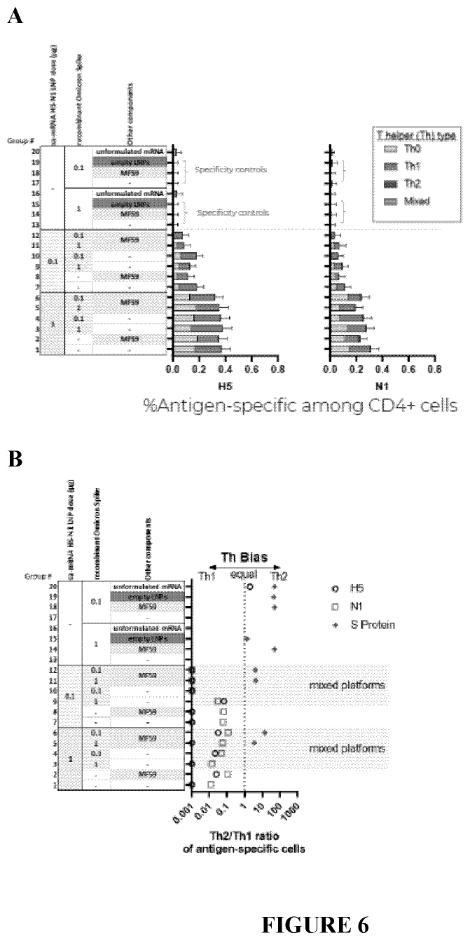

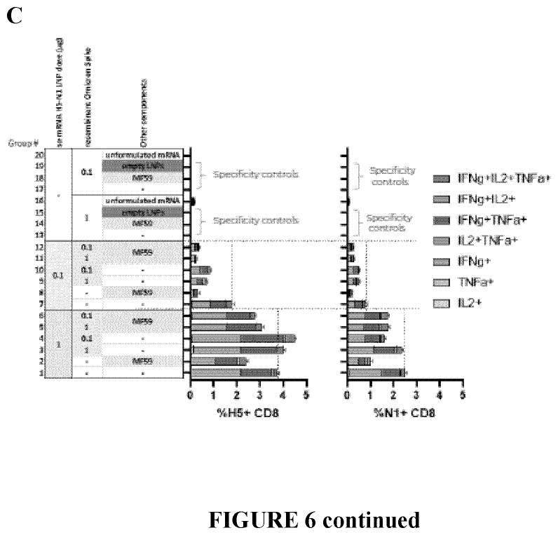

Moreover, in the presence of a high dose of sa-mRNA, a predominant Th1 response was observed (FIG. 6A). FIG. 6B demonstrates that T helper bias is towards Th1 whilst a Th2 predominant response is present in response to the protein based vaccine. FIG. 6B demonstrates that the CD8 T cell response is predominantly IFNg+. An equivalent CD8 T cell cytokine response was observed when the sa-mRNA-formulated in a LNP was combined with PBS or any dose of H1N1/MF59. The CD8 T cell cytokine response was sa-mRNA dose dependent.

### Example 5: Effect of Heterologous Antigen mRNA and Protein Composition on Immunogenicity in Mice

BALB/c mice were immunised with a coformulation of monovalent H1N1 sa-mRNA vaccine and heterologous monovalent cell derived RSV Pre F protein subunit vaccine according to Table 5.

TABLE 5
Experimental mouse groups

| | | sa-mRNA-H1- | |
|---|---|---|---|
| Group | Mice / Group | N1 (μg) F628 | Component #2 |
| 1 | 10 | 1 | — |
| 2 | | 1 | MF59 (1x) |
| 3 | | 1 | RSV Pre F (1 μg) |
| 4 | | 1 | RSV Pre F (0.1 μg) |
| 5 | | 1 | RSV Pre F (1 μg) + MF59 |
| 6 | | 1 | RSV Pre F (0.1 μg) + MF59 |
| 7 | 10 | 0.01 | — |
| 8 | | 0.01 | MF59 (1x) |
| 9 | | 0.01 | RSV Pre F (1 μg) |
| 10 | | 0.01 | RSV Pre F (0.1 μg) |
| 11 | | 0.01 | RSV Pre F (1 μg) + MF59 |
| 12 | | 0.01 | RSV Pre F (0.1 μg) + MF59 |

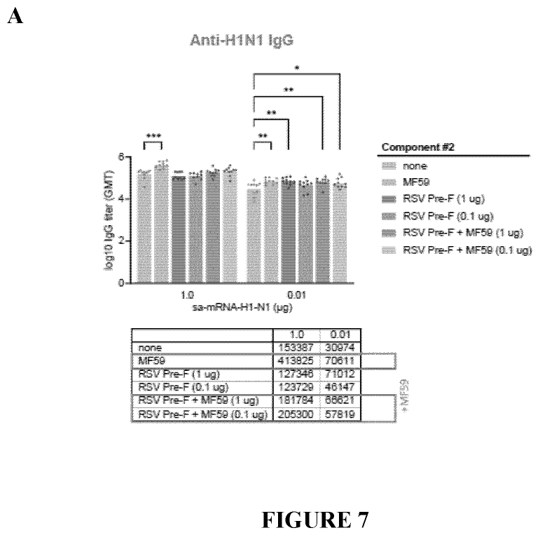

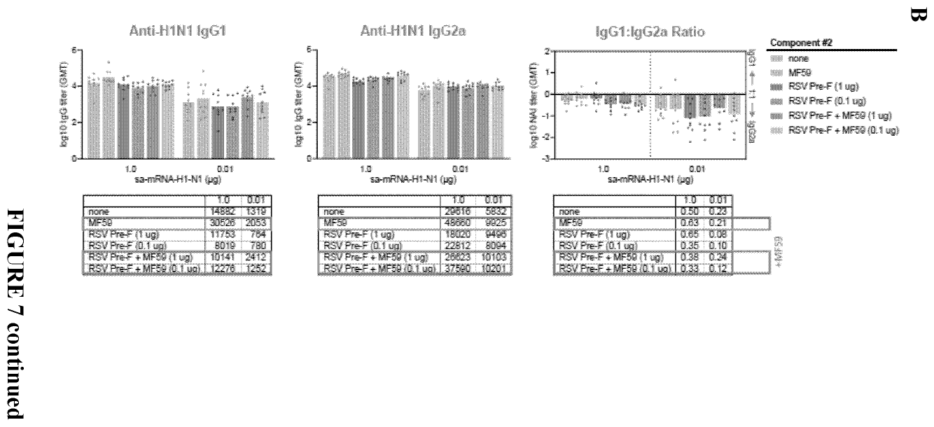

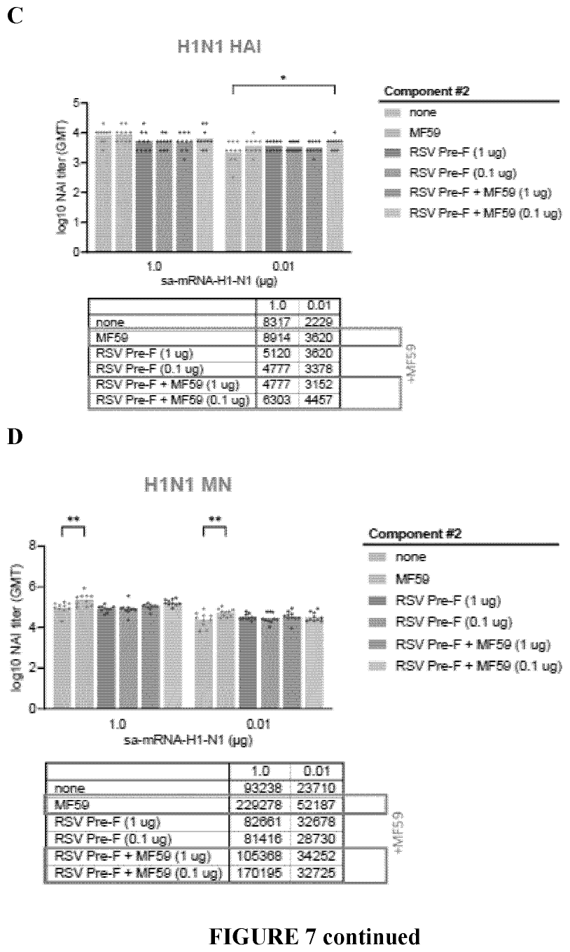

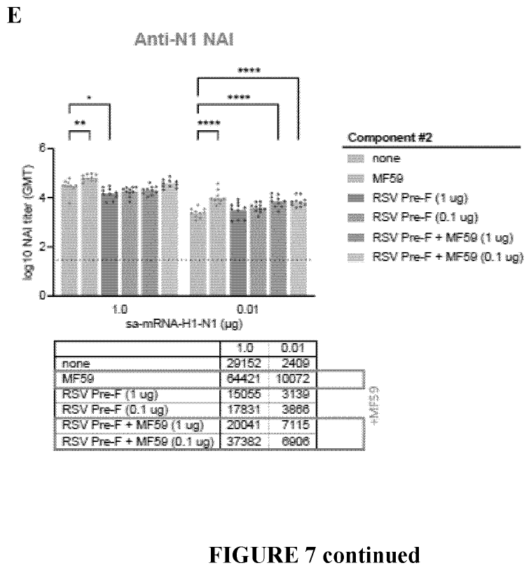

As shown in FIG. 7A, the H1N1 specific IgG response was enhanced when sa-mRNA was administered in the presence of the adjuvanted protein vaccine. IgG subclass was not however effected (FIG. 7B) nor was NAI titer (FIG. 7C). Neutralisation titer was modestly increased in the presence of sa-mRNA and the adjuvanted protein vaccine (FIG. 7D), however N1 inhibition was enhanced (FIG. 7E).

To assess the adjuvant activity of lipid nanoparticles on the protein based vaccine, BALB/c mice were immunised with a coformulation of monovalent H1N1 sa-mRNA vaccine and heterologous monovalent cell derived RSV Pre F protein subunit vaccine according to Table 6.

TABLE 6
Experimental mouse groups

| | | Rec. | | |
|---|---|---|---|---|
| | | RSV | | |
| | Mice / | Pre F | | |
| Group | Group | (μg) | Component #2 | MF59 |
| 13 | 10 | 1 | — | — |
| 16 | | 1 | Unformulated sa-mRNA (1 μg) | — |
| 15 | | 1 | Empty LNP (eq. 1 μg) | — |
| 3 | | 1 | sa-mRNA-H1N1 (1 μg) | — |
| 9 | | 1 | sa-mRNA-H1N1 (0.01 μg) | — |
| 5 | | 1 | sa-mRNA-H1N1 (1 μg) | MF59 (1x) |
| 11 | | 1 | sa-mRNA-H1N1 (0.01 μg) | MF59 (1x) |
| 14 | | 1 | — | MF59 (1x) |
| 17 | 10 | 0.1 | — | — |
| 20 | | 0.1 | Unformulated sa-mRNA (1 μg) | — |
| 19 | | 0.1 | Empty LNP (eq. 1 μg) | — |
| 4 | | 0.1 | sa-mRNA-H1N1 (1 μg) | — |
| 10 | | 0.1 | sa-mRNA H1N1 (0.01 μg) | — |
| 6 | | 0.1 | sa-mRNA-H1N1 (1 μg) | MF59 (1x) |
| 12 | | 0.1 | sa-mRNA-H1N1 (0.01 μg) | MF59 (1x) |
| 18 | | 0.1 | — | MF59 (1x) |

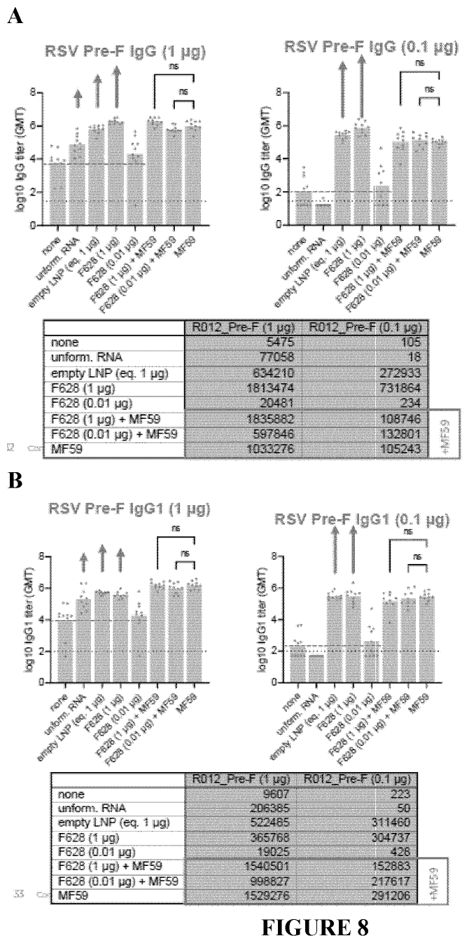

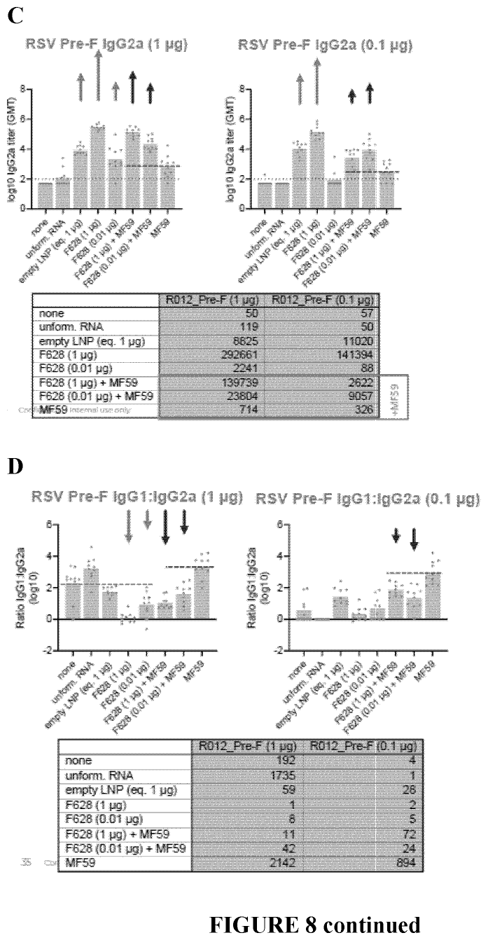

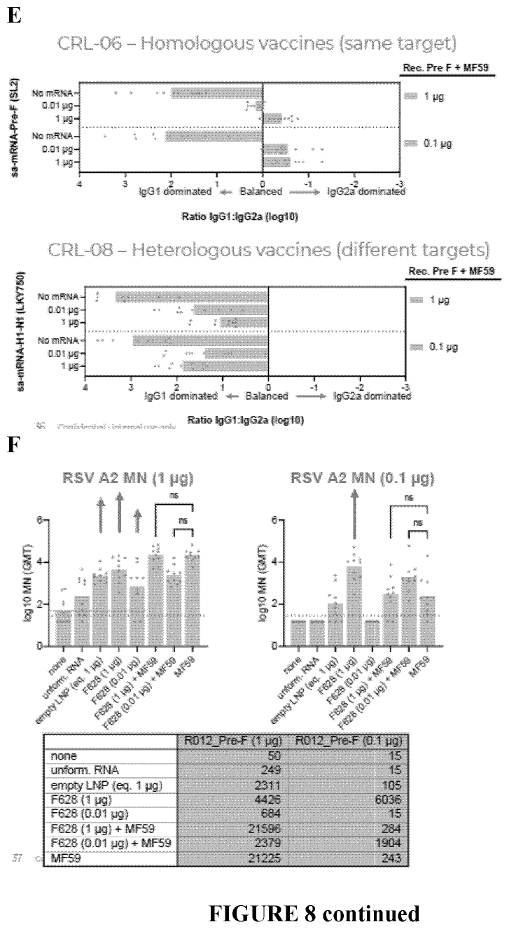

FIG. 8A demonstrates that the Pre F specific IgG response in enhanced when sa-mRNA was administered in the presence of the protein vaccine when adjuvanted with LNP (in the absence of MF59). Pre F specific IgG1 and IgG2a responses were also enhanced when sa-mRNA was administered in the presence of the protein vaccine when adjuvanted with LNP (in the absence of MF59) (FIG. 8B-C), although there was a reduction in the IgG1:IgG2a ratio (FIG. 8D). As shown in FIG. 8E, it appears that the IgG1:IgG2a ratio rebalance is stronger when the coformulation is homologous when compared to a heterologous formulation, although both are indicative of a Th1 predominant response. Lastly, neutralizing Ab responses were enhanced sa-mRNA was administered in the presence of the protein vaccine when adjuvanted with LNP (FIG. 8F).

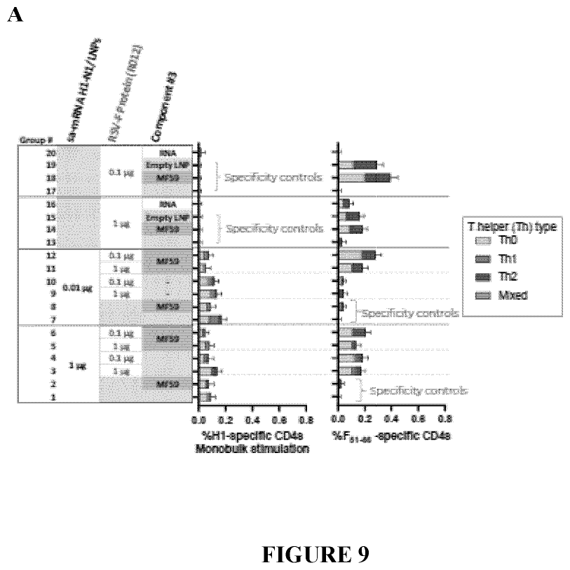

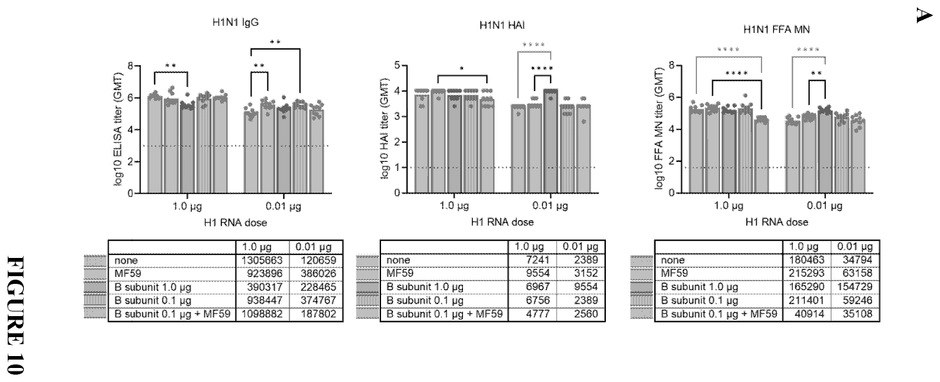

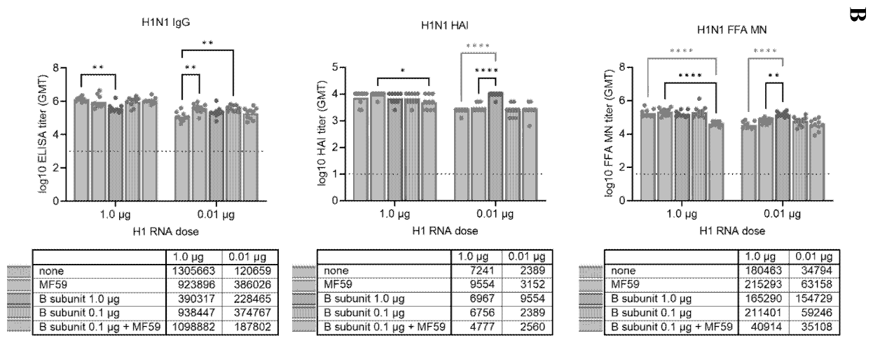

T cells were stimulated with a homemade peptide mix includes the following peptides: FYQSTCSAV (SEQ ID NO: 34; F32-40); WYTSVITI (SEQ ID NO: 35; F52-59); KYKNAVTEL (SEQ ID NO: 36; F85-92); EVNKIKSAL (SEQ ID NO: 37; F163-171); IKSALLSTN (SEQ ID NO: 38; F167-175) and TYMLTNSEL (SEQ ID NO: 39; F249-258). As shown in FIG. 9A, in the presence of the sa-mRNA alone, a CD4 Th1/Th0 response is induced, whilst in the presence of adjuvanted protein alone, a Th2/Th0 response is induced. In the presence of both adjuvanted protein and sa-mRNA, a predominant Th1 response is observed.

### Example 6: Effect of Influenza Antigen mRNA and Protein Composition on Immunogenicity in Mice

BALB/c mice were immunised with a coformulation of monovalent HA sa-mRNA vaccine and heterologous monovalent cell derived HA protein subunit vaccine according to Table 7.

TABLE 7
Experimental mouse groups

| | Mice/ | | Component 1 | | Component 2 |
|---|---|---|---|---|---|
| Group | Group | Component 1 | μg per animal | Component 2 | μg per animal |
| 1 | 10 | B/Singapore/INTT-16-0610/2016 | HA 10.0 | — | — |
| 2 | | (B Yam) monobulk lot# 257030 | HA 1.0 | — | — |
| 3 | | | HA 0.1 | — | — |
| 4 | | | HA 1.0 | MF59 | 1X |
| 5 | | | HA 0.1 | MF59 | |
| 6 | | | HA 1.0 | empty LNP | Equal to |
| 7 | | | HA 0.1 | empty LNP | RNA 1.0 μg |
| 8 | | A/Delaware/55/2019 (H1N1) | RNA 1.0 | — | — |
| 9 | | sa-mRNA monocistronic HA F641 | RNA 0.01 | — | |
| 10 | | | RNA 1.0 | MF59 | 1X |
| 11 | | | RNA 0.01 | MF59 | |
| 12 | | A/Delaware/55/2019 (H1N1) | RNA 1.0 | B/Singapore/INTT-16-0610/2016 (B Yam) | HA 1.0 |
| 13 | | | | | HA 0.1 |
| 14 | | | | B/Singapore/INTT-16-0610/2016 (B Yam) + MF59 | HA 0.1 |
| 15 | | | RNA 0.01 | B/Singapore/INTT-16-0610/2016 (B Yam) | HA 1.0 |
| 16 | | | | | HA 0.1 |
| 17 | | | | B/Singapore/INTT-16-0610/2016 (B Yam) + MF59 | HA 0.1 |

Mice were subjected to two vaccinations with sa-mRNA formulated in lipid nanoparticles according to the following.

Biophysical characterization of IMP in formulation

| | | | | | | | | | | sa- | Endo- | Vac. 1 | Vac. 2 |
|---|---|---|---|---|---|---|---|---|---|---|---|---|---|
| | | | | | | Encap- | | | | mRNA | toxin | Osmo- | Osmo- |
| | | LNP | | sa- | FACS | sulation | | | | Concen- | Level [symbol] | lality^{b} | lality^{b} |
| | | FD | LNP | mRNA | Potency | Effi- | | Size | | tration | (EU/ | (mmol/ | (mmol/ |
| Group # | Vacc. | Lot# | Type | vector | (ng − 1) | ciency(%) | Recovery | (nm) | PDI | (ug/mL) | mL) | kg) | kg) |
| Group | B Yam HA | — | — | — | — | — | — | — | — | — | — | 292, 288, | 287, 287 |
| #1, 2, 3 | | | | | | | | | | | | 278 | 284 |
| Group | B Yam HA + | — | — | — | — | — | — | — | — | — | — | 280, 306 | 288, 312 |
| # 4, 5 | MF59 | | | | | | | | | | | | |
| Group | B Yam HA + | CY010 | Empty | — | — | — | 41.5 | 98.11 | 0.205 | — | <1 | 281, 278 | 284, 287 |
| # 6, 7 | Empty LNP | | | | | | | | | | | | |
| Group | H1 LNP | GH173 | H1 | F641 | 3629 | 83.4 | 40.1 | 115.3 | 0.175 | 79.5 | <1 | 286, 290 | 287, 284 |
| # 8, 9 | | | | | | | | | | | | | |
| Group | H1 LNP + | GH173 | H1 | F641 | 3629 | 83.4 | 40.1 | 115.3 | 0.175 | 79.5 | <1 | 270, 308 | 280, 315 |
| # 10, 11 | MF59 | | | | | | | | | | | | |
| Group | H1 LNP + | GH173 | H1 | F641 | 3629 | 83.4 | 40.1 | 115.3 | 0.175 | 79.5 | <1 | 278, 288 | 285, 287 |
| # 12, 13 | B Yam HA | | | | | | | | | | | | |
| Group | H1 LNP + | GH173 | H1 | F641 | 3629 | 83.4 | 40.1 | 115.3 | 0.175 | 79.5 | <1 | 268, 312 | 278, 312 |
| # 14, 17 | B Yam HA + | | | | | | | | | | | | |
| | MF59 | | | | | | | | | | | | |
| Group | H1 LNP + | GH173 | H1 | F641 | 3629 | 83.4 | 40.1 | 115.3 | 0.175 | 79.5 | <1 | 280, 284 | 286, 287 |
| #15, 16 | B Yam HA | | | | | | | | | | | | |

[symbol]

indicates data missing or illegible when filed

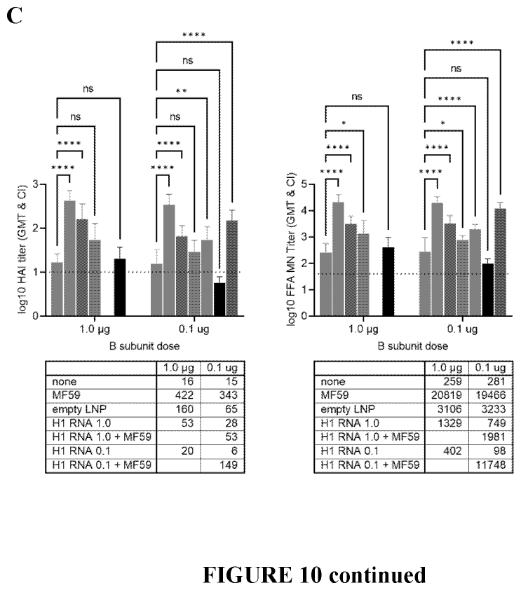

As shown in FIGS. 10A-C, there was a significant adjuvant effect in the presence of MF59 and a significant effect of empty LNPs. Moreover, adjuvant MF59 partially restores the effects inhibited by low dose LNPs.

In a further experiment, the inventors vaccinated C57BL/6 mice twice, 8 weeks apart with a trimeric H5 soluble protein, utilising the following T cell antigens.
H1: CD4: 10 ug/ml monobulk A/Delaware/55/2019 (H1N1) (HSP TD, Lot #296905); and CD8: 1 ug/ml of CD8 epitope “Peptide A” (IYSTVASSL, H1 residues 533-541).
B/Yamagata: CD4: 10 ug/ml monobulk B/Yamagata Singapore/INFTTI6 0610/2016 (HSP TD, Lot #288032); and CD8: 1 ug/ml of CD8 epitope “Peptide B” (YYSTAASSL, HA B residues 551-559).
VEE nSPs: CD4: 6 peptides from nSP1; and CD8: 6 peptides from nSP3.

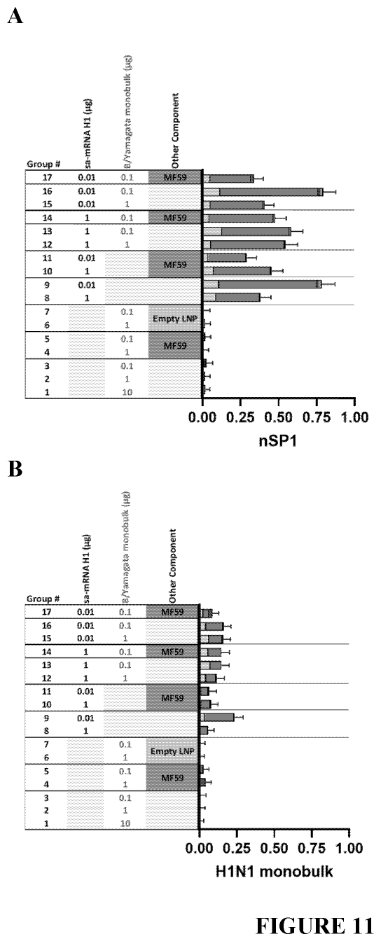

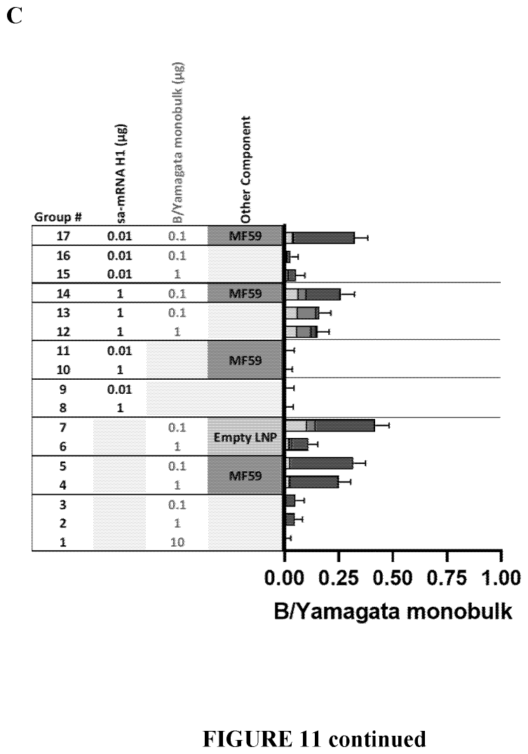

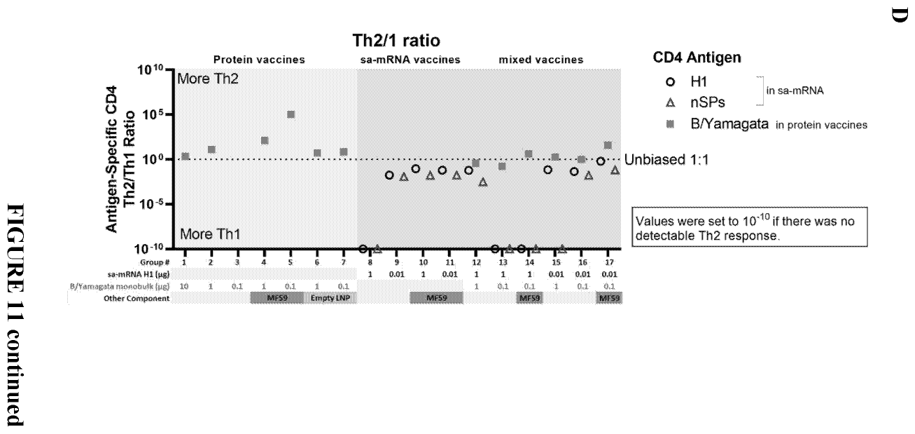

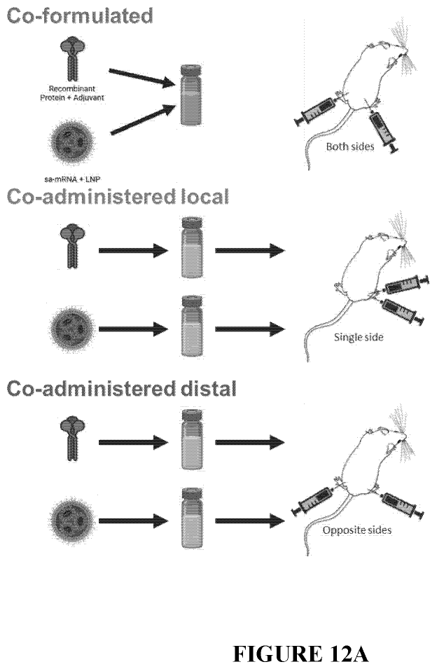

As shown in FIG. 11A-B, nSP1 and H1 specific CD4 responses are predominantly Th1/Th0 and the presence of MF59 does not change the Th bias. B/Yamagata responses in the presence of 1 g sa-mRNA H1/LNPs with B/Yamagata increased the adjuvants B/Yamagata CD4 specific response, and shifts some of the B/Yamagata response to Th1 (FIG. 11C). Whilst the sa-mRNA antigens (H1 & nSPs) consistently bias toward Th1 (below the mid-line), the protein vaccine (B/Yamagata) trends towards high Th2/Th1 ratio (above the mid-line) (FIG. 11D). The only exception is B/Yamagata in combination with a high dose sa-mRNA (groups 12 & 13), which trends towards unbiased (1:1 Th1:Th2 ratio), however this is not the case if MF59 is added (group 14).

In conclusion, antigens that are expressed intracellularly (in this case H1 and nSPs) result in Th0 & Th1 CD4 responses, regardless of the additional components. Adding MF59 (a Th2) adjuvant does not appear to overcome this Th1 bias against the intracellularly-expressed antigens. The adjuvant effect in the presence of sa-mRNA appears to be dose-dependent. Co-administration of protein with 1 g sa-mRNA shifts the Th2 response of protein vaccines towards a Th1 response, whilst adding MF59 restores the Th2 bias conferred by a protein vaccine. Finally, LNPs are capable of serving as an adjuvant for protein vaccines.

In conclusion, with respect to the use of compositions comprising homologous antigens, co-formulation of sa-mRNA/protein vaccines did not have a negative effect on neutralizing or IgG titers. The addition of sa-RNA modified a predominant Th2 response to a predominant Th1 response in a dose dependent manner. With respect to heterologous antigens, combining a sa-mRNA vaccine with a protein vaccine did not impact neutralizing or IgG response. The lipid component of the formulated RNA vaccine was capable of mediating adjuvant activity. As observed with the compositions comprising homologous antigens, the baseline Th2 response to the protein vaccine was shifted to a Th1 in a dose dependent manner in the presence of a sa-mRNA. These findings indicated that a mixed platform strategy has utility particular in circumstances where a Th1 predominant response is preferred or required.

### Example 7: Co-Formulation and Co-Administration of Heterologous sa-mRNA and Protein Vaccines have Differing Impacts on Immunogenicity

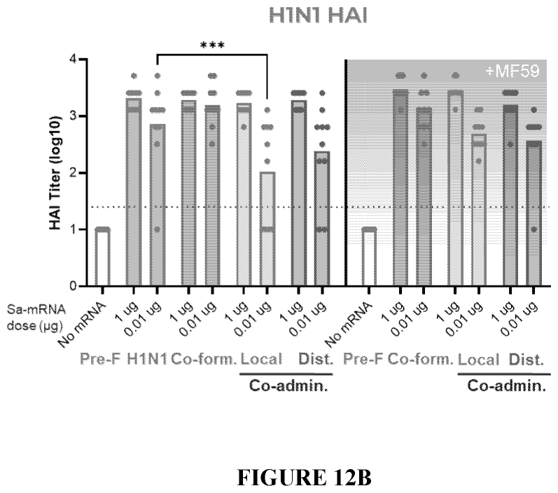

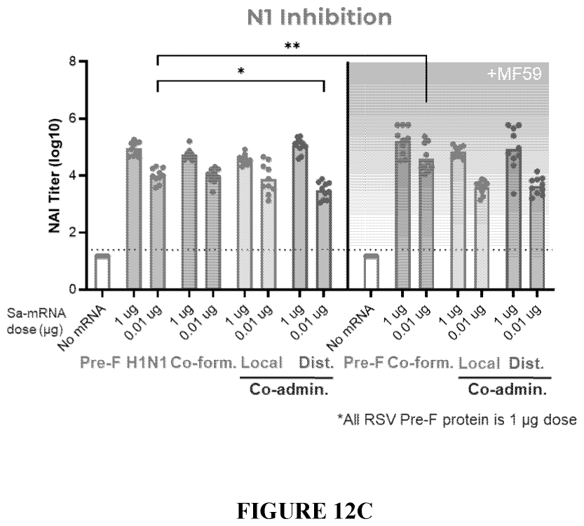

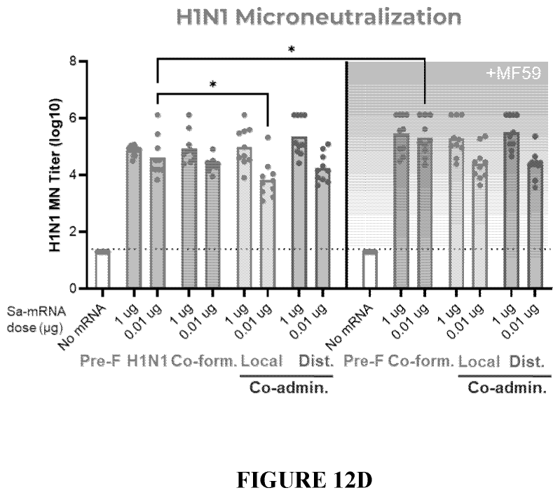

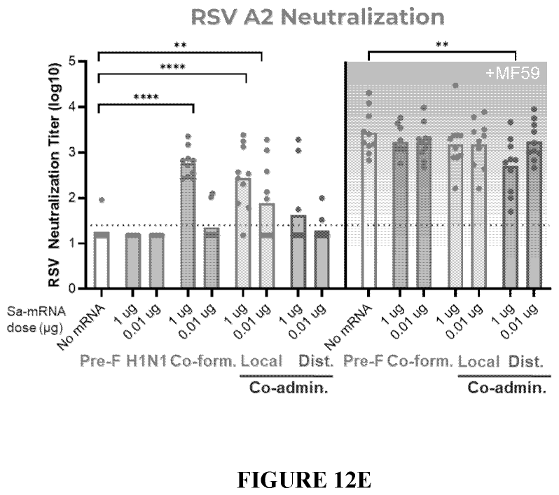

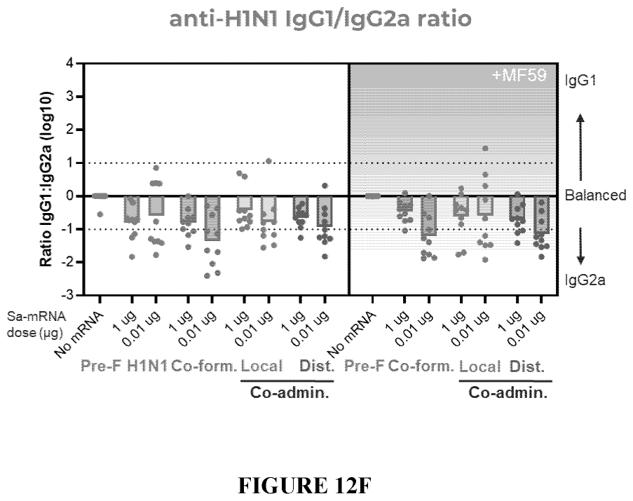

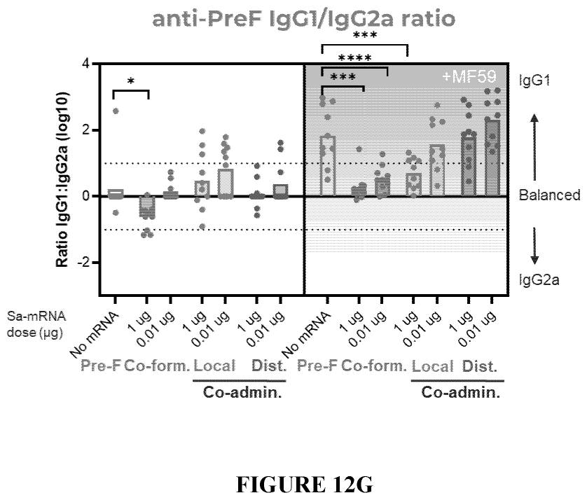

To investigate how responses to vaccines of different platforms would perform when combined we designed studies in which mice were given either sa-mRNA vaccine with protein vaccine with or without the MF59 adjuvant in one of three preparations: 1) co-formulated, with component vaccines mixed prior to immunization, 2) locally co-administered, with each vaccine prepared separately and administered to the same quadricep muscle separately, or 3) distally co-administered, with each vaccine prepared separately and administered bilaterally in opposite quadricep muscles (FIG. 12A). The inventors found that unlike co-formulation, local and distal co-administration of sa-mRNA with unadjuvanted or adjuvanted protein resulted no significant benefit in the immunogenicity of the sa-mRNA vaccine when measured by HAI (FIG. 12B), NAI (FIG. 12C), or MN (FIG. 12D). However, like co-formulation, local co-administration of sa-mRNA boosted the neutralizing antibody response to unadjuvanted protein vaccines (FIG. 12E). Further, we found that while neither co-formulation nor co-administration (local or distal) impacted the ratio of antigen-specific IgG1 to IgG2a induced against sa-mRNA vaccines (FIG. 12F), the subclass ratio of antigen-specific IgG induced by adjuvanted protein vaccine was significantly shifted towards a balanced IgG1:IgG2a response when either co-formulated or locally co-administered with sa-mRNA, but not when co-administered distally (FIG. 12G). Taken together our data suggests that either co-formulation or local co-administration of sa-mRNA vaccines with adjuvanted protein vaccines can enhance the neutralization titers against the targets of both vaccines, as well as influence the subclasses of antigen-specific IgG induced by recombinant protein vaccines.

1. An immunogenic composition comprising:
a) a RNA comprising a nucleotide sequence encoding an antigen selected from the group consisting of a respiratory syncytial virus (RSV), an influenza virus and a severe acute respiratory syndrome coronavirus (SARS-CoV-2), wherein the nucleotide sequence is operably linked to a regulatory element; and
b) an antigen polypeptide selected from the group consisting of a RSV, an influenza virus and a SARS-CoV-2.

2. The immunogenic composition of claim 1, comprising:
a) a RNA comprising a nucleotide sequence encoding an antigen from a RSV, wherein the nucleotide sequence is operably linked to a regulatory element; and
b) an antigen polypeptide from a SARS-CoV-2.

3. The immunogenic composition of claim 1, comprising:
a) a RNA comprising a nucleotide sequence encoding an antigen from a SARS-CoV-2, wherein the nucleotide sequence is operably linked to a regulatory element; and
b) an antigen polypeptide from a RSV.

4. The immunogenic composition of claim 1, comprising:
a) a RNA comprising a nucleotide sequence encoding an antigen from a SARS-CoV-2, wherein the nucleotide sequence is operably linked to a regulatory element; and
b) an antigen polypeptide from influenza.

5. The immunogenic composition of claim 1, comprising:
a) a RNA comprising a nucleotide sequence encoding an antigen from influenza, wherein the nucleotide sequence is operably linked to a regulatory element; and
b) an antigen polypeptide from a SARS-CoV-2.

6. The immunogenic composition of claim 1, comprising:
a) a RNA comprising a nucleotide sequence encoding an antigen from influenza, wherein the nucleotide sequence is operably linked to a regulatory element; and
b) an antigen polypeptide from a RSV.

7. The immunogenic composition of claim 1, comprising:
a) a RNA comprising a nucleotide sequence encoding an antigen from a RSV, wherein the nucleotide sequence is operably linked to a regulatory element; and
b) an antigen polypeptide from influenza.

8. The immunogenic composition of claim 1, further comprising a second RNA comprising a nucleotide sequence encoding an antigen selected from the group consisting of a RSV, an influenza virus and a SARS-CoV-2, wherein the nucleotide sequence is operably linked to a regulatory element.

9. The immunogenic composition of claim 1, further comprising a second antigen polypeptide selected from the group consisting of a RSV, an influenza virus and a SARS-CoV-2.

10. The immunogenic composition of claim 2 or 3, further comprising a second RNA comprising a nucleotide sequence encoding an antigen from influenza, operably linked to a regulatory element.

11. The immunogenic composition of claim 10, comprising, in order from 5′ to 3′:
(i) the RNA comprising a nucleotide sequence encoding an antigen from RSV or SARS-CoV-2;
(ii) the RNA comprising a nucleotide sequence encoding an antigen from influenza.

12. The immunogenic composition of claim 10, comprising, in order from 5′ to 3′:
(i) the RNA comprising a nucleotide sequence encoding an antigen from influenza; and
(ii) the RNA comprising a nucleotide sequence encoding an antigen from RSV or SARS-CoV-2.

13. The immunogenic composition of claim 2 or 3, further comprising a second antigen polypeptide from influenza.

14. The immunogenic composition of claim 4 or 5, further comprising a second RNA comprising a nucleotide sequence encoding an antigen from a RSV, operably linked to a regulatory element.

15. The immunogenic composition of 14, comprising, in order from 5′ to 3′:
(i) the RNA comprising a nucleotide sequence encoding an antigen from a SARS-CoV-2;
(ii) the RNA comprising a nucleotide sequence encoding an antigen from RSV.

16. The immunogenic composition of 14, comprising, in order from 5′ to 3′:
(i) the RNA comprising a nucleotide sequence encoding an antigen from RSV; and
(ii) the RNA comprising a nucleotide sequence encoding an antigen from a SARS-CoV-2.

17. The immunogenic composition of claim 4 or 5, further comprising a second antigen polypeptide from a RSV.

18. The immunogenic composition of claim 6 or 7, further comprising a second RNA comprising a nucleotide sequence encoding an antigen from a SARS-CoV-2, operably linked to a regulatory element.

19. The immunogenic composition of claim 18, comprising, in order from 5′ to 3′:
(i) the RNA comprising a nucleotide sequence encoding an antigen from a SARS-CoV-2;
(ii) the RNA comprising a nucleotide sequence encoding an antigen from influenza.

20. The immunogenic composition of claim 18, comprising, in order from 5′ to 3′:
(i) the RNA comprising a nucleotide sequence encoding an antigen from influenza; and
(ii) the RNA comprising a nucleotide sequence encoding an antigen from a SARS-CoV-2.

21. The immunogenic composition of claim 6 or 7, further comprising a second antigen polypeptide from a SARS-CoV-2.

22. The immunogenic composition of any one of claims 8, 10 to 12, 14 to 16 or 18 to 20, comprising a plurality of RNAs, wherein each self-replicating RNA encodes different polypeptide sequences.

23. The immunogenic composition of any one of claims 1 to 22, wherein the regulatory element is selected from the group consisting of a promoter, an internal ribosome entry site (IRES) or a Kozak consensus sequence or a combination thereof.

24. The immunogenic composition of claim 23, wherein the promoter is a subgenomic (SG) promoter.

25. The immunogenic composition of claim 24, wherein the SG promoter is a minimal SG promoter.

26. The immunogenic composition of claim 24, wherein the SG promoter is extended SG promoter.

27. The immunogenic composition of claim 25, wherein the minimal SG promoter is encoded by a sequence set forth in SEQ ID NO: 13.

28. The immunogenic composition of claim 26, wherein the extended SG promoter is encoded by a sequence set forth in SEQ ID NO: 14.

29. The immunogenic composition of claim 23, wherein the nucleotide sequence encoding an antigen is operably linked to an IRES located 5′ to the nucleotide sequence encoding the antigen.

30. The immunogenic composition of claim 23 or 29, wherein the IRES is an IRES from encephalomyocarditis virus (EMCV), poliovirus (PV), human enterovirus, foot-and-mouth disease virus (FMDV), hepatitis C virus (HCV), classical swine fever virus (CSFV), murine leukemia virus (MLV), simian immunodeficiency virus (SIV), Eukaryotic translation initiation factor 4G (elF4G), Death-associated protein 5 (DAP5), cellular Myc (c-Myc), NF-κB-repressing factor (NRF), vascular endothelial growth factor (VEGF), fibroblast growth factor (FGF-2), platelet-derived growth factor B (PDGF B), Antennapedia, X-linked inhibitor of apoptosis (XIAP or Apaf-1), immunoglobulin heavy-chain binding protein BiP, or fibroblast growth factor 1a (FGF1A), GTX, or a combination thereof.

31. The immunogenic composition of claim 30, wherein the EMCV IRES is a wild-type IRES encoded by a sequence set forth in SEQ ID NO: 16.

32. The immunogenic composition of any one of claims 1, 2, 7, 8, 11, 12 or 14 to 16, wherein the nucleotide sequence encoding an antigen from a RSV has a sequence at least 70%, at least 75%, at least 80%, at least 85%, at least 90% or at least 95% identical to SEQ ID NO:1 or 3.

33. The immunogenic composition of any one of claims wherein the nucleotide sequence encoding an antigen from a RSV has the sequence set forth in SEQ ID NO:1 or 3.

34. The immunogenic composition of any one of claims 1, 3, 4, 8, 12, 15, 16, 18, 19 or 20, wherein the nucleotide sequence encoding an antigen from a SARS-CoV-2 has a sequence at least 70%, at least 75%, at least 80%, at least 85%, at least 90% or at least 95% identical to SEQ ID NO: 9 or 11.

35. The immunogenic composition of any one of claims 1, 3, 4, 8, 12, 15, 16 or 18 to 20, wherein the nucleotide sequence encoding an antigen from a SARS-CoV-2 has the sequence set forth in SEQ ID NO: 9 or 11.

36. The immunogenic composition of any one of claims 1, 5, 6, 8, 10 to 12, 19 or 20, wherein the nucleotide sequence encoding an antigen from influenza has a sequence at least 70%, at least 75%, at least 80%, at least 85%, at least 90% or at least 95% identical to SEQ ID NO: 5 or 7.

37. The immunogenic composition of any one of claims 1, 5, 6, 8, 10 to 12, 19 or 20, wherein the nucleotide sequence encoding an antigen from influenza has the sequence set forth in SEQ ID NO: 5 or 7.

38. The immunogenic composition of any one of claims 1, 3, 6, 9 or 17, wherein the antigen polypeptide from a RSV has an amino acid sequence at least 70%, at least 75%, at least 80%, at least 85%, at least 90% or at least 95% identical to SEQ ID NO: 2 or 4.

39. The immunogenic composition of any one of claims 1, 3, 6, 9 or 17, wherein the antigen polypeptide from a RSV has the amino acid sequence set forth in SEQ ID NO: 2 or 4.

40. The immunogenic composition of any one of claims 1, 2, 5, 9 or 21, wherein the antigen polypeptide from a SARS-CoV-2 has an amino acid sequence at least 70%, at least 75%, at least 80%, at least 85%, at least 90% or at least 95% identical to SEQ ID NO: 10 or 12.

41. The immunogenic composition of any one of claims 1, 2, 5, 9 or 21, wherein the antigen polypeptide from a SARS-CoV-2 has the amino acid sequence set forth in SEQ ID NO: 10 or 12.

42. The immunogenic composition of any one of claims 1, 4, 7, 9 or 13, wherein the antigen polypeptide from influenza has an amino acid sequence at least 70%, at least 75%, at least 80%, at least 85%, at least 90% or at least 95% identical to SEQ ID NO: 6 or 8.

43. The immunogenic composition of any one of claims 1, 4, 7, 9 or 13, wherein the antigen polypeptide from influenza has the amino acid sequence set forth in SEQ ID NO: 6 or 8.

44. The immunogenic composition of any one of claims 1 to 43, wherein the RNA is selected from the group consisting of cRNA or a self-replicating RNA.

45. The immunogenic composition of claim 44, wherein the self-replicating RNA is from an alphavirus, optionally selected from the group consisting of Semliki Forest virus (SFV), Sindbis virus (SIN), and Venezuelan equine encephalitis virus (VEE) and combinations thereof.

46. The immunogenic composition of any one of claims 1 to 45, further comprising an adjuvant.

47. The immunogenic composition of claim 46, wherein the adjuvant is MF59.

48. The immunogenic composition of any one of claims 1 to 47, further comprising an additional RNA encoding, or a polypeptide selected from:
(i) one or more antigens;
(ii) one or more immunopotentiating agents;
(iii) one or more chemoattractants; and/or
(iv) one or more targeting molecules.

49. A pharmaceutical composition comprising an immunogenic composition of any one of claims 1 to 48 and a pharmaceutically acceptable carrier.

50. The pharmaceutical composition of claim 49, further comprising a lipid nanoparticle (LNP), a polymeric microparticle or an oil-in-water emulsion.

51. The pharmaceutical composition of claim 50, wherein the RNA is encapsulated in, bound to or adsorbed on a LNP, a polymeric microparticle or an oil-in-water emulsion.

52. The pharmaceutical composition of any one of claims 49 to 51, wherein each RNA is formulated together in the LNP.

53. The pharmaceutical composition of any one of claims 49 to 51, wherein each RNA is formulated separately in the LNP.

54. The immunogenic composition of any one of claims 1 to 48 or the pharmaceutical composition of any one of claims 49 to 53, for use as a vaccine.

55. A vaccine comprising the pharmaceutical composition of any one of claims 49 to 53 or the immunogenic composition of any one of claims 1 to 48.

56. A method of treating or preventing or delaying progression of a disease or condition in a subject in need thereof, the method comprising administering the immunogenic composition any one of claims 1 to 48, the pharmaceutical composition of any one of claims 49 to 53 or the vaccine of claim 55 to the subject.

57. Use of the immunogenic composition any one of claims 1 to 48, the pharmaceutical composition of any one of claims 49 to 53 or the vaccine of claim 55 in the manufacture of a medicament for treating or preventing or delaying progression of a disease or condition in a subject in need thereof.

58. The immunogenic composition any one of claims 1 to 48, the pharmaceutical composition of any one of claims 49 to 53 or the vaccine of claim 55 for use in treating or preventing or delaying progression of a disease or condition in a subject in need thereof.

59. The method of claim 56, the use of claim 57 or the pharmaceutical composition, the immunogenic composition or the vaccine for use of claim 58, wherein the disease or condition is selected from influenza, RSV, a SARS-CoV-2 infection and/or COVID-19.

60. A method of inducing an immune response in a subject, the method comprising administering the immunogenic composition any one of claims 1 to 48, the pharmaceutical composition of any one of claims 49 to 53 or the vaccine of claim 55 to a subject in need thereof.

61. Use of the immunogenic composition any one of claims 1 to 48, the pharmaceutical composition of any one of claims 49 to 53 or the vaccine of claim 55 in the manufacture of a medicament for inducing an immune response in a subject in need thereof.

62. The immunogenic composition any one of claims 1 to 48, the pharmaceutical composition of any one of claims 49 to 53 or the vaccine of claim 55 for use in inducing an immune response in a subject in need thereof.

63. The method of claim 60, the use of claim 61, or the immunogenic composition, the pharmaceutical composition or vaccine for use of claim 62, wherein the immune response is a cell-mediated immune response, optionally a Th2 response.

64. A method for reducing viral load in a subject comprising administering the immunogenic composition any one of claims 1 to 48, the pharmaceutical composition of any one of claims 49 to 53 or the vaccine of claim 55 to the subject in need thereof.

65. Use of the immunogenic composition any one of claims 1 to 48, the pharmaceutical composition of any one of claims 49 to 53 or the vaccine of claim 55 in the preparation of a medicament for reducing viral load in a subject in need thereof.

66. The immunogenic composition any one of claims 1 to 48, the pharmaceutical composition of any one of claims 49 to 53 or the vaccine of claim 55 for use in reducing viral load in a subject in need thereof.

67. A method for inducing a Th1 immune response in a subject in need thereof, comprising administering the pharmaceutical composition of any one of claims 49 to 53 or the vaccine of claim 55 to the subject.

68. Use of the pharmaceutical composition of any one of claims 49 to 53 or the vaccine of claim 55 in the preparation of a medicament for inducing a Th1 immune response in a subject.

69. The pharmaceutical composition of any one of claims 49 to 53 or the vaccine of claim 55 for use in inducing a Th1 immune response in a subject.

70. The method of any one of claims 56, 59, 60, 63, 64 or 67, the use of any one of claims 57, 59, 61, 63, 65 or 68, or the immunogenic composition, the pharmaceutical composition or vaccine for use of any one of claims 58, 59, 62, 63, 66 or 69, wherein the subject is a human of 18 years of age or older.

71. A method of treating, preventing or delaying progress of a respiratory syncytial virus (RSV) in an infant comprising administering the immunogenic composition of any one of claims 1, 2, 3, 6 to 9, 11, 12 or 14 to 17, or a pharmaceutical composition or vaccine thereof, to the infant.

72. Use of the immunogenic composition of any one of claims 1, 2, 3, 6 to 9, 11, 12 or 14 to 17, or a pharmaceutical composition or vaccine thereof, in the preparation of a medicament for treating, preventing or delaying progress of a respiratory syncytial virus (RSV) in an infant.

73. The immunogenic composition of any one of claim 1, 2, 3, 6 to 9, 11, 12 or 14 to 17, or a pharmaceutical composition or vaccine thereof for use in treating, preventing or delaying progress of a respiratory syncytial virus (RSV) in an infant.

74. A method of inducing an immune response in an infant comprising administering the immunogenic composition of any one of claims 1 to 48, the pharmaceutical composition of any one of claims 49 to 53 or the vaccine of claim 55 to the infant.

75. Use of the immunogenic composition of any one of claims 1 to 48, the pharmaceutical composition of any one of claims 49 to 53 or the vaccine of claim 55 in the preparation of a medicament for inducing an immune response in an infant.

76. The immunogenic composition of any one of claims 1 to 48, the pharmaceutical composition of any one of claims 49 to 53 or the vaccine of claim 55 for use in inducing an immune response in an infant.

77. The method of claim 74, the use of claim 75, or the pharmaceutical composition, immunogenic composition or vaccine for use of claim 76, wherein the immune response is a cell-mediated immune response, optionally a Th1 response.

78. The method of claim 71, the use of claim 72 or the immunogenic composition, pharmaceutical composition or vaccine for use of claim 73, wherein the infant is between about 1 month and 12 months of age.

79. The method of any one of claims 56, 59, 60, 63, 64, 67, 70, 71, 74 or 77, the use of any one of claims 57, 59, 61, 63, 65, 68, 70, 72, 75, 77 or 78 or the pharmaceutical composition, immunogenic composition or vaccine for use of any one of claims 58, 59, 62, 63, 66, 69, 70, 73, 76 to 78, wherein the vaccine or composition is administered in a one dose regimen.

80. The method of any one of claims 56, 59, 60, 63, 64, 67, 70, 71, 74 or 77, the use of any one of claims 57, 59, 61, 63, 65, 68, 70, 72, 75, 77 or 78 or the pharmaceutical composition, immunogenic composition or vaccine for use of any one of claims 58, 59, 62, 63, 66, 69, 70, 73, 76 to 78, wherein the composition or vaccine is administered in a two, three or four dose regimen, wherein the doses are administered about 1, 2 or 3 months apart.

81. A kit comprising:
(a) the immunogenic composition any one of claims 1 to 48, the pharmaceutical composition of any one of claims 49 to 53, or the vaccine of claim 55;
(b) instructions for use thereof; and optionally
(c) a diluent.

82. A method of treating or preventing or delaying progression of a disease or condition in a subject, the method comprising locally co-administering
(i) a polynucleotide comprising a nucleotide sequence encoding an antigen selected from the group consisting of a respiratory syncytial virus (RSV), influenza and a severe acute respiratory syndrome coronavirus (SARS-CoV-2), wherein the nucleotide sequence is operably linked to a regulatory element; and
(ii) an antigen polypeptide selected from the group consisting of a RSV, influenza and a SARS-CoV-2.


SEQUENCE LISTING (32 sequences)

SEQ ID NO: 1 - RNA, 1725 nt, synthetic construct
  source 1..1725: mol_type = other RNA
atggaactgctgatcctgaaggccaatgctattacaaccatcctgaccgccgtcaccttctgcttcgcctctggacagaacatcaccgaggaattttaccagagcacctgtagtgctgtttccaaaggctacctgagcgctctgcggacaggctggtatacatctgtgatcaccatcgagctctctaacatcaaaaagaacaaatgcaatggaacagatgccaaggtgaagctgatcaagcaagagctggacaaatacaagaatgccgtgaccgaactccagctgcttatgcagagcacacaggccaccaacaaccgggccagaagagagctgcctagattcatgaactacaccctgaacaacgccaagaaaacaaacgtcaccctgagcaagaaaagaaagcggcggttcctgggcttcctcctgggcgtgggatctgccatcgccagcggcgtggccgtgagcaaggtgctgcacctggaaggcgaagtgaacaagatcaagagcgccctgctgagcacaaacaaggccgtggtgagcctgtccaatggcgtcagcgtgctgacaagcaaagtgctggatctgaagaactacatcgacaagcagctgctgcctatcgtgaacaaacaaagctgtagcatcagcaacatcgagacagtgatcgagtttcagcagaagaacaacagactgctggaaatcaccagagaattcagcgtgaatgctggcgtgacaacacccgtgagcacctacatgctgacaaacagcgagctgctgtctctgatcaacgacatgcctattaccaatgatcagaaaaaactgatgagcaacaatgttcaaatcgtcagacagcagagctatagcatcatgtctatcattaaggaagaggtgctggcctacgtcgtgcagctgccactgtacggagtgatcgacaccccttgctggaagctgcacacctcccctctgtgtaccaccaataccaaggaaggctcgaatatctgcctgacccggaccgatagaggctggtactgcgacaacgccggcagcgtgtccttcttcccccaggccgagacctgcaaggtccaaagcaatagagtgttctgtgataccatgaacagcctgaccctgcccagcgaggtgaacctgtgcaacgtggacatcttcaaccccaagtacgactgcaaaatcatgacaagcaagaccgacgtgtccagcagcgtgatcacctctcttggcgccatcgtgagctgctacggcaagaccaagtgcaccgccagcaacaagaaccggggcatcatcaagacattcagcaacggatgtgactacgttagcaacaagggagtggacaccgtgtctgtgggcaacaccctgtactacgtgaacaagcaggagggcaagtctctgtacgtcaagggcgagcctatcatcaacttctacgatcctctggtgttcccaagcgacgagttcgacgcctctatcagccaggtgaacgagaagatcaaccagtctctggcctttatcagaaagagcgacgagctgctgcacaacgtgaatgccggcaaatccacaaccaacatcatgatcaccacaatcatcatcgtgatcattgtgatcctgctgagcctgatcgccgtgggcctgctgctgtattgcaaggctagaagcacacctgtgaccctgtctaaggaccagctgtcaggcatcaacaatatcgctttttctaactga

SEQ ID NO: 2 - protein, 574 aa, synthetic construct
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 3 - RNA, 1794 nt, synthetic construct
  source 1..1794: mol_type = other RNA
atggaactactgatcctgaaggccaacgccatcaccacaatcctgaccgctgtgacattttgcttcgccagcggacagaacattacggaggagttctaccagagcacctgtagcgccgtgagcaagggatacctgtctgccctgagaactggctggtacacaagcgtgatcaccatcgagctgagcaatatcaaggaaaacaagtgcaacggcaccgacgccaaggtgaagctgatcaagcaggaactggataagtacaagaacgccgtgaccgagctgcagctgctgatgcagagcacccccgctacaaacaacagagccagacgcgagctgcctcggttcatgaactatacactcaacaacgctaagaaaaccaacgtgacactgtctaaaaagagaaaaagacggttccttggcttcctgctgggggtgggcagtgctatcgcctctggcgtcgccgtgtgcaaggtcctgcacctggaaggcgaagtgaacaagatcaaaagcgccctgctgagcaccaacaaggccgttgtgtctctgagcaatggcgtctccgtgctgaccttcaaggtgctggacctgaagaattacatcgacaagcagctgctgcctatcctgaacaagcaaagctgcagcatttctaacatcgaaaccgtgatcgagttccagcagaaaaacaaccggctgctggaaatcaccagagagttcagcgtcaacgccggcgtgaccacccctgtgagtacctacatgttgaccaacagcgagctgctcagcctgatcaatgatatgccaatcacaaatgaccagaagaaactgatgagcaacaatgttcagatcgtgcggcagcagagctacagcatcatgtgcatcatcaaggaagaggtgctggcttacgtggtgcaactgcctctgtatggcgtgatcgatacgccttgctggaagctgcacacaagccctctgtgcacaacaaacacgaaggagggcagcaacatttgcctgaccagaaccgatagaggctggtattgtgataacgccggaagcgtgtccttctttccccaagccgagacatgcaaggtgcagtctaatagagtgttctgcgacaccatgaacagcctgacactgcctagcgaggtgaatctgtgtaatgtggacatcttcaaccccaagtacgactgcaagatcatgacctccaaaaccgatgtgtccagcagcgtgatcaccagcctgggcgccatagtgagctgttacggcaagacaaagtgcaccgccagcaacaaaaaccggggcatcatcaagaccttcagcaacggatgtgactacgtgagcaataagggcgttgacaccgtgagcgtgggcaacaccctgtactacgtgaacaaacaggagggaaagtctctgtacgtgaagggagaacctatcatcaacttctacgaccccctggtgtttccatctgacgagtttgatgcctccatcagccaggtgaacgagaaaatcaaccagtctctcgccttcatcagaaagagcgacgagctgctgtccgccattggcggctacatccctgaggcccctcgggacggccaggcctacgttagaaaggacggcgagtgggtgctgctgtccacctttctgggaggcctggtccccaggggcggctcagctggctctggatggtctcaccctcagttcgaaaaaggcggcggcagcggaggcggcagcggcggaggctcctggagccacccccagttcgagaagggtagcaagggcggccaccaccatcaccaccactgataa

SEQ ID NO: 4 - protein, 596 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFLGGLVPRGGSAGSGWSHPQFEKGGGSGGGSGGGSWSHPQFEKGSKGGHHHHHH

SEQ ID NO: 5 - RNA, 1701 nt, synthetic construct
  source 1..1701: mol_type = other RNA
atgaaggcaatactagtagttatgctgtatacatttacaaccgcaaatgcagacacattatgtataggttatcatgcgaacaattcaacagacactgtggacacagtactagaaaagaatgtaacagtaacacactctgtcaatcttctggaagacaagcataacggaaaactatgcaaactaagaggggtagccccattgcatttgggtaaatgtaacattgctggctggatcctgggaaatccagagtgtgaatcactctccacagcaagatcatggtcctacattgtggaaacatctaattcagacaatggaacgtgttacccaggagatttcatcaattatgaggagctaagagagcaattgagctcagtgtcatcatttgaaaggtttgaaatattccccaagacaagttcatggcctaatcatgactcggacaatggtgtaacggcagcatgtcctcacgctggagcaaaaagcttctacaaaaacttgatatggctggttaaaaaaggaaaatcatacccaaagatcaaccaaacctacattaatgataaagggaaagaagtcctcgtgctgtggggcattcaccatccacctactattgctgaccaacaaagtctctatcagaatgcagatgcatatgtttttgtggggacatcaagatacagcaagatgttcaagccggaaatagcaacaagacccaaagtgagggatcaagaagggagaatgaactattactggacactagtagaaccgggagacaaaataacattcgaagcaactggtaatctagtggcaccgagatatgcattcacaatggaaagagatgctggatctggtattatcatttcagatacaccagtccacgattgcaatacaacttgtcaaacacccgagggtgctataaacaccagcctcccatttcagaatgtacatccgatcacaattgggaaatgtccaaagtatgtaaaaagcacaaaattgagactggccacaggattgaggaatgtcccgtctattcaatctagaggcctattcggggccattgctggcttcatcgaaggggggtggacagggatggtagatggatggtacggttatcaccatcaaaatgagcaggggtcaggatatgcagccgatctgaagtccacacaaaatgccattgataagattactaacaaagtaaattctgttattgaaaagatgaatacacagttcacagcagttggtaaagagttcaaccaccttgaaaaaagaatagagaatctaaataaaaaggttgatgatggtttcctggacatttggacttacaatgccgaactgttggttctactggaaaacgaaagaactttggactatcacgattcaaatgtgaagaacttgtatgaaaaagtaagaaaccagttaaaaaacaatgccaaggaaattggaaacggctgctttgaattttaccacaaatgcgacaacacatgcatggaaagtgtcaagaatgggacttatgactacccaaaatactcagaggaagcaaaattaaacagagaaaaaatagatggagtaaagctggactcaacaaggatctaccagattttggcgatctattcaactgttgccagttcattggtactggtagtctccctgggggcaatcagcttctggatgtgctctaatgggtctctacagtgtagaatatgtatttaa

SEQ ID NO: 6 - protein, 566 aa, synthetic construct
MKAILVVMLYTFTTANADTLCIGYHANNSTDTVDTVLEKNVTVTHSVNLLEDKHNGKLCKLRGVAPLHLGKCNIAGWILGNPECESLSTARSWSYIVETSNSDNGTCYPGDFINYEELREQLSSVSSFERFEIFPKTSSWPNHDSDNGVTAACPHAGAKSFYKNLIWLVKKGKSYPKINQTYINDKGKEVLVLWGIHHPPTIADQQSLYQNADAYVFVGTSRYSKMFKPEIATRPKVRDQEGRMNYYWTLVEPGDKITFEATGNLVAPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLDSTRIYQILAIYSTVASSLVLVVSLGAISFWMCSNGSLQCRICI

SEQ ID NO: 7 - RNA, 1692 nt, synthetic construct
  source 1..1692: mol_type = other RNA
atggagaaaatagtgcttcttcttgcaatagtcagccttgttaaaagtgatcagatttgcattggttaccatgcaaacaactcgacagagcaggttgacacaataatggaaaagaacgtcactgttacacacgcccaagacatactggaaaagacacacaacgggaaactctgcgatctagatggagtgaagcctctaattttaagagattgtagtgtagctggatggctcctcgggaacccaatgtgtgacgaattcctcaatgtgccggaatggtcttacatagtggagaagatcaatccagccaatgacctctgttacccagggaatttcaacgactatgaagaactgaaacacctattgagcagaataaaccattttgagaaaattcagatcatccccaaaagttcttggtcagatcatgaagcctcagcaggggtgagctcagcatgtccataccagggaaggtcctccttttttagaaatgtggtatggcttatcaaaaaggacaatgcatacccaacaataaagagaagttacaataataccaaccaagaagatcttttggtattgtgggggattcaccatccaaatgatgcggcagagcagacaaggctctatcaaaacccaactacctatatttccgttgggacatcaacactaaaccagagattggtaccaaaaatagccactagatctaaggtaaacgggcaaagtggaaggatggagttcttttggacaattttaaaaccgaatgatgcaataaactttgagagtaatggaaatttcattgctccagaaaatgcatacaaaattgtcaagaaaggggactcaacaattatgaaaagtgagttggaatatggtaactgcaacaccaagtgtcaaactccaataggggcgataaactctagtatgccattccacaacatccaccctctcaccatcggggaatgccccaaatatgtgaaatcaagcagattagtccttgctactgggctcagaaatagccctcaacgagagacacgaggactatttggagctatagcaggttttatagagggaggatggggaatggtagatggttggtatgggtaccaccatagcaacgagcaggggagtgggtacgctgcagacaaagaatccactcaaaaggcaatagatggagtcaccaataaggtcaactcgatcattgacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagcttggtaacggttgtttcgagttctatcacagatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagtggcgagctccctagcactggcaatcatggtggctggtctatctttatggatgtgctccaatggatcgttacaatgcagaatttgcatttaa

SEQ ID NO: 8 - protein, 563 aa, synthetic construct
MEKIVLLLAIVSLVKSDQICIGYHANNSTEQVDTIMEKNVTVTHAQDILEKTHNGKLCDLDGVKPLILRDCSVAGWLLGNPMCDEFLNVPEWSYIVEKINPANDLCYPGNFNDYEELKHLLSRINHFEKIQIIPKSSWSDHEASAGVSSACPYQGRSSFFRNVVWLIKKDNAYPTIKRSYNNTNQEDLLVLWGIHHPNDAAEQTRLYQNPTTYISVGTSTLNQRLVPKIATRSKVNGQSGRMEFFWTILKPNDAINFESNGNFIAPENAYKIVKKGDSTIMKSELEYGNCNTKCQTPIGAINSSMPFHNIHPLTIGECPKYVKSSRLVLATGLRNSPQRETRGLFGAIAGFIEGGWGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHRCDNECMESVRNGTYDYPQYSEEARLKREEISGVKLESIGTYQILSIYSTVASSLALAIMVAGLSLWMCSNGSLQCRICI

SEQ ID NO: 9 - RNA, 3894 nt, synthetic construct
  source 1..3894: mol_type = other RNA
atgttcgtgttcctggtgttactgcctctggtgtcctcccaatgtgtcaacctgacaacaagaacgcagctgccaccagcttatacaaacagctttaccaggggcgtgtactaccccgataaagtgttccggtctagcgtcctgcacagcacccaggacctgttcctgcctttctttagcaacgtgacctggttccacgtgatctctggcaccaatggcaccaagagattcgacaaccccgtgctgcccttcaatgacggcgtgtacttcgcctctatcgagaagagtaacatcatccggggctggatcttcggaaccaccctggacagcaagacccaatctctgctgatcgtgaacaacgccacgaacgtcgtgattaaggtgtgcgagttccagttctgcaacgaccccttcctcgaccacaagaacaacaagtcttggatggaaagcgagttcagagtgtacagcagcgccaacaactgcacctttgagtacgtgagccaaccattcctcatggacctggaaggcaagcagggcaacttcaagaacctccgggaattcgtgtttaagaacatcgatggctattttaagatctactctaagcacacccctattatcgtgagagagcccgaggacctgcctcagggattctcggccctcgagcctctggtggacctgcctatcgggataaacatcaccagattccaaaccctgctggccctgcatagaagctacctgacaccaggcgatagcagcagcggctggaccgccggagcggctgcttattacgtgggctacctgcagcccagaaccttcctgctgaagtacaacgagaatggcacaatcaccgacgctgttgactgcgccctggatcctctgtctgagacaaagtgtaccctcaagagctttaccgtggaaaagggcatctaccagaccagcaatttcagagtgcagcccactgagtctatcgtgagatttccaaacatcactaacctgtgtccttttgatgaggtgttcaatgccacccgcttcgccagcgtgtatgcctggaaccggaagcggatctctaactgcgtggccgactacagcgtgctgtacaatctggccccatttttcacattcaagtgctacggcgtcagccctaccaagctgaatgatctgtgcttcaccaacgtgtacgccgattctttcgtgatcagaggcgacgaagtgcggcagattgcgcctggccagaccggcaatatcgccgattacaactacaagctgcctgacgacttcaccggctgcgtgatcgcttggaacagcaataagctggattccaaggtgagcggcaattacaattacctgtaccggctgttcagaaagagcaacctgaagcccttcgagagagacatcagcacggaaatctaccaggccggaaacaagccttgtaacggagtagccggcttcaattgttacttcccgctcagatcctacagcttcagacctacctatggagtcggccaccagccttaccgggtggtggtcctgagcttcgagctgctgcacgcccctgccacagtgtgcggccctaagaagtccacaaacctggtgaagaacaagtgcgtgaactttaattttaatggcctgaaaggcaccggagtgctgacggaaagcaacaagaaattcctgcccttccagcagttcggcagagacatcgccgacacaaccgatgccgtgcgggacccacaaaccctggaaatcctggatatcactccctgttctttcggcggcgtgtctgtgattacccctggaacaaatacatcgaaccaggtggccgtgctgtatcaaggcgtaaactgcacagaagtgcctgtggccatccacgctgaccagctgacccctacctggcgagtgtacagcaccggctccaacgtgttccagaccagagccggctgtctaattggagctgagtacgtgaacaactcttatgagtgcgacatccctatcggcgccggcatctgcgccagctaccagacacagaccaagagccacggcagcgccagctctgtggcctcccagtctatcatcgcctataccatgagcctcggcgcagagaactccgtggcctacagcaacaatagcatcgctatccccacaaacttcaccatcagcgtgaccacagagatcctgcctgtgagcatgaccaagaccagtgtggactgtacaatgtacatctgtggagattctaccgagtgcagcaacctgctgctgcagtacggctcattctgcacccagctgaaaagggccctgactggcatcgccgtggaacaggacaagaacacccaggaggtgtttgcacaagttaagcagatctacaagacccctcctatcaaatacttcggcggcttcaacttctcccagatcctgcctgatcctagtaaaccctcaaaacggagcttcatcgaagatctccttttcaacaaggtcaccctggccgacgccggctttatcaagcagtacggcgattgcctgggcgacatcgccgccagagatctgatctgcgcccagaagttcaaaggcctgaccgtgctgccacctttgctaacagacgagatgatcgcccagtacaccagcgctcttctggctggcaccatcacatctggctggacattcggcgccggcgccgctctgcagattcccttcgctatgcagatggcctacagattcaacggaatcggcgttacccagaatgtgctctacgagaaccagaagctgatcgctaatcagtttaacagcgccatcggaaaaatccaggattctctgtcctctaccgccagcgcgttggggaagctgcaggacgtggtgaaccacaacgcccaggccctgaacaccctggtgaaacaactgagcagcaagttcggagccatcagttctgtgctgaacgacatcttctcaagactggaccctcctgaggccgaagtgcagatcgacagactgatcaccggcagactgcagtctctgcagacctacgtgacacaacagctgatccgggccgccgagattagagccagcgccaacctggctgccacgaagatgtccgaatgtgtgctgggacagagcaaaagagtggatttctgcgggaaaggctaccacctgatgagctttcctcagagcgctccgcacggcgtggttttcctgcacgtgacctacgtccccgcccaggagaagaacttcaccaccgcccccgccatttgccacgacggcaaggcccacttccctcgggaaggggtgttcgtcagtaacggcacccactggttcgtgactcaaagaaacttctacgaaccccagatcatcactaccgataacaccttcgtcagcggaaactgcgatgtagtgattggtatcgtgaacaacaccgtgtacgaccctctgcagcctgagctggatagctttaaggaggaacttgacaagtacttcaagaaccacacatcccctgacgtggacctcggcgatataagcggcatcaacgcctcagtagttaatatccagaaagaaatcgacagactgaacgaggtggctaaaaatctgaacgagtccctgatcgaccttcaagagctgggaaaatacgagggcgggggcggcagcggcggcggcggcagcggcggcggtggctctggcgtgacacaaaacgtgctgtacgagaaccagaaactgatcgccaaccagttcaacagcgccatcggcaagatccaggacagcctgagctctacagcgtctgccttaggaaagctgcaggacgtggtgaatcacaacgcccaggccctgaacacactggttaagcagctgagcagcaaattcggcgccatcagcagcggctccagtggacaccaccaccatcatcaccaccactga

SEQ ID NO: 10 - protein, 1297 aa, synthetic construct
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEGGGGSGGGGSGGGGSGVTQNVLYENQKLIANQFNSAIGKIQDSLSSTASALGKLQDVVNHNAQALNTLVKQLSSKFGAISSGSSGHHHHHHHH

SEQ ID NO: 11 - RNA, 3813 nt, synthetic construct
  source 1..3813: mol_type = other RNA
atgttcgtgttcctggtgctgctgcccctcgttagcagccagtgcgtgaatctgaccacccgcacccagctgccaccagcctacacaaacagcttcaccagaggagtgtattaccctgataaggtctttagatcctccgtcctgcattctacgcaggatctcttcttgccattcttcagcaacgtgacatggttccacgtcatctctggcaccaacggcacaaagcgcttcgacaatcctgtgttgccgtttaacgacggcgtttacttcgccagcatcgaaaagagcaacatcatccggggctggatcttcggcaccaccctggacagcaaaacccaaagcctgctcatcgtgaacaacgccaccaacgtggtgatcaaggtgtgcgagttccagttctgcaatgatccttttctggaccacaagaacaacaagagctggatggaaagcgagttcagagtgtattctagcgccaacaactgcacctttgagtacgtgtcccagccctttcttatggacctggaaggcaagcagggcaacttcaagaatctgagagaattcgtgttcaagaacattgatggctacttcaagatctacagcaagcacacccctatcatcgttcgggagccagaagacctgccacaaggcttcagcgccctggaacctctggtggacctgcctatcggcatcaacatcacacggttccaaaccctgctggccctgcaccggagctacctgacccccggcgacagcagcagcggctggaccgccggcgctgccgcctattacgtgggctacctgcaacctagaaccttcctgctgaaatacaacgagaacggcacaatcaccgacgccgtggactgtgccctggaccccctgtctgagacaaagtgtaccctgaagtctttcaccgtggagaagggcatctaccagaccagcaacttccgggtgcagcctacagaatctatagtgcggttccctaacatcaccaacctgtgtccttttgacgaggtgttcaacgccactcggttcgcctctgtctacgcctggaaccggaaacggatctctaattgcgtggccgattacagcgtcctgtataacctcgcccctttcttcacattcaagtgctacggcgtgtcacccaccaagctgaacgatctgtgcttcaccaatgtgtacgccgatagtttcgtgatccggggcgatgaggtgcggcagatcgcccctggacagacaggcaacatcgccgactacaactacaagctgcctgacgacttcacaggctgtgtgatcgcatggaacagcaacaaactggacagcaaggtgagcggaaactacaactacctgtacagactgttcagaaagtccaacctgaagcctttcgagagagatatatctaccgagatctaccaggccggcaacaaaccctgtaatggagtggctggctttaactgctacttccctctgcggagctatagctttagacctacatatggggttggccaccagccttacagagtggtggtccttagcttcgagctgctccatgcccctgccaccgtgtgcggacctaagaagtccaccaacctggtgaaaaacaagtgcgtgaactttaattttaacggcctgaagggaacaggagtgctgacagaaagcaacaaaaagttcctgcctttccagcagttcggcagagacattgccgacaccacagatgctgttagagacccccagacgctggaaatcctggatatcaccccctgctcttttggcggcgtgagcgtgatcaccccaggcacaaacacaagcaaccaggtggctgtgctgtaccagggcgtgaactgtacagaggtccctgtggcaatccacgccgatcagctgacccctacatggcgggtgtactccactggatctaacgtgttccagacaagggccggatgcctcatcggcgctgagtacgtgaacaattcttacgagtgcgacatccctattggagcgggcatctgcgccagctaccagacacagaccaagagccatcagcaagccgctagcgtggcctcccagagcatcatcgcctacaccatgagcctgggagccgagaactctgtggcctacagcaacaacagcatcgctatccctaccaacttcaccatctctgtcaccaccgaaatcctgcccgtcagtatgaccaaaaccagcgtcgactgcaccatgtacatatgcggcgatagcaccgaatgcagcaacctgctgctgcagtatggctccttctgcacccaacttaagagagccctgactggcatcgccgtggagcaggacaagaatacccaggaggtgttcgcccaggtgaagcagatctacaagacacccccgatcaagtacttcggcggctttaatttctctcagatcctgccagacccatctaaaccctctaagcggagctttatcgaggacctgctgttcaacaaggtgactctggctgacgccggcttcatcaagcagtacggcgattgcctgggcgacattgctgctagagacctgatctgtgcccagaaattcaaaggtcttactgtgctgcctcctctgctgacggatgagatgatcgcccagtacaccagcgccctgctggccggcaccatcacatccggctggacattcggcgccggcgcagccctgcagatcccttttgccatgcagatggcctaccggttcaacggaatcggagtgacacagaacgtgctctacgaaaatcagaagttgatcgccaaccagttcaacagcgccatcggcaagattcaggatagtctgagttccaccgccagcgccctgggaaagctgcaggacgtggtcaatcacaatgcccaagccctgaacaccctggtgaagcagctgagcagcaagttcggcgccatcagctctgtgctgaacgacatcctgagtagattcgacaaggtggaagccgaagtgcagatcgacagattgatcaccggaagactgcaaagcctgcagacctacgtgacccagcagctgataagagctgctgaaatcagagccagcgctaatctggccgctaccaagatgagcgagtgcgttctgggccagtctaagagagtggacttctgcggaaaaggctaccacctgatgtcctttcctcagtctgccccccacggcgtggtgttcctgcacgtcacatacgtgcccgctcaagagaaaaacttcaccacggcccctgccatctgtcacgacggcaaggcccacttccccagagagggcgtgttcgtgagcaatggcacccactggtttgtgactcagagaaacttctacgagccacagattatcaccacagataacaccttcgtgtctggcaactgcgacgtggtgatcggcatcgtcaacaacacagtgtacgacccactgcaacctgagctggactcattcaaggaggaactggataagtacttcaagaatcacaccagccccgacgttgacctgggcgacatcagcggcattaacgcctctgtggtcaacatccagaaggaaatcgacagactgaatgaggtggccaagaatttgaacgagagcctgattgatctgcaggagctgggcaaatacgagcagtacatcaagtggccttggtacatctggctgggcttcatcgccgggctgatcgccatcgttatggtgacaatcatgctgtgttgcatgacaagctgttgtagctgcctgaaaggctgctgctcctgcggcagctgttgcaagtttgacgaagatgacagcgagcccgtgctgaaaggcgtcaagctgcactacacctga

SEQ ID NO: 12 - protein, 1270 aa, synthetic construct
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRFDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 13 - DNA, 49 bp, synthetic construct
  source 1..49: mol_type = other DNA
ctctctacggctaacctgaatggactacgacatagtctagtccgccaag

SEQ ID NO: 14 - DNA, 61 bp, synthetic construct
  source 1..61: mol_type = other DNA
gggcccctataactctctacggctaacctgaatggactacgacatagtctagtccgccaag

SEQ ID NO: 15 - DNA, 80 bp, synthetic construct
  source 1..80: mol_type = other DNA
tcattcagctacctgagaggggcccctataactctctacggctaacctgaatggactacgacatagtctagtccgccaag

SEQ ID NO: 16 - DNA, 575 bp, synthetic construct
  source 1..575: mol_type = other DNA
cccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaacc

SEQ ID NO: 17 - DNA, 101 bp, synthetic construct
  source 1..101: mol_type = other DNA
actctagctagcagtgttaaatcattcagctacctgagaggggcccctataactctctacggctaacctgaatggactacgacatagtctagtccgccaag

SEQ ID NO: 18-19 - skipped (2 entries)

SEQ ID NO: 20 - DNA, 10 bp, synthetic construct
  source 1..10: mol_type = other DNA
gcatatgact

SEQ ID NO: 21 - DNA, 94 bp, synthetic construct
  source 1..94: mol_type = other DNA
cccctccccgacacggattggctggtgtagtgggcgcggccagagaccacccagcgcccgaccccccctccccacaaacacggggggcgtccct

SEQ ID NO: 22 - DNA, 10 bp, synthetic construct
  source 1..10: mol_type = other DNA
ccccggcgcc

SEQ ID NO: 23 - skipped

SEQ ID NO: 24 - DNA, 26 bp, synthetic construct
  source 1..26: mol_type = other DNA
ccaaaggctcttttcagagccaccca

SEQ ID NO: 25 - DNA, 44 bp, synthetic construct
  source 1..44: mol_type = other DNA
ataggcggcgcatgagagaagcccagaccaattacctacccaaa

SEQ ID NO: 26 - DNA, 319 bp, synthetic construct
  source 1..319: mol_type = other DNA
ccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttc

SEQ ID NO: 27 - DNA, 70 bp, synthetic construct
  source 1..70: mol_type = other DNA
gggcccctataactctctacggctaacctgaatggacctgaatggactacgacatagtctagtccgccaa

SEQ ID NO: 28 - DNA, 36 bp, synthetic construct
  source 1..36: mol_type = other DNA
aaaaaaaaaaaaaaaaaaaaaaaaaaaaaaaaaaaa

SEQ ID NO: 29 - DNA, 9631 bp, synthetic construct
  source 1..9631: mol_type = other DNA
gataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggagaaaatagtgcttcttcttgcaatagtcagccttgttaaaagtgatcagatttgcattggttaccatgcaaacaactcgacagagcaggttgacacaataatggaaaagaacgtcactgttacacacgcccaagacatactggaaaagacacacaacgggaaactctgcgatctagatggagtgaagcctctaattttaagagattgtagtgtagctggatggctcctcgggaacccaatgtgtgacgaattcctcaatgtgccggaatggtcttacatagtggagaagatcaatccagccaatgacctctgttacccagggaatttcaacgactatgaagaactgaaacacctattgagcagaataaaccattttgagaaaattcagatcatccccaaaagttcttggtcagatcatgaagcctcagcaggggtgagctcagcatgtccataccagggaaggtcctccttttttagaaatgtggtatggcttatcaaaaaggacaatgcatacccaacaataaagagaagttacaataataccaaccaagaagatcttttggtattgtgggggattcaccatccaaatgatgcggcagagcagacaaggctctatcaaaacccaactacctatatttccgttgggacatcaacactaaaccagagattggtaccaaaaatagccactagatctaaggtaaacgggcaaagtggaaggatggagttcttttggacaattttaaaaccgaatgatgcaataaactttgagagtaatggaaatttcattgctccagaaaatgcatacaaaattgtcaagaaaggggactcaacaattatgaaaagtgagttggaatatggtaactgcaacaccaagtgtcaaactccaataggggcgataaactctagtatgccattccacaacatccaccctctcaccatcggggaatgccccaaatatgtgaaatcaagcagattagtccttgctactgggctcagaaatagccctcaacgagagacacgaggactatttggagctatagcaggttttatagagggaggatggggaatggtagatggttggtatgggtaccaccatagcaacgagcaggggagtgggtacgctgcagacaaagaatccactcaaaaggcaatagatggagtcaccaataaggtcaactcgatcattgacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagcttggtaacggttgtttcgagttctatcacagatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagtggcgagctccctagcactggcaatcatggtggctggtctatctttatggatgtgctccaatggatcgttacaatgcagaatttgcatttaaggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaa

SEQ ID NO: 30 - RNA, 9631 nt, synthetic construct
  source 1..9631: mol_type = other RNA
gataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggagaaaatagtgcttcttcttgcaatagtcagccttgttaaaagtgatcagatttgcattggttaccatgcaaacaactcgacagagcaggttgacacaataatggaaaagaacgtcactgttacacacgcccaagacatactggaaaagacacacaacgggaaactctgcgatctagatggagtgaagcctctaattttaagagattgtagtgtagctggatggctcctcgggaacccaatgtgtgacgaattcctcaatgtgccggaatggtcttacatagtggagaagatcaatccagccaatgacctctgttacccagggaatttcaacgactatgaagaactgaaacacctattgagcagaataaaccattttgagaaaattcagatcatccccaaaagttcttggtcagatcatgaagcctcagcaggggtgagctcagcatgtccataccagggaaggtcctccttttttagaaatgtggtatggcttatcaaaaaggacaatgcatacccaacaataaagagaagttacaataataccaaccaagaagatcttttggtattgtgggggattcaccatccaaatgatgcggcagagcagacaaggctctatcaaaacccaactacctatatttccgttgggacatcaacactaaaccagagattggtaccaaaaatagccactagatctaaggtaaacgggcaaagtggaaggatggagttcttttggacaattttaaaaccgaatgatgcaataaactttgagagtaatggaaatttcattgctccagaaaatgcatacaaaattgtcaagaaaggggactcaacaattatgaaaagtgagttggaatatggtaactgcaacaccaagtgtcaaactccaataggggcgataaactctagtatgccattccacaacatccaccctctcaccatcggggaatgccccaaatatgtgaaatcaagcagattagtccttgctactgggctcagaaatagccctcaacgagagacacgaggactatttggagctatagcaggttttatagagggaggatggggaatggtagatggttggtatgggtaccaccatagcaacgagcaggggagtgggtacgctgcagacaaagaatccactcaaaaggcaatagatggagtcaccaataaggtcaactcgatcattgacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagcttggtaacggttgtttcgagttctatcacagatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagtggcgagctccctagcactggcaatcatggtggctggtctatctttatggatgtgctccaatggatcgttacaatgcagaatttgcatttaaggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaa

SEQ ID NO: 31 - DNA, 11884 bp, synthetic construct
  source 1..11884: mol_type = other DNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggagaaaatagtgcttcttcttgcaatagtcagccttgttaaaagtgatcagatttgcattggttaccatgcaaacaactcgacagagcaggttgacacaataatggaaaagaacgtcactgttacacacgcccaagacatactggaaaagacacacaacgggaaactctgcgatctagatggagtgaagcctctaattttaagagattgtagtgtagctggatggctcctcgggaacccaatgtgtgacgaattcctcaatgtgccggaatggtcttacatagtggagaagatcaatccagccaatgacctctgttacccagggaatttcaacgactatgaagaactgaaacacctattgagcagaataaaccattttgagaaaattcagatcatccccaaaagttcttggtcagatcatgaagcctcagcaggggtgagctcagcatgtccataccagggaaggtcctccttttttagaaatgtggtatggcttatcaaaaaggacaatgcatacccaacaataaagagaagttacaataataccaaccaagaagatcttttggtattgtgggggattcaccatccaaatgatgcggcagagcagacaaggctctatcaaaacccaactacctatatttccgttgggacatcaacactaaaccagagattggtaccaaaaatagccactagatctaaggtaaacgggcaaagtggaaggatggagttcttttggacaattttaaaaccgaatgatgcaataaactttgagagtaatggaaatttcattgctccagaaaatgcatacaaaattgtcaagaaaggggactcaacaattatgaaaagtgagttggaatatggtaactgcaacaccaagtgtcaaactccaataggggcgataaactctagtatgccattccacaacatccaccctctcaccatcggggaatgccccaaatatgtgaaatcaagcagattagtccttgctactgggctcagaaatagccctcaacgagagacacgaggactatttggagctatagcaggttttatagagggaggatggggaatggtagatggttggtatgggtaccaccatagcaacgagcaggggagtgggtacgctgcagacaaagaatccactcaaaaggcaatagatggagtcaccaataaggtcaactcgatcattgacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagcttggtaacggttgtttcgagttctatcacagatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagtggcgagctccctagcactggcaatcatggtggctggtctatctttatggatgtgctccaatggatcgttacaatgcagaatttgcatttaaggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaggcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 32 - DNA, 38 bp, Synthetic construct
  source 1..38: mol_type = other DNA
taacctgaatggactacgacatagtctagtccgccaag